(19) United States
(12) Patent Application Publication
(10) Pub. No.: US 2024/0368168 A1
(43) Pub. Date: Nov. 7, 2024

(54) SARM1 ENZYME ACTIVITY INHIBITOR AND APPLICATION THEREOF

(71) Applicant: ARTIVILA BIOPHARMA, Wuxi, Jiangsu (CN)

(72) Inventors: Deqiang NIU, Wuxi, Jiangsu (CN); Zhendong ZHU, Wuxi, Jiangsu (CN); Ming FAN, Wuxi, Jiangsu (CN); Xuke LI, Wuxi, Jiangsu (CN)

(73) Assignee: ARTIVILA BIOPHARMA, Wuxi, Jiangsu (CN)

(21) Appl. No.: 18/686,299

(22) PCT Filed: Aug. 25, 2022
(86) PCT No.: PCT/CN2022/114815

§ 371 Date: Feb. 23, 2024

(30) Foreign Application Priority Data
Aug. 26, 2021 (CN) 202110990705.2

(51) Int. Cl. C07D 487/04; A61K 31/437; A61K 31/444; A61K 31/4545; A61K 31/4709; A61K 31/4725; A61K 31/496; A61K 31/501; A61K 31/5025; A61K 31/506; A61K 31/5377; A61K 31/5383; C07D 471/04; C07D 498/14; C07D 515/16; C07D 519/00

(52) U.S. Cl. CPC C07D 487/04; A61K 31/437; A61K 31/444; A61K 31/4545; A61K 31/4709; A61K 31/4725; A61K 31/496; A61K 31/501; A61K 31/5025; A61K 31/506; A61K 31/5377; A61K 31/5383; C07D 471/04; C07D 498/14; C07D 515/16; C07D 519/00

(57) ABSTRACT

The present invention provides an application of an SARM1 enzyme activity inhibitor in the treatment of neurodegenerative diseases or neurological diseases or conditions. The present invention particularly provides a compound of formula I as an SARM1 enzyme activity inhibitor and a pharmaceutical composition thereof.

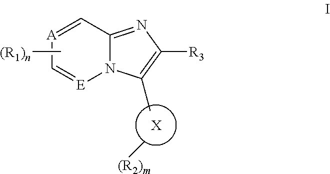

### CROSS REFERENCE TO RELATED APPLICATIONS

This application claims the priority of Chinese Patent Application No. 202110990705.2, filed with the China National Intellectual Property Administration on Aug. 26, 2021, and titled with “SARM1 ENZYME ACTIVITY INHIBITOR AND APPLICATION THEREOF”, which is hereby incorporated by reference in its entirety.

### FIELD

The present disclosure relates to compounds for inhibiting SARM1 enzymatic activity, and/or use of the compound in treating and/or preventing a neurodegenerative or neurological disease or condition associated with SARM1 enzymatic activity.

### BACKGROUND

Neurodegenerative diseases are a type of disease that can seriously harm humans, which can be a progressive disease that causes devastating damage, such as death of nerve cells. As the primary neurodegenerative diseases, central nervous system diseases such as Alzheimer's disease, Parkinson's disease, amyotrophic lateral sclerosis (ALS) and Huntington's disease, and peripheral neurological diseases such as diabetes are already known. Most of these are related to aging, and the onset of these diseases increases with age, although they also occur in middle-aged people or even younger people.

As a result of studies on brain structure and function, the roles of neurotransmitters and neurotrophic factors have been gradually elucidated, but many local causes of neurodegeneration remain unclear. Only for Parkinson's disease, the relationship between the disease and dopamine, a special neurotransmitter, has been clarified, and L-dopa, a precursor of dopamine, has been used as a drug to reduce neurological symptoms and restore neurological functions. However, L-dopa cannot inhibit the development of neurodegeneration, and gradually loses its effect as the disease progresses, that is, the degeneration and defects of dopamine-based nerve cells. Similarly, Alzheimer's disease is also caused by the degeneration and defects of various nerve cells, such as acetylcholine-based nerve cells and monoamine-based nerve cells. As drugs to treat this disease, cholinesterase inhibitors have been put on the market or are in the process of being developed. However, L-dopa for treating Parkinson's disease is still limited to symptomatic treatment to temporarily improve neurological symptoms.

Therefore, there is currently a lack of effective therapeutic drugs for neurodegenerative diseases.

It has been found from studies that nerve axon damage occurs in a variety of neurological diseases such as neurodegenerative diseases and accidental injuries. Axonal degeneration can cause structural necrosis and dysfunction of the peripheral nervous system, ultimately leading to acquired or inherited degenerative diseases of the central nervous system.

Although there is currently no very effective pharmacological method that can accurately assess the weight of incidence rate caused by axonal degeneration, it has been found in histopathological studies that significant axonal damage and degradation are observed in the early stages of various neuropathies such as Alzheimer's disease, Parkinson's disease, multiple sclerosis, amyotrophic lateral sclerosis and peripheral neuropathy, indicating that axonal degeneration plays an important role in the development of neuropathy (Fischer et al., Neuro-degenerative Diseases, 2007, 4:431-442). Therefore, maintaining the integrity of neuronal structure and function by attenuating or even blocking axonal degeneration may be a therapeutic method that can benefit a variety of neurological diseases.

In the absence of effective therapeutic drugs for neurodegenerative diseases, the research and development of new compounds is urgently required in the prior art, especially small chemical molecules, including compounds that have effect on neural axon degeneration.

### SUMMARY

After extensive research, the inventors unexpectedly found a class of compounds with significant inhibitory effect on SARM1 enzymatic activity, and found that the compounds can improve axonal degeneration and be used to treat or prevent neurodegenerative diseases and related conditions.

SARM1 consists of three domains, namely Armadillo/HEAT repeat (ARM) domain at the N-terminal, two tandem Sterile alpha motif (SAM) domains, and Toll/Interleukin receptor (TIR) domain at the C-terminal. In addition, there is also a mitochondrial localization signal peptide at the N-terminal.

It is known that in wild-type neurons, axonal injury induces NAD⁺ depletion and axonal degeneration. Knocking out SARM1 inhibits axonal degeneration, and maintains NAD⁺ at normal levels, indicating that SARM1 promotes NAD⁺ consumption and aggravates axonal degeneration.

Milbrandt's research group at Washington University School of Medicine prepared TIR domain of SARM1 (SARM1-TIR) and found that it has NAD⁺ hydrolase activity. High-purity SARM1-TIR was further obtained through strict E. coli expression and purification experiments and a cell-free expression system. It has been finally proven that SARM1-TIR can catalyze NAD⁺ to produce adenosine 5′-diphosphate ribose (ADPR) and cyclic adenosine 5′-diphosphate ribose (cADPR).

SARM1 is a multifunctional signaling enzyme that can catalyze a variety of substrates NAD⁺, NADP⁺ and NA etc. to generate signal molecules such as cADPR, ADPR and NAADP. In a variety of neurodegenerative diseases, SARM1 is activated, leading to NAD⁺ depletion, thereby initiating a new cell death mechanism. Knocking out SARM1 can inhibit axonal degeneration and disease progression. Therefore, SARM1 is considered a potential drug target for related neurological diseases, including TBI, AD, CIPN, ASL, etc.

In the present disclosure, the inventor prepared full-length SARM1, which was used in NAD enzymatic activity experiments, and used to screen and discover herein compounds with inhibitory SARM1 enzymatic activity.

Therefore, based on the above findings, in a first aspect, the present disclosure provides use of a SARM1 enzymatic activity inhibitor in the manufacture of a medicament for treating or preventing a neurodegenerative disease or a neurological disease or condition.

In another aspect, the present disclosure provides use of a SARM1 enzymatic activity inhibitor in the manufacture of a medicament for treating or preventing an axonal degeneration-related disease or condition.

In particular, the present disclosure provides a compound represented by formula I that serves as an inhibitor of SARM1 enzymatic activity:

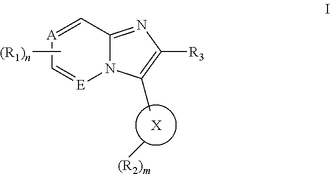

or a pharmaceutically acceptable salt or stereoisomer thereof,
wherein,
A represents CH or N;
E represents CH or N;
R₁ is independently selected from the group consisting of hydrogen, halogen, CF₃, CN, C₁-C₆ alkyl, C₁-C₆ alkoxy, C₃-C₆ cycloalkyl, C₃-C₆ heterocycloalkyl, amino, CF₃C(O)—NH—, CF₃C(O)—N(CH₃)—, C₁-C₆ alkylamino, C₃-C₆ cycloalkylamino, C₆-C₁₄ aryl, C₅-C₁₄ heteroaryl, C₆-C₁₄ arylamino, C₆-C₁₄ heteroarylamino, —OH, C₆-C₁₄ aryloxy, —CONH₂, —SO₂NH₂, C₁-C₆ alkyl-C(O)NR₅—, C₃-C₆ cycloalkyl-C(O)NR₅—, C₃-C₆ heterocycloalkyl-C(O)NR₅—, C₁-C₆ alkyl-OC(O)NR₅—, C₃-C₆ cycloalkyl-OC(O)NR₅—, C₃-C₆ heterocycloalkyl-OC(O)NR₅—, C₁-C₆ alkyl-OC(O)NR₅—(C₁-C₄ alkyl)-, C₁-C₆ alkyl-C(O)NR₅—(C₁-C₄ alkyl)-, C₃-C₆ cycloalkyl-OC(O)NR₅—(C₁-C₄ alkyl)-, C₃-C₆ cycloalkyl-C(O)NR₅—(C₁-C₄ alkyl)-, (C₆-C₁₄ aryl)-(C₁-C₆ alkyl)-CO—N(R₅)—, (C₆-C₁₄ aryl)-(C₃-C₆ alkyl)-N(R₅)—, and (C₆-C₁₄ aryl)-(C₃-C₆ alkenyl)-N(R₅)—; wherein, in the above C₁-C₆ alkyl, one carbon atom can be replaced by a heteroatom selected from the group consisting of N, O and S atoms; preferably, R₁ is independently selected from the group consisting of C₁-C₆ alkylamino, C₃-C₆ heterocycloalkylamino, C₁-C₆ alkylacylamino, 1-morpholinyl, and C₁-C₆ alkyl-OC(O)NR₅—;
X represents a cyclic structure, selected from the group consisting of C₃-C₆ cycloalkyl, C₃-C₆ cycloalkenyl, C₃-C₆ heterocycloalkyl, C₆-C₁₄ aryl and C₅-C₁₄ heteroaryl, or X is absent; preferably, X is selected from the group consisting of phenyl, pyridyl, methoxy-substituted pyridyl, thiazolyl, cyclohexyl and cyclohexenyl, wherein the phenyl can be substituted by the following substituents: —SO₂—NH₂, —NH—COCH₃, —NH₂, —CO—NH₂, —OCH₃, halogen, C₁-C₄ alkyl, —SO₂—N(BoC)CH₃ or C₁-C₄ alkyl-NH—SO₂—;
R₂ is independently selected from the group consisting of hydrogen, halogen, —NH₂, —N(R₅)—CO—R, —CO—N(R₅)—R, —N(R₅)—SO₂—R, —SO₂—N(R₅)—R, —COOR, —COR, —(C₁-C₄ alkyl)-OR, —(C₁-C₄ alkyl)-N(CH₃)₂, NH—(C₁-C₄ alkyl)R—, —N(R₅)—R, —NHCO—(C₃-C₆ cycloalkyl)-(C₃-C₆ heterocycloalkyl), —OR, —O—(C₁-C₄ alkyl)-R and R;
   R is selected from the group consisting of C₁-C₄ alkoxy, C₁-C₁₂ alkyl, —CONH₂, —SO₂—NH—R, C₃-C₆ cycloalkyl, C₃-C₆ heterocycloalkyl, —(C₁-C₁₂ alkyl)-(C₆-C₁₄) aryl, C₆-C₁₄ aryl and C₅-C₁₄ heteroaryl, wherein the C₁-C₁₂ alkyl, C₃-C₆ cycloalkyl, C₃-C₆ heterocycloalkyl, C₆-C₁₄ aryl and C₅-C₁₄ heteroaryl are optionally substituted by 1, 2 or 3 halogens, and 1 to 4 —CH₂— units in the C₁-C₁₂ alkyl are optionally replaced by O atom, S atom, —CO— or —NH—;
R₅ is selected from the group consisting of H, C₁-C₄ alkyl, C₃-C₆ cycloalkyl, C₃-C₆ heterocycloalkyl, C₁-C₄ alkoxy, C₆-C₁₄ aryl and C₅-C₁₄ heteroaryl;
R₃ is independently selected from the group consisting of hydrogen, C₁-C₄ alkyl and C₁-C₄ alkoxycarbonyl;
wherein the above C₃-C₆ heterocycloalkyl and C₅-C₁₄ heteroaryl contain 1 or 2 heteroatoms selected from the group consisting of N, O and S atoms;
R₁ and R₂ can be connected through a carbon-carbon bond or an ether bond to form a 14- to 16-membered ring, which contains 1-4 heteroatoms selected from the group consisting of N, O and S, preferably, the ring contains 3-4 N atoms and 1-2 O or S atoms;
m and n are positive integers selected from the group consisting of 1, 2 and 3.

In a preferred aspect, the compound represented by formula I of the present disclosure has the following structure of formula II:

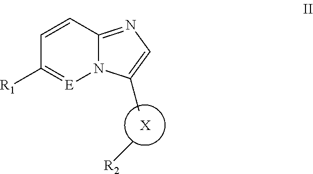

wherein E, R₁, R₂ and X are defined as above.

In a preferred aspect, the compound represented by formula I of the present disclosure has the following structure of formula III:

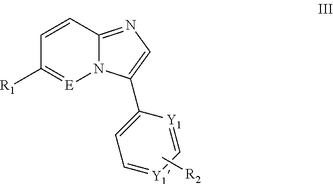

wherein E, R₁ and R₂ are defined as above;
Y₁ and Y₁′ are independently CH or N.

In another preferred aspect, the compound represented by formula I of the present disclosure has the following structure of formula IV:

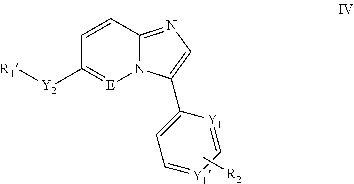

wherein E and R₂ are defined as above;
Y₁ and Y₁′ are independently CH or N;
Y₂ is selected from the group consisting of —O—, —NH—, —NR₅—, —NR₅—(C₁-C₄ alkyl)- and —NR₅ (C₃-C₆ cycloalkyl)-, or Y₂ does not exist;
R₁′ is selected from the group consisting of R, —C(═O)—R, —SO₂—R, —C(═O)—OR and —SO₂NHR; wherein R₅ and R are defined as above.

In yet another preferred aspect, the compound represented by formula I of the present disclosure has the following structure of formula V:

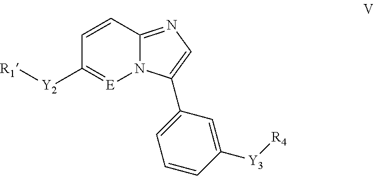

wherein,
E, R₁′ and Y₂ are defined as above;
Y₃ is selected from the group consisting of N(R₅)CO—, —CO—N(R₅)—, —N(R₅)—SO₂—, —SO₂—N(R₅)—, —CO₂—, —CO—, —NH—(C₁-C₄ alkyl)-, —N(R₅)—, —O—(C₁-C₄ alkyl)- and —O—, or Y₃ does not exist;
R₄ is selected from the group consisting of C₁-C₁₂ alkyl, C₃-C₆ cycloalkyl, C₃-C₆ heterocycloalkyl, C₆-C₁₄ aryl and C₅-C₁₄ heteroaryl, wherein the C₁-C₁₂ alkyl, C₃-C₆ cycloalkyl, C₃-C₆ heterocycloalkyl, C₆-C₁₄ aryl and C₅-C₁₄ heteroaryl are optionally substituted by 1, 2 or 3 halogens; the C₃-C₆ heterocycloalkyl and C₅-C₁₄ heteroaryl contain 1 or 2 heteroatoms selected from the group consisting of N, O and S atoms; and 1 to 4 —CH₂— units in the C₁-C₁₂ alkyl are optionally replaced by O atom, S atom, —CO— or —NH—.

In yet another preferred aspect, the compound of the present disclosure has the following structure of formula VI:

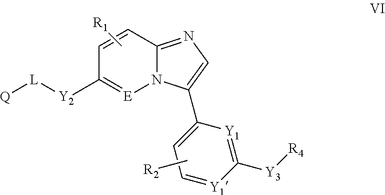

wherein,
E, R₁, R₂, Y₁, Y₁′, Y₂, Y₃ and R₄ are defined as above;
L is C₂-C₁₂ alkylene, wherein 1, 2, 3 or 4 —CH₂— units in the C₂-C₁₂ alkylene are optionally replaced by 1, 2, 3 or 4 O atoms, N atoms, —CO—, —CONH— or —NHCO—;
Q is an E3 ligase ligand, preferably a VHL ligand

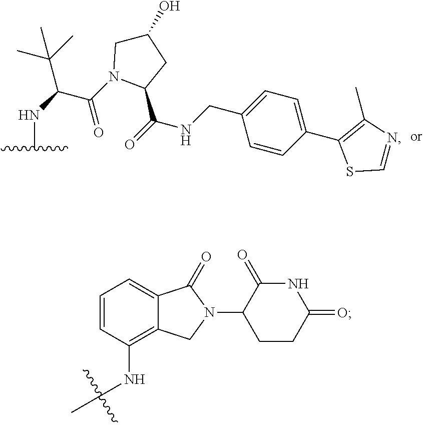 [SMILES: *N[C@H](C(=O)N1C[C@H](O)C[C@H]1C(=O)NCc1ccc(-c2scnc2C)cc1)C(C)(C)C ; "or" ; *Nc1cccc2c1CN(C1CCC(=O)NC1=O)C2=O]

or Q is a structural unit selected from the group consisting of:

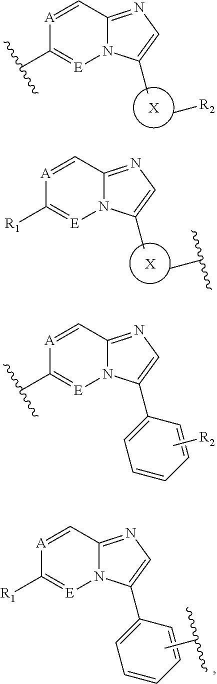

wherein the variables A, E, X, R₁ and R₂ are defined as above;
or Q is

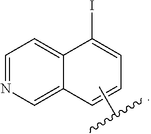

In another preferred aspect, the compound of the present disclosure has the following structure of formula VII:

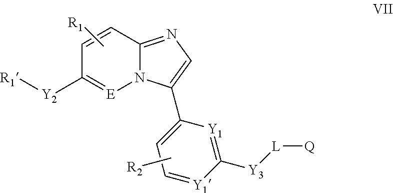

wherein,
E, R₁, R₂, R₁′, Y₁, Y₁′ and Y₂ are defined as above;
Y₃ is selected from the group consisting of —N(R₅)CO—, —CO—N(R₅)—, —N(R₅)—SO₂—, —SO₂—N(R₅)—, —CO₂—, —CO—, —NH—(C₁-C₄ alkyl)-, —N(R₅)—, —O—(C₁-C₄ alkyl)-, and —O—, or Y₃ does not exist;
L is C₂-C₁₂ alkylene, wherein 1, 2, 3 or 4 —CH₂— units in the C₂-C₁₂ alkylene are optionally replaced by 1, 2, 3 or 4 O atoms, N atoms, —CO—, —CONH— or —NHCO—;
Q is an E3 ligase ligand, preferably a VHL ligand

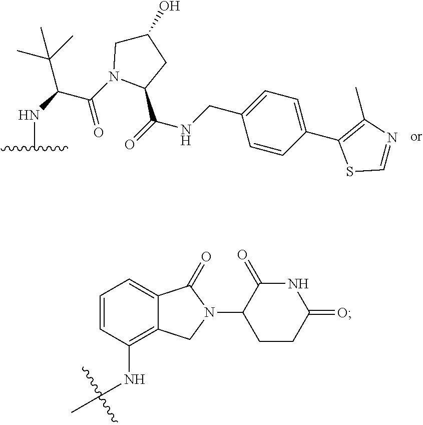 [SMILES: *N[C@H](C(=O)N1C[C@H](O)C[C@H]1C(=O)NCc1ccc(-c2scnc2C)cc1)C(C)(C)C ; "or" ; *Nc1cccc2c1CN(C1CCC(=O)NC1=O)C2=O]

or Q is a structural unit selected from the group consisting of:

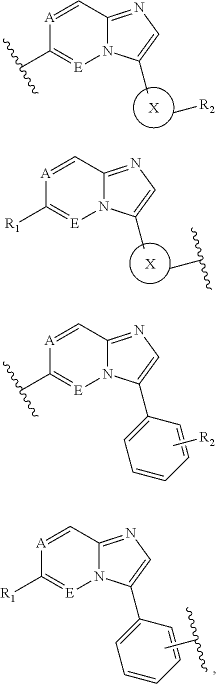

wherein, the variables A, E, X, R₁ and R₂ are defined as above;
or Q is

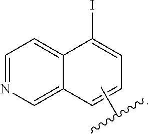

In some preferred embodiments, the compound of the present disclosure is selected from the group consisting of the following compounds, or pharmaceutically acceptable salts or stereoisomers thereof:

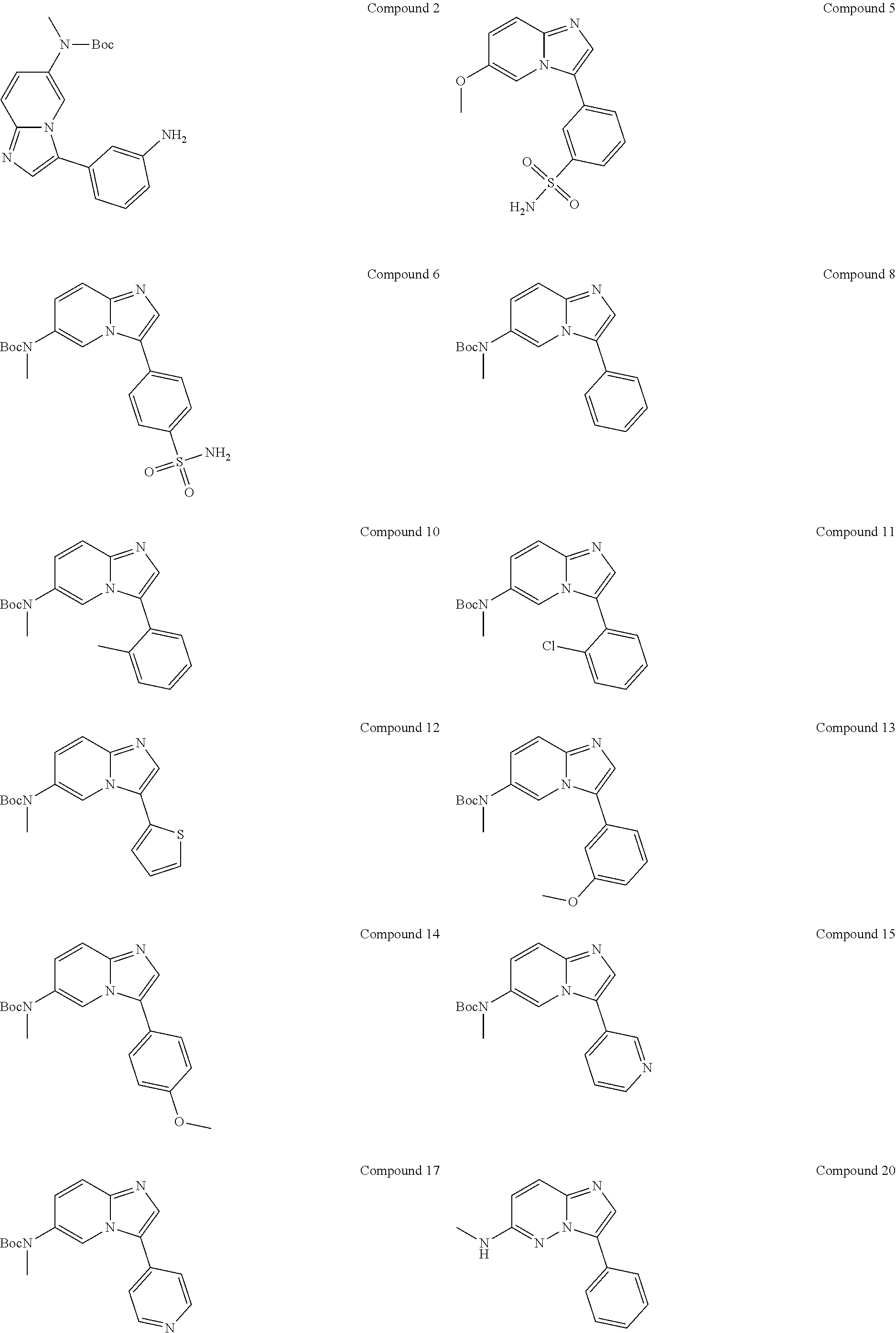

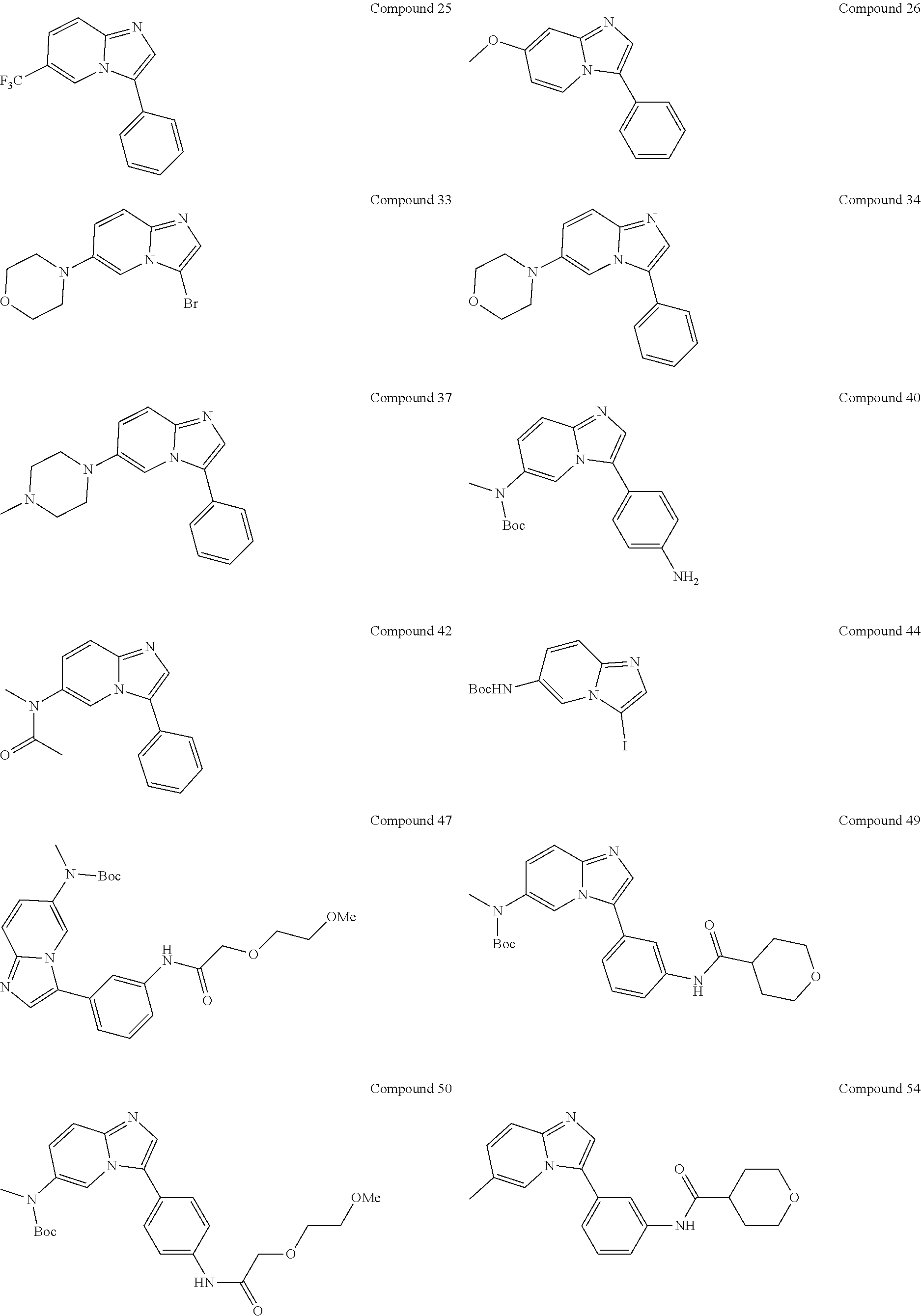

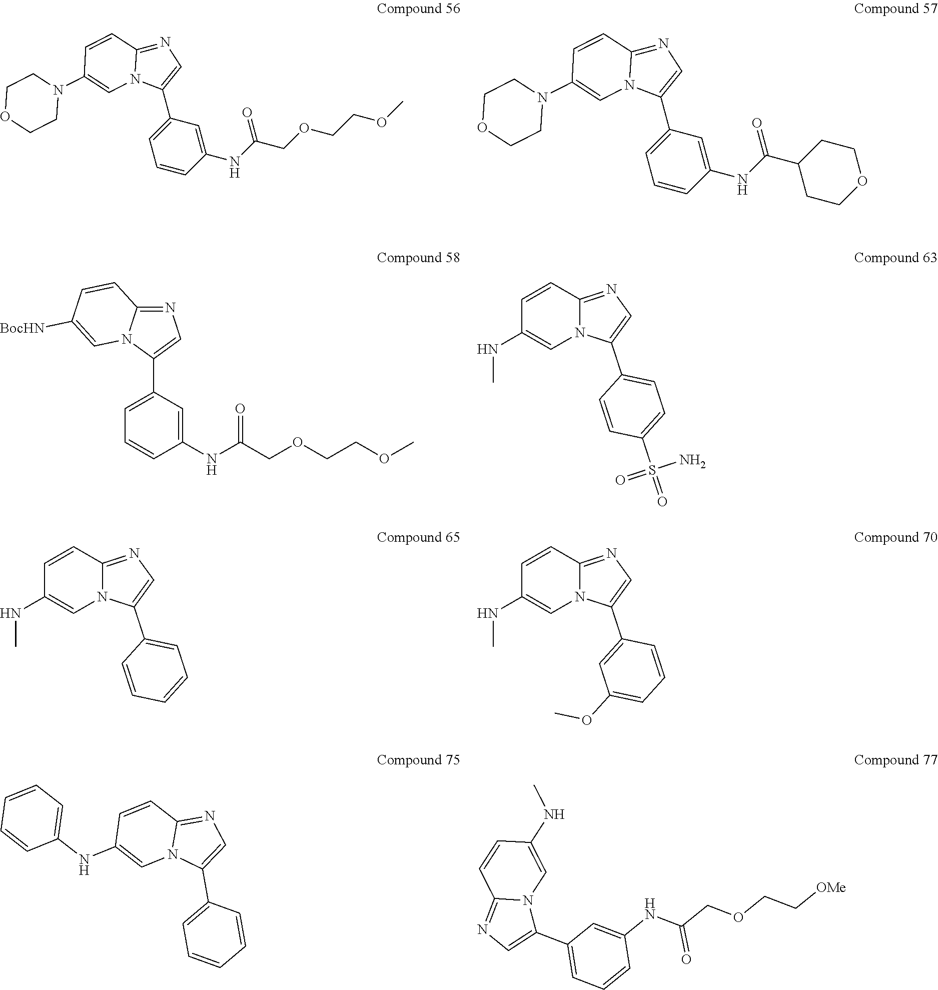

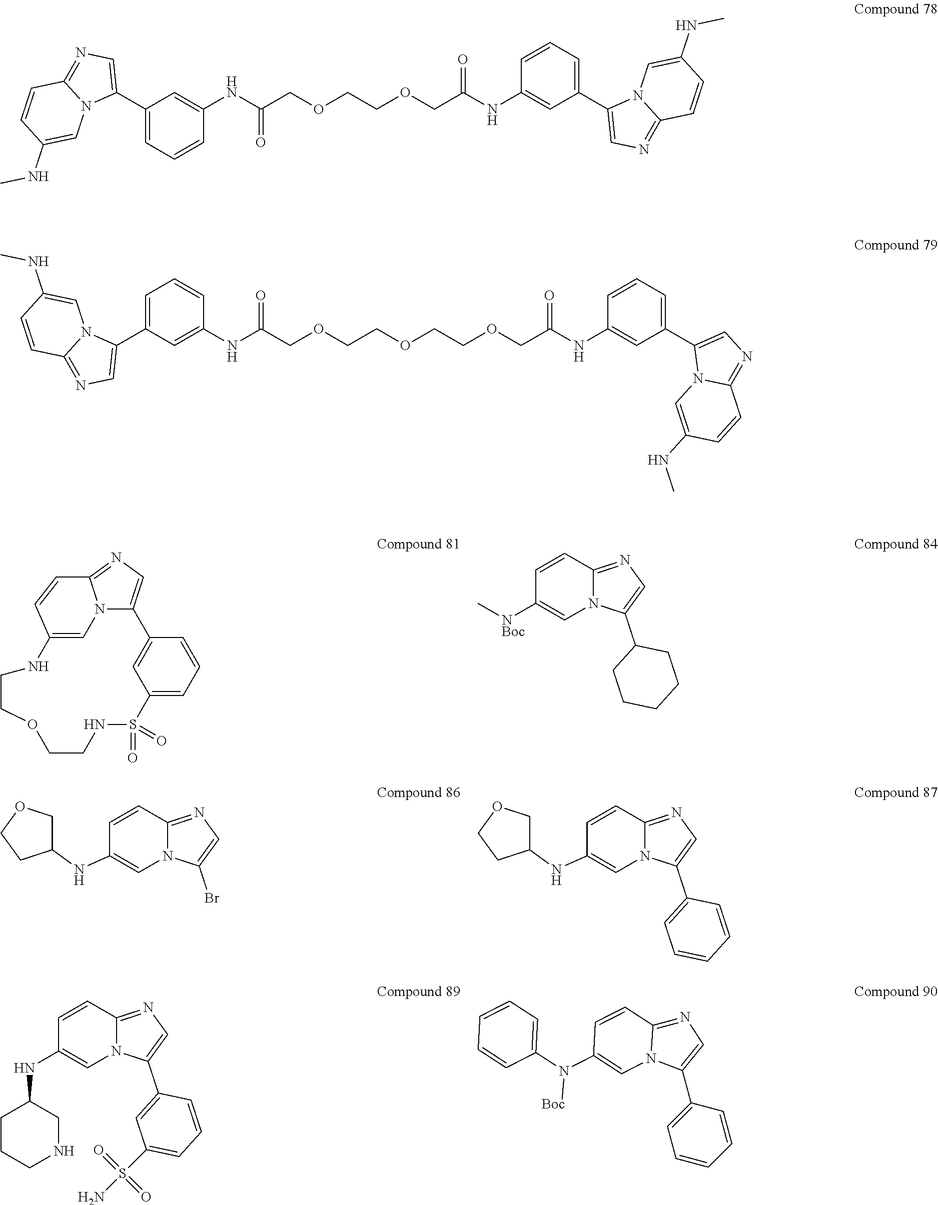

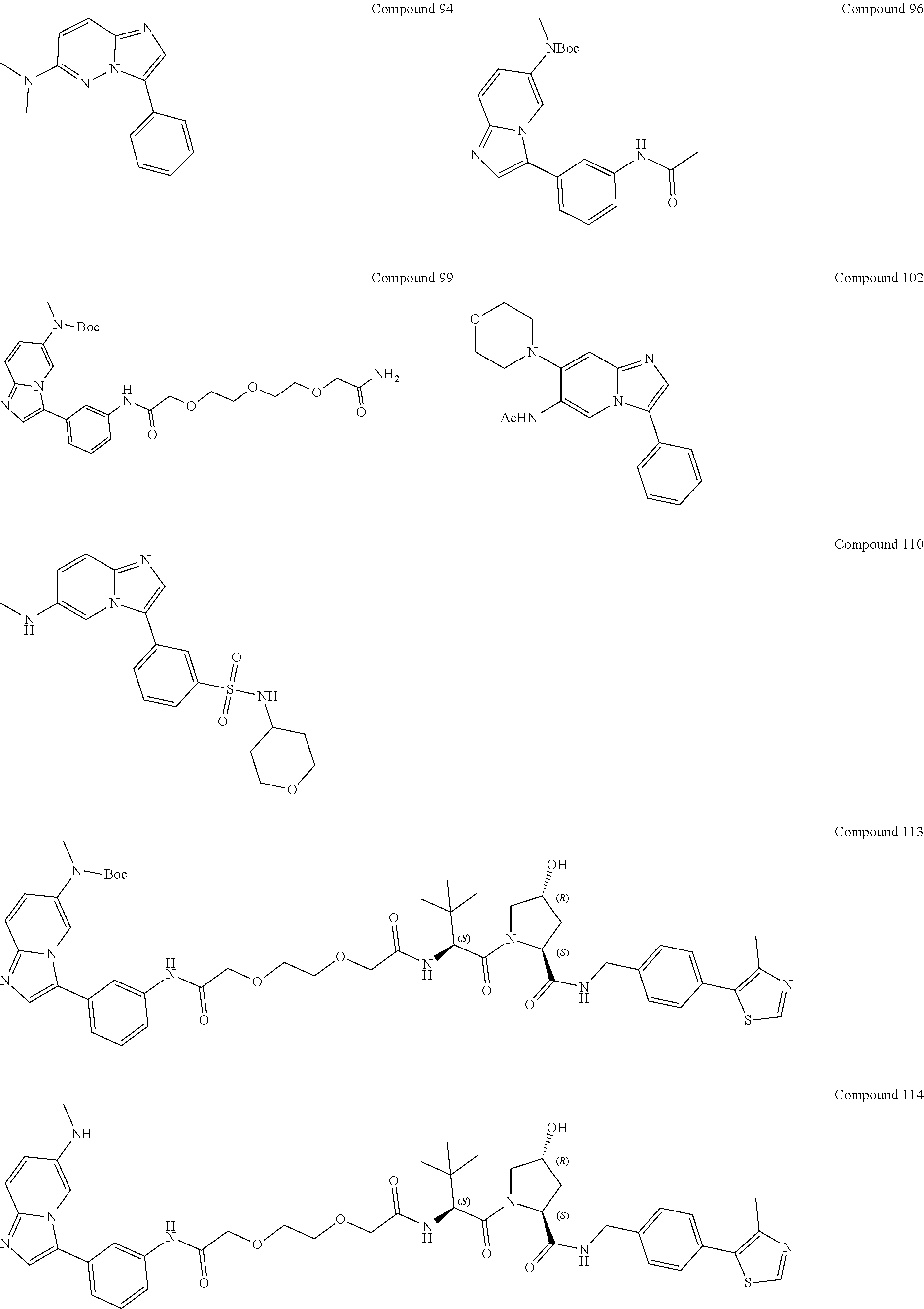

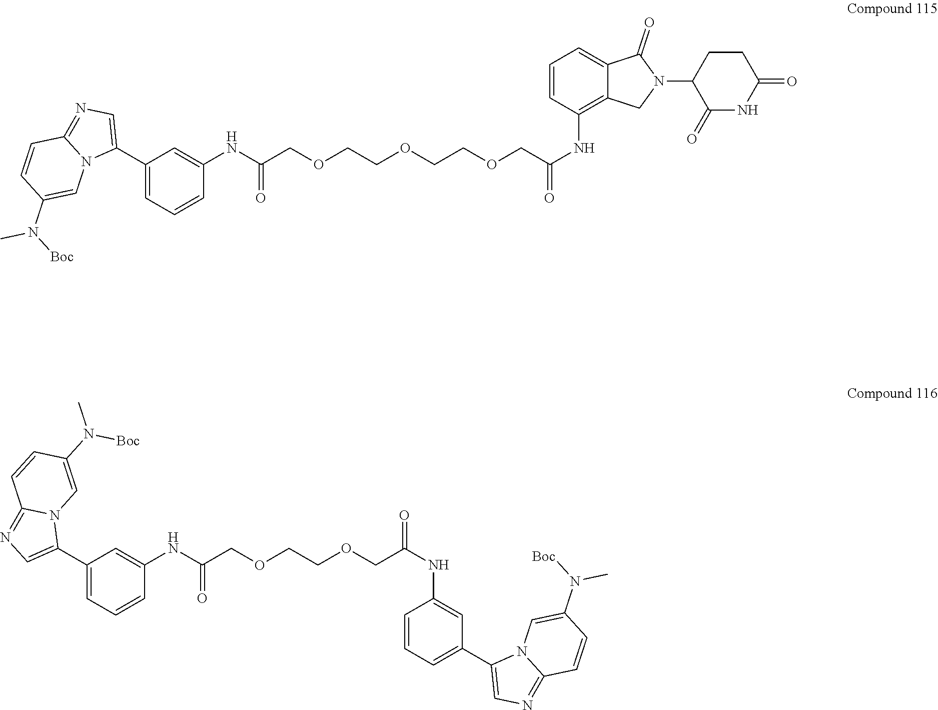 [SMILES: CN(C(=O)OC(C)(C)C)c1ccc2ncc(-c3cccc(NC(=O)COCCOCCOCC(=O)Nc4cccc5c4CN(C4CCC(=O)NC4=O)C5=O)c3)n2c1 ; CN(C(=O)OC(C)(C)C)c1ccc2ncc(-c3cccc(NC(=O)COCCOCC(=O)Nc4cccc(-c5cnc6ccc(N(C)C(=O)OC(C)(C)C)cn56)c4)c3)n2c1]

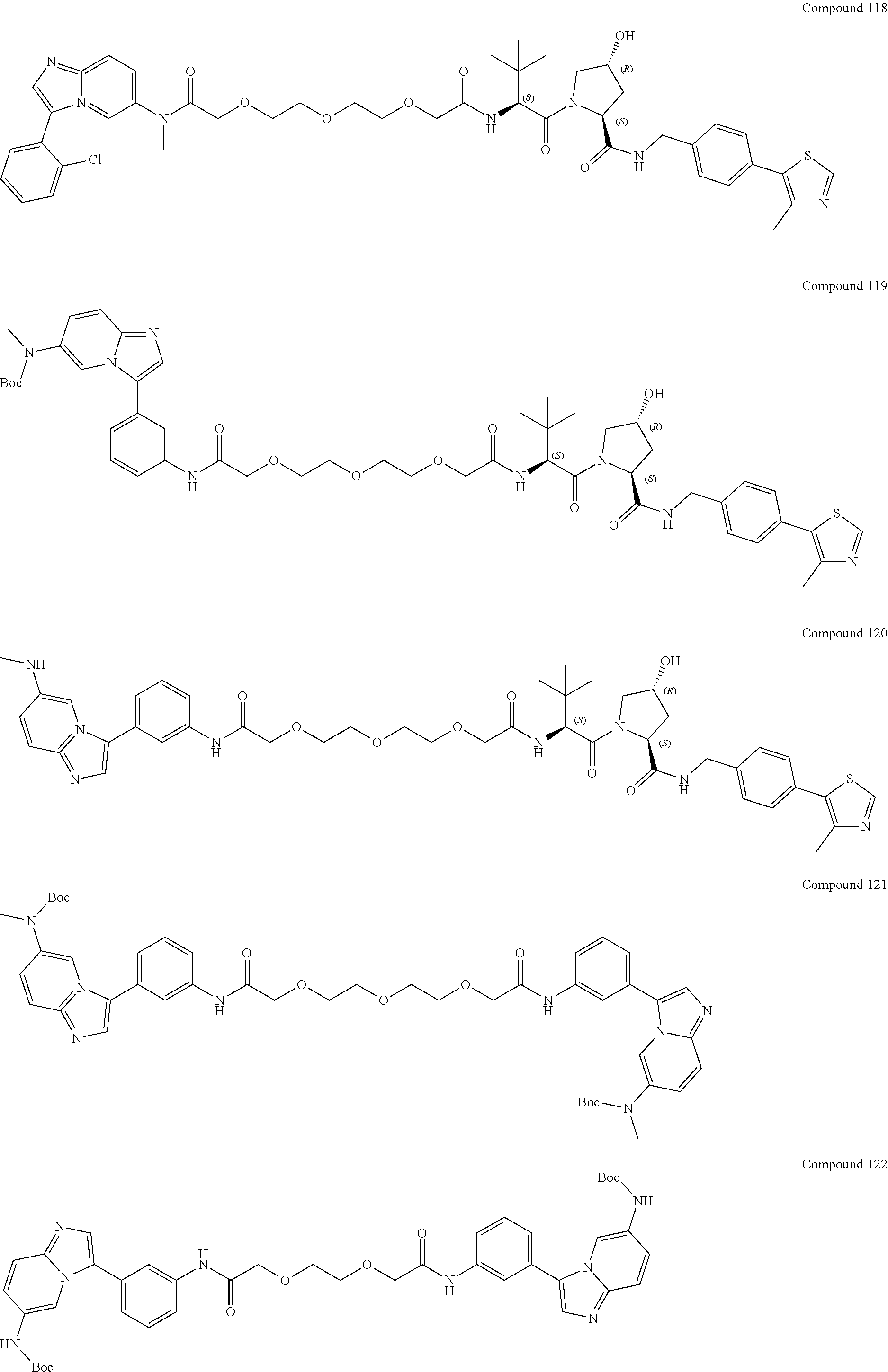

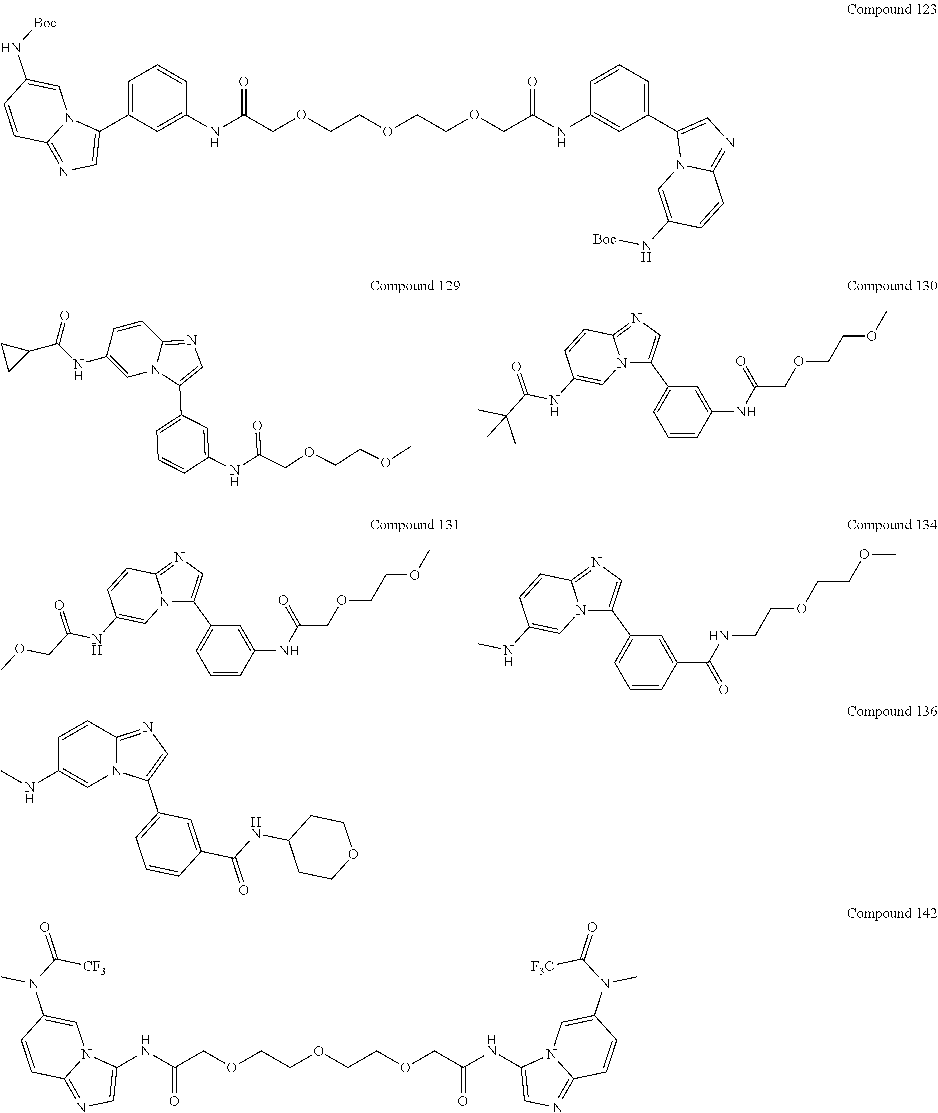

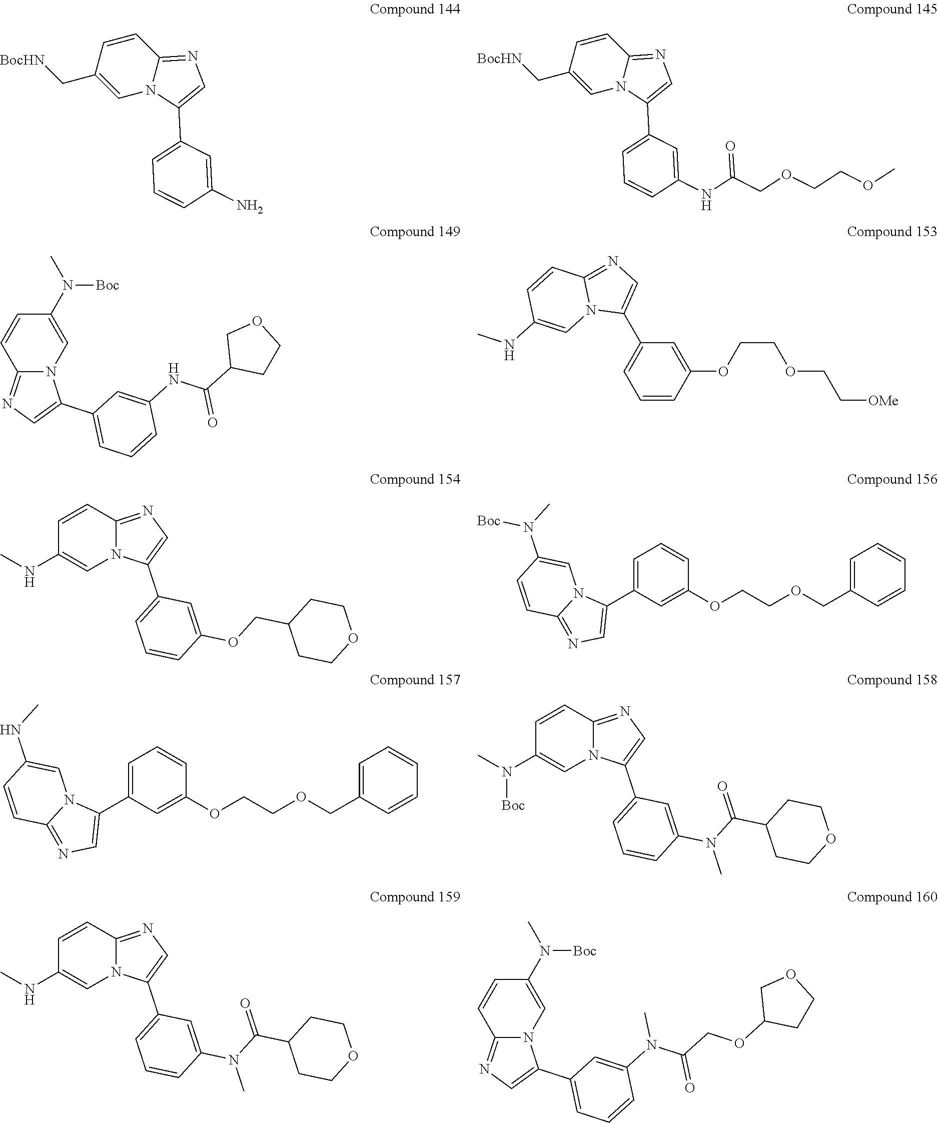

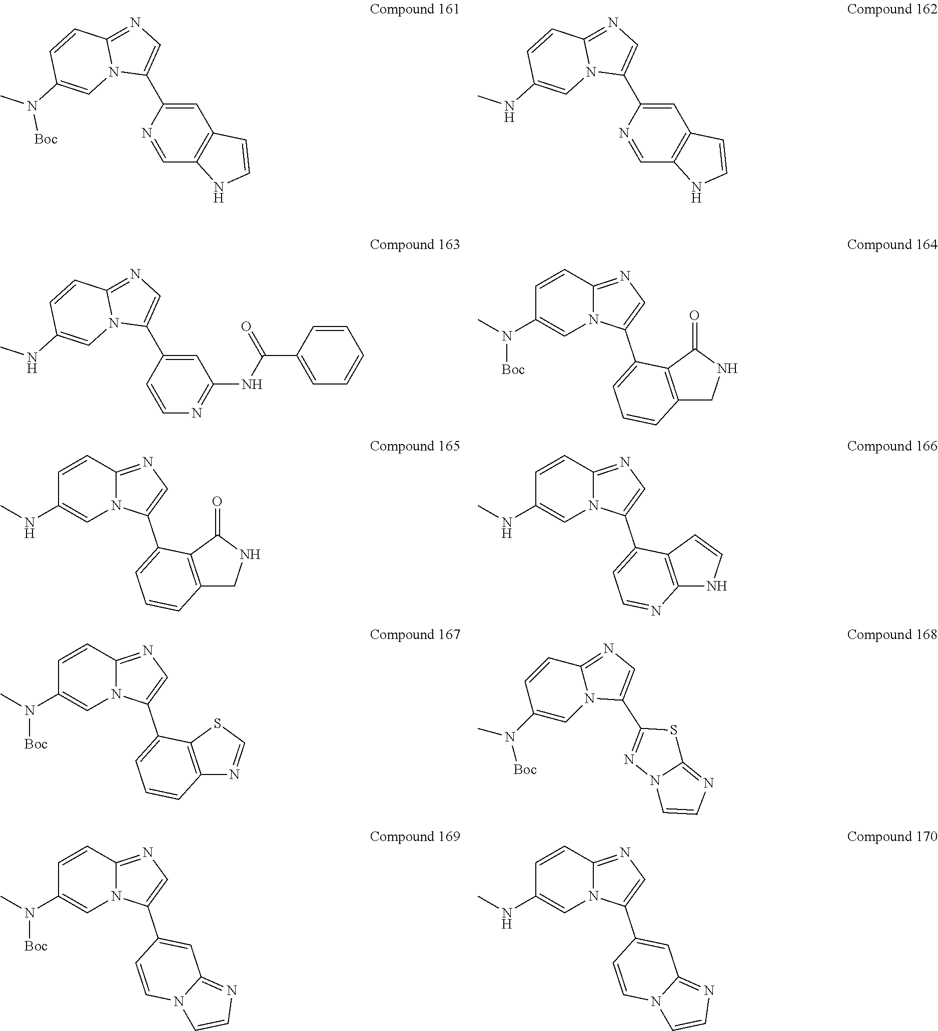

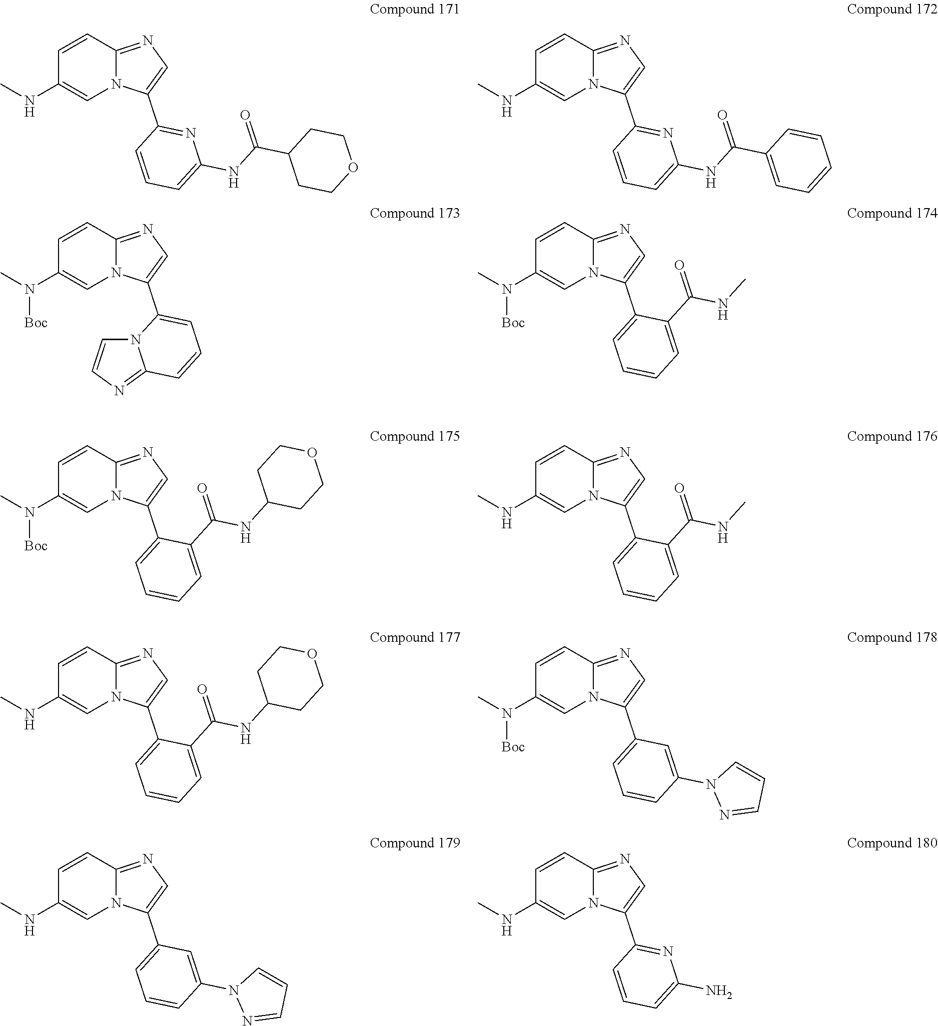

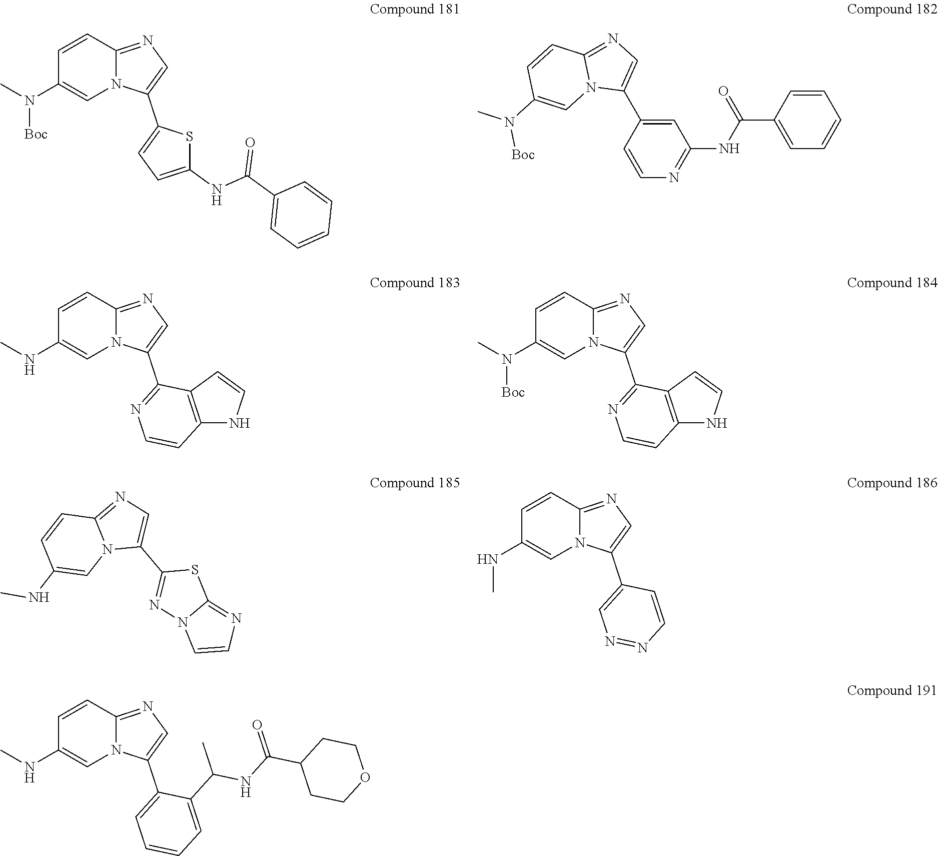

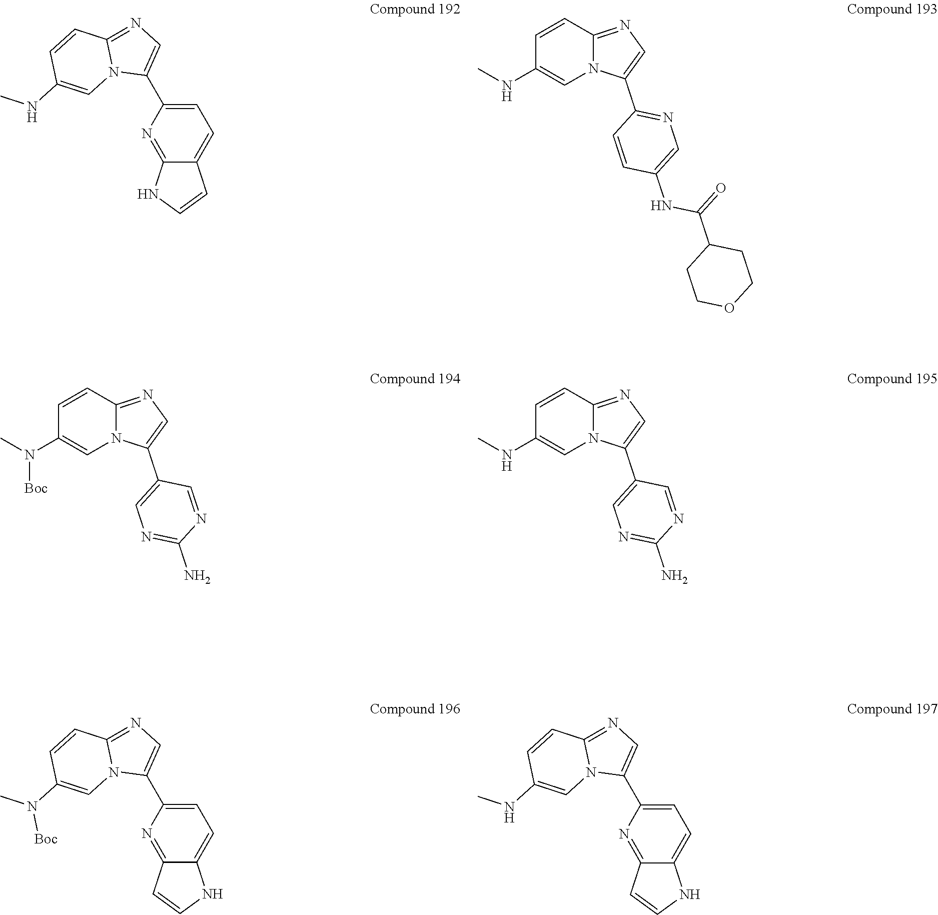 [SMILES: CNc1ccc2ncc(-c3ccc4cc[nH]c4n3)n2c1 ; CNc1ccc2ncc(-c3ccc(NC(=O)C4CCOCC4)cn3)n2c1 ; CN(C(=O)OC(C)(C)C)c1ccc2ncc(-c3cnc(N)nc3)n2c1 ; CNc1ccc2ncc(-c3cnc(N)nc3)n2c1 ; CN(C(=O)OC(C)(C)C)c1ccc2ncc(-c3ccc4[nH]ccc4n3)n2c1 ; CNc1ccc2ncc(-c3ccc4[nH]ccc4n3)n2c1]

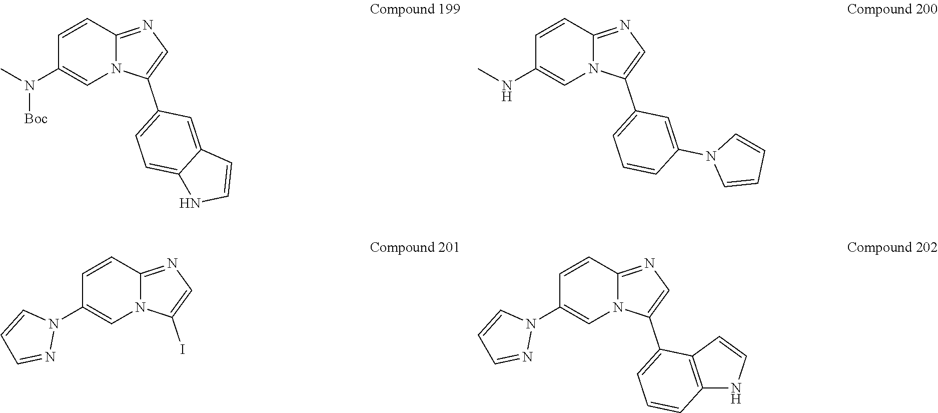 [SMILES: CN(C(=O)OC(C)(C)C)c1ccc2ncc(-c3ccc4[nH]ccc4c3)n2c1 ; CNc1ccc2ncc(-c3cccc(-n4cccc4)c3)n2c1 ; Ic1cnc2ccc(-n3cccn3)cn12 ; c1cc(-c2cnc3ccc(-n4cccn4)cn23)c2cc[nH]c2c1]

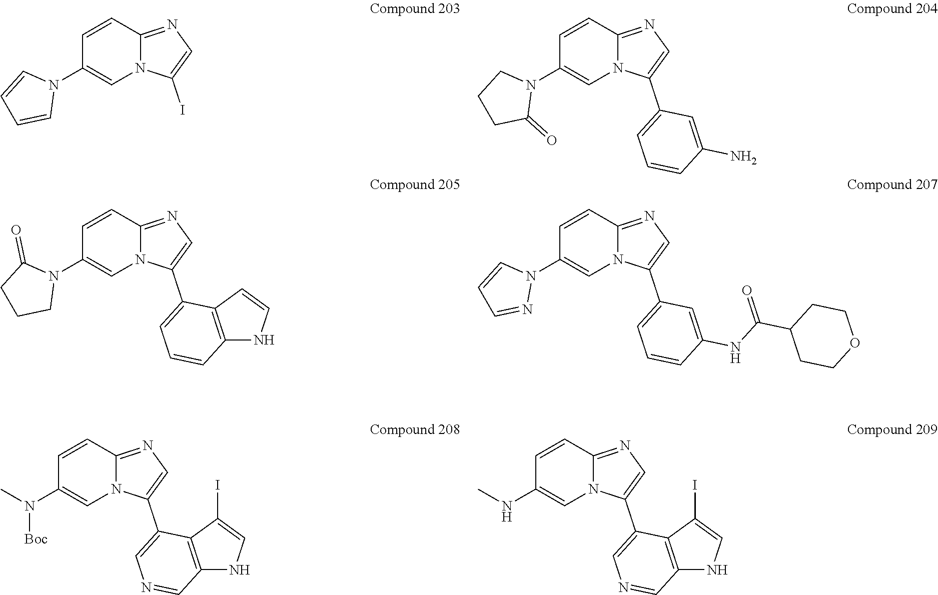 [SMILES: Ic1cnc2ccc(-n3cccc3)cn12 ; Nc1cccc(-c2cnc3ccc(N4CCCC4=O)cn23)c1 ; O=C1CCCN1c1ccc2ncc(-c3cccc4[nH]ccc34)n2c1 ; O=C(Nc1cccc(-c2cnc3ccc(-n4cccn4)cn23)c1)C1CCOCC1 ; CN(C(=O)OC(C)(C)C)c1ccc2ncc(-c3cncc4[nH]cc(I)c34)n2c1 ; CNc1ccc2ncc(-c3cncc4[nH]cc(I)c34)n2c1]

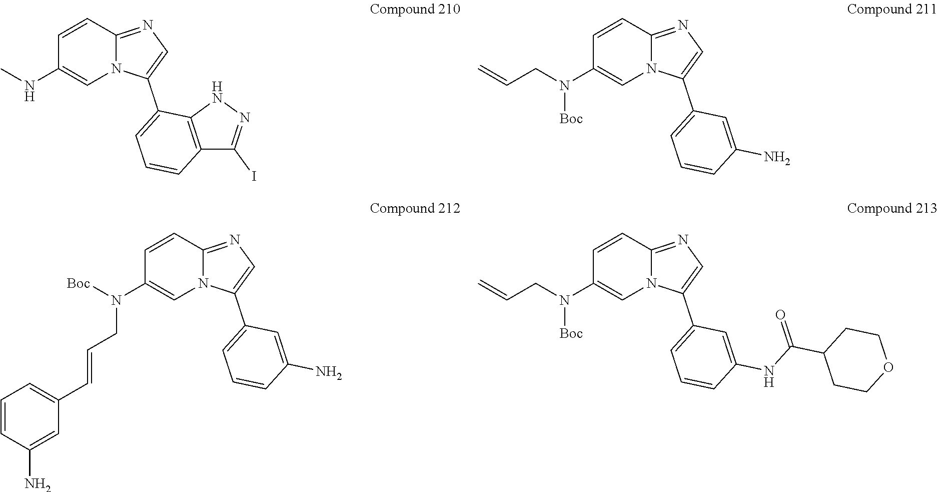 [SMILES: CNc1ccc2ncc(-c3cccc4c(I)n[nH]c34)n2c1 ; C=CCN(C(=O)OC(C)(C)C)c1ccc2ncc(-c3cccc(N)c3)n2c1 ; CC(C)(C)OC(=O)N(C/C=C/c1cccc(N)c1)c1ccc2ncc(-c3cccc(N)c3)n2c1 ; C=CCN(C(=O)OC(C)(C)C)c1ccc2ncc(-c3cccc(NC(=O)C4CCOCC4)c3)n2c1]

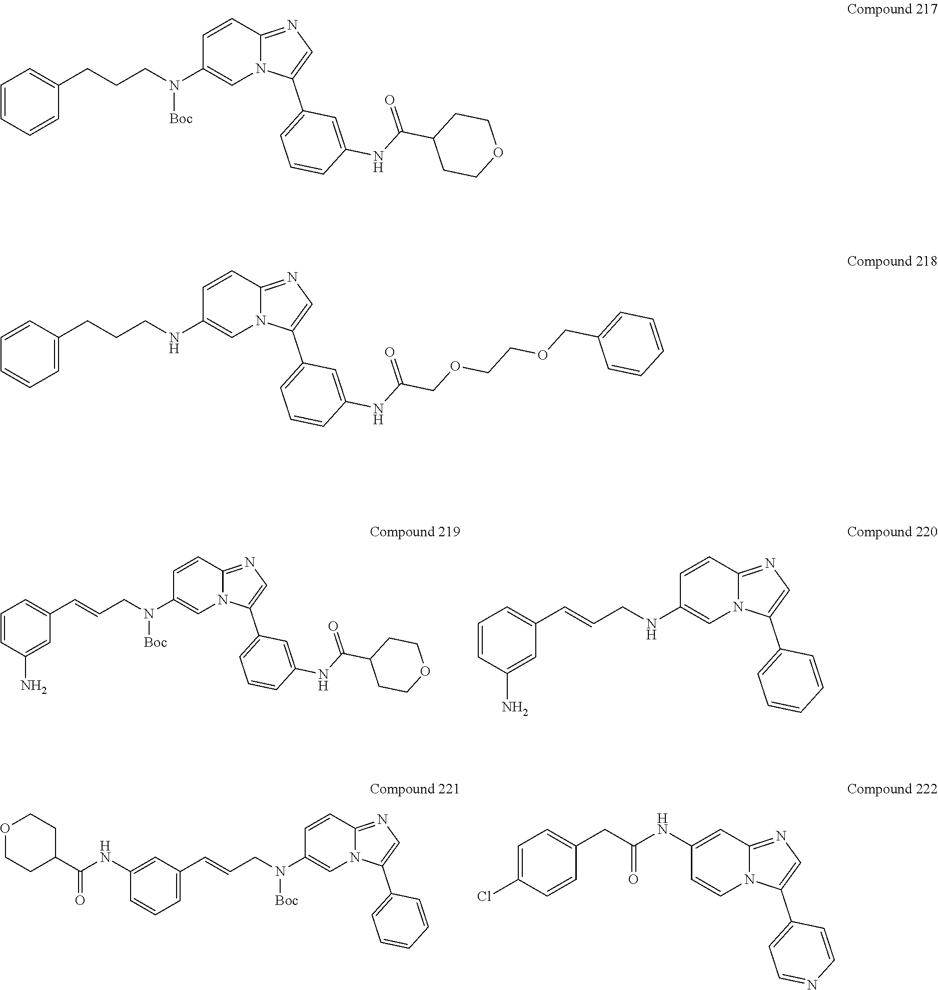 [SMILES: CC(C)(C)OC(=O)N(CCCc1ccccc1)c1ccc2ncc(-c3cccc(NC(=O)C4CCOCC4)c3)n2c1 ; O=C(COCCOCc1ccccc1)Nc1cccc(-c2cnc3ccc(NCCCc4ccccc4)cn23)c1 ; CC(C)(C)OC(=O)N(C/C=C/c1cccc(N)c1)c1ccc2ncc(-c3cccc(NC(=O)C4CCOCC4)c3)n2c1 ; Nc1cccc(/C=C/CNc2ccc3ncc(-c4ccccc4)n3c2)c1 ; CC(C)(C)OC(=O)N(C/C=C/c1cccc(NC(=O)C2CCOCC2)c1)c1ccc2ncc(-c3ccccc3)n2c1 ; O=C(Cc1ccc(Cl)cc1)Nc1ccn2c(-c3ccncc3)cnc2c1]

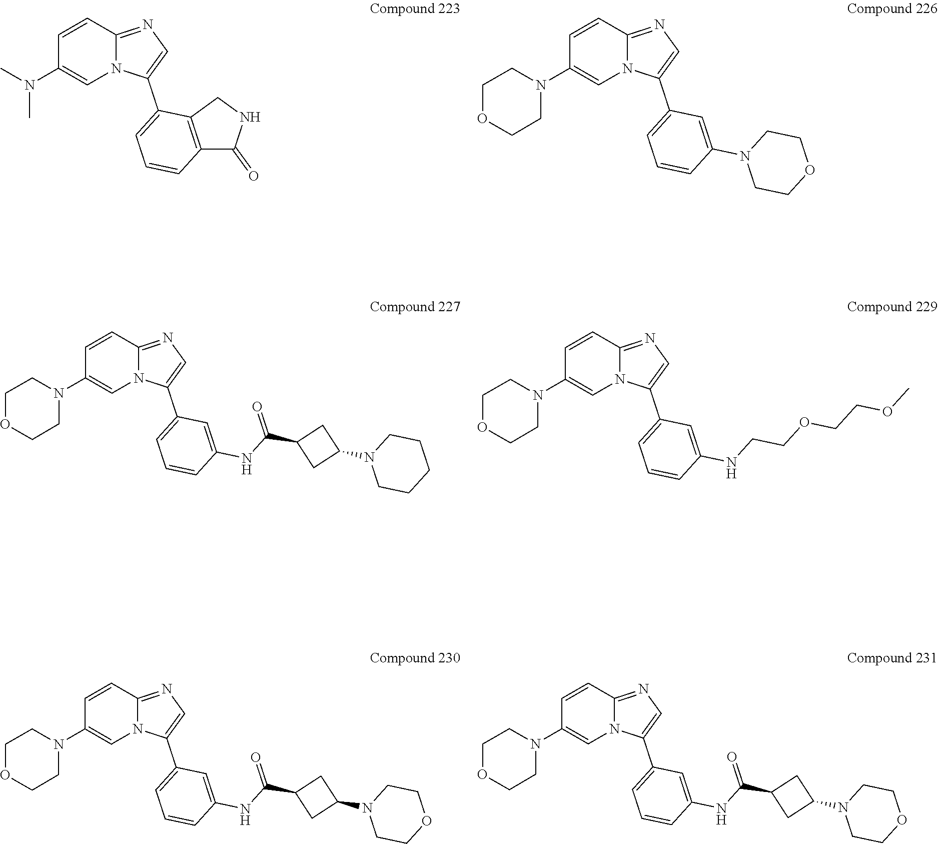 [SMILES: CN(C)c1ccc2ncc(-c3cccc4c3CNC4=O)n2c1 ; c1cc(-c2cnc3ccc(N4CCOCC4)cn23)cc(N2CCOCC2)c1 ; O=C(Nc1cccc(-c2cnc3ccc(N4CCOCC4)cn23)c1)[C@H]1C[C@H](N2CCCCC2)C1 ; COCCOCCNc1cccc(-c2cnc3ccc(N4CCOCC4)cn23)c1 ; O=C(Nc1cccc(-c2cnc3ccc(N4CCOCC4)cn23)c1)[C@H]1C[C@@H](N2CCOCC2)C1 ; O=C(Nc1cccc(-c2cnc3ccc(N4CCOCC4)cn23)c1)[C@H]1C[C@H](N2CCOCC2)C1]

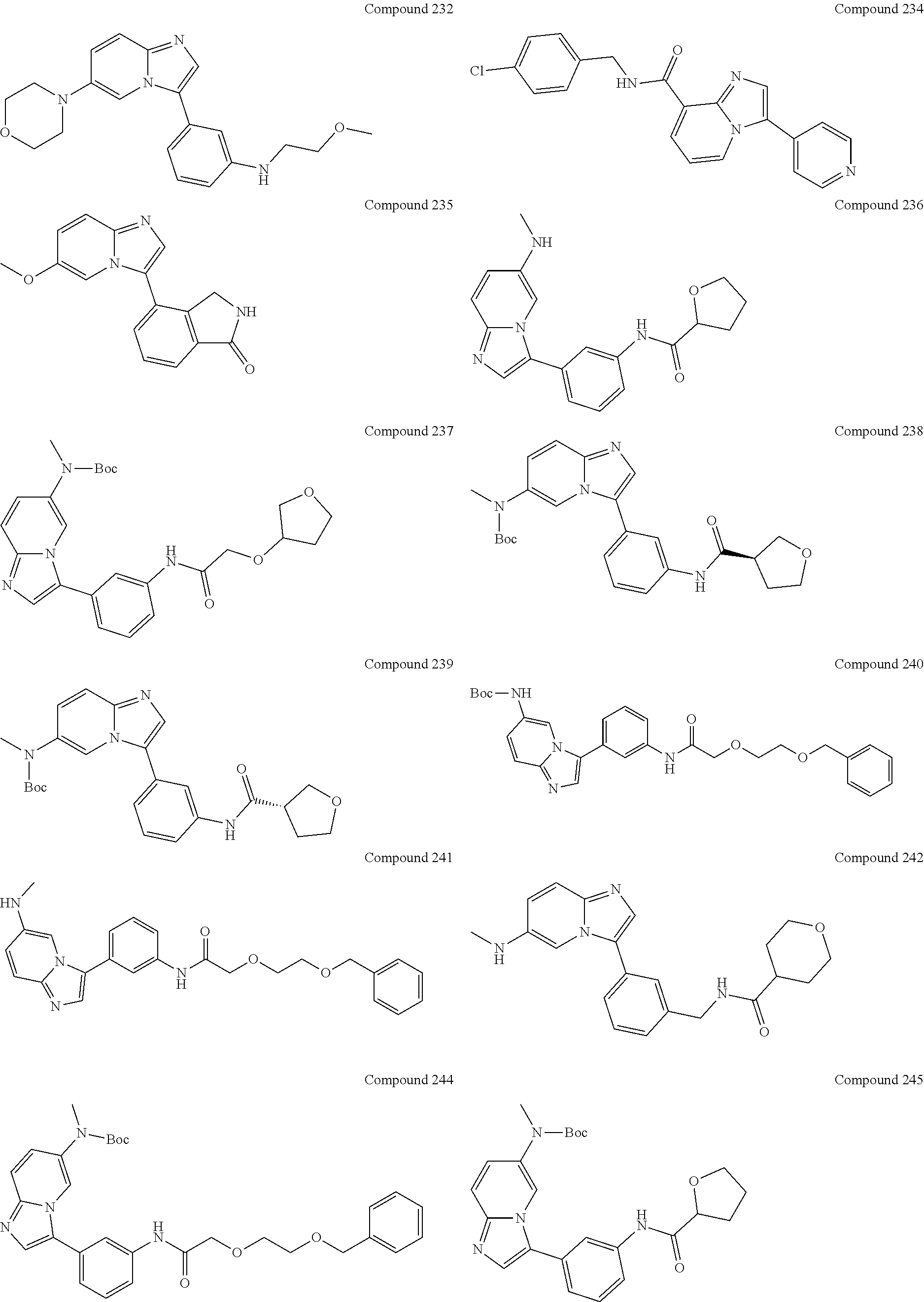

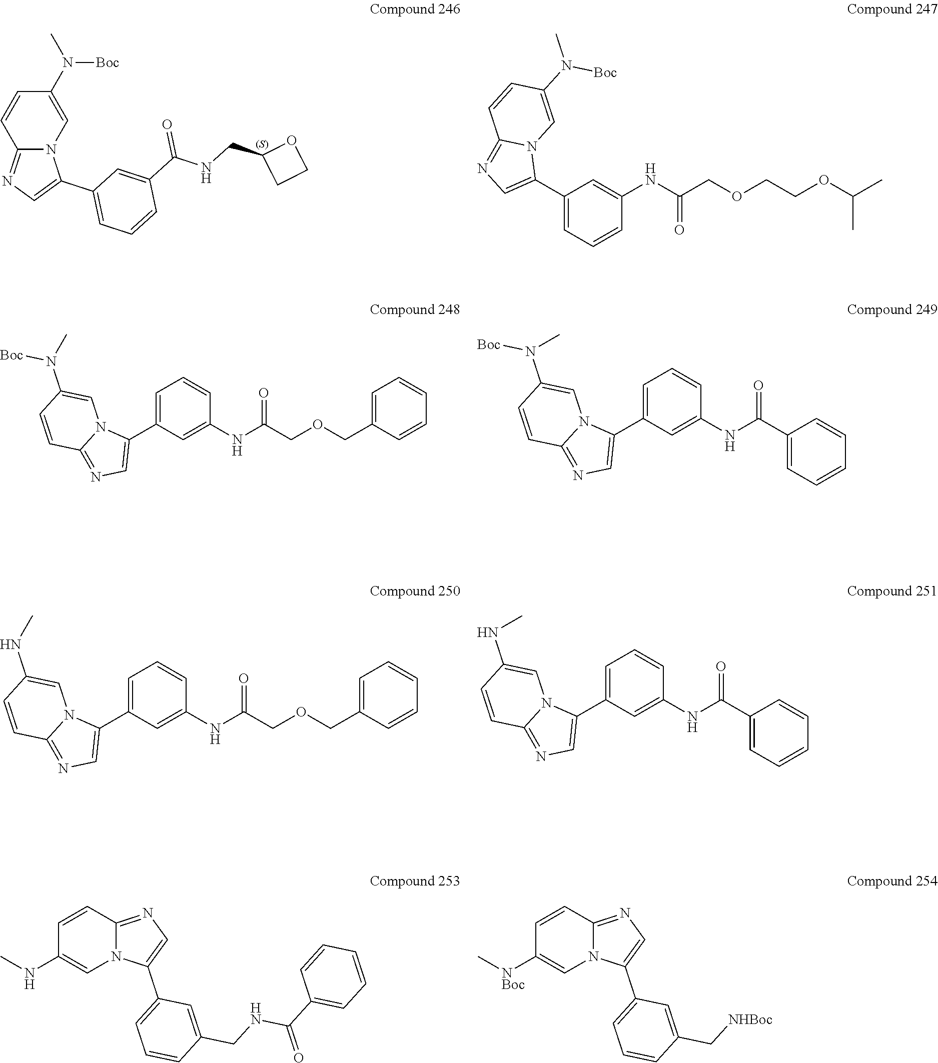

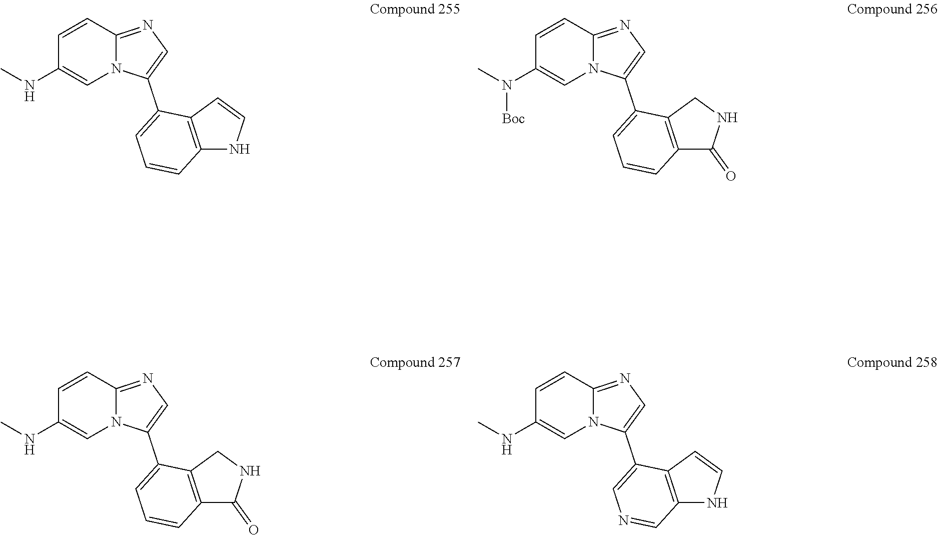 [SMILES: CNc1ccc2ncc(-c3cccc4[nH]ccc34)n2c1 ; CN(C(=O)OC(C)(C)C)c1ccc2ncc(-c3cccc4c3CNC4=O)n2c1 ; CNc1ccc2ncc(-c3cccc4c3CNC4=O)n2c1 ; CNc1ccc2ncc(-c3cncc4[nH]ccc34)n2c1]

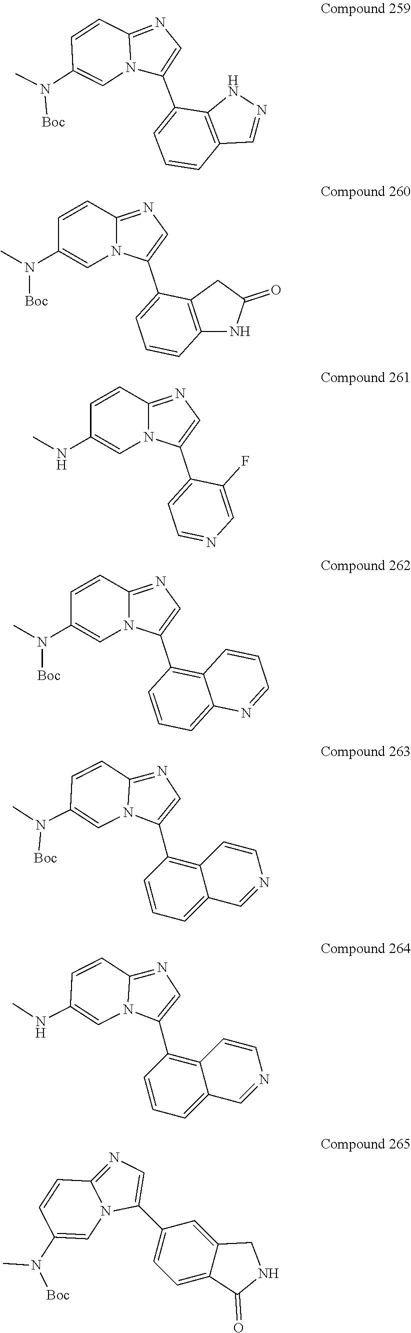

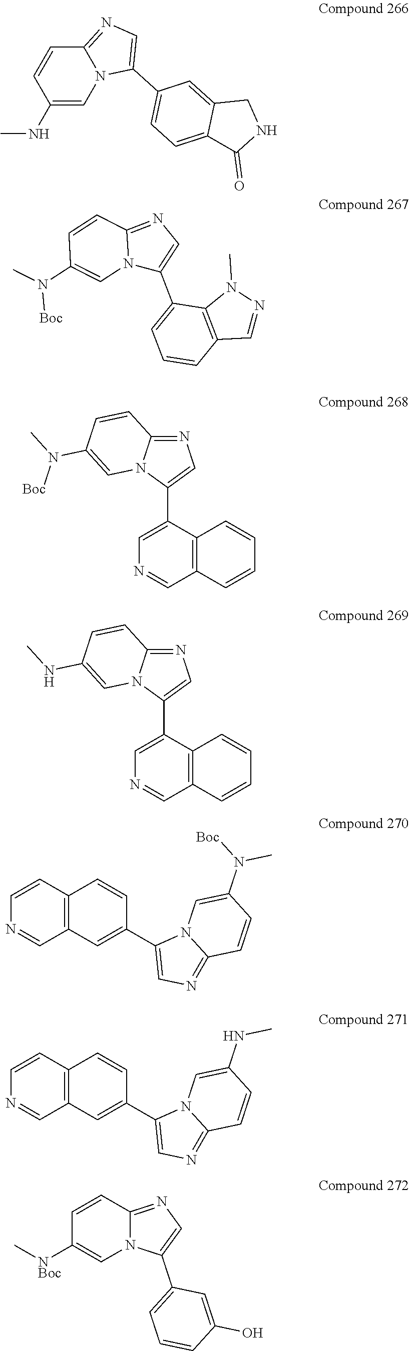

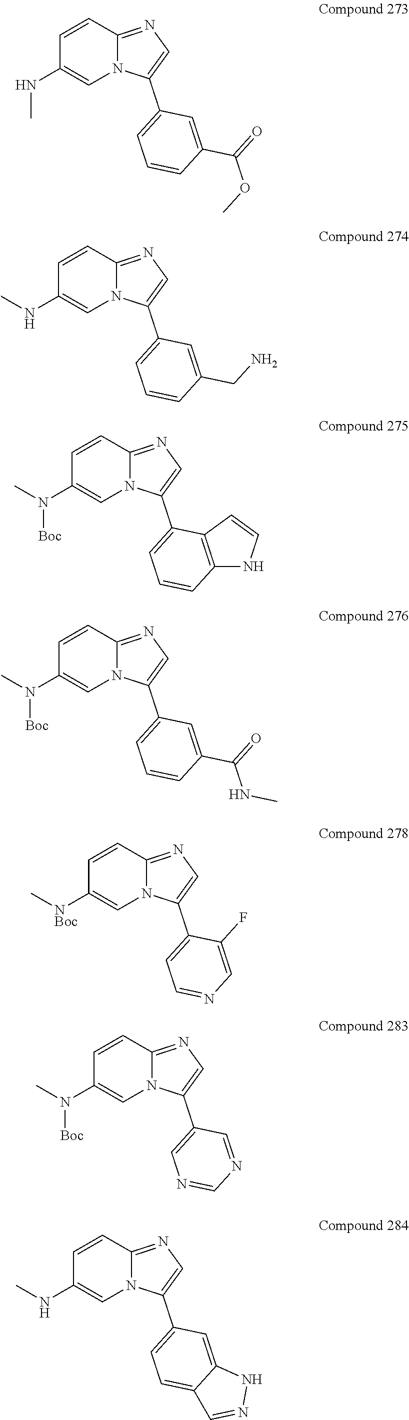

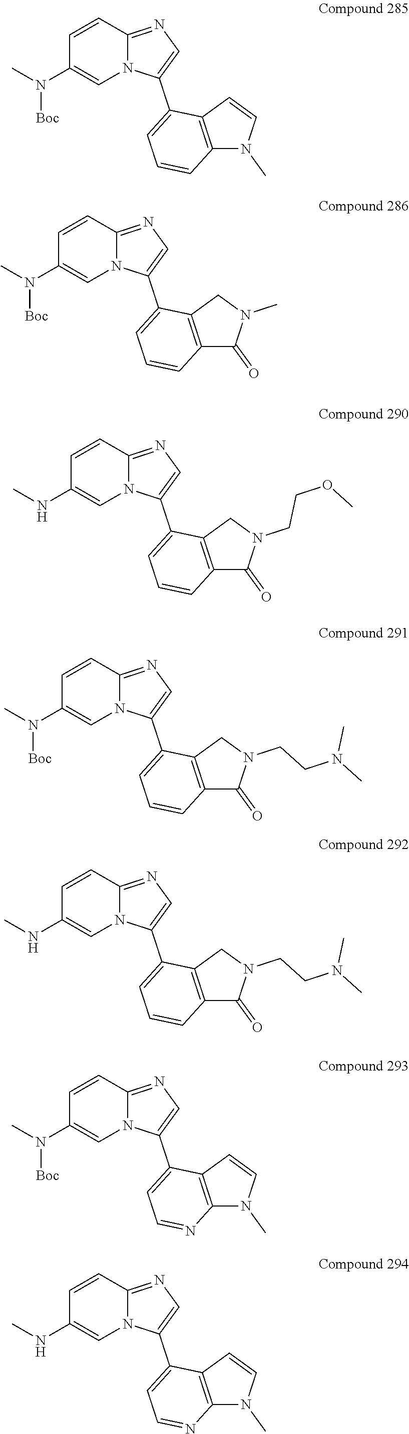

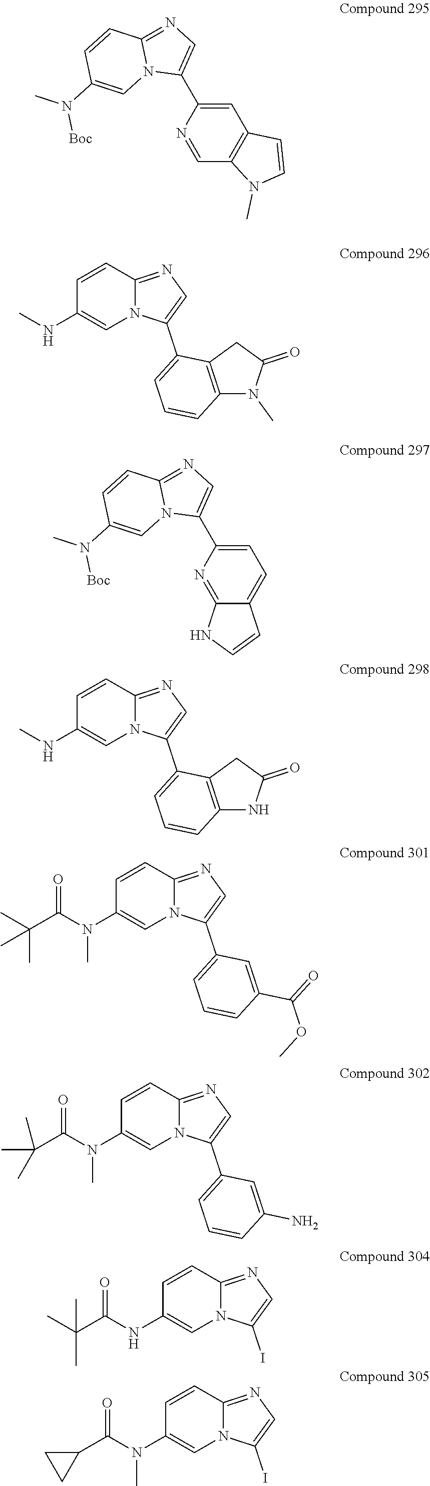

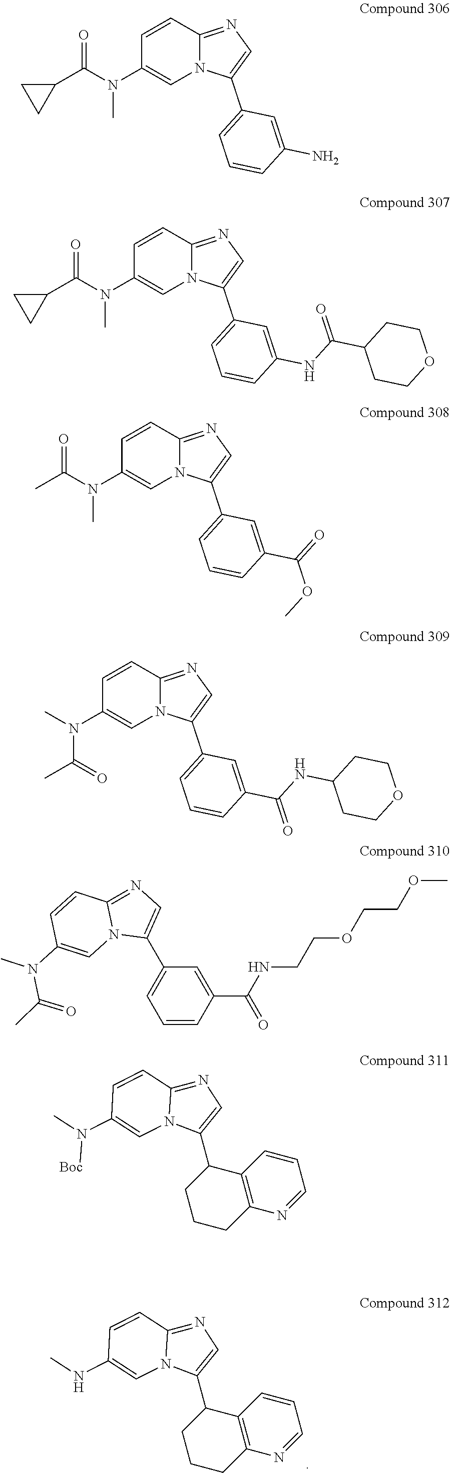

As used herein, when reference is made to the compounds represented by formula I to formula VII, it also includes pharmaceutically acceptable salts of the compounds represented by formula I to formula VII or stereoisomers thereof.

The present disclosure further relates to a method of treating or preventing a neurodegenerative disease or a neurological disease or condition associated therewith, comprising administering to a subject in need thereof the compound of the present disclosure as an inhibitor of SARM1 enzymatic activity. In particular, the present disclosure relates to a method of treating or preventing an axonal degeneration-related disease or condition, comprising administering to a subject in need thereof the compound of the present disclosure as an inhibitor of SARM1 enzymatic activity. More particularly, the present disclosure relates to a method of inhibiting SARM1 enzymatic activity, comprising administering to a subject in need thereof the compound of the present disclosure. More particularly, the present disclosure relates to a method of inhibiting axonal degeneration, comprising administering to a subject in need thereof the compound of the present disclosure. The compound or composition of the present disclosure can be administered to a subject or patient in need thereof in an effective amount.

Accordingly, the present disclosure further relates to use of the compound of the present disclosure or a pharmaceutically acceptable salt or stereoisomer thereof in the manufacture of a medicament for treating or preventing a neurodegenerative disease or a neurological disease or condition. The present disclosure further relates to use of the compound of the present disclosure or a pharmaceutically acceptable salt or stereoisomer thereof in the manufacture of an inhibitor of SARM1 enzymatic activity. The present disclosure further relates to use of the compound of the present disclosure or a pharmaceutically acceptable salt or stereoisomer thereof in in the manufacture of a medicament for treating or preventing an axonal degeneration-related disease or condition. Preferably, the neurodegenerative disease or neurological disease or condition or axonal degeneration-related disease or condition is selected from the group consisting of Alzheimer's disease, Parkinson's disease, multiple sclerosis, amyotrophic lateral sclerosis and peripheral neuropathy.

### DETAILED DESCRIPTION

### Terms

As used herein, reference to a “compound” having a particular structural formula generally also encompasses pharmaceutically acceptable salts, stereoisomers, diastereoisomers, enantiomers, racemic mixtures and isotope derivatives thereof.

It is known to those skilled in the art that in addition to the salts of compounds, solvates and hydrates are alternative forms of compounds, which can all be converted into the compounds under certain conditions. Therefore, when referring to a compound herein, the solvate and hydrate thereof are generally included.

The pharmaceutically acceptable salt of the present disclosure may be formed using, for example, the following inorganic or organic acids. “Pharmaceutically acceptable salts” refer to salts which, within the scope of reasonable medical judgment, are suitable for contact with humans and mammals tissue without undue toxicity, irritation, allergic reactions, etc., and have a reasonable benefit/risk ratio. The salts may be prepared in situ during the final isolation and purification of the compounds of the present disclosure, or prepared separately by reacting a free base or free acid with a suitable reagent. For example, a free base can be reacted with a suitable acid. Examples of pharmaceutically acceptable acid addition salts are salts formed by amino (amine) with inorganic acids (such as hydrochloric acid, hydrobromic acid, phosphoric acid, sulfuric acid, and perchloric acid) or organic acids (such as acetic acid, oxalic acid, maleic acid, tartaric acid, citric acid, succinic acid or malonic acid), or salts formed by using other methods in the art such as ion exchange. Other pharmaceutically acceptable salts include sodium alginate, ascorbate, benzenesulfonate, adipate, camphorsulfonate, aspartate, benzoate, bisulfate, borate, butyrate, camphorate, citrate, lauryl sulfate, ethanesulfonate, formate, fumarate, glucoheptonate, glycerol phosphate, gluconate, enanthate, hexanoate, hydroiodide, lactobionate, lactate, laurate, lauryl sulfate, malate, maleate, malonate, methanesulfonate, 2-naphthalenesulfonate, niacinate, nitrate, oleate, oxalate, palmitate, pamate, pectate, persulfate, 3-phenylpropionate, phosphate, picrate, pivalate, propionate, stearate, succinate, sulfate, tartrate, thiocyanate, p-toluenesulfonate, undecanoate, valerate, etc.

The pharmaceutically acceptable salt of the present disclosure can be prepared by conventional methods, for example, by dissolving the compound of the present disclosure in a water-miscible organic solvent (such as methanol, ethanol, acetone, and acetonitrile), adding an excess amount of organic acid or inorganic acid aqueous solution thereto, to precipitate salt from the resulting mixture, removing the solvent and the remaining free acid therefrom, and isolating the precipitated salt.

“Solvate” as used herein refers to a physical association of the compound of the present disclosure with one or more solvent molecules, whether organic or inorganic. This physical association includes a hydrogen bond. In certain circumstances, for example, when one or more solvent molecules are incorporated into the crystal lattice of a crystalline solid, the solvate will be able to be isolated. The solvent molecules in a solvate may exist in regular and/or disordered arrangements. A solvate may contain stoichiometric or non-stoichiometric amounts of solvent molecules. “Solvate” encompasses both solution phase and isolable solvate. Exemplary solvates include, but are not limited to, hydrates, ethanolates, metholates, and isopropolates. Solvation methods are well known in the art.

“Stereoisomerism” as used herein is divided into conformational isomerism and configurational isomerism. Configurational isomerism can further be divided into cis-trans isomerism and optical isomerism. Conformational isomerism refers to a stereoisomerism phenomenon in which each atom or group of atoms of an organic molecule with a certain configuration are arranged differently in space due to the rotation or distortion of carbon-carbon single bonds. Common structures include alkanes and cycloalkanes, such as the chair conformation and boat conformation that appear in the cyclohexane structure. “Stereoisomer” means that the compound of the present disclosure contains one or more asymmetric centers, which is thus available to be a racemate and racemic mixture, single enantiomer, diastereomeric mixture and single diastereomer. The compound of the present disclosure can have an asymmetric center, and each asymmetric center will produce two optical isomers. The scope of the present disclosure includes all possible optical isomers and diastereomeric mixtures and pure or partially pure compounds.

In particular, the compound described in the present disclosure may exist as a tautomer having different points of attachment of hydrogens by displacement of one or more double bonds. For example, a ketone and its enol form are keto-enol tautomers. Each tautomer and mixtures thereof are included in the compound of the present disclosure. All enantiomers, diastereomers, racemates, mesomers, cis-trans isomers, tautomers, geometric isomers, epimers, mixtures thereof, etc. are included in the scope of the present disclosure.

“Isotopic derivative” as used herein refers to a molecule in which the compound of the present disclosure is isotopically labeled. Isotopes commonly used as isotope labels include hydrogen isotopes: 2H and 3H; carbon isotopes: 11C, 13C and 14C; chlorine isotopes: 35Cl and 37Cl; fluorine isotope: 18F; iodine isotopes: 123I and 125I; nitrogen isotopes: 13N and 15N; oxygen isotopes: 15O, 17O and 18O, and sulfur isotope: 35S. The isotopically labeled compounds can be used to study the distribution of pharmaceutical molecules in tissues. Substitution by certain heavy isotopes, such as deuterium (2H), can enhance metabolic stability and extend half-life, thereby reducing administration amount and providing therapeutic advantages. Isotopically labeled compounds are generally synthesized starting from labeled starting materials using known synthetic techniques as for non-isotopically labeled compounds.

The compound of the present disclosure may be used in combination with an additional SARM1 enzymatic activity inhibitor for treating or preventing a neurodegenerative disease or related neurological disease or condition, or may be used in combination with an additional active drug for treating or preventing a neurodegenerative disease or related neurodegenerative disease or condition, for treating or preventing a neurodegenerative disease or related neurodegenerative disease or condition.

The compound of the present disclosure or pharmaceutically acceptable salt thereof can be administered orally or parenterally as an active ingredient, at an effective amount ranging from 0.1 to 2000 mg/kg body weight/day, preferably, 0.1 to 100 mg/kg body weight/day in the case of mammals including humans (body weight about 70 kg), in single or divided doses per day, or with/without following a predetermined time. The administration amount of the active ingredient may be adjusted based on a number of relevant factors such as the condition of the subject to be treated, the type and severity of the disease, the rate of administration and physician opinion. In some cases, amounts less than the above amounts may be appropriate.

The pharmaceutical composition of the present disclosure can be formulated into a dosage form for oral administration or parenteral administration (including intramuscular administration, intravenous and subcutaneous administration, and intratumoral injection) according to any of conventional methods, such as tablets, granules, powders, capsules, syrups, emulsions, microemulsions, solutions or suspensions.

The pharmaceutical composition of the present disclosure for oral administration can be prepared by mixing the active ingredient with a carrier, for example, selected from the group consisting of cellulose, calcium silicate, magnesium stearate, calcium stearate, corn starch, lactose, sucrose, dextrose, calcium phosphate, stearic acid, surfactants, suspending agents, gelatin, talc, emulsifiers and diluents. Examples of carriers used in the injectable compositions of the present disclosure include water, glycerides, salt solutions, alcohols, glycols, glucose solutions, ethers (such as polyethylene glycol 400), oils, fatty acids, fatty acid esters, surfactants, suspending agents and emulsifiers.

Unless otherwise stated, mass spectrometry, nuclear magnetic resonance, HPLC, protein chemistry, biochemistry, recombinant DNA technology, and pharmacological conventional methods are used. In the present disclosure, “or” or “and” are used to mean “and/or” unless stated otherwise.

In the description and claims, a given chemical formula or name will cover all stereo and optical isomers as well as racemates in which the above isomers are present. Unless otherwise indicated, all chiral (enantiomeric and diastereomeric) and racemic forms are within the scope of the present disclosure. Many geometric isomers of C═C double bonds, C═N double bonds, ring systems, etc. may also exist in the compounds, and all the above stable isomers are encompassed by the present disclosure. The present disclosure describes cis- and trans- (or E- and Z-) geometric isomers of the compounds of the present disclosure, which may be isolated into mixtures of isomers or separate isomeric forms.

The compounds of the present disclosure can be isolated in optically active or racemic forms. All methods for preparing the compounds of the present disclosure and the intermediates prepared therein are considered to be part of the present disclosure. In the preparation of enantiomeric or diastereomeric products, they can be isolated by conventional methods, for example by chromatography or fractional crystallization. It should be understood that all tautomeric forms which may exist are included in the present disclosure. The compounds of the present disclosure are commercially available as they are known in the art.

Unless otherwise defined, when a substituent is designated as “optionally substituted”, the substituent is selected from, for example, substituents such as alkyl, cycloalkyl, aryl, heterocyclyl, halogen, hydroxyl, alkoxy, nitro, cyano, oxo, alkanoyl, aryloxy, alkanoyloxy, amino, alkylamino, arylamino, and alkylthio.

The term “alkyl” or “alkylene” as used herein is intended to include both branched and straight chain saturated aliphatic hydrocarbon groups having the specified number of carbon atoms. The alkyl in the present disclosure is preferably C₁-C₁₂ alkyl, C₁-C₁₀ alkyl, C₁-C₈ alkyl, more preferably C₁-C₆ alkyl, particularly preferably C₁-C₄ alkyl, particularly C₁-C₃ alkyl. For example, “C₁-C₆ alkyl” represents an alkyl having 1 to 6 carbon atoms. Examples of alkyl include, but are not limited to, methyl (Me), ethyl (Et), propyl (such as n-propyl and isopropyl), butyl (such as n-butyl, isobutyl, tert-butyl), and pentyl (such as n-pentyl, isopentyl, neopentyl). In the C₁-C₁₂ alkyl in the present disclosure, 1 to 4 —CH₂— units are optionally replaced by O atoms, S atoms or —NH—.

The term “alkoxy” or “alkyloxy” refers to —O-alkyl. For example, “C₁-C₆ alkoxy” (or alkyloxy) is intended to include C₁, C₂, C₃, C₄, C₅, C₆ alkoxy. Preferred alkoxy includes C₁-C₁₀ alkoxy, C₁-C₈ alkoxy, more preferably C₁-C₆ alkoxy, particularly preferably C₁-C₄ alkoxy, particularly C₁-C₃ alkoxy. Examples of alkoxy include, but are not limited to, methoxy, ethoxy, propoxy (such as n-propoxy and isopropoxy), and tert-butoxy. Similarly, “alkylthio” or “thioalkoxy” represents a sulfur-bridged alkyl defined as above having the specified number of carbon atoms; for example, methyl-S— and ethyl-S—. Similarly, preferred alkylthio includes C₁-C₁₀ alkylthio, C₁-C₈ alkylthio, more preferably C₁-C₆ alkylthio, particularly preferably C₁-C₄ alkylthio, particularly C₁-C₃ alkylthio.

The term “carbonyl” refers to an organic functional group consisting of two atoms of carbon and oxygen connected by a double bond (C═O or C(O)).

The term “aryl”, alone or as part of a large moiety such as “aralkyl”, “aralkoxy” or “aryloxyalkyl”, refers to a monocyclic, bicyclic or tricyclic ring system having a total of 6 to 14 ring members, wherein at least one ring in the system is aromatic and wherein each ring in the system contains 3 to 7 ring members. In certain embodiments of the present disclosure, “aryl” refers to an aromatic ring system including, but not limited to, phenyl, naphthyl, biphenyl, indanyl, 1-naphthyl, 2-naphthyl, and tetrahydronaphthyl. The aryl of the present disclosure is preferably C₆-C₁₀ aryl. The term “aralkyl” or “arylalkyl” refers to an alkyl residue attached to an aryl ring. Non-limiting examples include benzyl and phenethyl.

The term “cycloalkyl” refers to cyclic alkyl, which may be monocyclic or bicyclic. The cycloalkyl of the present disclosure is preferably C₃-C₈ cycloalkyl, including but not limited to cyclopropyl, cyclobutyl, cyclopentyl, cyclohexyl and norbornyl.

“Halo” or “halogen” includes fluorine, chlorine, bromine and iodine. “Haloalkyl” is intended to include branched and straight chain saturated aliphatic hydrocarbon groups having the specified number of carbon atoms substituted with one or more halogens. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl and heptachloropropyl. Examples of haloalkyl further include fluoroalkyl intended to include branched and straight chain saturated aliphatic hydrocarbon groups having the specified number of carbon atoms substituted with one or more fluorine atoms, particularly preferably trifluoromethyl.

Haloalkoxy represents haloalkyl defined as above having the specified number of carbon atoms linked via an oxygen bridge. For example, “C₁-C₆ haloalkoxy” is intended to include C₁, C₂, C₃, C₄, C₅, C₆ haloalkoxy. Examples of haloalkoxy include, but are not limited to, trifluoromethoxy, 2,2,2-trifluoroethoxy and pentafluoroethoxy. Similarly, “haloalkylthio” or “thiohaloalkoxy” represents a sulfur-bridged haloalkyl defined as above having the specified number of carbon atoms; for example, trifluoromethyl-S— and pentafluoroethyl —S—.

In the present disclosure, one or more halogens may be each independently selected from the group consisting of fluorine, chlorine, bromine and iodine.

The term “heteroaryl” refers to a stable 3-membered, 4-membered, 5-membered, 6-membered, or 7-membered aromatic monocyclic ring or a 7-membered, 8-membered, 9-membered, or 10-membered aromatic bicyclic ring or aromatic polycyclic heterocyclic ring, which is fully unsaturated or partially unsaturated, and contains carbon atoms and 1, 2, 3 or 4 heteroatoms independently selected from the group consisting of N, O and S. Nitrogen and sulfur heteroatoms may optionally be oxidized. Nitrogen atoms are substituted or unsubstituted (i.e., N or NR, where R is H or another substituent if defined). Heterocycles can be attached to their pendant groups at any heteroatom or carbon atom that results in a stable structure. If the resulting compound is stable, the heterocyclyl described herein may be substituted on the carbon or nitrogen atom. The nitrogen in the heterocycle may optionally be quaternized. Preferably, when the total number of S and O atoms in the heterocycle exceeds 1, then these heteroatoms are not adjacent to each other. Preferably, the total number of S and O atoms in the heterocycle is not greater than 1. When the term “heterocycle” is used, it is intended to include heteroaryl. Examples of heteroaryl include, but are not limited to, acridinyl, azetidinyl, azecinyl, benzimidazolyl, benzofuryl, benzothiofuranyl, benzothienyl, benzoxazolyl, benzoxazolinyl, benzothiazolyl, benzotriazolyl, benzotetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolyl, 2H,6H-1,5,2-dithiazinyl, dihydrofuro[2,3-b]tetrahydrofuranyl, furanyl, furoxanyl, imidazolidinyl, imidazolinyl, imidazolyl, 1H-indazolyl, imidazopyridyl, indolenyl, dihydroindolyl, indolizinyl, indolyl, 3H-indolyl, isatinoyl, isobenzofuranyl, isochromanyl, isoindazolyl, isodihydroindolyl, isoindolyl, isoquinolinyl, isothiazolyl, isothiazopyridyl, isoxazolyl, isoxazolopyridyl, methylenedioxyphenyl, morpholinyl, diazanaphthyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridyl, oxazolidinyl, perimidyl, oxindolyl, pyrimidinyl, phenanthridinyl, phenanthrolinyl, phenazinyl, phenothiazinyl, phenoxathiyl, phenoxazinyl, phthalazinyl, piperazinyl, piperidinyl, piperidinonyl, 4-piperidinonyl, piperonyl, pteridinyl, purinyl, pyranyl, pyrazinyl, pyrazolidinyl, pyrazolinyl, pyrazopyridyl, pyrazolyl, pyridazinyl, pyridoxazolyl, pyridimidazolyl, pyridothiazolyl, pyridyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrazolyl, tetrahydrofuryl, tetrahydroisoquinolyl, tetrahydroquinolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridyl, thienothiazolyl, thienoxazolyl, thienoimidazolyl, thienyl, triazinyl, 1,2,3-triazolyl, 1,2,4-triazolyl, 1,2,5-triazolyl, 1,3,4-triazolyl and xanthyl, quinolyl, isoquinolyl, phthalazinyl, quinazolinyl, indolyl, isoindolyl, dihydroindolyl, 1H-indazolyl, benzimidazolyl, 1,2,3,4-tetrahydroquinolyl, 1,2,3,4-tetrahydroisoquinolyl, 5,6,7,8-tetrahydro-quinolyl, 2,3-dihydro-benzofuryl, chromanyl, 1,2,3,4-tetrahydro-quinoxalinyl and 1,2,3,4-tetrahydro-quinazolinyl. The term “heteroaryl” may further include a biaryl structure formed by the above-defined “aryl” and a monocyclic “heteroaryl”, such as but not limited to “-phenylbipyridyl-”, “-phenylbipyrimidinyl”, “-pyridylbiphenyl”, “-pyridylbipyrimidinyl-”, “-pyrimidinylbiphenyl-”; wherein the present disclosure further includes fused-ring and spirocyclic compounds containing, for example, the above heterocycles.

The term “substituted”/“substitution” as used herein means that at least one hydrogen atom is replaced by a non-hydrogen group, provided that normal valency is maintained and that the substitution results in a stable compound. As used herein, a cyclic double bond is a double bond formed between two adjacent ring atoms (such as C═C, C═N, or N═N).

When any variable occurs more than once in any composition or formula of a compound, its definition on each occurrence is independent of its definition on every other occurrence. Thus, for example, if a group is shown substituted with 0-3 R, then the group may be optionally substituted with up to three R groups, with R on each occurrence being independently selected from the definition of R. Furthermore, combinations of substituents and/or variables are permitted only if such combinations result in stable compounds.

As used herein, the term “effective amount” refers to an amount of a drug or agent (i.e., the compound of the present disclosure) that will elicit the biological or medical response in a tissue, system, animal, or human, for example, that is sought by a researcher or clinician. Furthermore, the term “therapeutically effective amount” refers to an amount that results in improved treatment, cure, prevention, or alleviation of a disease, condition, or side effect, or reduction in rate at which the disease or condition progresses, compared to a corresponding subject that does not receive such amount. An effective amount may be administered in one or more administrations, applications or doses and is not intended to be limited by a particular formulation or route of administration. The term further includes an amount effective to enhance normal physiological functions within its scope.

The term “treatment” as used herein includes any effect resulting in amelioration of a condition, disease, disorder, etc., such as alleviation, reduction, regulation, amelioration or elimination, or compromise of symptoms thereof.

The term “pharmaceutically acceptable” is used herein to refer to those compounds, substances, compositions and/or dosage forms which, within the scope of reasonable medical judgment, are suitable for use in contact with human and animal tissue without excessive toxicity, irritation, allergic reactions and/or other problems or complications, and proportionate to a reasonable benefit/risk ratio.

The phrase “pharmaceutically acceptable carrier” as used herein refers to a pharmaceutical substance, composition or vehicle such as a liquid or solid filler, diluent, excipient, manufacturing auxiliary (e.g., lubricant, talc, magnesium stearate, calcium stearate or zinc stearate or stearic acid) or a solvent encapsulated substance, which involves carrying or transporting the subject compound from one organ or part of the body to another. Each carrier must be “acceptable” in the sense of being compatible with the other ingredients of the formulation and harmless to the patient.

The term “pharmaceutical composition” refers to a composition comprising a compound of the present disclosure and at least one other pharmaceutically acceptable carrier. “Pharmaceutically acceptable carrier” refers to a medium generally accepted in the art for the delivery of biologically active agents to animals, particularly mammals, including (i.e.) adjuvants, excipients or vehicles such as diluents, preservatives, fillers, flow regulators, disintegrants, wetting agents, emulsifiers, suspending agents, sweeteners, flavoring agents, aromatics, antibacterial agents, antifungal agents, lubricants and dispersants, depending on the mode of administration and characteristics of dosage form.

As used herein, a compound or pharmaceutical composition, when administered, can ameliorate a disease, symptom or condition, especially the severity thereof, delay the onset of the disease, slow down the progression of the disease, or reduce the duration of the disease. Whether the administration is fixed or temporary, continuous or intermittent may be attributed to or related to the administration circumstances.

### Route of Administration

Suitable routes of administration include, but are not limited to, oral, intravenous, rectal, aerosol, parenteral, ocular, pulmonary, transdermal, vaginal, ear canal, nasal and topical administration. In addition, by way of example only, parenteral administration includes intramuscular injection, subcutaneous injection, intravenous injection, intramedullary injection, ventricular injection, intraperitoneal injection, intralymphatic injection, and intranasal injection.

In an aspect, the compounds described herein are administered locally rather than systemically. In a certain embodiment, a long-acting formulation is administered by implantation (e.g., subcutaneously or intramuscularly) or by intramuscular injection. Furthermore, in another particular embodiment, the drug is administered via a targeted drug delivery system, for example, a liposome coated with organ-specific antibodies. In this particular embodiment, the liposome is selectively targeted to specific organs for uptake.

In the pharmaceutical composition of the present disclosure, pharmaceutical carriers can be formulated according to many factors within the scope of knowledge of those skilled in the art. These factors include, but are not limited to, the type and characteristics of the active agent formulated; the subject to whom the composition containing the active agent is to be administered; the intended route of administration of the composition; and the targeted therapeutic indication. Pharmaceutical carriers include aqueous and non-aqueous liquid media and various solid and semi-solid dosage forms.

The carriers described above may include many other different ingredients and additives in addition to the active agent. The other ingredients are included in the formulation for various reasons known to those skilled in the art, such as an activity-stabilizing agent and a binding agent. Descriptions of suitable pharmaceutical carriers and factors involved in carrier selection can be found in several readily available sources, such as Allen L. V Jr. et al. Remington: The Science and Practice of Pharmacy (2 Volumes), 22^{nd} Edition (2012), Pharmaceutical Press.

The compound is typically administered in the form of a mixture formulated with a suitable pharmaceutical diluent, excipient or carrier (herein, collectively referred to as pharmaceutical carriers), which is appropriately selected in accordance with the intended mode of administration (such as oral tablets, capsules, elixirs, and syrups) and consistent with conventional pharmacy practice.

Although the compound of the present disclosure can be administered alone, it is preferred to be administered in the form of a pharmaceutical formulation (composition).

### Kit/Product Packaging

For use in the treatment of the above indications, the kit/product packaging is also described herein. These kits can be composed of a conveyor, a drug pack or a container box, wherein the container box can be divided into multiple compartments to accommodate one or more containers, such as a vial, a test tube and the like, and each container contains a single ingredient in the method described. Suitable containers include bottles, vials, syringes and test tubes. Containers are made of acceptable materials such as glass or plastic.

For example, a container may contain one or more compounds described herein, where the compound exits either as a pharmaceutical component or in a mixture with other ingredients described herein. The container may have a sterile outlet (for example, the container may be an IV bag or bottle, and the stopper may be pierced by a hypodermic needle). Such kit may contain a compound and an instruction for the use method as described herein, a label, or an operation instruction.

A typical kit may include one or more containers. Each container contains one or more materials (such as a reagent, or a concentrated stock solution, and/or equipment) to accommodate commercial promotion and demand of users for the use of the compound. These materials include, but are not limited to, a buffer, a diluent, a filter, a needle, a syringe, a delivery device, a bag, a container, a bottle and/or a test tube, accompanied by a list of contents and/or an instruction for use, as may the built-in packaging. The entire set of instruction must be included.

The above features described in the present disclosure or the features described in the embodiments may be combined in any combination. All features disclosed in the specification of the present disclosure may be used in conjunction with any combination form. Each feature disclosed in the specification may be replaced by any alternative feature that serves the same, equivalent or similar purpose. Therefore, unless otherwise stated, the features disclosed are only general examples of equivalent or similar features.

The present disclosure will be further described below in conjunction with specific examples. It should be understood that these examples are only used to illustrate the present disclosure and are not intended to limit the scope of the present disclosure. In the following examples, experimental methods without specifying specific conditions are usually conducted under conventional conditions or conditions recommended by the manufacturer. Unless otherwise stated, all percentages, ratios, proportions, or parts are by weight.

The unit of weight-volume percentage in the present disclosure is well known to those skilled in the art, for example, referring to the weight of the solute in 100 ml of solution. Unless otherwise defined, all technical and scientific terms used herein have the same meaning as familiar to those skilled in the art. In addition, any methods and materials similar or equivalent to those described can be used in the method of the present disclosure. The preferred implementation methods and materials described herein are for demonstration purposes only.

In the terms used in the present disclosure, “neurodegenerative disease” and “neural degenerative disease” have the same meaning; “axonal degeneration” and “axon degeneration” have the same meaning. Those skilled in the art will understand that the terms have commonly understood meanings.

### EXAMPLES

The present teachings include the description provided in the examples, which is not intended to limit the scope of any claim. The following non-limiting examples are provided to further illustrate the present disclosure. In light of the present disclosure, those skilled in the art will appreciate that many changes can be made to the specific embodiments disclosed and the same or similar results can still be obtained without departing from the spirit and scope of the present teachings.

Unless otherwise stated, all materials/reagents were obtained from commercial suppliers and used without further purification. Reactions were monitored during the experiments by LC-MS and/or thin layer chromatography (TLC) on silica 60 F254 (0.2 mm) precoated glass backings and visualized using UV light. The structures of the compounds in the following examples were characterized by nuclear magnetic resonance (NMR) and/or mass spectrometry (MS).

¹HNMR (400 MHz) spectra were recorded on Bruker spectrometer at room temperature with TMS or residual solvent peaks as internal standards. Chemical shift values or peak shape multiples are given in (6), and the coupling constant (J) is given as an absolute value in Hertz (Hz). The abbreviations for multiplicity in ¹HNMR spectra are as follows: s (singlet), d (doublet), t (triplet), q (quartet), m (multiplet), br or broadt (broadened).

Preparative HPLC purification was performed on Shimadzu LC-6AD. All purification work was performed using Shim-pack PREP-DDS(H)KIT columns. The mobile phase was water (containing 0.1% HCO2H) and acetonitrile; all reagents used were HPLC grade. The flow rate was 10 ml/min.

LC-MS was performed on Agilent 1260 infinityII under the following conditions: mobile phase: A: water (0.1% trifluoroacetic acid), B: ACN; 3.5 min of running program; column: YMC-Triart C18, 50*3 mm, 3 m; flow rate: 1.8 ml/min; oven temperature: 40° C.; gradient: 5-100 (ACN %).

Preparative TLC was performed on a Whatman LK6F Silica Gel 60A plate with a size of 20×20 cm and a thickness of 500 μm.

The following examples are intended to illustrate embodiments of the present disclosure without limiting it in any way.

### Synthesis of Intermediates

### Synthesis of Intermediate BB1

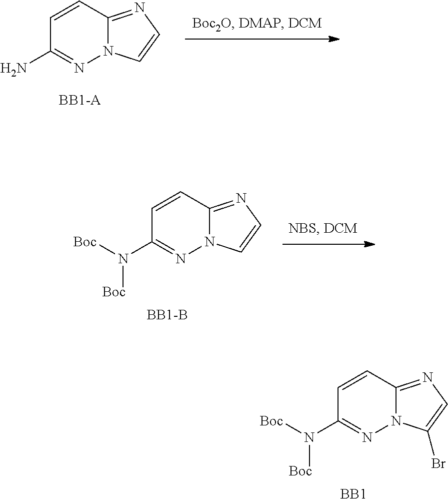

### Step 1: Compound BB1-B bis-tert-butylimidazo[1,2-b]pyridazin-6-yl aminodicarboxylate

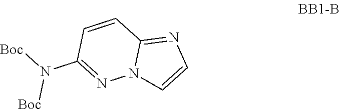 [SMILES: CC(C)(C)OC(=O)N(C(=O)OC(C)(C)C)c1ccc2nccn2n1]

A dichloromethane solution containing compound BB1-A imidazo[1,2-b]pyridazin-6-amine (50 mg, 0.373 mmol), di-tert-butyl dicarbonate (89.55 mg, 0.41 mmol), 4-dimethylaminopyridine (9.1 mg, 0.0745 mmol) and triethylamine (75.49 mg, 0.746 mmol) was stirred at room temperature for 14 hours. The reaction was monitored by TLC to be completed. The reaction mixture was extracted with water (10 ml) and dichloromethane (10 ml×2). The organic layer was dried over anhydrous sodium sulfate and concentrated under reduced pressure. The obtained residue was separated and purified by preparative TLC (eluted with dichloromethane containing 4.76% methanol) to obtain compound BB1-B bis-tert-butylimidazo[1,2-b]pyridazin-6-ylaminodicarboxylate (85 mg) as a white solid.

LC_MS: (ES⁺): m/z 335.1 [M+H]⁺

### Step 2: Compound BB1 Tert-butyl (3-bromoimidazo [1,2-b]pyridazin-6-yl)aminodicarboxylate

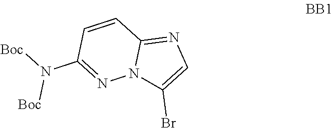 [SMILES: CC(C)(C)OC(=O)N(C(=O)OC(C)(C)C)c1ccc2ncc(Br)n2n1]

A dichloromethane solution (2.5 ml) containing the compound BB1-B bis-tert-butylimidazo[1,2-b]pyridazin-6-ylaminodicarboxylate (60 mg, 0.256 mmol) and N-bromosuccinimide (50.17 mg, 0.282 mmol) was stirred at room temperature for 5 hours. The reaction was monitored by TLC to be completed. The reaction mixture was extracted with water (10 ml) and dichloromethane (10 ml×2). The organic layer was dried over anhydrous sodium sulfate and concentrated under reduced pressure. The obtained residue was separated and purified by preparative TLC (eluted with dichloromethane containing 2.38% methanol) to obtain compound BB1 tert-butyl (3-bromoimidazo [1,2-b]pyridazin-6-yl)aminodicarboxylate (55 mg) as a white solid.

LC_MS: (ES⁺): m/z 335.1 [M+H]⁺

### Synthesis of Intermediate BB2

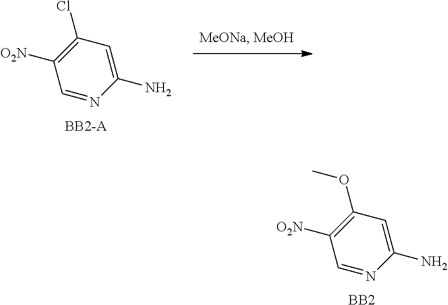

### Step 1: Compound BB2 4-methoxy-5-nitropyridin-2-amine

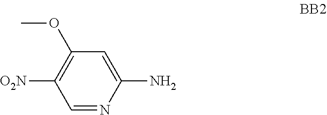

A methanol solution (20 mL) containing compound BB2-A (500 mg, 2.88 mmol) was added with sodium methoxide (1.56 g, 8.64 mmol). The mixture was stirred at 20° C. for 12 hours of reaction, and then heated to 60° C. and stirred for 1 hour of reaction. The compound BB2-A (Rf=0.7) was monitored to be completely consumed by TLC (DCM:MeOH=10:1), and a new spot (Rf=0.5) was generated. The reaction solution was concentrated. The residue was diluted with water (20 mL) and extracted with ethyl acetate (20 mL×2). The organic layers were combined, washed with saturated brine (20 mL), dried over anhydrous sodium sulfate, filtered and concentrated. The residue was subjected to silica gel column chromatography (eluted with dichloromethane containing 8% methanol, Rf=0.5) to obtain compound BB2 4-methoxy-5-nitropyridin-2-amine (420 mg) as a yellow solid.

LC_MS: (ES⁺): m/z 170.1 [M+H]⁺.

### Synthesis of Intermediate BB3

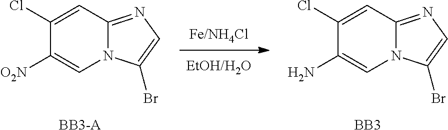

### Step 1: Compound BB3 3-bromo-7-chloroimidazole[1,2-a]pyridin-6-amine

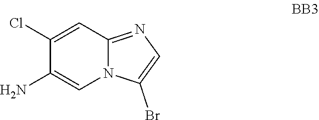 [SMILES: Nc1cn2c(Br)cnc2cc1Cl]

Ammonium chloride (19.4 mg) and iron powder (20.2 mg) were added to a mixed solution of ethanol (2 mL) and water (0.2 mL) containing compound BB3-A (20 mg, 72.34 μmol). The reaction solution was stirred at 80° C. for 3 hours of reaction. Compound BB3-A (Rf=0.7) was monitored by TLC (PE:EtOAc=1:2) to be completely consumed, and two new spots (Rf=0.5, 0.4) were generated. The reaction solution was filtered, and the mother liquor was concentrated. The residue was diluted with water (10 mL) and extracted with ethyl acetate (10 mL×2). The organic layers were combined, washed with saturated brine (20 mL), dried over anhydrous sodium sulfate, filtered and concentrated. The residue was used directly in the next step without further purification. Thus, a yellow solid crude compound BB-3 3-bromo-7-chloroimidazole[1,2-a]pyridin-6-amine (17.8 mg) was obtained.

LC_MS: (ES⁺): m/z 245.9 [M+H]⁺.

### Synthesis of Intermediate BB4

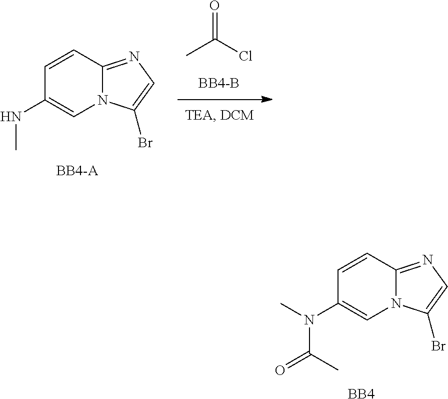

### Step 1: Intermediate BB4 N-(3-bromoimidazo[1,2-a]pyridin-6-yl)-N-methylacetamide

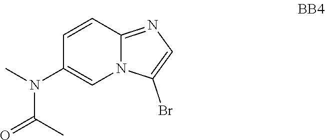

A dichloromethane (4 ml) solution containing compound BB4-A 3-bromo-N-methylimidazo[1,2-a]pyridin-6-amine (80 mg, 0.354 mmol) and triethylamine (35.75 mg, 0.354 mmol) was added with compound BB4-B acetyl chloride (33.3 mg, 0.425 mmol) under nitrogen at 0° C. The reaction solution was stirred at 0° C. for 5 hours of reaction. The reaction was monitored by TLC to be completed. The reaction solution was added with water and extracted with dichloromethane. The combined organic layers were dried over anhydrous sodium sulfate and concentrated under reduced pressure. The residue was subjected to preparative TLC (eluted with dichloromethane containing 2.4% methanol) to obtain brown solid compound BB4 N-(3-bromoimidazo[1,2-a]pyridin-6-yl)-N-methylacetamide (43 mg).

LC_MS: (ES⁺): m/z 269.9 [M+H]⁺.

### Synthesis of Intermediates BB5 and BB6

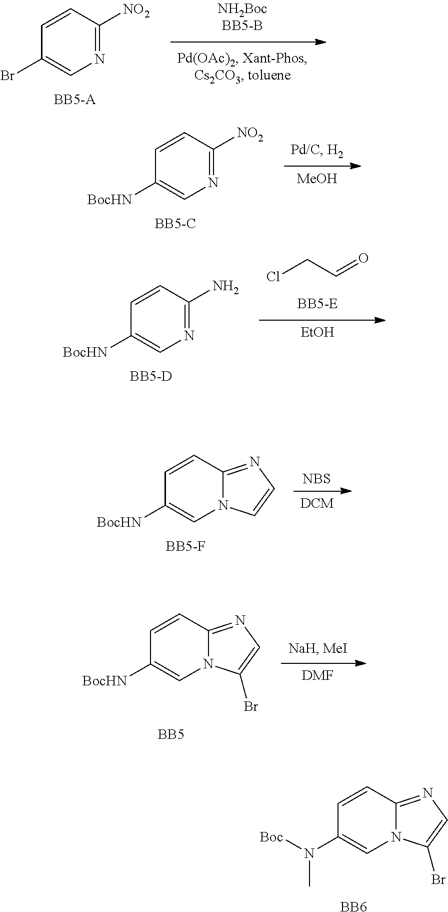

### Step 1: Compound BB5-C tert-butyl (6-nitropyridin-3-yl)carbamate

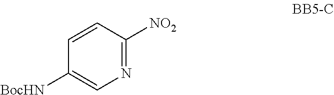 [SMILES: CC(C)(C)OC(=O)Nc1ccc([N+](=O)[O-])nc1]

A toluene solution containing compound BB5-A 5-bromo-2-nitropyridine (500 mg, 2.46 mmol) and compound BB5-B (577.1 mg, 4.93 mmol) was added with palladium acetate (55.3 mg, 246.31 μmol), Xant-Phos (142.5 mg, 246.31 μmol) and cesium carbonate (2.41 g, 7.39 mmol). The reaction solution was stirred under a nitrogen atmosphere at 100° C. for 12 hours of reaction. The reaction was monitored by TLC to be completed (PE:EtOAc=2:1). The reaction solution was diluted with water (20 mL), and extracted with ethyl acetate (20 mL×2). The organic layers were combined, washed with saturated brine (20 mL), dried over anhydrous sodium sulfate, filtered, and concentrated. The residue was separated and purified by silica gel column chromatography (eluted with a petroleum ether solution containing 33% ethyl acetate) to obtain a yellow oil compound BB5-C tert-butyl (6-nitropyridin-3-yl)carbamate (580 mg).

LC_MS: (ES⁺): m/z 240.1 [M+H]⁺.

### Step 2: Compound BB5-D tert-butyl (6-aminopyridin-3-yl)carbamate

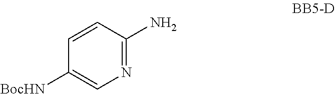 [SMILES: CC(C)(C)OC(=O)Nc1ccc(N)nc1]

A methanol (10 mL) solution containing compound BB5-C (580 mg, 2.42 mmol) was added with Pd/C. The reaction solution was stirred under a hydrogen atmosphere at 20° C. for 12 hours of reaction. Compound 3 (Rf=0.9) was monitored by TLC (DCM:MeOH=10:1) to be completely consumed, and a new spot (Rf=0.3) was generated. The reaction solution was filtered through diatomite, and the mother liquor was concentrated. The residue was used directly in the next step without further purification. Thus, a yellow solid compound BB5-D tert-butyl (6-aminopyridin-3-yl)carbamate (540 mg) was obtained.

LC_MS: (ES⁺): m/z 210.1 [M+H]⁺.

### Step 3: Compound BB5-F tert-butylimidazo[1,2-a]pyridin-6-yl carbamate

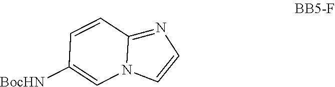 [SMILES: CC(C)(C)OC(=O)Nc1ccc2nccn2c1]

Compound BB5-E (1.01 g, 5.16 mmol, 40% mass fraction in water) was added to the ethanol solution containing compound BB5-D (540 mg, 2.58 mmol). The reaction solution was stirred and reacted at 80° C. for 3 hours. TLC (DCM:MeOH=10:1) monitoring showed that the reaction was complete. The reaction solution was concentrated, then added with saturated sodium bicarbonate solution (20 mL), and extracted with dichloromethane (20 mL×2). The organic layers were combined, washed with saturated brine (20 mL), dried over anhydrous sodium sulfate, filtered and concentrated. The residue was subjected to silica gel column chromatography (eluted with a dichloromethane solution containing 10% methanol) to obtain a yellow solid compound BB5-F tert-butylimidazo[1,2-a]pyridin-6-yl carbamate (400 mg).

LC_MS: (ES⁺): m/z 234.1 [M+H]⁺.

### Step 4: Compound BB5 tert-butyl (3-bromoimidazo[1,2-a]pyridin-6-yl)carbamate

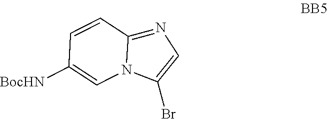 [SMILES: CC(C)(C)OC(=O)Nc1ccc2ncc(Br)n2c1]

A dichloromethane (5 ml) solution containing compound BB5-F (300 mg, 1.29 mmol) was added with N-bromosuccinimide (229 mg, 1.29 mmol). The reaction solution was stirred at room temperature for 2 hours of reaction. The reaction was monitored by TLC to be completed. The reaction mixture was concentrated under reduced pressure, and the resulting residue was subjected to silica gel column chromatography (eluted with petroleum ether containing 0%-50% ethyl acetate) to obtain yellow solid compound BB5 tert-butyl (3-bromoimidazo[1,2-a]pyridin-6-yl)carbamate (370 mg).

LC_MS: (ES⁺): m/z 311.9 [M+H]⁺.

### Step 5: Compound BB6 tert-butyl (3-bromoimidazo[1,2-a]pyridin-6-yl)(methyl)carbamate

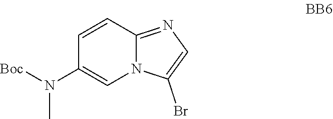 [SMILES: CN(C(=O)OC(C)(C)C)c1ccc2ncc(Br)n2c1]

A DMF (10 ml) solution containing compound BB5 (800 mg, 2.563 mmol) was added with sodium hydride (102.51 mg, 2.563 mmol, 60%) and methyl iodide (727.58 mg, 5.126 mmol) under a nitrogen atmosphere at 0° C. The reaction solution was stirred at 0° C. for 1 hour of reaction. The reaction was monitored by TLC to be completed. The reaction solution was poured into saturated ammonium chloride solution (30 mL), and extracted with ethyl acetate (25 mL×2).

The organic phases were combined, washed with saturated brine (50 mL), dried over anhydrous sodium sulfate, and concentrated under reduced pressure. The obtained crude product was separated and purified by silica gel column chromatography (eluent: a dichloromethane solution containing 0.024%-0.062% methanol) to obtain white solid compound BB6 tert-butyl (3-bromoimidazo[1,2-a]pyridin-6-yl)(methyl)carbamate (840 mg).

LC_MS: (ES⁺): m/z 327.9 [M+H]⁺.

### Synthesis of Intermediate BB7

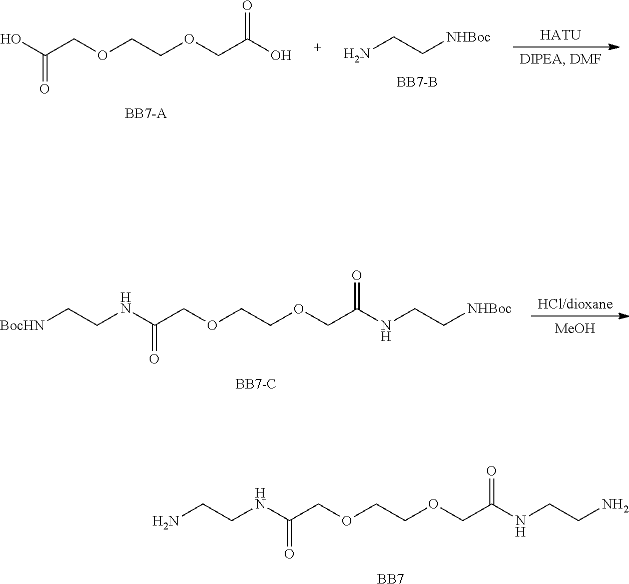

### Step 1: Compound BB7-C di-tert-butyl (4,11-dioxo-6,9-dioxin-3,12-diazatetradecane-1,14-diyl)dicarbamate

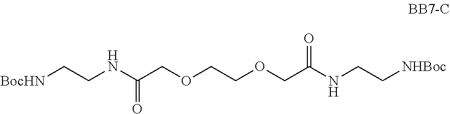 [SMILES: CC(C)(C)OC(=O)NCCNC(=O)COCCOCC(=O)NCCNC(=O)OC(C)(C)C]

A DMF solution containing compound BB7-A 2,2′-(ethane-1,2-diylbis(oxy))diacetic acid (300 mg, 1.7 mmol), compound BB7-B tert-butyl (2-aminoethyl)carbamate (546 mg, 3.4 mmol) and N,N-diisopropylethylamine (1.1 g, 8.5 mmol) was added with HATU (1.94 g, 5.1 mmol) at 0° C. The reaction solution was warmed to room temperature and stirred for 2 hours of reaction. The reaction was monitored by TLC to be completed. The reaction solution was partitioned between ethyl acetate (30 ml) and water (50 ml). The organic layer was collected, washed with saturated brine (30 ml), dried over anhydrous sodium sulfate, and concentrated under reduced pressure. The crude product was separated and purified by silica gel column chromatography (eluted with dichloromethane containing 1.5% methanol) to obtain white solid compound BB7-C di-tert-butyl (4,11-dioxo-6,9-dioxin-3,12-diazatetradecane-1,14-diyl)dicarbamate (480 mg, 61%).

¹H NMR (400 MHz, CDCl₃): δ 1.43 (s, 18H), 3.26-3.32 (m, 4H), 3.41-3.45 (m, 4H), 3.70 (s, 4H), 4.02 (s, 4H)¹H NMR (400 MHz, CDCl₃): δ 1.43 (s, 18H), 3.26-3.32 (m, 4H), 3.41-3.45 (m, 4H), 3.70 (s, 4H), 4.02 (s, 4H).

### Step 2: Compound BB7 2,2′-(ethane-1,2-diylbis(oxy))bis(N-(2-aminoethyl)acetamide

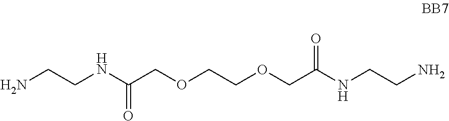 [SMILES: NCCNC(=O)COCCOCC(=O)NCCN]

A methanol (2 ml) solution containing compound BB7-C di-tert-butyl (4,11-dioxo-6,9-dioxin-3,12-diazatetradecane-1,14-diyl)dicarbamate (100 mg) was added with 4M hydrogen chloride-dioxane solution (5 ml). The reaction solution was stirred at room temperature for 3 hours of reaction. The reaction was monitored by TLC to be completed. The reaction solution was concentrated under reduced pressure to obtain crude compound BB7 2,2′-(ethane-1,2-diylbis(oxy))bis(N-(2-aminoethyl)acetamide (80 mg) as a black oil, which was used directly in the next step without further purification.

¹H NMR (400 MHz, DMSO-d6): δ 2.86-2.91 (m, 4H), 3.36-3.41 (m, 4H), 3.66 (s, 4H), 3.94 (s, 4H), 8.08-8.11 (m, 6H).

### Example 1: Synthesis of Compound 1

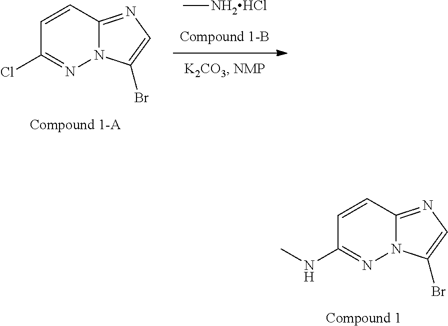

### Step 1: Compound 1 3-bromo-N-methylimidazo[1,2-b]pyridazin-6-amine

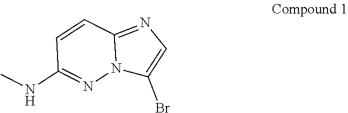

A N-methylpyrrolidone (1 ml) solution containing compound 1-A 3-bromo-6-chloroimidazo[1,2-b]pyridazine (40 mg, 0.172 mmol), compound 1-B methylamine hydrochloride (34.84 mg, 0.516 mmol) and potassium carbonate (83.07 mg, 0.602 mmol) was stirred at 100° C. for 18 hours of reaction. The reaction was monitored by TLC to be completed. The reaction solution was extracted with water and ethyl acetate. The combined organic layers were washed with saturated brine, dried over anhydrous sodium sulfate, and concentrated under reduced pressure. The resulting residue was separated and purified by preparative TLC (eluted with dichloromethane containing 4.76% methanol) to obtain compound 1 3-bromo-N-methylimidazo[1,2-b]pyridazin-6-amine (30 mg) as a white solid.

LC_MS: (ES⁺): m/z 227.0 [M+H]⁺.

### Example 2: Synthesis of Compound 2

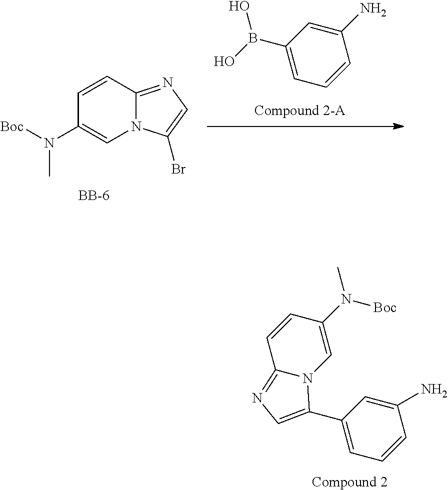

### Step 1: Compound 2 tert-butyl 3-(3-aminophenyl)imidazo[1,2-a]pyridin-6-yl)(methyl)carbamate

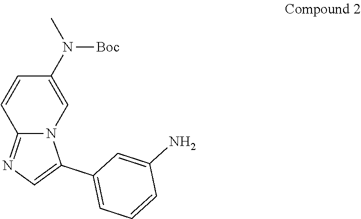 [SMILES: CN(C(=O)OC(C)(C)C)c1ccc2ncc(-c3cccc(N)c3)n2c1]

A 1,4-dioxane solution containing compound BB-6 (530 mg, 1.625 mmol), compound 2-A (244.75 mg, 1.787 mmol) and saturated aqueous sodium carbonate solution (3 ml) was added with Pd(dppf)Cl₂—CH₂Cl₂ (132.3 mg, 0.162 mmol) at room temperature under nitrogen atmosphere. The reaction solution was stirred at 90° C. for 12 hours of reaction. The reaction was monitored by TLC to be completed. The reaction solution was cooled to room temperature, and extracted with water (20 mL) and ethyl acetate (30 ml×2). The organic layers were combined, dried over anhydrous sodium sulfate, and concentrated under reduced pressure. The obtained crude product was subjected to silica gel column chromatography (eluted with a petroleum ether solution containing 0%-90% ethyl acetate) to obtain compound 2 tert-butyl 3-(3-aminophenyl)imidazo[1,2-a]pyridin-6-yl)(methyl)carbamate as a yellow solid (432 mg).

LC_MS: (ES⁺): m/z 339.2 [M+H]⁺.

¹H NMR (400 MHz, DMSO) δ 8.42 (d, J=1.2 Hz, 1H), 7.66 (s, 2H), 7.30 (d, J=9.2 Hz, 1H), 7.17 (t, J=7.8 Hz, 1H), 6.76 (dd, J=18.1, 4.7 Hz, 2H), 6.62 (dd, J=8.0, 1.4 Hz, 1H), 5.30 (d, J=4.9 Hz, 2H), 3.18 (s, 3H), 1.35 (s, 9H).

The synthesis of the following compounds referred to the synthesis of compound 2 and intermediate BB5 or BB6:

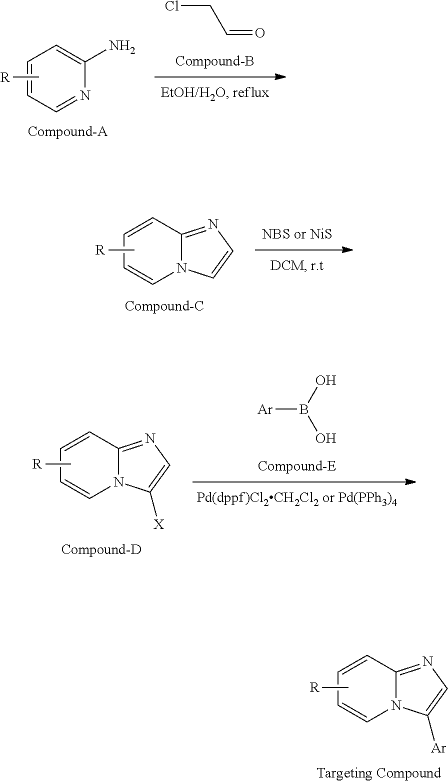

### Synthesis of Compound 3

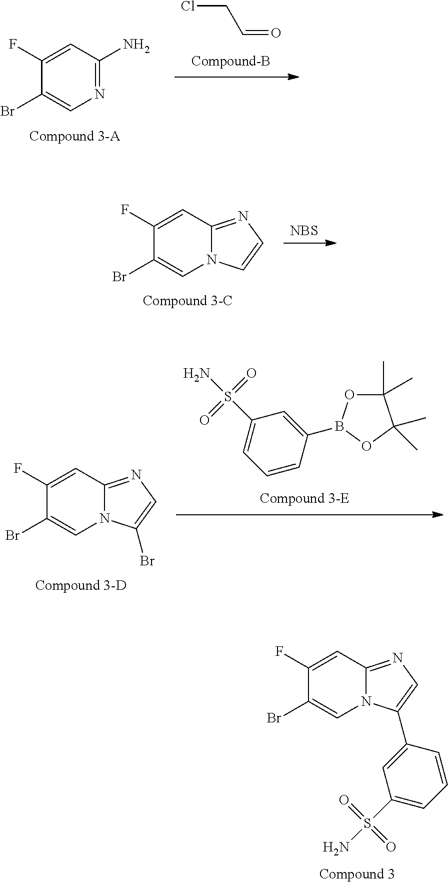

Step 1: The synthesis of compound 3-C was performed referring to the third step of the synthesis method of intermediate BB5.

Step 2: The synthesis of compound 3-D was performed referring to the fourth step of the synthesis method of intermediate BB5.

Step 3: The synthesis of compound 3 was performed referring to the first step of the synthesis method of compound 2.

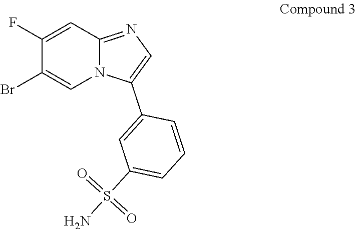 [SMILES: NS(=O)(=O)c1cccc(-c2cnc3cc(F)c(Br)cn23)c1]

LC_MS: (ES⁺): m/z 369.8 [M+H]⁺.

### Synthesis of Compound 4

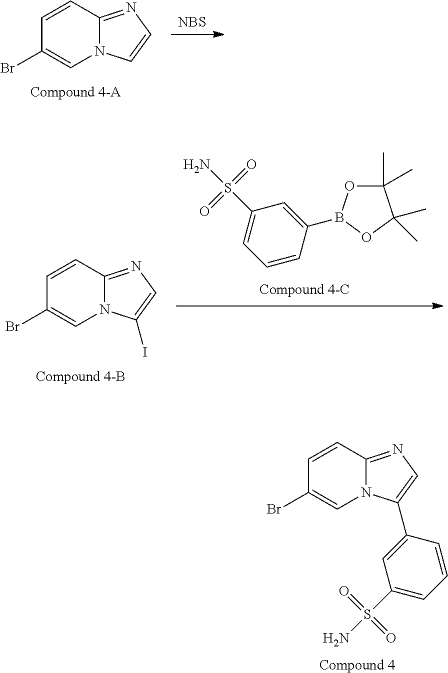

Step 1: The synthesis of compound 4-B using compound 4-A and NIS as raw materials, was performed referring to the fourth step of the synthesis method of intermediate BB5.

Step 2: The synthesis of compound 4 was performed referring to the first step of the synthesis method of compound 2.

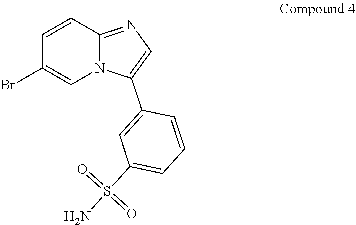 [SMILES: NS(=O)(=O)c1cccc(-c2cnc3ccc(Br)cn23)c1]

LC_MS: (ES⁺): m/z 369.8 [M+H]⁺.

### Synthesis of Compound 5

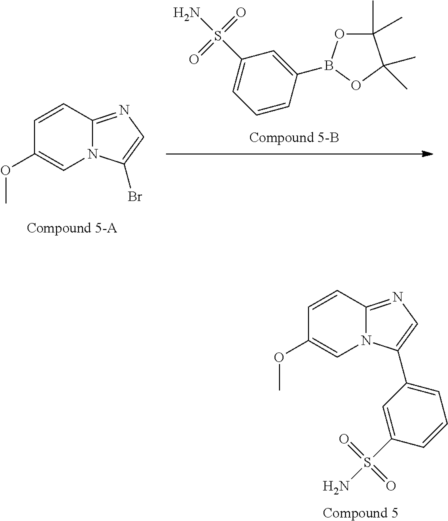

Step 1: The synthesis of compound 5 was performed referring to the first step of the synthesis method of compound 2.

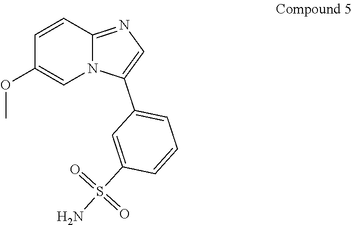 [SMILES: COc1ccc2ncc(-c3cccc(S(N)(=O)=O)c3)n2c1]

LC_MS: (ES⁺): m/z 303.9 [M+H]⁺.

¹H NMR (400 MHz, DMSO) δ 8.09 (s, 1H), 8.00 (d, J=1.5 Hz, 1H), 7.95 (d, J=7.7 Hz, 1H), 7.85 (d, J=7.9 Hz, 1H), 7.80-7.73 (m, 2H), 7.73-7.67 (m, 1H), 7.47 (s, 2H), 7.21 (d, J=9.6 Hz, 1H), 3.79 (s, 3H).

### Synthesis of Compound 6

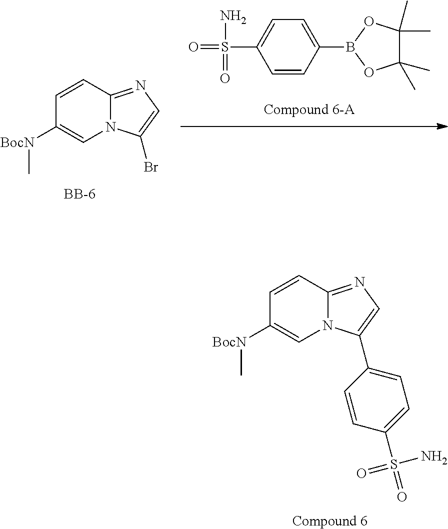

Step 1: The synthesis of compound 6 was performed referring to the first step of the synthesis method of compound 2.

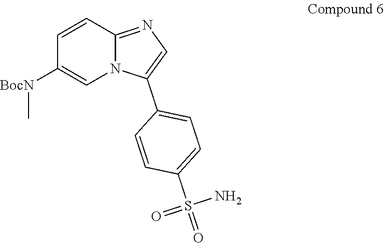 [SMILES: CN(C(=O)OC(C)(C)C)c1ccc2ncc(-c3ccc(S(N)(=O)=O)cc3)n2c1]

LC_MS: (ES⁺): m/z 403.0 [M+H]⁺.

### Synthesis of Compound 7

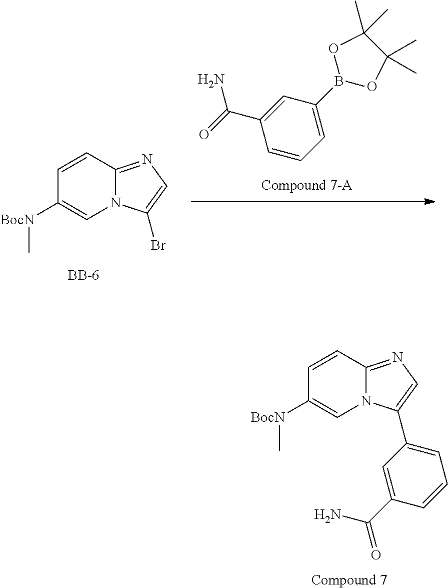

Step 1: The synthesis of compound 7 was performed referring to the first step of the synthesis method of compound 2.

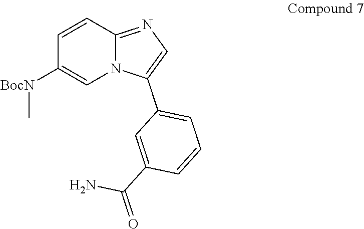 [SMILES: CN(C(=O)OC(C)(C)C)c1ccc2ncc(-c3cccc(C(N)=O)c3)n2c1]

LC_MS: (ES⁺): m/z 367.0 [M+H]⁺.

### Synthesis of Compound 8

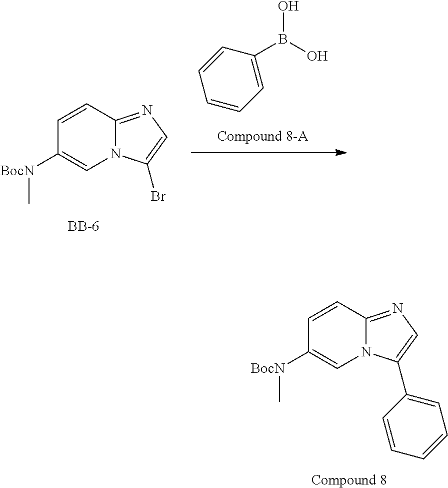

Step 1: The synthesis of compound 8 was performed referring to the first step of the synthesis method of compound 2.

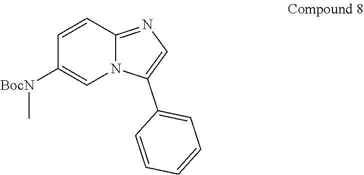 [SMILES: CN(C(=O)OC(C)(C)C)c1ccc2ncc(-c3ccccc3)n2c1]

LC_MS: (ES⁺): m/z 324.0 [M+H]⁺.

### Synthesis of Compound 9

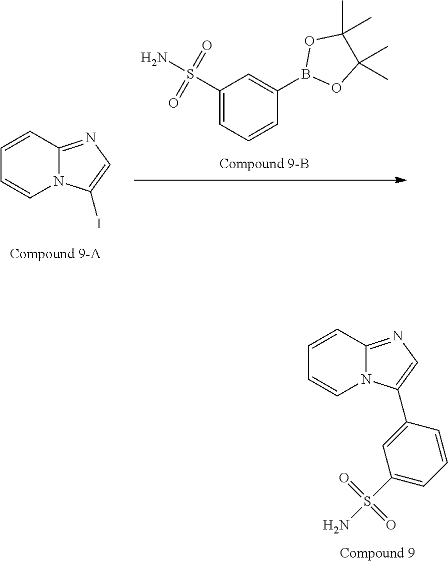

Step 1: The synthesis of compound 9 was performed referring to the first step of the synthesis method of compound 2.

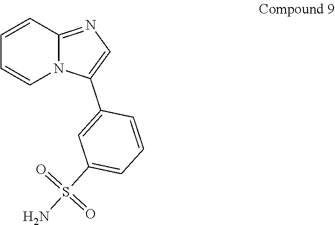 [SMILES: NS(=O)(=O)c1cccc(-c2cnc3ccccn23)c1]

LC_MS: (ES⁺): m/z 273.9 [M+H]⁺.

¹H NMR (400 MHz, DMSO) δ 8.60-8.56 (m, 1H), 8.06 (t, J=1.6 Hz, 1H), 7.93-7.89 (m, 1H), 7.87-7.83 (m, 2H), 7.72 (dd, J=16.4, 8.5 Hz, 2H), 7.44 (s, 2H), 7.35 (ddd, J=9.0, 6.7, 1.0 Hz, 1H), 7.03 (td, J=6.8, 1.1 Hz, 1H).

### Synthesis of Compound 10

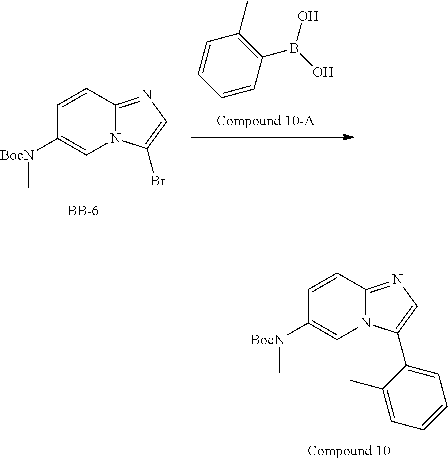

Step 1: The synthesis of compound 10 was performed referring to the first step of the synthesis method of compound 2.

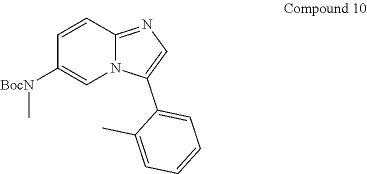 [SMILES: Cc1ccccc1-c1cnc2ccc(N(C)C(=O)OC(C)(C)C)cn12]

LC_MS: (ES⁺): m/z 338.0 [M+H]⁺.

### Synthesis of Compound 11

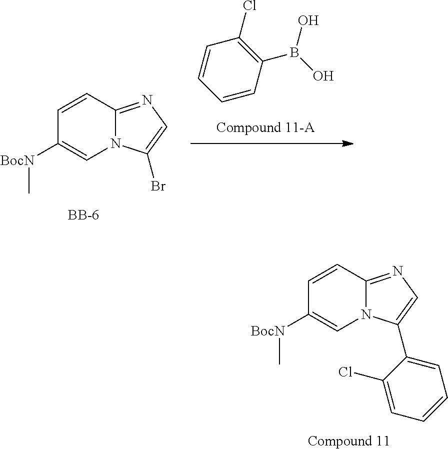

Step 1: The synthesis of compound 11 was performed referring to the first step of the synthesis method of compound 2.

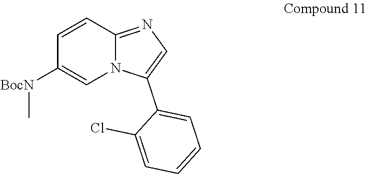 [SMILES: CN(C(=O)OC(C)(C)C)c1ccc2ncc(-c3ccccc3Cl)n2c1]

LC_MS: (ES⁺): m/z 358.0 [M+H]⁺.

### Synthesis of Compound 12

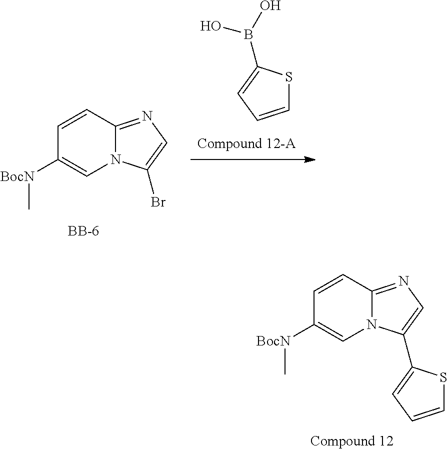

Step 1: The synthesis of compound 12 was performed referring to the first step of the synthesis method of compound 2.

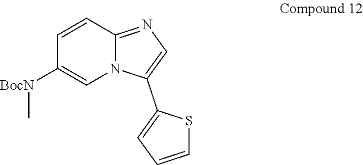 [SMILES: CN(C(=O)OC(C)(C)C)c1ccc2ncc(-c3cccs3)n2c1]

LC_MS: (ES⁺): m/z 330.0 [M+H]⁺.

### Synthesis of Compound 13

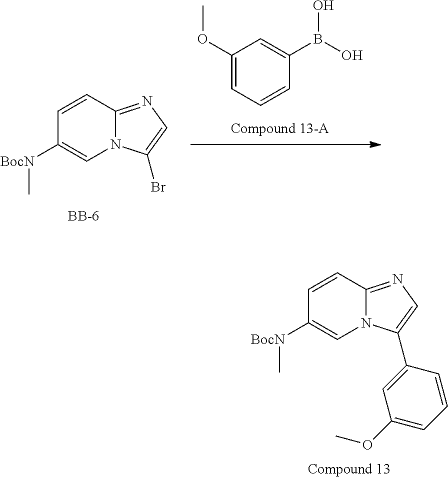

Step 1: The synthesis of compound 13 was performed referring to the first step of the synthesis method of compound 2.

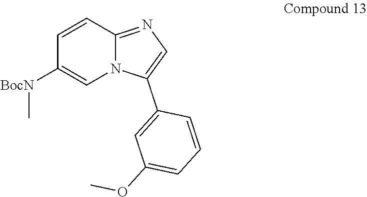 [SMILES: COc1cccc(-c2cnc3ccc(N(C)C(=O)OC(C)(C)C)cn23)c1]

LC_MS: (ES⁺): m/z 354.0 [M+H]⁺.

### Synthesis of Compound 14

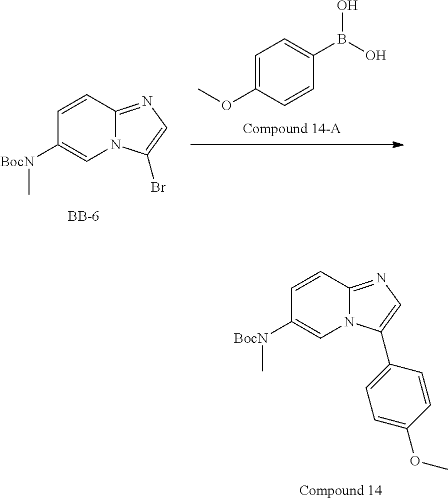

Step 1: The synthesis of compound 14 was performed referring to the first step of the synthesis method of compound 2.

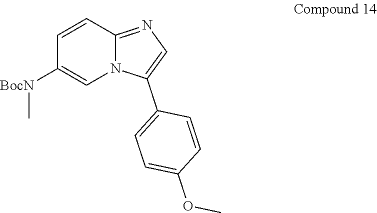 [SMILES: COc1ccc(-c2cnc3ccc(N(C)C(=O)OC(C)(C)C)cn23)cc1]

LC_MS: (ES⁺): m/z 354.0 [M+H]⁺.

### Synthesis of Compound 15

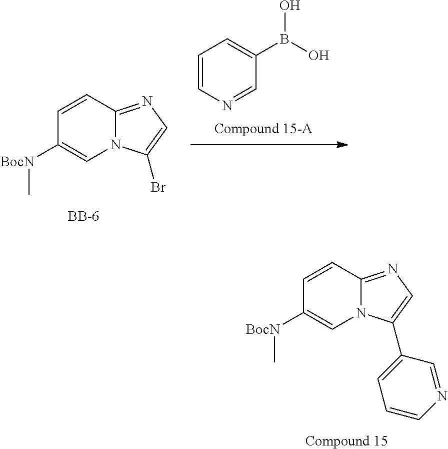

Step 1: The synthesis of compound 15 was performed referring to the first step of the synthesis method of compound 2.

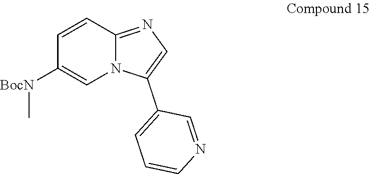 [SMILES: CN(C(=O)OC(C)(C)C)c1ccc2ncc(-c3cccnc3)n2c1]

LC_MS: (ES⁺): m/z 325.0 [M+H]⁺.

### Synthesis of Compound 16

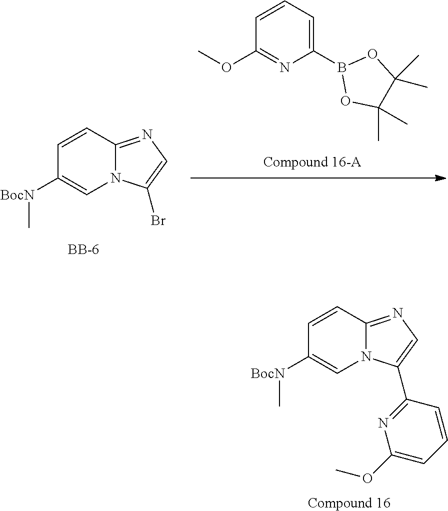

Step 1: The synthesis of compound 16 was performed referring to the first step of the synthesis method of compound 2.

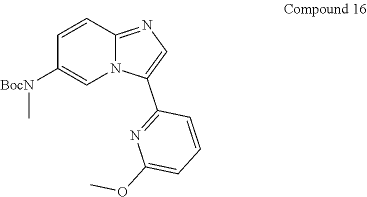 [SMILES: COc1cccc(-c2cnc3ccc(N(C)C(=O)OC(C)(C)C)cn23)n1]

LC_MS: (ES⁺): m/z 355.0 [M+H]⁺.

### Synthesis of Compound 17

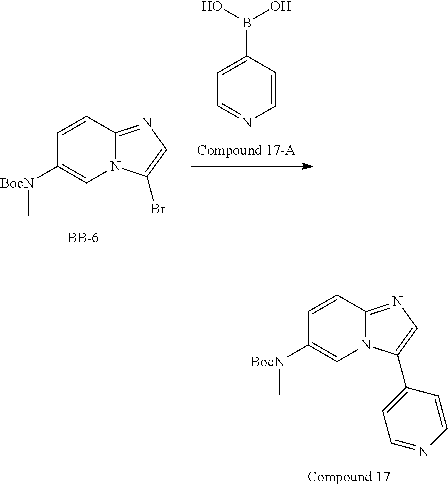

Step 1: The synthesis of compound 17 was performed referring to the first step of the synthesis method of compound 2.

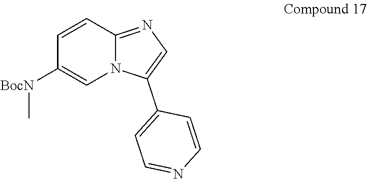 [SMILES: CN(C(=O)OC(C)(C)C)c1ccc2ncc(-c3ccncc3)n2c1]

LC_MS: (ES⁺): m/z 325.0 [M+H]⁺.

### Synthesis of Compound 18

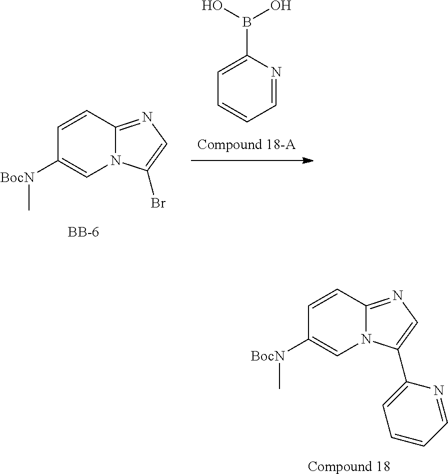

Step 1: The synthesis of compound 18 was performed referring to the first step of the synthesis method of compound 2.

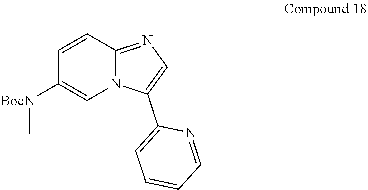 [SMILES: CN(C(=O)OC(C)(C)C)c1ccc2ncc(-c3ccccn3)n2c1]

LC_MS: (ES⁺): m/z 325.0 [M+H]⁺.

### Synthesis of Compound 19

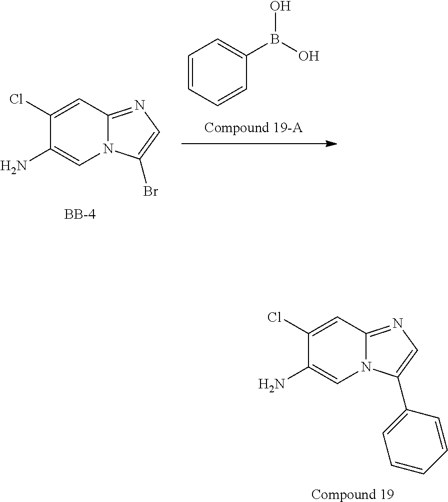 [SMILES: Nc1cn2c(Br)cnc2cc1Cl ; OB(O)c1ccccc1 ; "BB-4" ; Nc1cn2c(-c3ccccc3)cnc2cc1Cl ; "Compound 19"]

Step 1: The synthesis of compound 19 was performed referring to the first step of the synthesis method of compound 2.

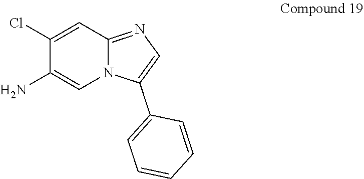 [SMILES: Nc1cn2c(-c3ccccc3)cnc2cc1Cl]

LC_MS: (ES⁺): m/z 244.0 [M+H]⁺.

### Synthesis of Compound 20

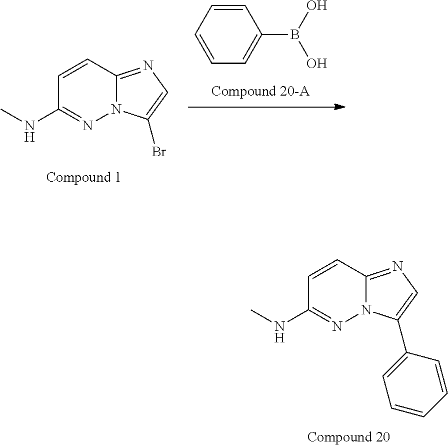 [SMILES: CNc1ccc2ncc(Br)n2n1 ; OB(O)c1ccccc1 ; "Compound 1" ; CNc1ccc2ncc(-c3ccccc3)n2n1 ; "Compound 20"]

Step 1: The synthesis of compound 20 was performed referring to the first step of the synthesis method of compound 2.

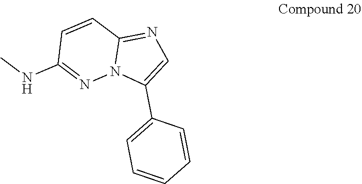 [SMILES: CNc1ccc2ncc(-c3ccccc3)n2n1]

LC_MS: (ES⁺): m/z 225.0 [M+H]⁺.

¹H NMR (500 MHz, DMSO) δ 8.20 (dd, J=8.4, 1.1 Hz, 2H), 7.87 (s, 1H), 7.77 (d, J=9.7 Hz, 1H), 7.47 (dd, J=10.7, 4.9 Hz, 2H), 7.31 (dd, J=10.5, 4.3 Hz, 1H), 7.11 (d, J=4.7 Hz, 1H), 6.71 (d, J=9.7 Hz, 1H), 2.85 (d, J=4.8 Hz, 3H).

### Synthesis of Compound 21

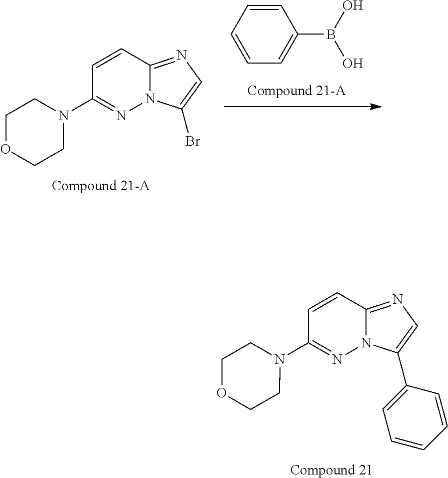 [SMILES: Brc1cnc2ccc(N3CCOCC3)nn12 ; OB(O)c1ccccc1 ; "Compound 21-A" ; c1ccc(-c2cnc3ccc(N4CCOCC4)nn23)cc1 ; "Compound 21"]

Step 1: The synthesis of compound 21 was performed referring to the first step of the synthesis method of compound 2.

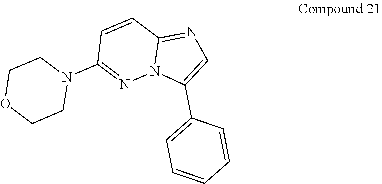 [SMILES: c1ccc(-c2cnc3ccc(N4CCOCC4)nn23)cc1]

LC_MS: (ES⁺): m/z 281.1 [M+H]⁺.

¹H NMR (400 MHz, DMSO) δ 8.12 (d, J=7.4 Hz, 2H), 7.99-7.93 (m, 2H), 7.47 (t, J=7.8 Hz, 2H), 7.32 (t, J=7.4 Hz, 1H), 7.23 (d, J=9.9 Hz, 1H), 3.77-3.72 (m, 4H), 3.50-3.45 (m, 4H).

### Synthesis of Compound 22

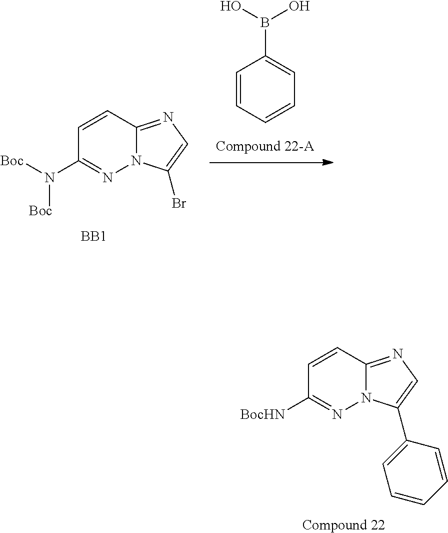 [SMILES: CC(C)(C)OC(=O)N(C(=O)OC(C)(C)C)c1ccc2ncc(Br)n2n1 ; OB(O)c1ccccc1 ; "BB1" ; CC(C)(C)OC(=O)Nc1ccc2ncc(-c3ccccc3)n2n1 ; "Compound 22"]

Step 1: The synthesis of compound 22 was performed referring to the first step of the synthesis method of compound 2.

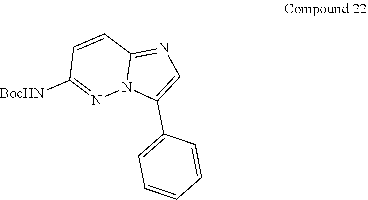 [SMILES: CC(C)(C)OC(=O)Nc1ccc2ncc(-c3ccccc3)n2n1]

LC_MS: (ES⁺): m/z 311.1 [M+H]⁺.

¹H NMR (500 MHz, DMSO) δ 10.37 (s, 1H), 8.29 (dd, J=8.4, 1.1 Hz, 2H), 8.19 (s, 1H), 8.11 (d, J=9.8 Hz, 1H), 7.78-7.75 (m, 1H), 7.69 (d, J=9.8 Hz, 1H), 7.47 (dd, J=10.7, 4.9 Hz, 2H), 1.50 (s, 9H).

### Synthesis of Compound 23

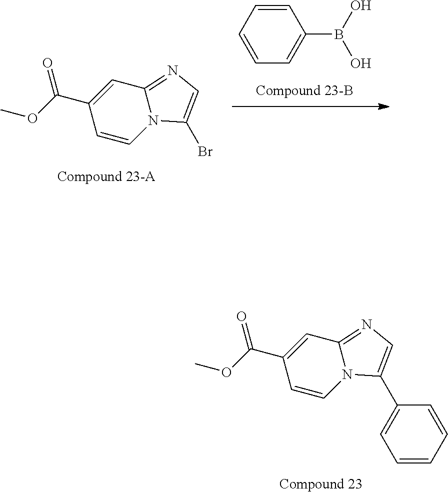

Step 1: The synthesis of compound 23 was performed referring to the first step of the synthesis method of compound 2.

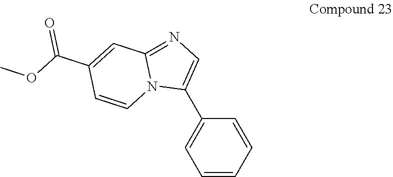 [SMILES: COC(=O)c1ccn2c(-c3ccccc3)cnc2c1]

LC_MS: (ES⁺): m/z 253.0 [M+H]⁺.

### Synthesis of Compound 24

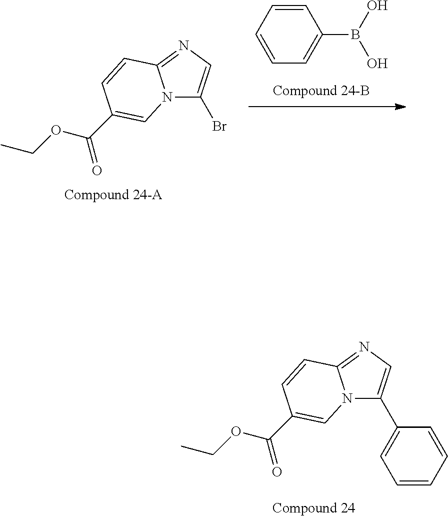

Step 1: The synthesis of compound 24 was performed referring to the first step of the synthesis method of compound 2.

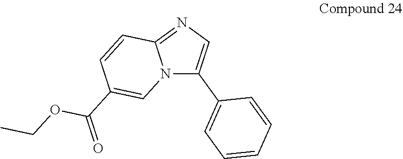 [SMILES: CCOC(=O)c1ccc2ncc(-c3ccccc3)n2c1]

LC_MS: (ES⁺): m/z 267.1 [M+H]⁺.

### Synthesis of Compound 25

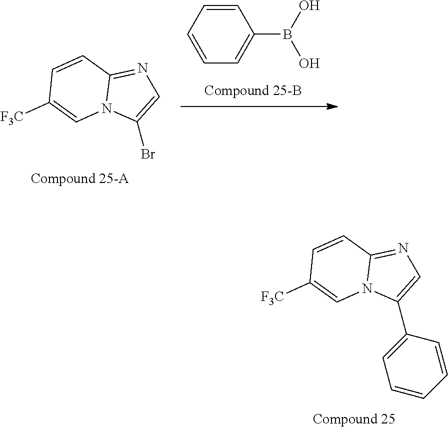 [SMILES: FC(F)(F)c1ccc2ncc(Br)n2c1 ; OB(O)c1ccccc1 ; "Compound 25-A" ; FC(F)(F)c1ccc2ncc(-c3ccccc3)n2c1 ; "Compound 25"]

Step 1: The synthesis of compound 25 was performed referring to the first step of the synthesis method of compound 2.

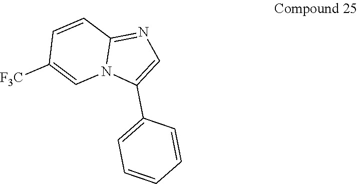 [SMILES: FC(F)(F)c1ccc2ncc(-c3ccccc3)n2c1]

LC_MS: (ES⁺): m/z 263.0 [M+H]⁺.

¹H NMR (500 MHz, DMSO) δ 8.78 (s, 1H), 8.03 (s, 1H), 7.88 (d, J=9.5 Hz, 1H), 7.77 (dd, J=8.0, 1.4 Hz, 1H), 7.72 (dd, J=8.2, 1.1 Hz, 2H), 7.59 (d, J=7.5 Hz, 2H), 7.55-7.53 (m, 1H).

### Synthesis of Compound 26

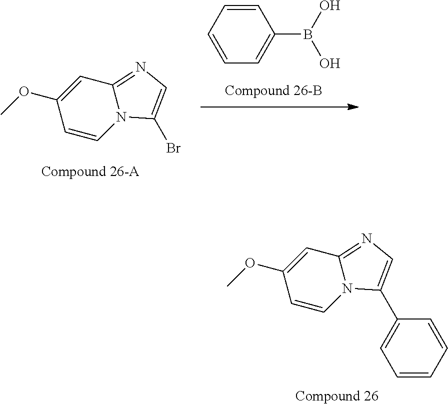

Step 1: The synthesis of compound 26 was performed referring to the first step of the synthesis method of compound 2.

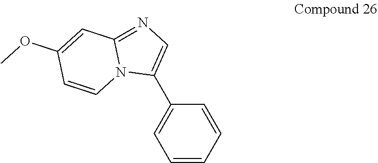 [SMILES: COc1ccn2c(-c3ccccc3)cnc2c1]

LC_MS: (ES⁺): m/z 225.1 [M+H]⁺.

¹H NMR (500 MHz, DMSO) δ 8.42 (d, J=7.6 Hz, 1H), 7.63-7.60 (m, 3H), 7.55-7.50 (m, 2H), 7.43-7.39 (m, 1H), 7.10 (s, 1H), 6.71 (dd, J=7.5, 2.4 Hz, 1H), 3.85 (s, 3H).

### Synthesis of Compound 27

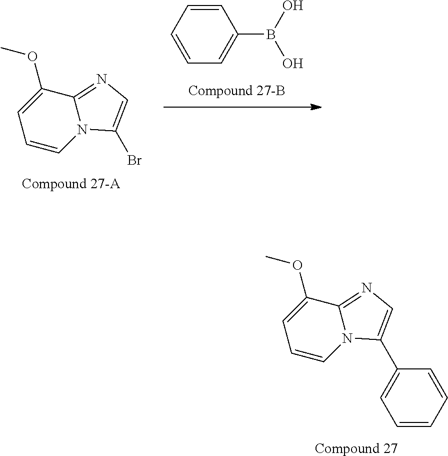

Step 1: The synthesis of compound 27 was performed referring to the first step of the synthesis method of compound 2.

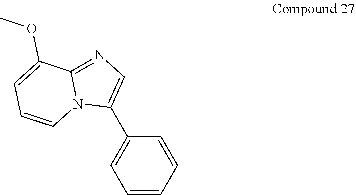 [SMILES: COc1cccn2c(-c3ccccc3)cnc12]

LC_MS: (ES⁺): m/z 225.0 [M+H]⁺.

¹H NMR (500 MHz, DMSO) δ 8.42 (d, J=7.6 Hz, 1H), 7.63-7.60 (m, 3H), 7.55-7.50 (m, 2H), 7.43-7.39 (m, 1H), 7.10 (s, 1H), 6.71 (dd, J=7.5, 2.4 Hz, 1H), 3.85 (s, 3H).

### Synthesis of Compound 28

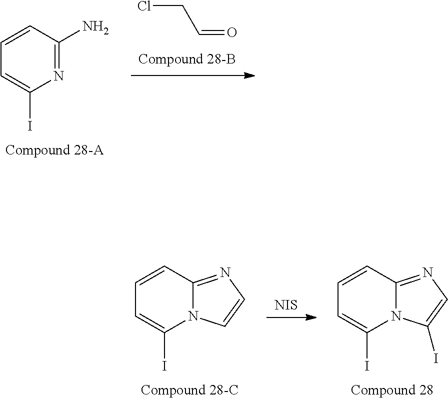

Step 1: The synthesis of compound 28-C was performed referring to the third step of the synthesis method of intermediate BB5.

Step 2: The synthesis of compound 28 was performed referring to the fourth step of the synthesis method of intermediate BB5.

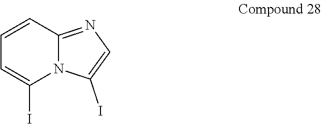 [SMILES: Ic1cccc2ncc(I)n12]

LC_MS: (ES⁺): m/z 370.9 [M+H]⁺.

¹H NMR (500 MHz, DMSO) δ 7.72 (s, 1H), 7.66 (dd, J=8.9, 1.2 Hz, 1H), 7.50 (dd, J=7.1, 1.1 Hz, 1H), 6.92 (dd, J=8.9, 7.1 Hz, 1H).

### Synthesis of Compound 29

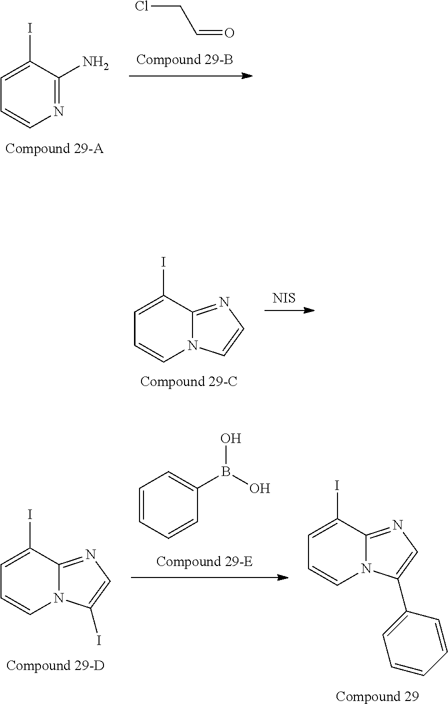

Step 1: The synthesis of compound 29-C was performed referring to the third step of the synthesis method of intermediate BB5.

Step 2: The synthesis of compound 29-D was performed referring to the fourth step of the synthesis method of intermediate BB5.

Step 3: The synthesis of compound 29 was performed referring to the first step of the synthesis method of compound 2.

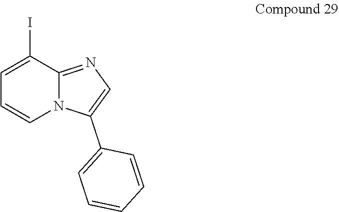 [SMILES: Ic1cccn2c(-c3ccccc3)cnc12]

LC_MS: (ES⁺): m/z 321.0 [M+H]⁺.

### Synthesis of Compound 30

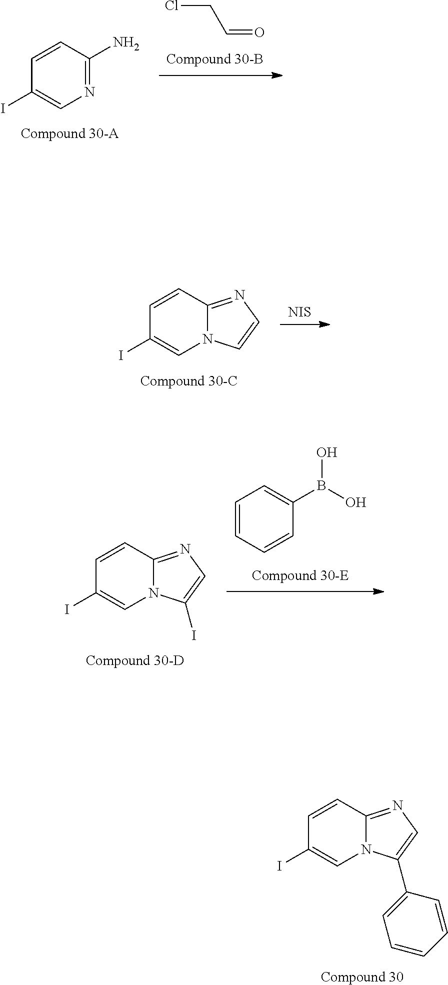

Step 1: The synthesis of compound 30-C was performed referring to the third step of the synthesis method of intermediate BB5.

Step 2: The synthesis of compound 30-D was performed referring to the fourth step of the synthesis method of intermediate BB5.

Step 3: The synthesis of compound 30 was performed referring to the first step of the synthesis method of compound 2.

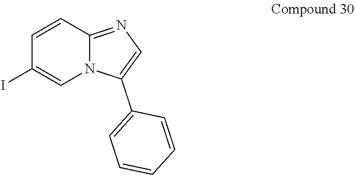 [SMILES: Ic1ccc2ncc(-c3ccccc3)n2c1]

LC_MS: (ES⁺): m/z 321.0 [M+H]⁺.

### Synthesis of Compound 31

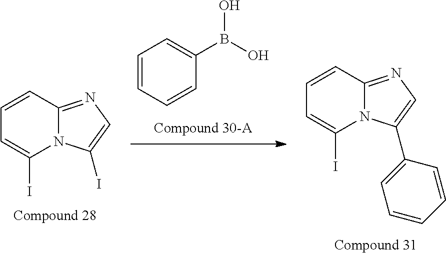

Step 1: The synthesis of compound 31 was performed referring to the first step of the synthesis method of compound 2.

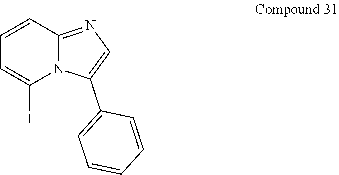 [SMILES: Ic1cccc2ncc(-c3ccccc3)n12]

LC_MS: (ES⁺): m/z 320.9 [M+H]⁺.

### Synthesis of Compound 32

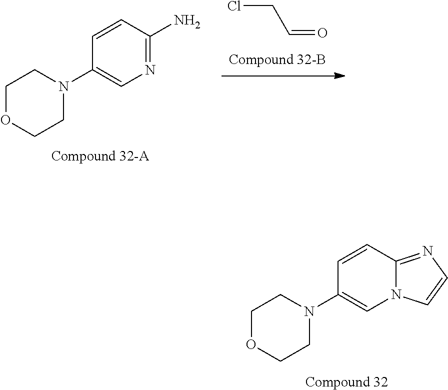

Step 1: The synthesis of compound 32 was performed referring to the third step of the synthesis method of intermediate BB5.

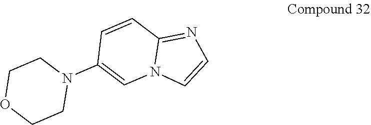 [SMILES: c1cn2cc(N3CCOCC3)ccc2n1]

LC_MS: (ES⁺): m/z 204.1 [M+H]⁺.

### Synthesis of Compound 33

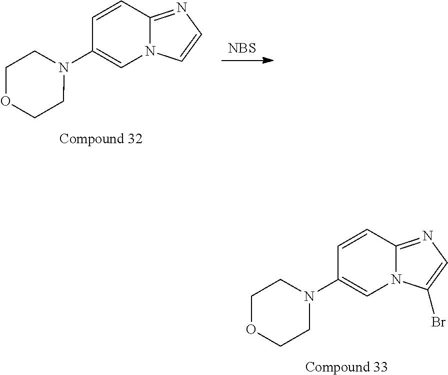

Step 1: The synthesis of compound 32 was performed referring to the fourth step of the synthesis method of intermediate BB5.

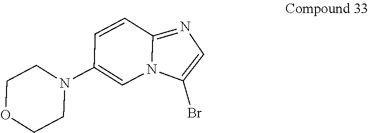 [SMILES: Brc1cnc2ccc(N3CCOCC3)cn12]

LC_MS: (ES⁺): m/z 283.0 [M+H]⁺.

¹H NMR (500 MHz, DMSO) δ 7.62 (s, 1H), 7.58 (d, J=9.8 Hz, 1H), 7.49 (d, J=1.9 Hz, 1H), 7.41 (dd, J=9.8, 2.2 Hz, 1H), 3.77-3.74 (m, 4H), 3.12-3.07 (m, 4H).

### Synthesis of Compound 34

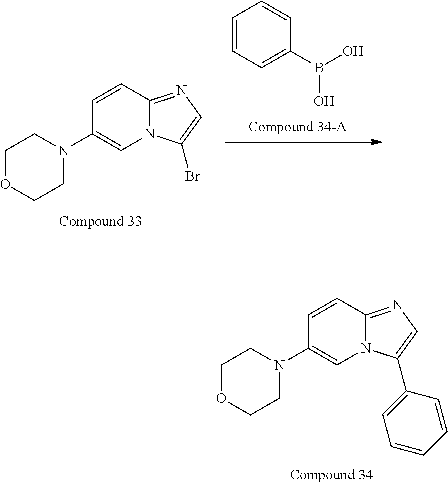

Step 1: The synthesis of compound 34 was performed referring to the first step of the synthesis method of compound 2.

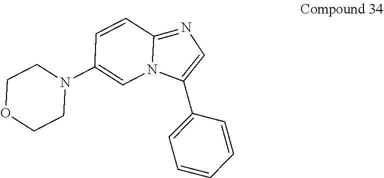 [SMILES: c1ccc(-c2cnc3ccc(N4CCOCC4)cn23)cc1]

LC_MS: (ES⁺): m/z 280.1 [M+H]⁺.

¹H NMR (500 MHz, DMSO) δ 8.02 (s, 1H), 7.76 (d, J=1.3 Hz, 1H), 7.69 (d, J=7.1 Hz, 2H), 7.67 (s, 1H), 7.55 (t, J=7.7 Hz, 2H), 7.45 (t, J=7.4 Hz, 2H), 3.80-3.69 (m, 4H), 3.10-2.99 (m, 4H).

### Synthesis of Compound 35

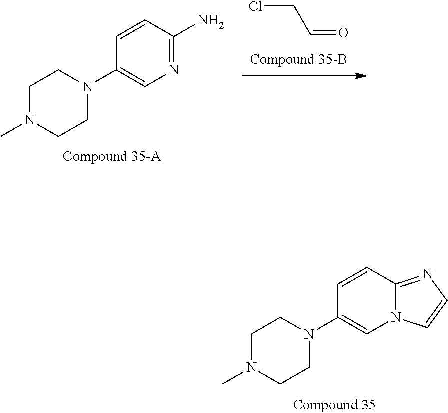

Step 1: The synthesis of compound 35 was performed referring to the third step of the synthesis method of intermediate BB5.

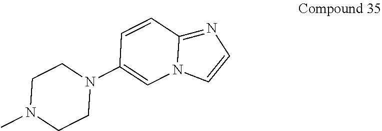 [SMILES: CN1CCN(c2ccc3nccn3c2)CC1]

LC_MS: (ES⁺): m/z 217.1 [M+H]⁺.

### Synthesis of Compound 36

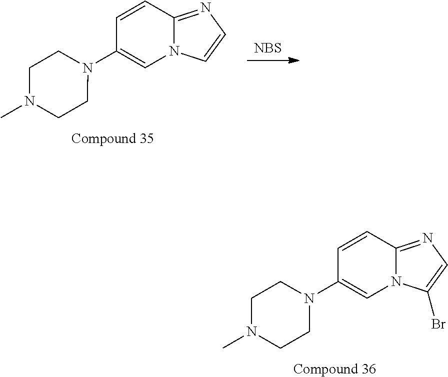

Step 1: The synthesis of compound 36 was performed referring to the fourth step of the synthesis method of intermediate BB5.

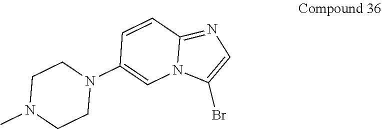 [SMILES: CN1CCN(c2ccc3ncc(Br)n3c2)CC1]

LC_MS: (ES⁺): m/z 295.0 [M+H]⁺.

¹H NMR (500 MHz, DMSO) δ 7.59 (s, 1H), 7.51 (d, J=9.8 Hz, 1H), 7.45 (d, J=1.8 Hz, 1H), 7.35 (dd, J=9.8, 2.2 Hz, 1H), 3.13-3.06 (m, 4H), 2.48 (d, J=5.9 Hz, 4H), 2.23 (s, 3H).

### Synthesis of Compound 37

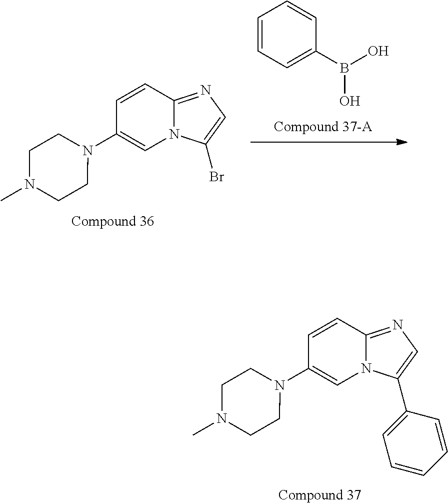

Step 1: The synthesis of compound 37 was performed referring to the first step of the synthesis method of compound 2.

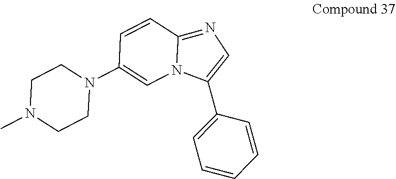 [SMILES: CN1CCN(c2ccc3ncc(-c4ccccc4)n3c2)CC1]

LC_MS: (ES⁺): m/z 293.1 [M+H]⁺.

¹H NMR (500 MHz, DMSO) δ 7.75 (d, J=1.7 Hz, 1H), 7.67 (d, J=7.2 Hz, 2H), 7.64 (s, 1H), 7.56 (dd, J=17.8, 10.2 Hz, 3H), 7.43 (t, J=7.4 Hz, 1H), 7.36 (d, J=8.5 Hz, 1H), 3.08 (s, 4H), 2.58 (s, 4H), 2.29 (s, 3H).

### Synthesis of Compound 38

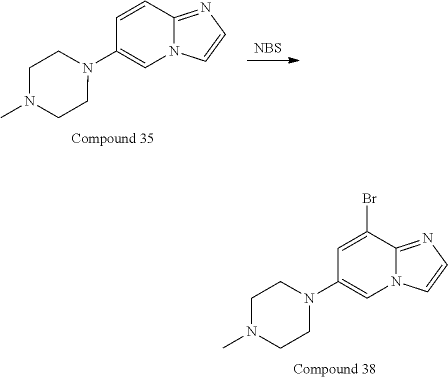

Step 1: The synthesis of compound 38 was performed referring to the fourth step of the synthesis method of intermediate BB5.

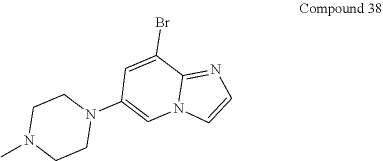 [SMILES: CN1CCN(c2cc(Br)c3nccn3c2)CC1]

LC_MS: (ES⁺): m/z 297.0 [M+H]⁺.

¹H NMR (500 MHz, DMSO) δ 7.95 (s, 1H), 7.67 (d, J=1.2 Hz, 1H), 7.62 (d, J=9.5 Hz, 1H), 7.40 (d, J=9.5 Hz, 1H), 2.94 (t, J=4.7 Hz, 4H), 2.51 (d, J=8.9 Hz, 4H), 2.25 (s, 3H).

### Synthesis of Compound 39

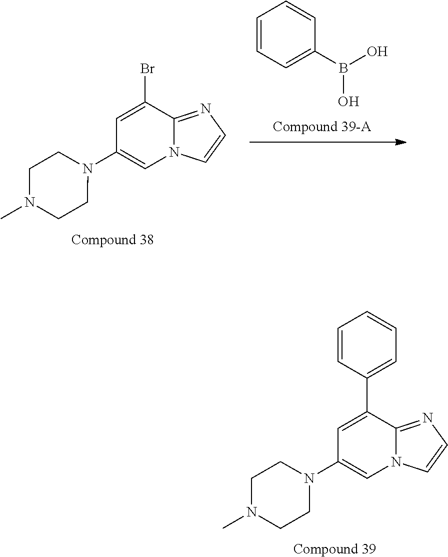

Step 1: The synthesis of compound 39 was performed referring to the first step of the synthesis method of compound 2.

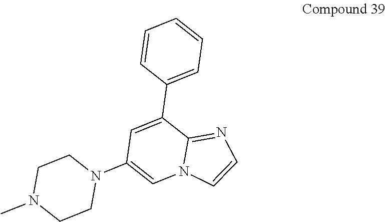 [SMILES: CN1CCN(c2cc(-c3ccccc3)c3nccn3c2)CC1]

LC_MS: (ES⁺): m/z 293.1 [M+H]⁺.

### Synthesis of Compound 40

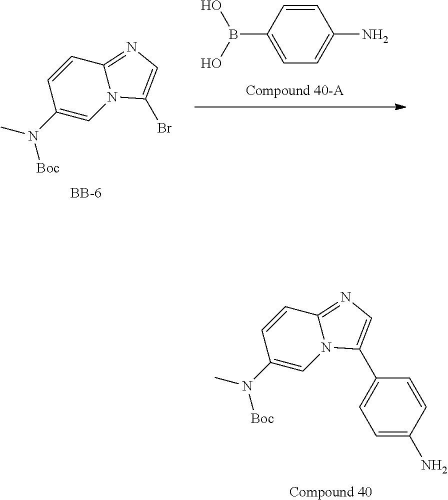

Step 1: The synthesis of compound 40 was performed referring to the first step of the synthesis method of compound 2.

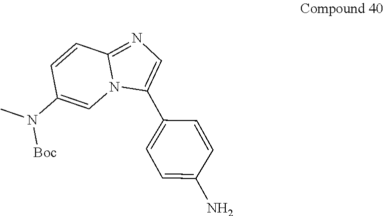 [SMILES: CN(C(=O)OC(C)(C)C)c1ccc2ncc(-c3ccc(N)cc3)n2c1]

LC_MS: (ES⁺): m/z 339.1 [M+H]⁺.

### Synthesis of Compound 41

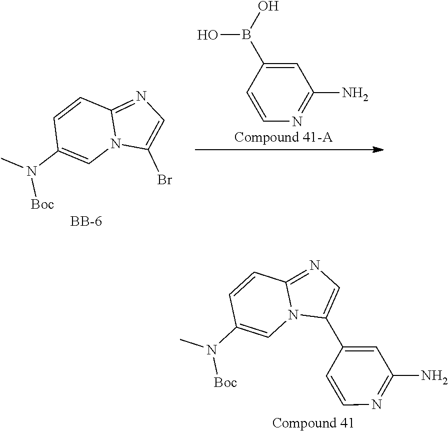

Step 1: The synthesis of compound 41 was performed referring to the first step of the synthesis method of compound 2.

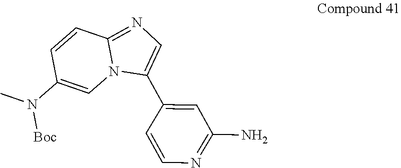 [SMILES: CN(C(=O)OC(C)(C)C)c1ccc2ncc(-c3ccnc(N)c3)n2c1]

LC_MS: (ES⁺): m/z 340.1 [M+H]⁺.

### Synthesis of Compound 42

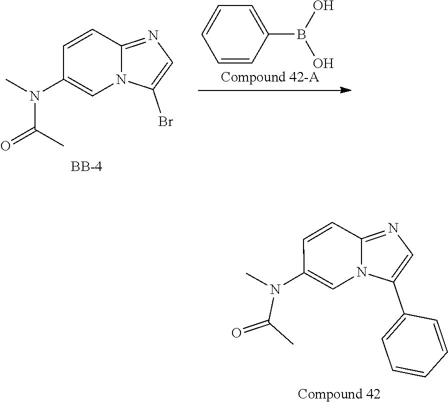

Step 1: The synthesis of compound 42 was performed referring to the first step of the synthesis method of compound 2.

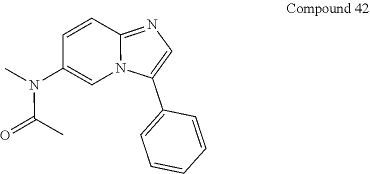 [SMILES: CC(=O)N(C)c1ccc2ncc(-c3ccccc3)n2c1]

LC_MS: (ES⁺): m/z 266.0 [M+H]⁺.

¹H NMR (500 MHz, CDCl₃) δ 8.27 (s, 1H), 7.83 (s, 1H), 7.60-7.47 (m, 6H), 7.18 (d, J=8.0 Hz, 1H), 3.27 (s, 3H), 1.95 (s, 3H).

### Synthesis of Compound 43

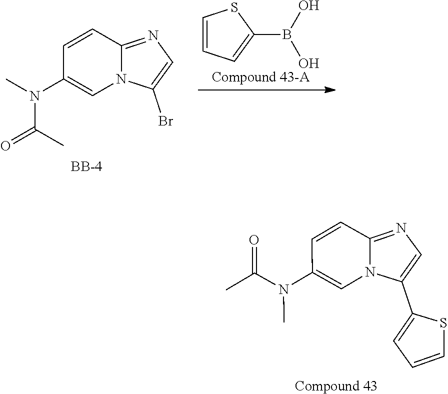

Step 1: The synthesis of compound 43 was performed referring to the first step of the synthesis method of compound 2.

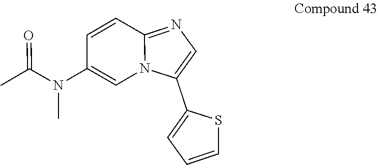 [SMILES: CC(=O)N(C)c1ccc2ncc(-c3cccs3)n2c1]

LC_MS: (ES⁺): m/z 271.9 [M+H]⁺.

¹H NMR (500 MHz, DMSO) δ 8.68 (s, 1H), 7.86 (s, 1H), 7.74 (d, J=9.2 Hz, 1H), 7.70 (d, J=5.1 Hz, 1H), 7.60 (s, 1H), 7.36 (d, J=9.3 Hz, 1H), 7.27 (dd, J=5.1, 3.6 Hz, 1H), 3.16 (s, 3H), 1.83 (s, 3H).

### Synthesis of Compound 44

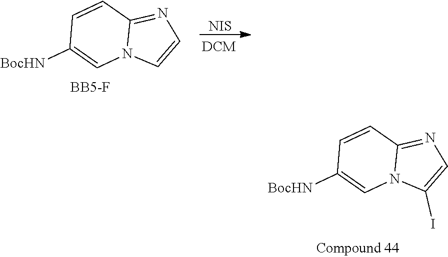

Step 1: The synthesis of compound 44 using BB5-F and NIS as raw materials, was performed referring to the first step of the synthesis method of compound 2.

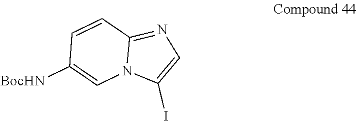 [SMILES: CC(C)(C)OC(=O)Nc1ccc2ncc(I)n2c1]

LC_MS: (ES⁺): m/z 360.0 [M+H]⁺.

¹H NMR (500 MHz, DMSO) δ 9.63 (s, 1H), 8.80 (s, 1H), 7.63 (s, 1H), 7.58-7.45 (m, 1H), 7.23 (dd, J=9.6, 1.9 Hz, 1H), 1.50 (s, 9H).

### Synthesis of Compound 45

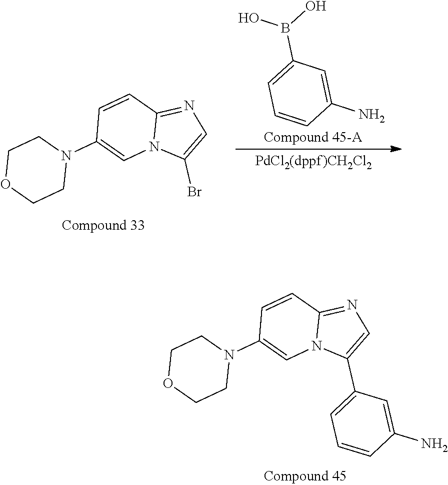

Step 1: The synthesis of compound 45 was performed referring to the first step of the synthesis method of compound 2.

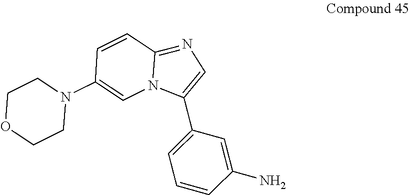 [SMILES: Nc1cccc(-c2cnc3ccc(N4CCOCC4)cn23)c1]

LC_MS: (ES⁺): m/z 295.2 [M+H]⁺.

### Synthesis of Compound 46

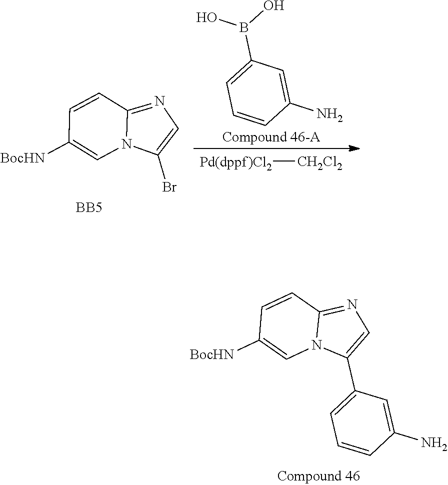

Step 1: The synthesis of compound 46 was performed referring to the first step of the synthesis method of compound 2.

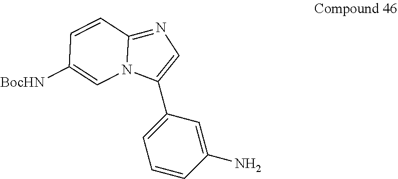 [SMILES: CC(C)(C)OC(=O)Nc1ccc2ncc(-c3cccc(N)c3)n2c1]

LC_MS: (ES⁺): m/z 339.1 [M+H]⁺.

### Example 3: Synthesis of Compound 47

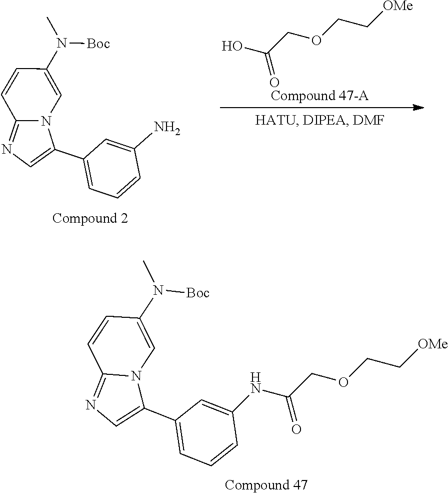

### Step 1: Compound 47 tert-butyl (3-(3-(2-(2-methoxyethoxy)acetamido)phenyl)imidazo[1,2-a]pyridin-6-yl)(methyl) carbamate

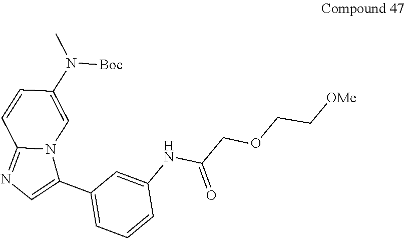 [SMILES: COCCOCC(=O)Nc1cccc(-c2cnc3ccc(N(C)C(=O)OC(C)(C)C)cn23)c1]

A solution of compound 47 (40 mg, 0.118 mmol), compound 47-A (15.85 mg, 0.118 mmol), HATU (44.84 mg, 0.118 mmol) and DIPEA (30.44 mg, 0.236 mmol) in DMF (2 mL) was stirred at room temperature for 12 hours of reaction. The reaction was monitored by TLC to be completed. The reaction mixture was extracted with water (20 ml) and ethyl acetate (30 ml×3). The organic layers were combined, dried over anhydrous sodium sulfate, and concentrated under reduced pressure. The crude product obtained was separated and purified by prep-TLC (eluted with dichloromethane containing 4.76% methanol) to obtain yellow solid compound 47 tert-butyl (3-(3-(2-(2-methoxyethoxy)acetamido)phenyl)imidazo[1,2-a]pyridin-6-yl)(methyl) carbamate (30 mg).

LC_MS: (ES⁺): m/z 455.2 [M+H]⁺.

¹H NMR (400 MHz, CDCl₃) δ 9.08 (s, 1H), 8.33 (s, 1H), 7.85 (d, J=33.6 Hz, 3H), 7.60 (d, J=8.0 Hz, 1H), 7.49 (t, J=7.7 Hz, 1H), 7.29 (s, 1H), 4.12 (s, 2H), 3.78 (s, 2H), 3.63 (s, 2H), 3.47 (s, 3H), 3.27 (s, 3H), 1.42 (s, 9H).

The following compounds were synthesized using the corresponding carboxylic acid, referring to the synthesis of compound 47:

### Synthesis of Compound 48

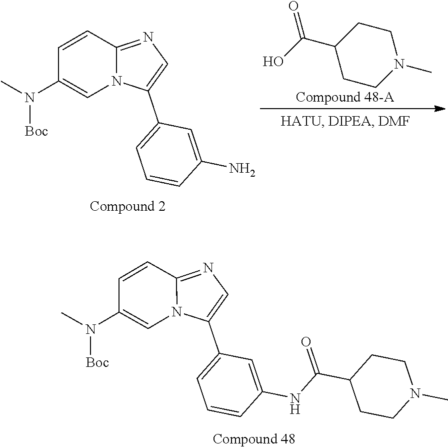

Step 1: The synthesis of compound 48 was performed referring to the first step of the synthesis method of compound 47.

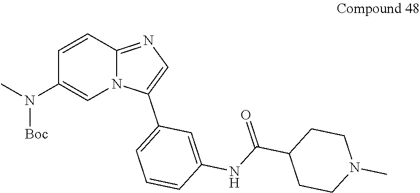

LC_MS: (ES⁺): m/z 464.2 [M+H]⁺.

¹H NMR (500 MHz, DMSO) δ 10.22 (s, 1H), 8.47 (d, J=1.3 Hz, 1H), 7.92 (s, 1H), 7.75 (s, 1H), 7.62 (t, J=9.3 Hz, 2H), 7.48 (t, J=7.9 Hz, 1H), 7.35-7.28 (m, 2H), 3.43 (s, 4H), 3.20 (s, 3H), 2.64-2.57 (m, 3H), 1.98 (s, 1H), 1.95 (d, J=11.4 Hz, 2H), 1.83 (s, 2H), 1.36 (s, 9H).

### Synthesis of Compound 49

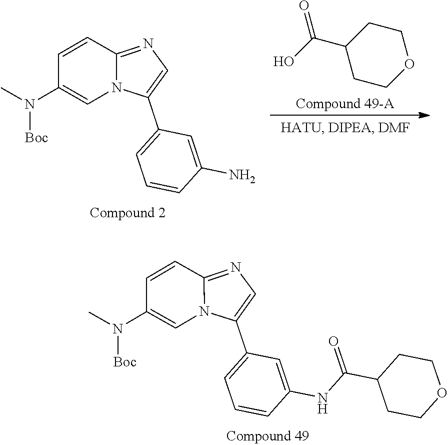

Step 1: The synthesis of compound 49 was performed referring to the first step of the synthesis method of compound 47.

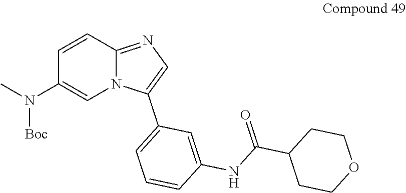 [SMILES: CN(C(=O)OC(C)(C)C)c1ccc2ncc(-c3cccc(NC(=O)C4CCOCC4)c3)n2c1]

LC_MS: (ES⁺): m/z 451.2 [M+H]⁺.

¹H NMR (500 MHz, DMSO) δ 10.07 (s, 1H), 8.47 (d, J=1.2 Hz, 1H), 7.91 (s, 1H), 7.75 (s, 1H), 7.63 (dd, J=11.8, 9.0 Hz, 2H), 7.47 (s, 1H), 7.31 (d, J=6.6 Hz, 2H), 3.92-3.88 (m, 2H), 3.20 (s, 3H), 3.16 (d, J=4.9 Hz, 2H), 2.63-2.58 (m, 1H), 1.73-1.64 (m, 4H), 1.36 (s, 9H).

### Synthesis of Compound 50

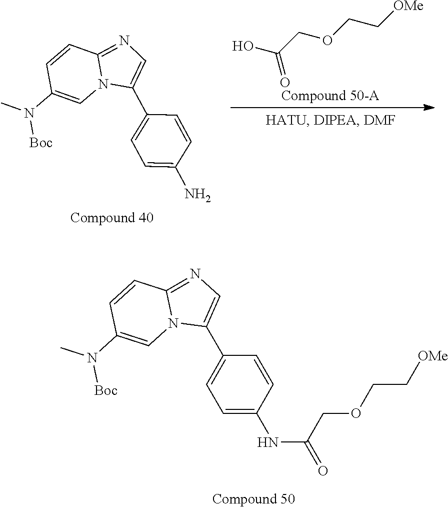

Step 1: The synthesis of compound 50 was performed referring to the first step of the synthesis method of compound 47.

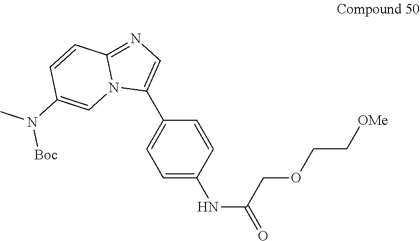 [SMILES: COCCOCC(=O)Nc1ccc(-c2cnc3ccc(N(C)C(=O)OC(C)(C)C)cn23)cc1]

LC_MS: (ES⁺): m/z 455.2 [M+H]⁺.

### Synthesis of Compound 51

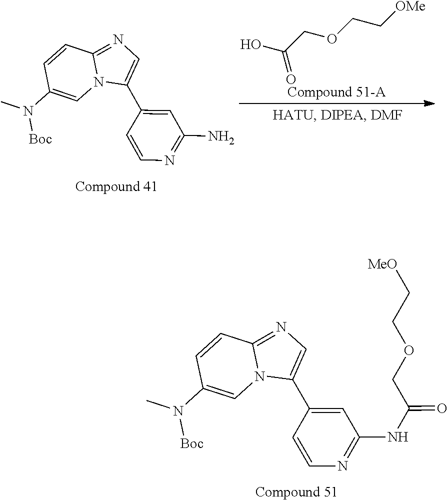

Step 1: The synthesis of compound 51 was performed using compound 41 and compound 51-A as raw materials, referring to the first step of the synthesis method of compound 47

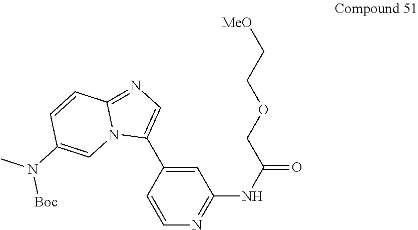 [SMILES: COCCOCC(=O)Nc1cc(-c2cnc3ccc(N(C)C(=O)OC(C)(C)C)cn23)ccn1]

LC_MS: (ES⁺): m/z 456.2 [M+H]⁺.

### Synthesis of Compound 52

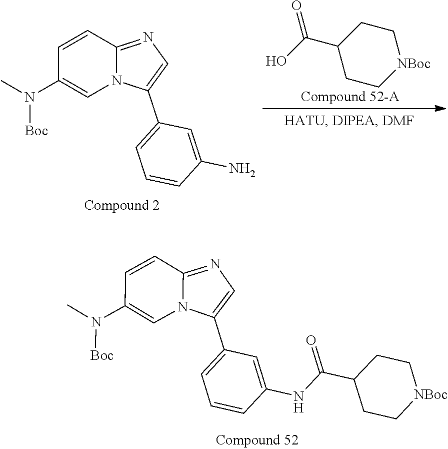

Step 1: The synthesis of compound 52 was performed referring to the first step of the synthesis method of compound 47.

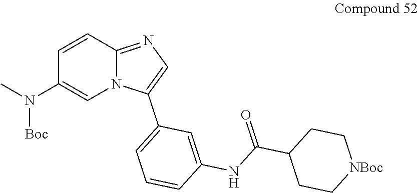 [SMILES: CN(C(=O)OC(C)(C)C)c1ccc2ncc(-c3cccc(NC(=O)C4CCN(C(=O)OC(C)(C)C)CC4)c3)n2c1]

LC_MS: (ES⁺): m/z 550.3 [M+H]⁺.

### Synthesis of Compound 53

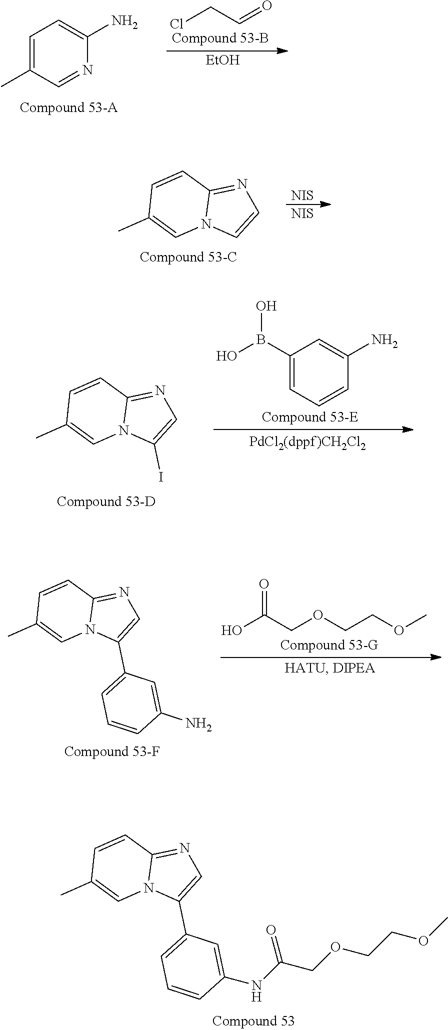

Step 1: The synthesis of compound 53-C was performed referring to the third step of the synthesis method of intermediate BB5.

Step 2: The synthesis of compound 53-D was performed referring to the fourth step of the synthesis method of intermediate BB5.

Step 3: The synthesis of compound 53-F was performed referring to the first step of the synthesis method of compound 2.

Step 4: The synthesis of compound 53 using compound 53-F and compound 53-G as raw materials, was performed referring to the first step of the synthesis method of compound 47

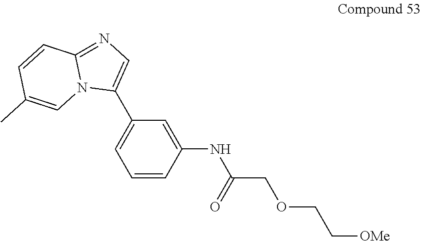 [SMILES: COCCOCC(=O)Nc1cccc(-c2cnc3ccc(C)cn23)c1]

LC_MS: (ES⁺): m/z 340.1 [M+H]⁺.

### Synthesis of Compound 54

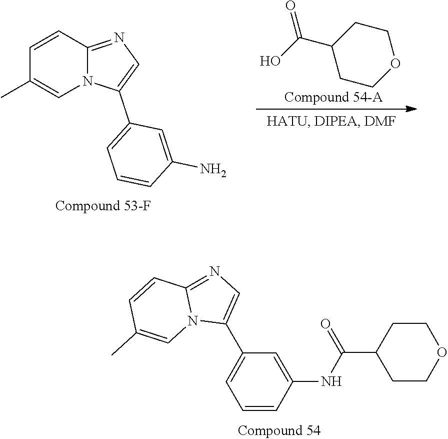

Step 1: The synthesis of compound 54 using compound 53-F and compound 54-A as raw materials, was performed referring to the first step of the synthesis method of compound 47.

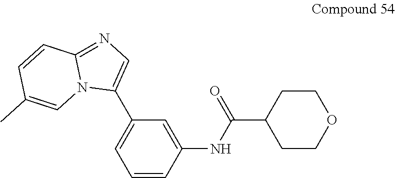 [SMILES: Cc1ccc2ncc(-c3cccc(NC(=O)C4CCOCC4)c3)n2c1]

LC_MS: (ES⁺): m/z 336.4 [M+H]⁺.

### Synthesis of Compound 55

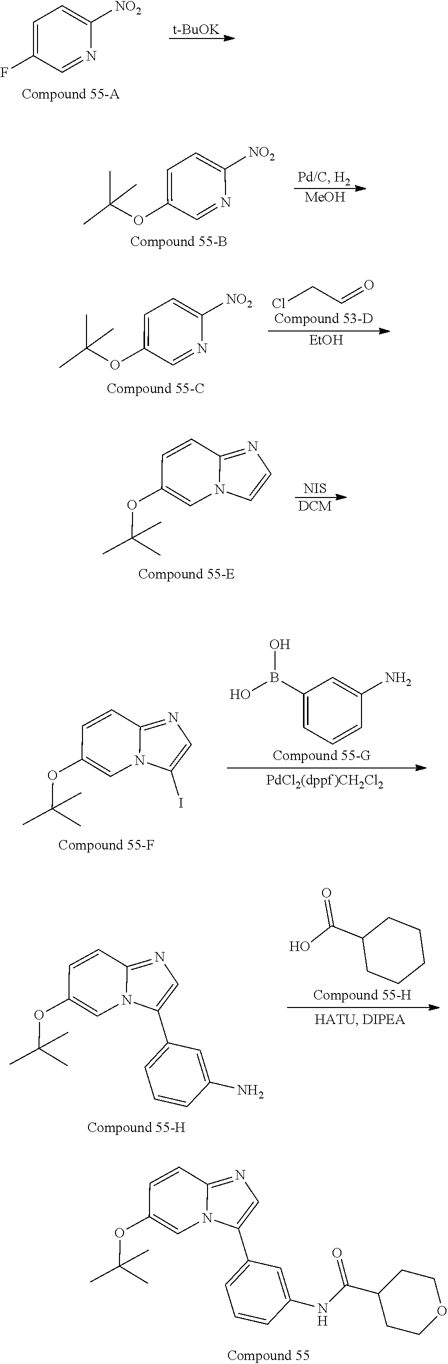

### Step 1: Compound 55-A 5-(tert-butoxy)-2-nitropyridine

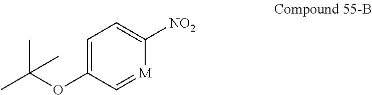

A tetrahydrofuran solution containing compound 55-A (450 mg, 3.17 mmol) was added with potassium tert-butoxide (426.5 mg, 3.80 mmol) under a nitrogen atmosphere at 0° C. The reaction solution was stirred at 20° C. for 12 hours of reaction. The reaction was monitored by LCMS to be completed. The reaction solution was diluted with water (20 mL), and extracted with ethyl acetate (20 mL×2). The organic layers were combined, washed with saturated brine (20 mL), dried over anhydrous sodium sulfate, filtered and concentrated. The residue was separated and purified by silica gel column chromatography (eluted with ethyl acetate solution containing 10% petroleum ether) to obtain compound 55-A (253 mg) as a colorless oil.

LC_MS: (ES⁺): m/z 197.1 [M+H]⁺.

Step 2: The synthesis of compound 55-C was performed referring to the second step of the synthesis method of intermediate BB5.

Step 3: The synthesis of compound 55-E was performed referring to the third step of the synthesis method of intermediate BB5.

Step 4: The synthesis of compound 55-F was performed referring to the fourth step of the synthesis method of intermediate BB5.

Step 5: The synthesis of compound 55-H was performed referring to the first step of the synthesis method of compound 2.

Step 6: The synthesis of compound 55 using compound 55-H and compound 55-I as raw materials, was performed referring to the first step of the synthesis method of compound 47

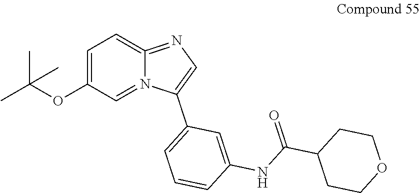

LC_MS: (ES⁺): m/z 394.2 [M+H]⁺.

### Synthesis of Compound 56

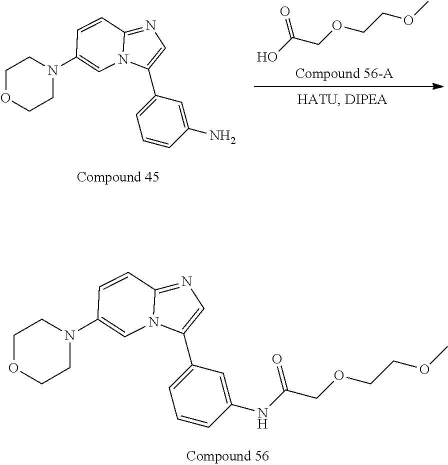

Step 1: The synthesis of compound 56 was performed using compound 45 and compound 56-A as raw materials, referring to the first step of the synthesis method of compound 47.

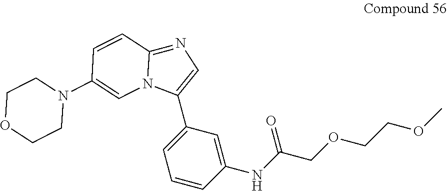 [SMILES: COCCOCC(=O)Nc1cccc(-c2cnc3ccc(N4CCOCC4)cn23)c1]

LC_MS: (ES⁺): m/z 411.1 [M+H]⁺.

### Synthesis of Compound 57

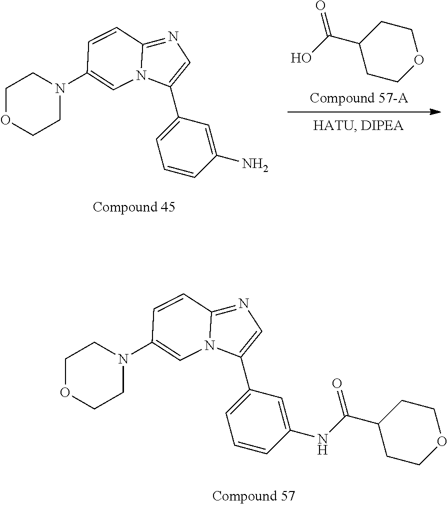 [SMILES: Nc1cccc(-c2cnc3ccc(N4CCOCC4)cn23)c1 ; O=C(O)C1CCOCC1 ; O=C(Nc1cccc(-c2cnc3ccc(N4CCOCC4)cn23)c1)C1CCOCC1]

Step 1: The synthesis of compound 57 was performed using compound 45 and compound 57-A as raw materials, referring to the first step of the synthesis method of compound 47.

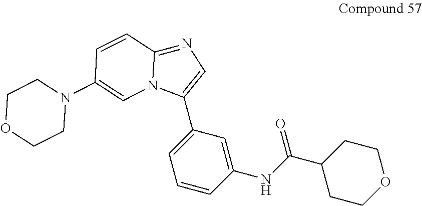 [SMILES: O=C(Nc1cccc(-c2cnc3ccc(N4CCOCC4)cn23)c1)C1CCOCC1]

LC_MS: (ES⁺): m/z 407.1 [M+H]⁺.

### Synthesis of Compound 58

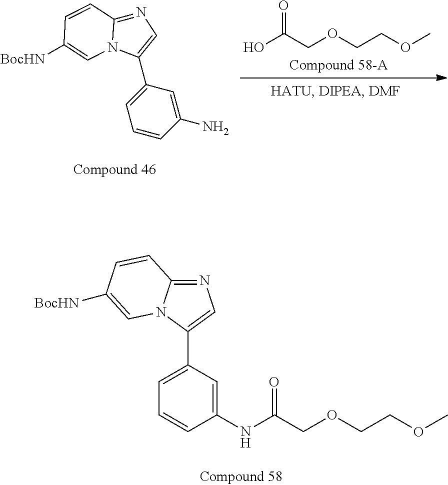

Step 1: The synthesis of compound 58 was performed using compound 46 and compound 58-A as raw materials, referring to the first step of the synthesis method of compound 47.

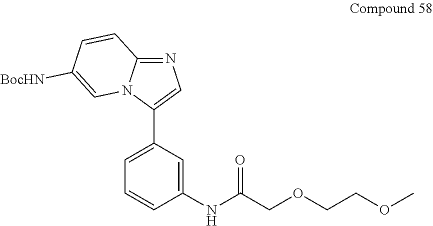 [SMILES: COCCOCC(=O)Nc1cccc(-c2cnc3ccc(NC(=O)OC(C)(C)C)cn23)c1]

LC_MS: (ES⁺): m/z 441.2 [M+H]⁺.

### Example 4: Synthesis of Compound 59 and Compound 60

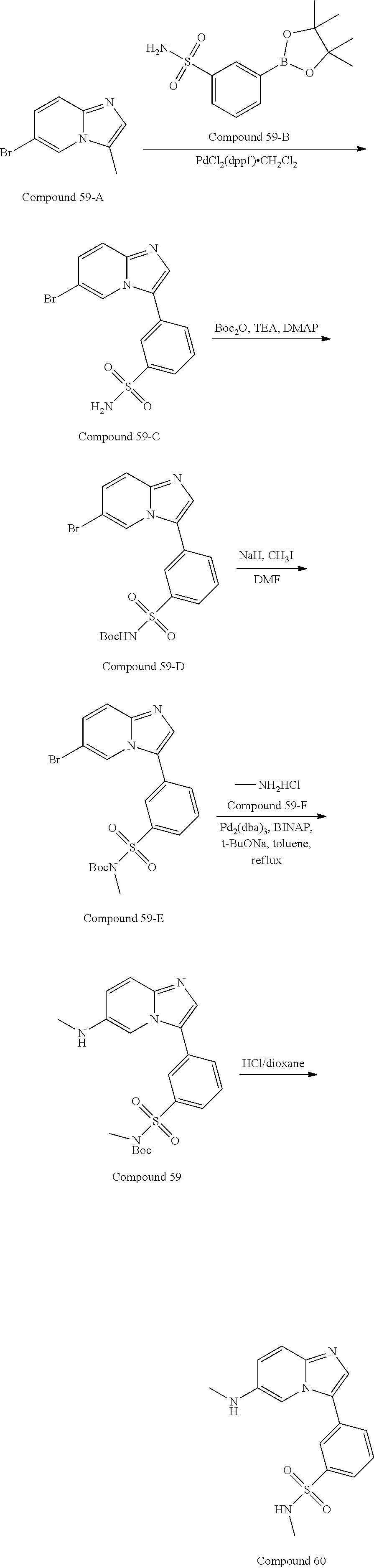

Step 1: The synthesis of compound 59-C was performed referring to the first step of the synthesis method of compound 2.

### Step 2: Compound 59-D tert-butyl ((3-(6-bromoimidazo[1,2-a]pyridin-3-yl)phenyl)sulfonyl)carbamate

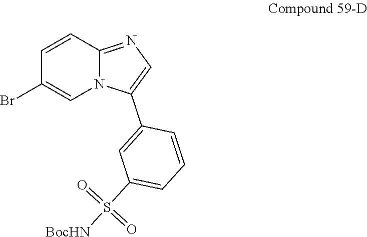 [SMILES: CC(C)(C)OC(=O)NS(=O)(=O)c1cccc(-c2cnc3ccc(Br)cn23)c1]

A solution of compound 59-C (400 mg, 1.14 mmol), triethylamine (230.3 mg, 1.37 mmol) and 4-dimethylaminopyridine (55.6 mg, 0.45 mmol) in tetrahydrofuran (6 ml) was added with di-tert-butyl succinate (298.1 mg, 1.37 mmol). The reaction solution was stirred at room temperature for 4 hours of reaction. The reaction was monitored by TLC to be completed. The reaction solution was extracted with water and dichloromethane. The combined organic layers were dried over anhydrous sodium sulfate and concentrated under reduced pressure. The residue was separated and purified by silica gel column chromatography (eluted with dichloromethane containing 2.4%-4.7% methanol) to obtain compound 59-D tert-butyl ((3-(6-bromoimidazo[1,2-a]pyridin-3-yl)phenyl)sulfonyl) carbamate (370 mg) as a yellow solid.

LC_MS: (ES⁺): m/z 453.9 [M+H]⁺

### Step 3: Compound 59-E tert-butyl ((3-(6-bromoimidazo[1,2-a]pyridin-3-yl)phenyl)sulfonyl)(methyl)carbamate

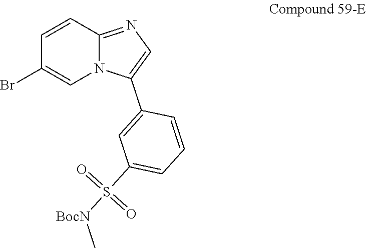 [SMILES: CN(C(=O)OC(C)(C)C)S(=O)(=O)c1cccc(-c2cnc3ccc(Br)cn23)c1]

A N,N-dimethylformamide solution containing sodium hydride (25.7 mg, 0.643 mmol, 60% dispersed in mineral oil) was added with compound 59-D (290 mg, 0.643 mmol) and methyl iodide (182.55 mg, 1.29 mmol) at 0° C. under nitrogen. The reaction solution was stirred at 0° C. for 4 hours of reaction. The reaction was monitored by TLC to be completed. The reaction solution was extracted with water and ethyl acetate. The combined organic layers were washed with saturated brine, dried over anhydrous sodium sulfate, and concentrated under reduced pressure. The obtained residue was separated and purified by silica gel column chromatography (eluted with dichloromethane containing 1.9%-4.7% methanol) to obtain a yellow solid compound 59-E tert-butyl ((3-(6-bromoimidazo[1,2-a]pyridin-3-yl)phenyl)sulfonyl)(methyl)carbamate (220 mg).

LC_MS: (ES⁺): m/z 465.9 [M+H]⁺.

### Step 4: Compound 59 tert-butyl methyl ((3-(6-(methylamino)imidazo[1,2-a]pyridin-3-yl)phenyl)sulfonyl)carbamate

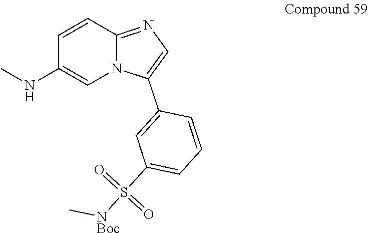 [SMILES: CNc1ccc2ncc(-c3cccc(S(=O)(=O)N(C)C(=O)OC(C)(C)C)c3)n2c1]

A solution of compound 59-E (140 mg, 0.3 mmol), compound 59-F methylamine hydrochloride (101.28 mg, 1.5 mmol), and cesium carbonate (688.8 mg, 2.1 mmol) in 1,4-dioxane (4 ml) was added with BrettPhos-Pd-G3 (54.39 mg, 0.2 mmol) at room temperature under nitrogen atmosphere. The reaction mixture was replaced with nitrogen three times, and stirred at 110° C. for 48 hours of reaction. The reaction was monitored by TLC to be completed. The reaction solution was extracted with water and dichloromethane. The combined organic layers were dried over anhydrous sodium sulfate and concentrated under reduced pressure. The obtained crude product was separated and purified by preparative TLC (eluted with dichloromethane containing 4.76% methanol) to obtain a brown solid compound 59 tert-butyl methyl ((3-(6-(methylamino)imidazo[1,2-a]pyridin-3-yl)phenyl)sulfonyl)carbamate (15 mg).

LC_MS: (ES⁺): m/z 417.0 [M+H]⁺.

### Step 5: Compound 60 N-methyl-3-(6-(methylamino)imidazo[1,2-a]pyridin-3-yl)benzenesulfonamide

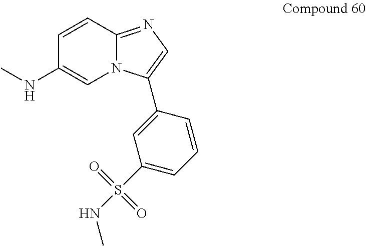 [SMILES: CNc1ccc2ncc(-c3cccc(S(=O)(=O)NC)c3)n2c1]

A solution of compound 59 tert-butyl methyl ((3-(6-(methylamino)imidazo[1,2-a]pyridin-3-yl)phenyl)sulfonyl)carbamate (15 mg, 0.036 mmol) in methanol (250 l) was added with hydrochloric acid/dioxane (1 ml, 4.0 mol/L) solution. The reaction mixture was stirred at room temperature for 4 hours of reaction. The reaction was monitored by TLC to be completed. The reaction solution was concentrated under reduced pressure to obtain a brown solid compound 60 N-methyl-3-(6-(methylamino)imidazo[1,2-a]pyridin-3-yl)benzenesulfonamide (8 mg).

LC_MS: (ES⁺): m/z 317.0 [M+H]⁺.

### Example 5: Synthesis of Compound 61 and Compound 62

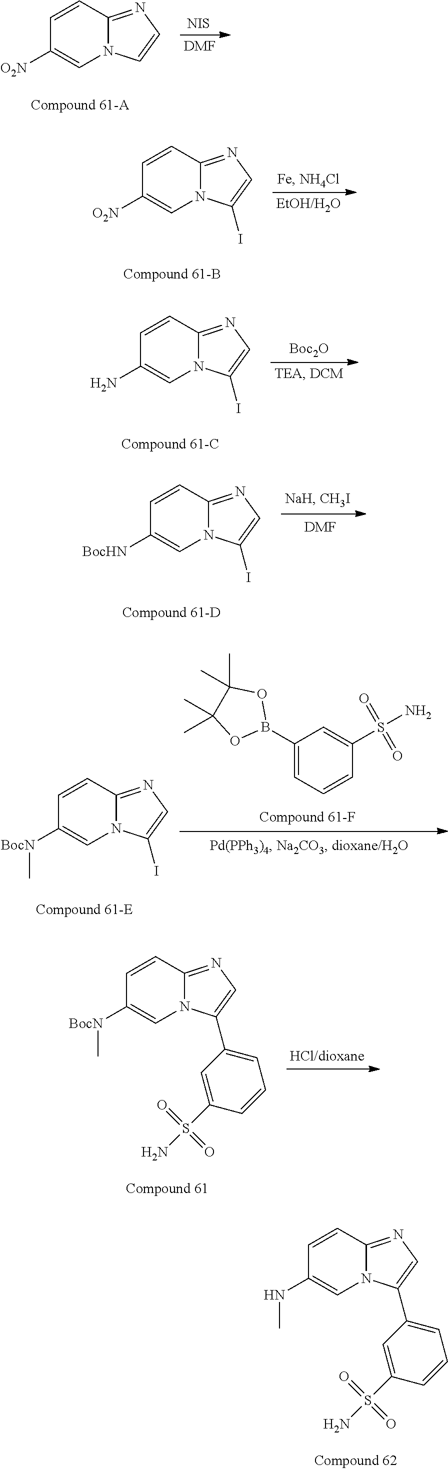

### Step 1: Compound 61-B 3 iodo-6-nitroimidazo[1,2-a]pyridine

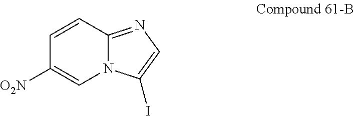 [SMILES: O=[N+]([O-])c1ccc2ncc(I)n2c1]

A solution of compound 61-A (4.85 g, 29.73 mmol) in N,N-dimethylformamide (30 ml) was added with N-iodosuccinimide (8 g, 35.67 mmol). The reaction mixture was stirred at room temperature for 14 hours of reaction. The reaction was monitored by TLC to be completed. The reaction mixture was added with water (30 ml) while being stirred at room temperature. A large amount of white solid was generated, which was then filtered, collected, and dried to obtain white solid compound 61-B 3-iodo-6-nitroimidazo[1,2-a]pyridine (8 g, crude product).

Step 2: Compound 61-C 3-iodoimidazo[1,2-a]pyridin-6-amine

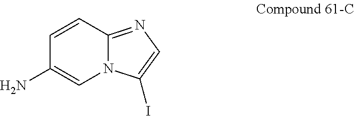 [SMILES: Nc1ccc2ncc(I)n2c1]

A solution containing compound 61-B (500 mg, 1.55 mmol) and ammonium chloride (1.85 g, 34.6 mmol) in ethanol (35 ml) and water (10 ml) was added with iron powder (1.93 g, 34.6 mmol) under nitrogen at room temperature. The reaction mixture was stirred at room temperature for 1 hour of reaction. The reaction was monitored by TLC to be completed. The reaction mixture was filtered through diatomite, and the filtrate was extracted with water and dichloromethane. The combined organic layers were dried over anhydrous sodium sulfate and concentrated under reduced pressure. The obtained crude product was separated and purified by silica gel column chromatography (eluted with ethyl acetate containing 9% dichloromethane) to obtain brown solid compound 61-C 3-iodoimidazo[1,2-a]pyridin-6-amine (1 g).

LC_MS: (ES⁺): m/z 259.8.0 [M+H]⁺.

### Step 3: Compound 61-D tert-butyl (3-iodoimidazo[1,2-a]pyridin-6-yl)carbamate

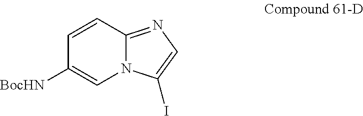 [SMILES: CC(C)(C)OC(=O)Nc1ccc2ncc(I)n2c1]

A solution containing compound 61-C (1 g, 3.86 mmol), N,N-diisopropylethylamine (1.49 g, 11.58 mmol) and 4-dimethylaminopyridine (47.1 mg, 0.39 mmol) in dichloromethane (15 ml) and N,N-dimethylformamide (4 ml) was added with di-tert-butyl succinate (925.6 mg, 4.25 mmol). The reaction mixture was stirred at room temperature for 18 hours of reaction. The reaction was monitored by TLC to be completed. The reaction mixture was extracted with water and dichloromethane. The combined organic layers were dried over anhydrous sodium sulfate and concentrated under reduced pressure. The obtained crude product was separated and purified by silica gel column chromatography (eluted with petroleum ether containing 50%-100% ethyl acetate) to obtain brown solid compound 61-D tert-butyl (3-iodoimidazo[1,2-a]pyridin-6-yl)carbamate (330 mg).

LC_MS: (ES⁺): m/z 359.9 [M+H]⁺.

### Step 4: Compound 61-E tert-butyl (3-iodoimidazo[1,2-a]pyridin-6-yl)(methyl)carbamate

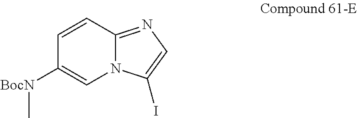 [SMILES: CN(C(=O)OC(C)(C)C)c1ccc2ncc(I)n2c1]

A solution of sodium hydride (36.76 mg, 0.92 mmol, 60% dispersed in mineral oil) in N,N-dimethylformamide (2 ml) was added with compound 61-D (330 mg, 0.92 mmol) and methyl iodide (260.94 mg, 1.84 mmol) at 0° C. under nitrogen. The reaction mixture was stirred at 0° C. for 3 hours of reaction. The reaction was monitored by TLC to be completed. The reaction solution was extracted with water and ethyl acetate. The combined organic layers were washed with saturated brine, dried over anhydrous sodium sulfate, and concentrated under reduced pressure. The obtained crude product was separated and purified by silica gel column chromatography (eluted with dichloromethane containing 2.4%-7.7% methanol) to obtain a brown solid compound 61-E tert-butyl (3-iodoimidazo[1,2-a]pyridin-6-yl)(methyl)carbamate (270 mg).

LC_MS: (ES⁺): m/z 373.9 [M+H]⁺.

### Step 4: Compound 61 tert-butyl methyl (3-(3-sulfamoylphenyl)imidazo[1,2-a]pyridin-6-yl)carbamate

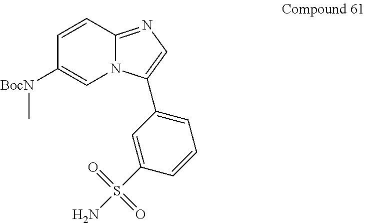 [SMILES: CN(C(=O)OC(C)(C)C)c1ccc2ncc(-c3cccc(S(N)(=O)=O)c3)n2c1]

A solution containing compound 61-E (50 mg, 0.134 mmol), compound 61-F (94.83 mg, 0.335 mmol), and sodium carbonate (42.61 mg, 0.402 mmol) in 1,4-dioxane (2 ml) and water (600 l) was added with Pd(PPh₃)₄ (10.84 mg, 0.01 mmol) at room temperature under nitrogen atmosphere. The reaction mixture was replaced with nitrogen three times, and stirred at 90° C. for 18 hours of reaction. The reaction was monitored by TLC to be completed. The reaction solution was extracted with water and dichloromethane. The combined organic layers were dried over anhydrous sodium sulfate and concentrated under reduced pressure. The resulting crude product was separated and purified by preparative TLC (eluted with dichloromethane containing 8.3% methanol) to obtain a white solid compound 61 tert-butyl methyl (3-(3-sulfamoylphenyl)imidazo[1,2-a]pyridin-6-yl)carbamate (27 mg).

LC_MS: (ES⁺): m/z 403.0 [M+H]⁺.

### Step 5: Compound 62 3-(6-(methylamino)imidazo[1,2-a]pyridin-3-yl) benzenesulfonamide

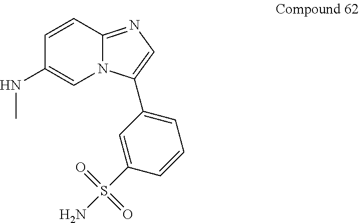 [SMILES: CNc1ccc2ncc(-c3cccc(S(N)(=O)=O)c3)n2c1]

A solution of compound 61 (24 mg, 0.035 mmol) in methanol (250 l) and hydrochloric acid/dioxane (1 ml, 4.0 mol/L) was stirred at room temperature for 4 hours of reaction. The reaction was monitored by TLC to be completed. The reaction solution was concentrated under reduced pressure to obtain compound 62 3-(6-(methylamino)imidazo[1,2-a]pyridin-3-yl)benzenesulfonamide (16.7 mg) as a yellow solid.

LC_MS: (ES⁺): m/z 302.9 [M+H]⁺.

¹H NMR (400 MHz, MeOD) δ 8.23 (t, J=1.6 Hz, 1H), 8.12 (ddd, J=7.9, 1.8, 1.1 Hz, 1H), 8.04 (s, 1H), 7.94 (dd, J=5.3, 4.1 Hz, 1H), 7.83 (t, J=7.8 Hz, 1H), 7.74 (d, J=9.7 Hz, 1H), 7.56 (dd, J=9.7, 2.0 Hz, 1H), 7.49 (d, J=1.9 Hz, 1H), 2.76 (s, 3H).

### Example 6: Synthetic Route of Compound 63

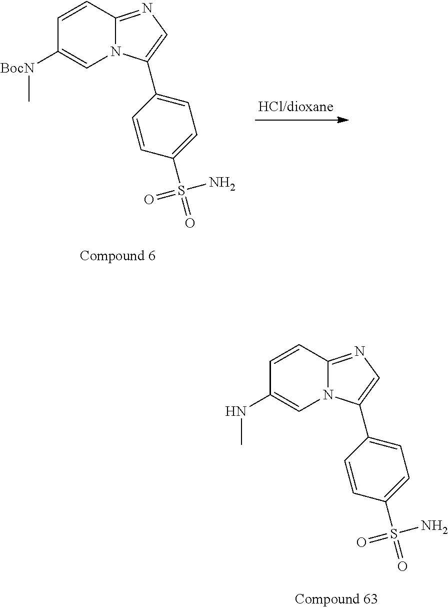 [SMILES: CN(C(=O)OC(C)(C)C)c1ccc2ncc(-c3ccc(S(N)(=O)=O)cc3)n2c1 ; CNc1ccc2ncc(-c3ccc(S(N)(=O)=O)cc3)n2c1]

### Step 1: Compound 63 4-(6-(methylamino)imidazo[1,2-a]pyridin-3-yl)benzenesulfonamide

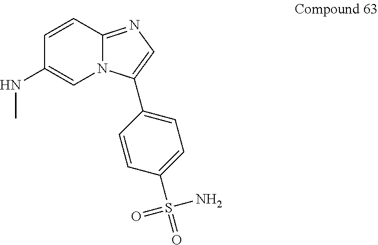 [SMILES: CNc1ccc2ncc(-c3ccc(S(N)(=O)=O)cc3)n2c1]

A solution of compound 6 (40 mg, 0.099 mmol) in methanol (0.5 ml) and hydrochloric acid/dioxane (1 ml, 4.0 mol/L) was stirred at room temperature for 2 hours of reaction. The reaction was monitored by TLC to be completed. The reaction solution was concentrated under reduced pressure to obtain compound 63 (30 mg) as a yellow solid.

LC_MS: (ES⁺): m/z 303.0 [M+H]⁺.

¹H NMR (500 MHz, MeOD) δ 8.16 (d, J=7.4 Hz, 2H), 8.09 (s, 1H), 7.94 (d, J=7.8 Hz, 2H), 7.78 (d, J=9.3 Hz, 1H), 7.62 (d, J=10.2 Hz, 2H), 2.80 (s, 3H).

The following compounds were synthesized referring to the synthesis of compound 63:

### Synthetic Route of Compound 64

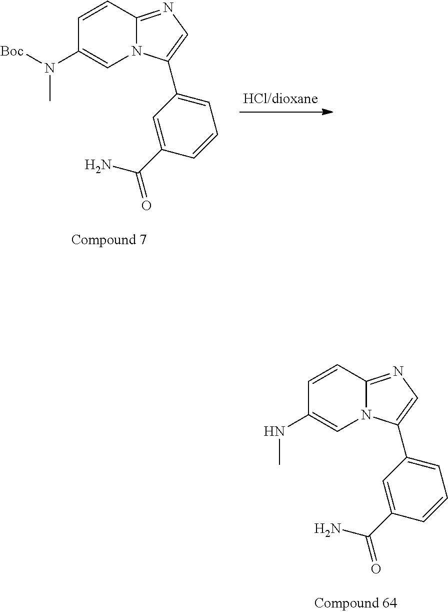 [SMILES: CN(C(=O)OC(C)(C)C)c1ccc2ncc(-c3cccc(C(N)=O)c3)n2c1 ; CNc1ccc2ncc(-c3cccc(C(N)=O)c3)n2c1]

Step 1: The synthesis of compound 64 was performed referring to the first step of the synthesis method of compound 63.

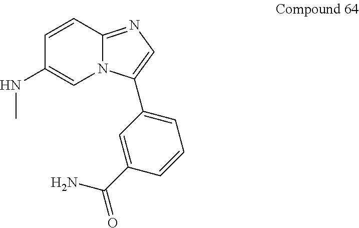 [SMILES: CNc1ccc2ncc(-c3cccc(C(N)=O)c3)n2c1]

LC_MS: (ES⁺): m/z 267.0 [M+H]⁺.

¹H NMR (500 MHz, MeOD) δ 8.22 (s, 1H), 8.13-8.09 (m, 1H), 8.04 (s, 1H), 7.93 (d, J=7.7 Hz, 1H), 7.82-7.75 (m, 2H), 7.70-7.57 (m, 2H), 2.80 (s, 3H).

### Synthesis of Compound 65

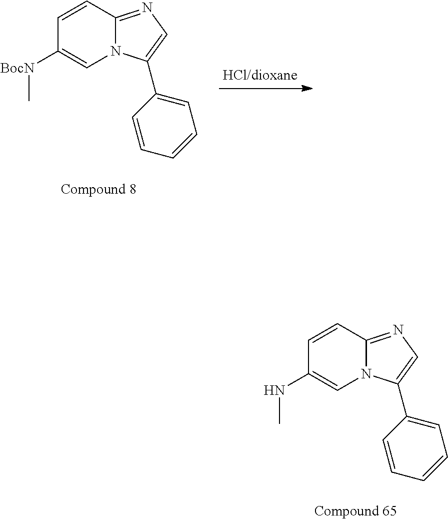 [SMILES: CN(C(=O)OC(C)(C)C)c1ccc2ncc(-c3ccccc3)n2c1 ; CNc1ccc2ncc(-c3ccccc3)n2c1]

Step 1: The synthesis of compound 65 was performed referring to the first step of the synthesis method of compound 63.

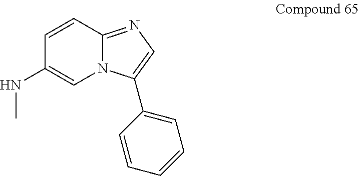 [SMILES: CNc1ccc2ncc(-c3ccccc3)n2c1]

LC_MS: (ES⁺): m/z 224.0 [M+H]⁺.

¹H NMR (500 MHz, MeOD) δ 7.98 (s, 1H), 7.79 (d, J=9.6 Hz, 1H), 7.73 (d, J=7.2 Hz, 1H), 7.68-7.65 (m, 1H), 7.64 (d, J=4.0 Hz, 1H), 7.62 (d, J=2.7 Hz, 1H), 2.80 (s, 1H).

### Synthesis of Compound 66

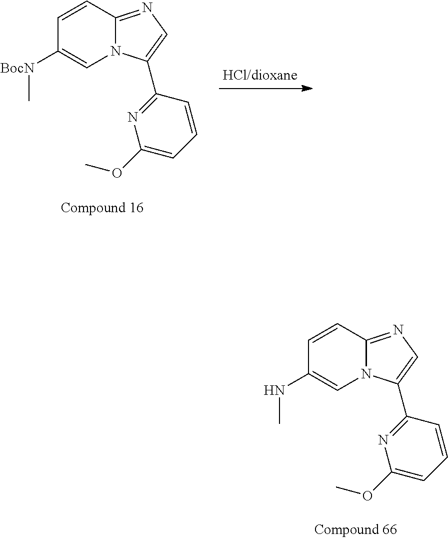 [SMILES: COc1cccc(-c2cnc3ccc(N(C)C(=O)OC(C)(C)C)cn23)n1 ; CNc1ccc2ncc(-c3cccc(OC)n3)n2c1]

Step 1: The synthesis of compound 66 was performed referring to the first step of the synthesis method of compound 63.

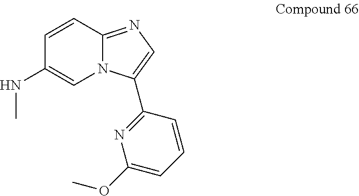 [SMILES: CNc1ccc2ncc(-c3cccc(OC)n3)n2c1]

LC_MS: (ES⁺): m/z 255.0 [M+H]⁺.

¹H NMR (400 MHz, DMSO) δ 9.09 (d, J=1.9 Hz, 1H), 8.79 (s, 1H), 7.93-7.87 (m, 1H), 7.80 (d, J=9.6 Hz, 1H), 7.65 (d, J=7.4 Hz, 1H), 7.51 (d, J=2.9 Hz, 1H), 6.90 (d, J=8.2 Hz, 1H), 4.04 (s, 3H), 2.74 (s, 3H).

### Synthesis of Compound 67

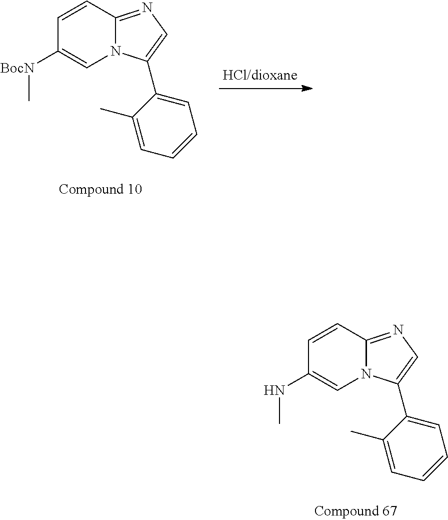 [SMILES: Cc1ccccc1-c1cnc2ccc(N(C)C(=O)OC(C)(C)C)cn12 ; CNc1ccc2ncc(-c3ccccc3C)n2c1]

Step 1: The synthesis of compound 67 was performed referring to the first step of the synthesis method of compound 63.

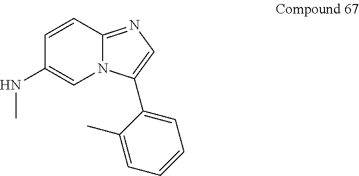 [SMILES: CNc1ccc2ncc(-c3ccccc3C)n2c1]

LC_MS: (ES⁺): m/z 238.0 [M+H]⁺.

¹H NMR (500 MHz, DMSO) δ 8.15 (s, 1H), 7.82 (d, J=9.6 Hz, 1H), 7.54 (d, J=2.1 Hz, 1H), 7.52-7.48 (m, 3H), 7.43 (d, J=7.3 Hz, 1H), 6.87 (d, J=1.9 Hz, 1H), 2.58 (s, 3H), 2.18 (s, 3H).

### Synthesis of Compound 68

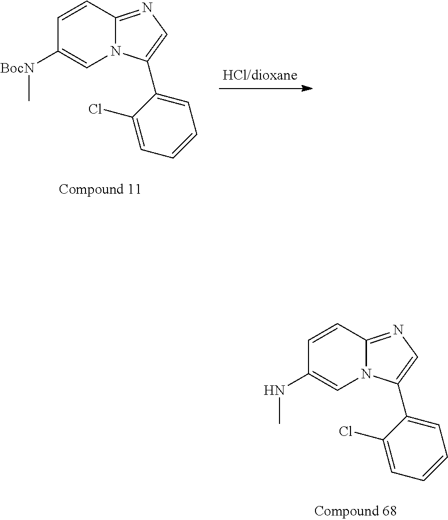 [SMILES: CN(C(=O)OC(C)(C)C)c1ccc2ncc(-c3ccccc3Cl)n2c1 ; CNc1ccc2ncc(-c3ccccc3Cl)n2c1]

Step 1: The synthesis of compound 68 was performed referring to the first step of the synthesis method of compound 63.

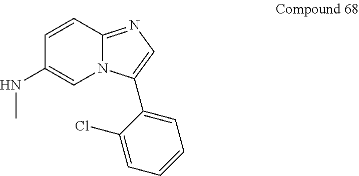 [SMILES: CNc1ccc2ncc(-c3ccccc3Cl)n2c1]

LC_MS: (ES⁺): m/z 257.9 [M+H]⁺.

¹H NMR (500 MHz, DMSO) δ 8.25 (s, 1H), 7.83 (d, J=9.6 Hz, 1H), 7.78 (d, J=8.1 Hz, 1H), 7.69 (t, J=7.3 Hz, 2H), 7.61 (t, J=7.4 Hz, 1H), 7.55 (dd, J=9.7, 1.9 Hz, 1H), 6.97 (s, 1H), 2.60 (s, 3H).

### Synthesis of Compound 69

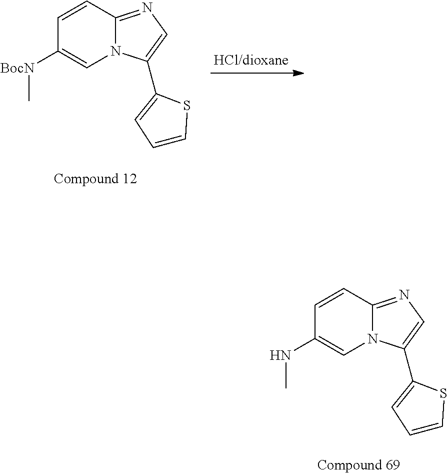 [SMILES: CN(C(=O)OC(C)(C)C)c1ccc2ncc(-c3cccs3)n2c1 ; CNc1ccc2ncc(-c3cccs3)n2c1]

Step 1: The synthesis of compound 69 was performed referring to the first step of the synthesis method of compound 63.

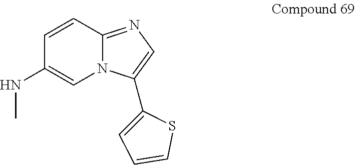 [SMILES: CNc1ccc2ncc(-c3cccs3)n2c1]

LC_MS: (ES⁺): m/z 229.9 [M+H]⁺.

¹H NMR (400 MHz, DMSO) δ 8.30 (s, 1H), 7.89 (dd, J=5.1, 1.1 Hz, 1H), 7.79 (dd, J=9.6, 0.4 Hz, 1H), 7.67 (dd, J=3.6, 1.1 Hz, 1H), 7.54 (dd, J=9.6, 2.1 Hz, 1H), 7.50 (d, J=1.8 Hz, 1H), 7.34 (dd, J=5.1, 3.6 Hz, 1H), 2.68 (s, 3H).

### Synthesis of Compound 70

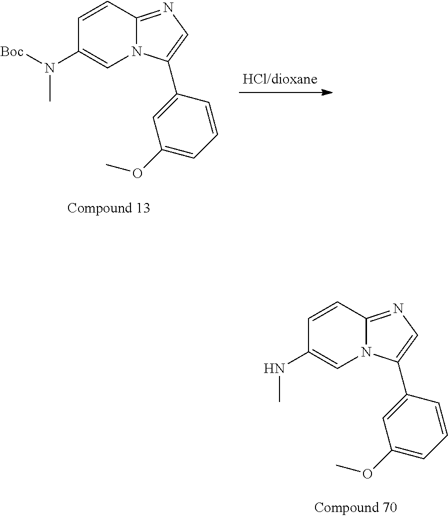 [SMILES: COc1cccc(-c2cnc3ccc(N(C)C(=O)OC(C)(C)C)cn23)c1 ; CNc1ccc2ncc(-c3cccc(OC)c3)n2c1]

Step 1: The synthesis of compound 70 was performed referring to the first step of the synthesis method of compound 63.

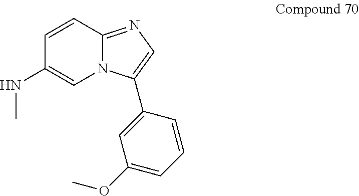 [SMILES: CNc1ccc2ncc(-c3cccc(OC)c3)n2c1]

LC_MS: (ES⁺): m/z 254.0 [M+H]⁺.

¹H NMR (400 MHz, DMSO) δ 8.20 (s, 1H), 7.78 (d, J=9.6 Hz, 1H), 7.56-7.49 (m, 2H), 7.47 (d, J=1.6 Hz, 1H), 7.31-7.26 (m, 2H), 7.17-7.12 (m, 1H), 3.83 (s, 3H), 2.66 (s, 3H).

### Synthesis of Compound 72

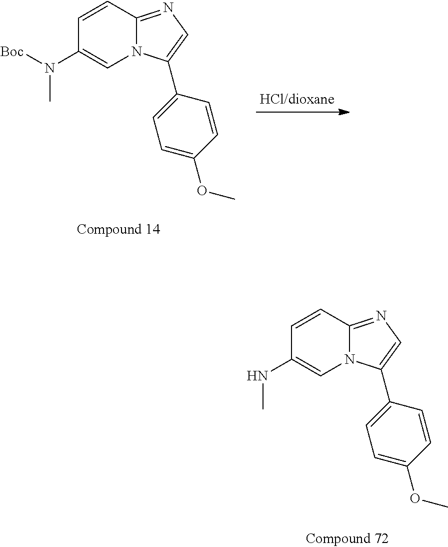 [SMILES: COc1ccc(-c2cnc3ccc(N(C)C(=O)OC(C)(C)C)cn23)cc1 ; CNc1ccc2ncc(-c3ccc(OC)cc3)n2c1]

Step 1: The synthesis of compound 72 was performed referring to the first step of the synthesis method of compound 63.

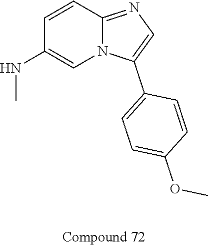 [SMILES: CNc1ccc2ncc(-c3ccc(OC)cc3)n2c1]

LC_MS: (ES⁺): m/z 254.0 [M+H]⁺.

¹H NMR (400 MHz, DMSO) δ 8.11 (s, 1H), 7.78 (d, J=9.6 Hz, 1H), 7.65-7.62 (m, 1H), 7.52-7.51 (m, 1H), 7.36 (d, J=1.8 Hz, 1H), 7.19-7.14 (m, 2H), 3.84 (s, 3H), 2.64 (s, 3H).

### Synthesis of Compound 73

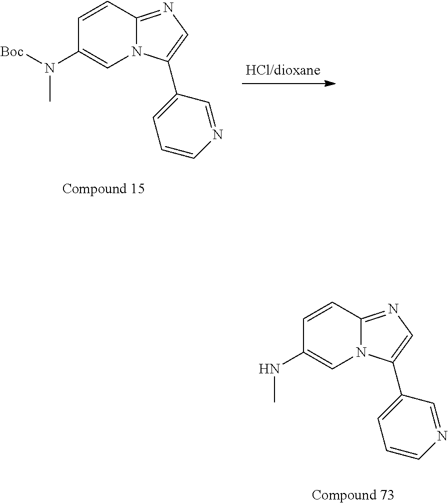 [SMILES: CN(C(=O)OC(C)(C)C)c1ccc2ncc(-c3cccnc3)n2c1 ; CNc1ccc2ncc(-c3cccnc3)n2c1]

Step 1: The synthesis of compound 73 was performed referring to the first step of the synthesis method of compound 63.

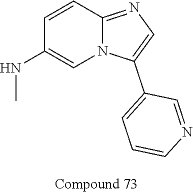 [SMILES: CNc1ccc2ncc(-c3cccnc3)n2c1]

LC_MS: (ES⁺): m/z 225.0 [M+H]⁺.

¹H NMR (500 MHz, DMSO) δ 9.11 (s, 1H), 8.93 (d, J=4.3 Hz, 1H), 8.54 (d, J=7.9 Hz, 1H), 8.38 (s, 1H), 7.95 (dd, J=7.8, 5.3 Hz, 1H), 7.86 (d, J=9.6 Hz, 1H), 7.59 (dd, J=9.7, 2.0 Hz, 1H), 7.53 (d, J=1.6 Hz, 1H), 2.70 (s, 3H).

### Synthesis of Compound 74

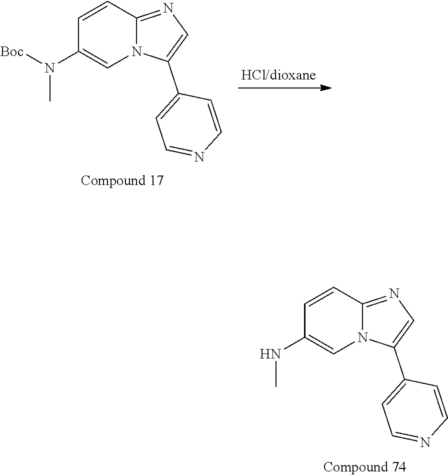 [SMILES: CN(C(=O)OC(C)(C)C)c1ccc2ncc(-c3ccncc3)n2c1 ; CNc1ccc2ncc(-c3ccncc3)n2c1]

Step 1: The synthesis of compound 74 was performed referring to the first step of the synthesis method of compound 63.

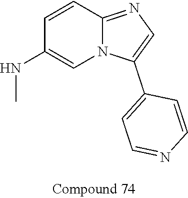 [SMILES: CNc1ccc2ncc(-c3ccncc3)n2c1]

LC_MS: (ES⁺): m/z 225.0 [M+H]⁺.

¹H NMR (400 MHz, DMSO) δ 8.98 (d, J=6.6 Hz, 2H), 8.64 (s, 1H), 8.26 (d, J=6.6 Hz, 2H), 7.85 (d, J=9.7 Hz, 1H), 7.74 (d, J=1.7 Hz, 1H), 7.59 (dd, J=9.7, 2.0 Hz, 1H), 2.72 (s, 3H).

### Synthesis of Compound 75

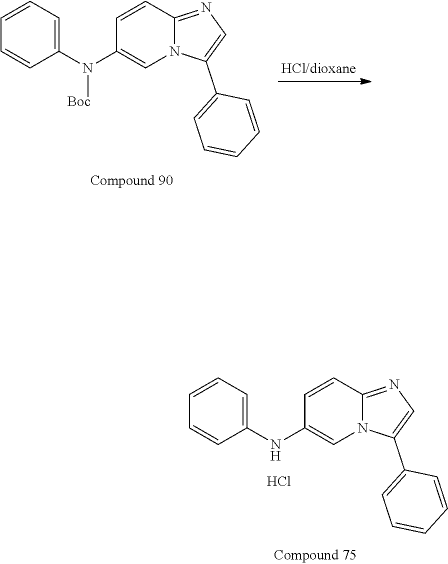 [SMILES: CC(C)(C)OC(=O)N(c1ccccc1)c1ccc2ncc(-c3ccccc3)n2c1 ; c1ccc(Nc2ccc3ncc(-c4ccccc4)n3c2)cc1 ; Cl]

Step 1: The synthesis of compound 75 was performed referring to the first step of the synthesis method of compound 63.

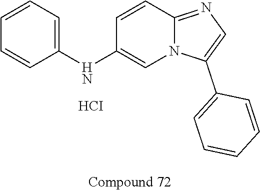

LC_MS: (ES⁺): m/z 286.1 [M+H]⁺.

### Synthesis of Compound 76

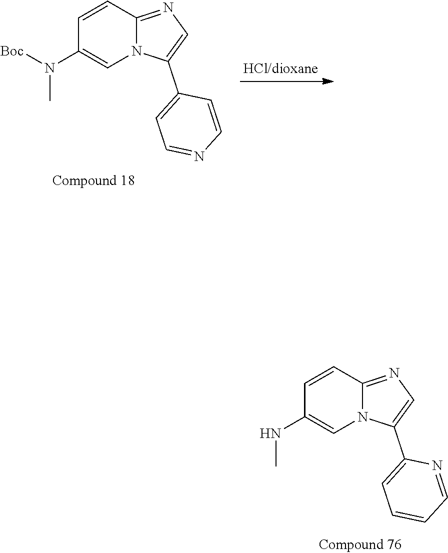 [SMILES: CN(C(=O)OC(C)(C)C)c1ccc2ncc(-c3ccncc3)n2c1 ; CNc1ccc2ncc(-c3ccccn3)n2c1]

Step 1: the synthesis of compound 76 was performed referring to the first step of the synthesis method of compound 63.

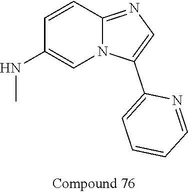 [SMILES: CNc1ccc2ncc(-c3ccccn3)n2c1]

LC_MS: (ES⁺): m/z 225.0 [M+H]⁺.

¹H NMR (400 MHz, DMSO) δ 9.21 (d, J=1.9 Hz, 1H), 8.83 (s, 1H), 8.77 (d, J=4.7 Hz, 1H), 8.06 (d, J=8.0 Hz, 1H), 8.03-7.98 (m, 1H), 7.82 (d, J=9.6 Hz, 1H), 7.57 (dd, J=9.6, 2.1 Hz, 1H), 7.49-7.43 (m, 1H), 2.74 (s, 3H).

### Synthesis of Compound 77

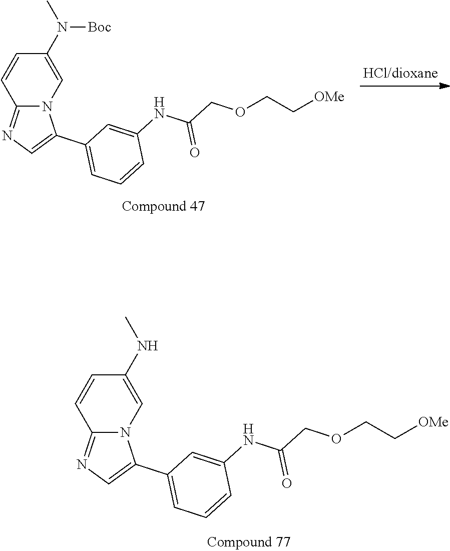 [SMILES: COCCOCC(=O)Nc1cccc(-c2cnc3ccc(N(C)C(=O)OC(C)(C)C)cn23)c1 ; CNc1ccc2ncc(-c3cccc(NC(=O)COCCOC)c3)n2c1]

Step 1: The synthesis of compound 77 was performed referring to the first step of the synthesis method of compound 63.

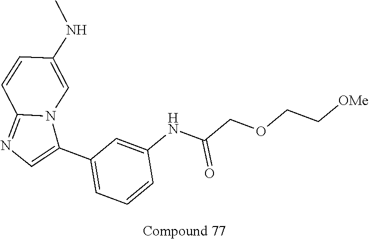 [SMILES: CNc1ccc2ncc(-c3cccc(NC(=O)COCCOC)c3)n2c1]

LC_MS: (ES⁺): m/z 355.1 [M+H]⁺.

### Synthesis of Compound 78

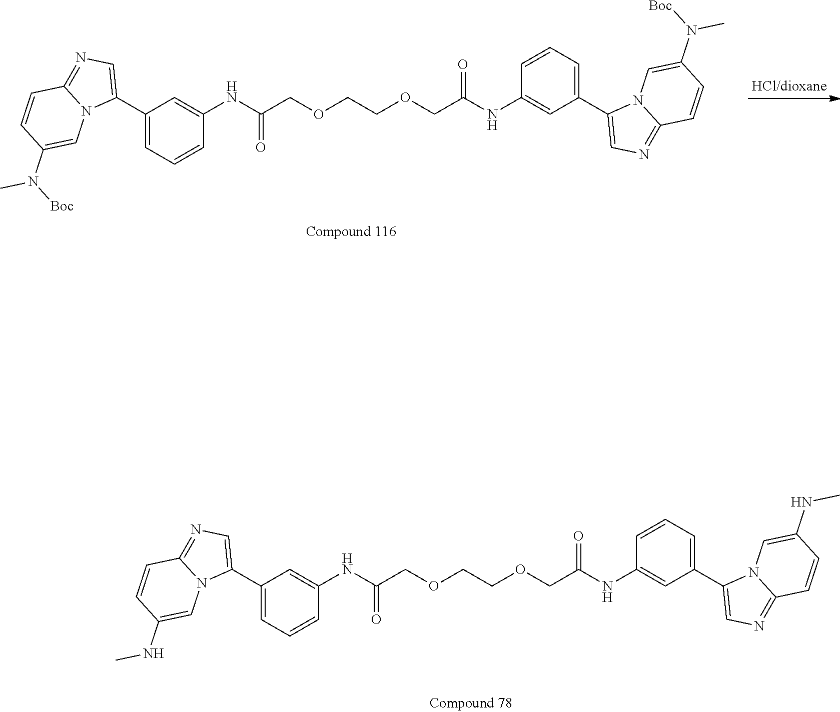

Step 1: The synthesis of compound 78 was performed referring to the first step of the synthesis method of compound 63.

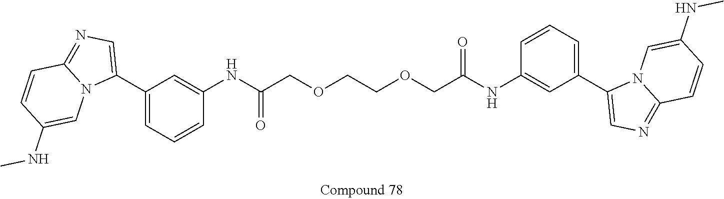

LC_MS: (ES⁺): m/z 619.2 [M+H]⁺.

### Synthesis of Compound 79

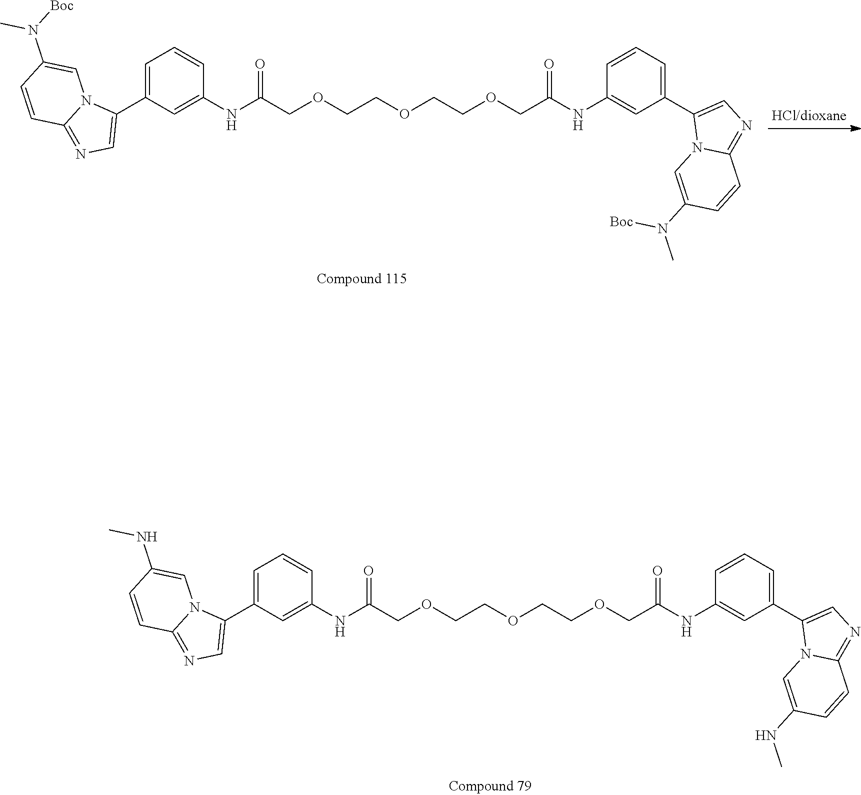 [SMILES: CN(C(=O)OC(C)(C)C)c1ccc2ncc(-c3cccc(NC(=O)COCCOCCOCC(=O)Nc4cccc(-c5cnc6ccc(N(C)C(=O)OC(C)(C)C)cn56)c4)c3)n2c1 ; CNc1ccc2ncc(-c3cccc(NC(=O)COCCOCCOCC(=O)Nc4cccc(-c5cnc6ccc(NC)cn56)c4)c3)n2c1]

Step 1: The synthesis of compound 79 was performed referring to the first step of the synthesis method of compound 63.

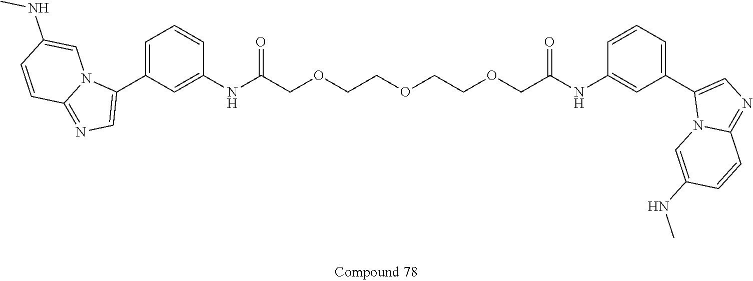 [SMILES: CNc1ccc2ncc(-c3cccc(NC(=O)COCCOCCOCC(=O)Nc4cccc(-c5cnc6ccc(NC)cn56)c4)c3)n2c1]

LC_MS: (ES⁺): m/z 663.1 [M+H]⁺.

### Example 7: Synthesis of Compound 80 and Compound 81

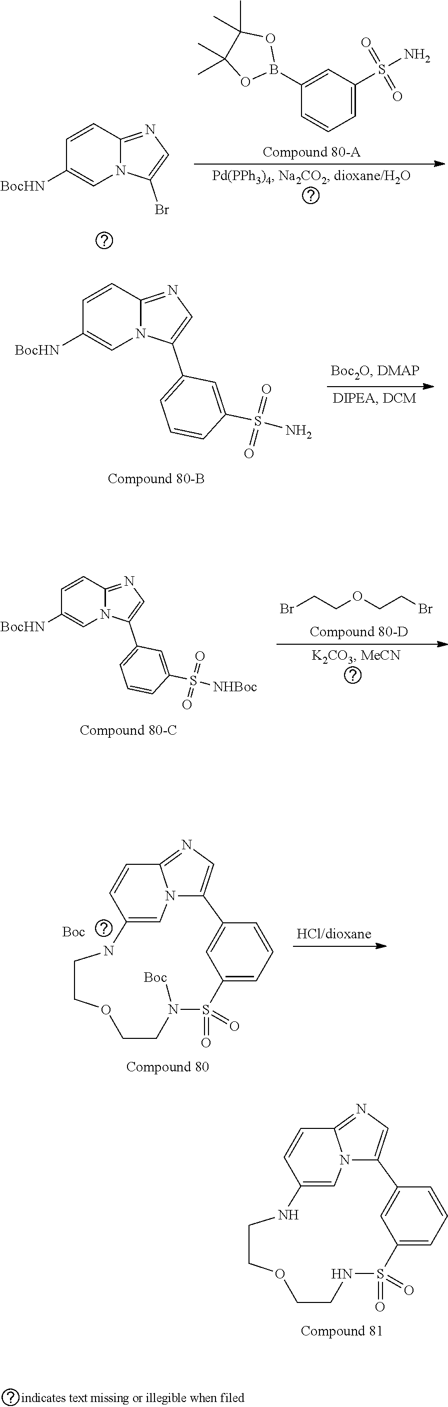

### Step 1: Compound 80-B tert-butyl (3-(3-sulfamoylphenyl)imidazo[1,2-a]pyridin-6-yl)carbamate

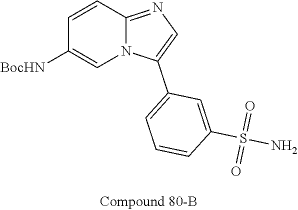 [SMILES: CC(C)(C)OC(=O)Nc1ccc2ncc(-c3cccc(S(N)(=O)=O)c3)n2c1]

A solution containing compound BB5 (100 mg, 320.34 μmol) and compound 80-A (272 mg, 961.02 μmol) in 1,4-dioxane (3 mL)/water (1 mL) was added with Pd(PPh₃)₄ (18.51 mg, 16.02 μmol) and sodium carbonate (102 mg, 961.02 μmol). The reaction solution was stirred under nitrogen at 90° C. for 12 hours of reaction. The reaction was monitored by LCMS to be completed. The reaction solution was diluted with water (20 mL), and extracted with ethyl acetate (10 mL×2). The combined organic layers were washed with saturated brine (20 mL), dried over anhydrous sodium sulfate, filtered, and concentrated. The crude product was separated and purified by preparative TLC (dichloromethane:methanol=10:1, Rf=0.5) to obtain yellow solid compound 80-B tert-butyl (3-(3-sulfamoylphenyl)imidazo[1,2-a]pyridin-6-yl)carbamate tert-butyl (3-(3-sulfamoylphenyl)imidazo[1,2-a]pyridin-6-yl)carbamate (100 mg).

LC_MS: (ES⁺): m/z 389.0 [M+H]⁺.

### Step 2: Compound 80-C tert-butyl 3-(3-(N-(tert-butoxycarbonyl)sulfamoyl)phenyl)imidazo[1,2-a]pyridin-6-yl)carbamate

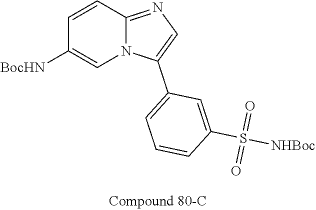 [SMILES: CC(C)(C)OC(=O)Nc1ccc2ncc(-c3cccc(S(=O)(=O)NC(=O)OC(C)(C)C)c3)n2c1]

A solution of compound 80-B (100 mg, 257.44 μmol) in dichloromethane (5 mL) was added with triethylamine (78.15 mg, 772.32 μmol), 4-dimethylaminopyridine (3.2 mg, 25.74 mol) and di-tert-butyl dicarbonate (56.2 mg, 257.44 μmol). The reaction solution was stirred at 20° C. for 12 hours of reaction. Compound 80-B (Rf=0.5) was monitored by TLC (dichloromethane:methanol=10:1) to be reacted completely, and a new spot (Rf=0.6) was generated. The reaction solution was diluted with water (20 mL), and extracted with dichloromethane (10 mL×2). The combined organic layers were washed with saturated brine (20 mL), dried over anhydrous sodium sulfate, filtered, and concentrated. The crude product was separated and purified by preparative TLC (dichloromethane:methanol=10:1, Rf=0.6) to obtain compound 80-C tert-butyl 3-(3-(N-(tert-butoxycarbonyl)sulfamoyl)phenyl)imidazo[1,2-a]pyridin-6-yl)carbamate as a colorless oil (54 mg).

LC_MS: (ES⁺): m/z 489.0 [M+H]⁺.

### Step 3: Compound 80 di-tert-butyl(E)-7-oxa-3-thio-4,10-diaza-1(3,6)-imidazol[1,2-a]pyridin-2(1,3)-phenylcyclodecane-4,10-dicarboxylate 3,3-dioxide

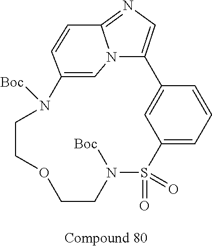 [SMILES: CC(C)(C)OC(=O)N1CCOCCN(C(=O)OC(C)(C)C)S(=O)(=O)c2cccc(c2)-c2cnc3ccc1cn23]

A solution containing compound 80-C (44 mg, 90.06 μmol) and potassium carbonate (62 mg, 450.60 μmol) in acetonitrile (20 mL) was added with compound 80-D (147 mg, 633.84 mol). The reaction solution was stirred at 80° C. for 12 hours of reaction. Most product was detected by LCMS. The reaction solution was diluted with water (30 mL), and extracted with ethyl acetate (30 mL×2). The combined organic layers were washed with saturated brine (50 mL), dried over anhydrous sodium sulfate, filtered, and concentrated. The resulting crude product was separated and purified by preparative TLC (petroleum ether:ethyl acetate=0:1, Rf=0.8) to obtain yellow solid compound 80 di-tert-butyl(E)-7-oxa-3-thio-4,10-diaza-1(3,6)-imidazol[1,2-a]pyridin-2(1,3)-phenylcyclodecane-4,10-dicarboxylate 3,3-dioxide (34 mg).

LC_MS: (ES⁺): m/z 559.1 [M+H]⁺.

### Step 4: Compound 81 (E)-7-oxa-3-thia-4,10-diaza-1(3,6)-imidazo[1,2-a]pyridine-2(1,3)-benzocyclodecane 3,3-dioxide

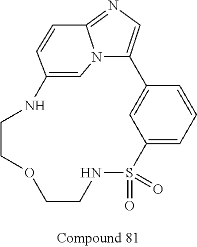 [SMILES: O=S1(=O)NCCOCCNc2ccc3ncc(n3c2)-c2cccc1c2]

A solution of compound 80 (28 mg, 50.12 μmol) in methanol (1.5 mL) was added with hydrochloric acid/dioxane (1.5 mL). The reaction solution was stirred at 20° C. for 1 hour of reaction. The reaction was monitored by LCMS to be completed. The reaction solution was concentrated to obtain compound 81 (10.3 mg) as a yellow solid.

LC_MS: (ES⁺): m/z 359.0 [M+H]⁺.

¹H NMR (500 MHz, DMSO) δ 8.31 (d, J=9.0 Hz, 2H), 8.03-7.91 (m, 3H), 7.85 (d, J=25.9 Hz, 4H), 7.53 (d, J=9.6 Hz, 1H), 7.37-7.04 (m, 1H), 3.78-3.59 (m, 4H), 3.29-3.19 (m, 4H).

### Example 8: Synthesis of Compound 82

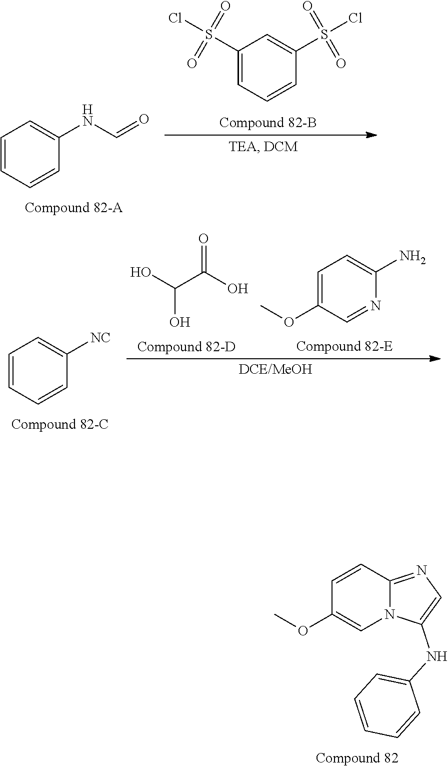

### Step 1: Compound 82-C isocyanobenzene

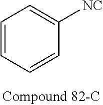

A solution of compound 82-A (400 mg, 3.30 mmol) and triethylamine (2 mL) in dichloromethane (5 mL) was added with compound 82-B (454.2 mg, 1.65 mmol). The reaction solution was stirred at 0° C. for 3 hours of reaction. A new spot (Rf=0.6) was generated by TLC (petroleum ether:ethyl acetate=2:1) monitoring. The reaction mixture was poured into ice water and extracted with dichloromethane (30 mL×2). The combined organic layers were dried over anhydrous sodium sulfate, filtered, and concentrated. The crude product was separated and purified by silica gel column chromatography (SiO₂, petroleum ether:ethyl acetate=5:1-1:1, Rf=0.6) to obtain compound 82-C (38 mg) as a yellow oil.

### Step 2: Compound 82 6-methoxy-N-phenylimidazo[1,2-a]pyridin-3-amine

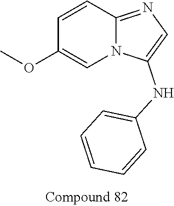 [SMILES: COc1ccc2ncc(Nc3ccccc3)n2c1]

A solution containing compound 82-D (38 mg, 306.10 μmol) and compound 82-E (34 mg, 367.21 μmol) in 1,2-dichloroethane (2 ml)/methanol (1 ml) was stirred at 20° C. for 30 minutes of reaction. Then compound 82-C (38 mg, 367.32 μmol) was added to the reaction solution, and the mixed reaction solution was stirred at 20° C. for 12 hours of reaction. Finally, the mixed reaction solution was added with dichloromethane and sodium bicarbonate solid, and stirred at 20° C. for 15 minutes. The generation of the product was monitored by LCMS. The reaction mixture was filtered through diatomite, and the filtrate was concentrated, diluted with water (10 mL), and extracted with ethyl acetate (20 mL×2). The combined organic layers were washed with saturated brine (20 mL), dried over anhydrous sodium sulfate, filtered, and concentrated. The crude product was separated and purified by preparative TLC (dichloromethane:methanol=10:1, Rf=0.5) and preparative TLC (petroleum ether:ethyl acetate=1:2.5, Rf=0.3) to obtain compound 82 6-methoxy-N-phenylimidazo[1,2-a]pyridin-3-amine (34 mg, 367.21 μmol) as a brown oil.

LC_MS: (ES⁺): m/z 240.0 [M+H]⁺.

¹H NMR (500 MHz, CDCl₃) δ 7.49 (d, J=10.3 Hz, 1H), 7.40 (d, J=1.9 Hz, 1H), 7.27 (s, 1H), 7.21 (dd, J=8.4, 7.5 Hz, 2H), 7.06 (s, 1H), 6.99 (d, J=9.8 Hz, 1H), 6.87 (d, J=7.3 Hz, 1H), 6.68-6.50 (m, 2H), 3.74 (s, 3H).

### Example 9: Synthesis of Compound 83, Compound 84 and Compound 85

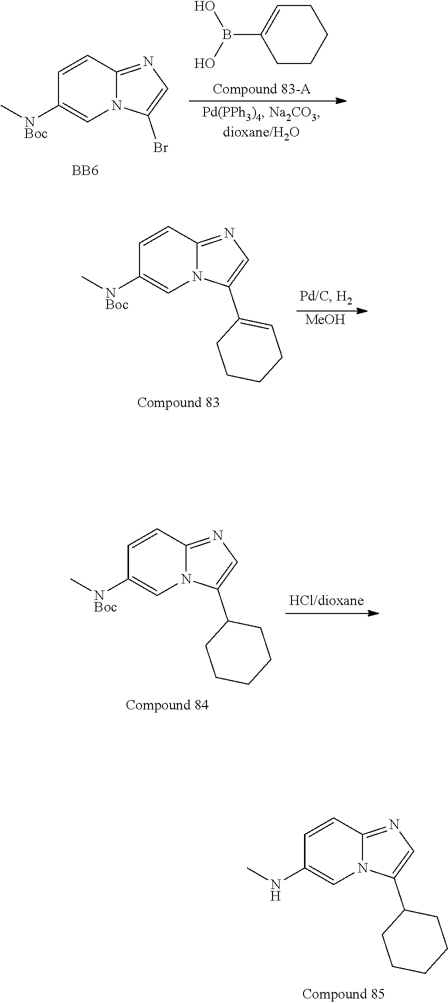

### Step 1: Compound 83 tert-butyl 3-(cyclohex-1-en-1-yl)imidazo[1,2-a]pyridin-6-yl)(methyl)carbamate

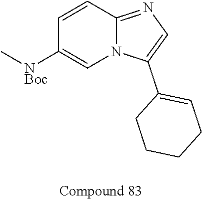 [SMILES: CN(C(=O)OC(C)(C)C)c1ccc2ncc(C3=CCCCC3)n2c1]

A solution containing intermediate BB6 (90 mg, 275.94 μmol) and compound 83-A (244.75 mg, 1.787 mmol) in 1,4-dioxane (6 ml) and water (1 mL) was added with Pd(PPh₃)₄ (16 mg, 13.80 μmol) and sodium carbonate (87.7 mg, 827.73 μmol) under nitrogen atmosphere. The reaction solution was heated to 90° C. under nitrogen protection and stirred for 12 hours of reaction. The reaction was monitored by TLC to be completed. The reaction mixture was extracted with water (10 ml) and ethyl acetate (10 ml×2). The combined organic layers were washed with saturated brine (20 mL), dried over anhydrous sodium sulfate, and concentrated under reduced pressure. The resulting crude product was separated and purified by silica gel column chromatography (eluted with petroleum ether containing 0%-90% ethyl acetate) to obtain compound 83 tert-butyl 3-(cyclohex-1-en-1-yl)imidazo[1,2-a]pyridin-6-yl)(methyl)carbamate (108 mg) as a yellow solid.

LC_MS: (ES⁺): m/z 328.1 [M+H]⁺.

### Step 2: Compound 84 tert-butyl (3-cyclohexylimidazo[1,2-a]pyridin-6-yl)(methyl)carbamate

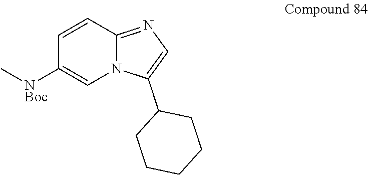 [SMILES: CN(C(=O)OC(C)(C)C)c1ccc2ncc(C3CCCCC3)n2c1]

Compound 83 (88 mg, 268.8 μmol) was dissolved in methanol (5 mL), and then added with palladium carbon (10 mg). The reaction solution was stirred at 20° C. under a hydrogen atmosphere for 48 hours. The reaction was monitored by TLC to be completed. The reaction solution was filtered through diatomite, and the filtrate was concentrated under reduced pressure. The resulting residue was separated and purified by preparative TLC (eluted with petroleum ether containing 50% ethyl acetate) to obtain compound 84 tert-butyl (3-cyclohexylimidazo [1,2-a]pyridin-6-yl)(methyl)carbamate (25 mg).

LC_MS: (ES⁺): m/z 330.1 [M+H]⁺.

### Step 3: Compound 85 3-cyclohexyl-N-methylimidazo[1,2-a]pyridin-6-amine

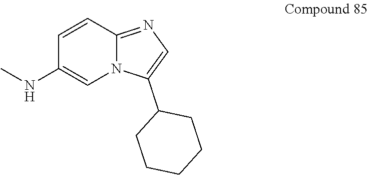 [SMILES: CNc1ccc2ncc(C3CCCCC3)n2c1]

Compound 84 (20 mg, 60.71 μmol) was dissolved in methanol (1 mL), and added with hydrochloric acid in dioxane (1 mL, 4M). The reaction solution was stirred at room temperature for 1 hour. The reaction was monitored by LC-MS to be completed. The reaction solution was concentrated under reduced pressure to obtain brown solid compound 85 3-cyclohexyl-N-methylimidazo[1,2-a]pyridin-6-amine (9.9 mg).

LC_MS: (ES⁺): m/z 230.0 [M+H]⁺.

¹H NMR (400 MHz, DMSO) δ 7.80 (d, J=5.6 Hz, 1H), 7.68 (t, J=6.6 Hz, 1H), 7.62 (s, 1H), 7.47-7.44 (m, 1H), 7.44-7.40 (m, 1H), 3.11 (d, J=11.4 Hz, 1H), 2.74 (s, 2H), 2.01 (t, J=10.1 Hz, 2H), 1.84-1.70 (m, 3H), 1.50 (dd, J=25.1, 12.4 Hz, 2H), 1.41-1.29 (m, 3H).

### Example 10: Synthesis of Compound 86 and Compound 87

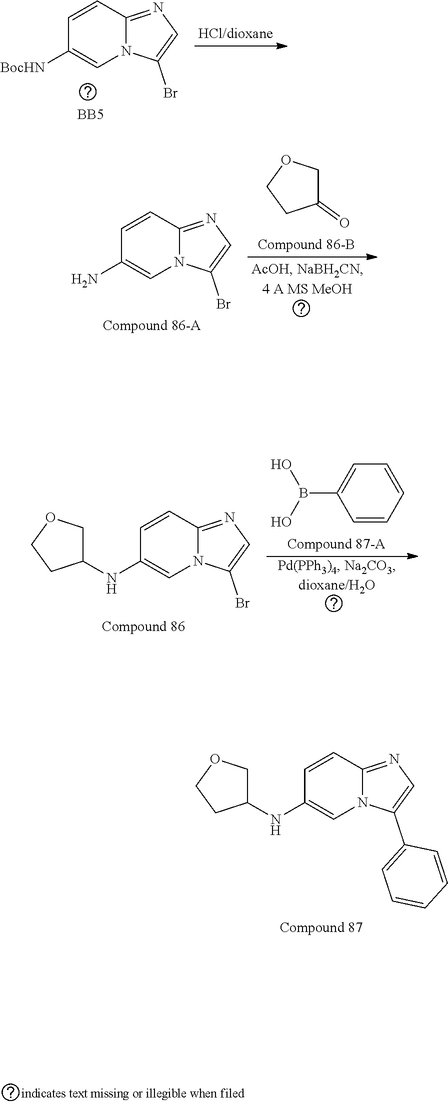

### Step 1: Compound 86-A 3-bromoimidazo[1,2-a]pyridin-6-amine

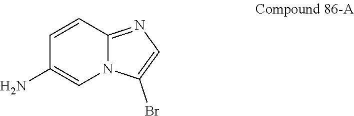 [SMILES: Nc1ccc2ncc(Br)n2c1]

Intermediate BB5 (100 mg, 320.34 μmol) was dissolved in a solution of hydrochloric acid in dioxane (5 mL, 4M). The reaction solution was stirred at room temperature for 1 hour. The reaction was monitored by LC-MS to be completed. The reaction solution was concentrated under reduced pressure to obtain compound 86-A (80 mg) as a white solid.

LC_MS: (ES⁺): m/z 211.9 [M+H]⁺.

### Step 2: Compound 86 3-bromo-N-(tetrahydrofuran-3-yl)imidazo[1,2-a]pyridin-6-amine

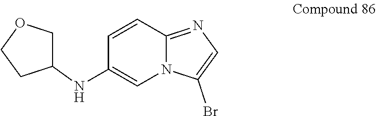 [SMILES: Brc1cnc2ccc(NC3CCOC3)cn12]

Compound 86-A (48 mg, 226.36 μmol) and compound 86-B (29.2 mg, 339.54 μmol) and a drop of acetic acid were dissolved in methanol (1 mL), and added with NaBH₃CN (28.5 mg, 452.72 μmol) and 4A molecular sieve (50 mg). The reaction solution was stirred at 20° C. for 12 hours. The reaction was monitored by TLC to be completed. The reaction solution was filtered, concentrated, and extracted with water (10 ml) and dichloromethane (10 ml×2). The combined organic layers were washed with saturated brine (20 mL), dried over anhydrous sodium sulfate, filtered and concentrated under reduced pressure. The resulting crude product was separated and purified by silica gel column chromatography (eluted with a dichloromethane solution containing 10% methanol) to obtain compound 86 3-bromo-N-(tetrahydrofuran-3-yl)imidazo[1,2-a]pyridin-6-amine (45 mg) as a yellow oil.

LC_MS: (ES⁺): m/z 281.9 [M+H]⁺.

### Step 3: Compound 87 3-phenyl-N-(tetrahydrofuran-3-yl)imidazo[1,2-a]pyridin-6-amine

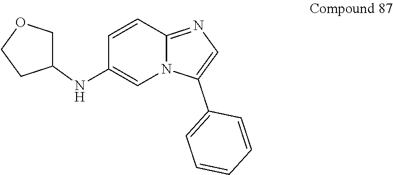 [SMILES: c1ccc(-c2cnc3ccc(NC4CCOC4)cn23)cc1]

A solution containing compound 86 (36 mg, 127.60 μmol) and compound 87-A (31.1 mg, 255.19 μmol) in 1,4-dioxane (3 ml) and water (1 mL) was added with Pd(PPh₃)₄ (6.1 mg, 6.38 μmol) and sodium carbonate (40.6 mg, 382.79 μmol) under nitrogen atmosphere. The reaction solution was heated to 90° C. under nitrogen protection and stirred for 30 minutes of reaction. The reaction was monitored by LCMS to be not completed. The reaction solution was subsequently added with compound 87-A phenylboronic acid (31.1 mg, 255.19 μmol), tetrakis(triphenylphosphine)palladium (6.1 mg, 6.38 μmol) and sodium carbonate (40.6 mg, 382.79 μmol), heated to 100° C. under nitrogen protection and stirred for 1.5 hours of reaction. The reaction mixture was extracted with water (10 ml) and ethyl acetate (10 ml×2). The combined organic layers were washed with saturated brine (20 mL), dried over anhydrous sodium sulfate, filtered and concentrated under reduced pressure. The resulting crude product was separated and purified by silica gel column chromatography (eluted with a dichloromethane solution containing 10% methanol) to obtain compound 87 3-phenyl-N-(tetrahydrofuran-3-yl)imidazo[1,2-a]pyridin-6-amine (11.6 mg) as a yellow oil.

LC_MS: (ES⁺): m/z 280.0 [M+H]⁺.

¹H NMR (500 MHz, MeOD) δ 7.72-7.39 (m, 9H), 7.18 (dd, J=38.7, 9.3 Hz, 1H), 4.06-3.53 (m, 5H), 2.31-2.06 (m, 1H), 1.90 (s, 1H).

### Example 11: Synthesis of Compound 88 and Compound 89

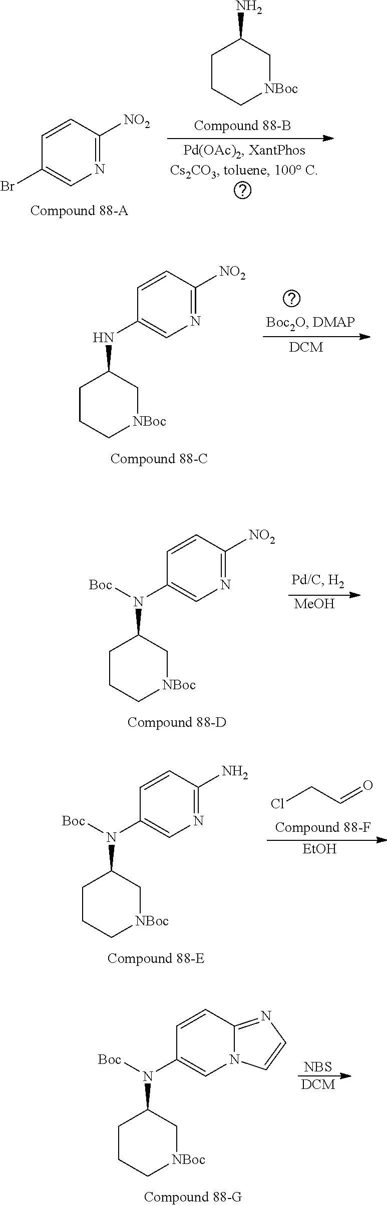

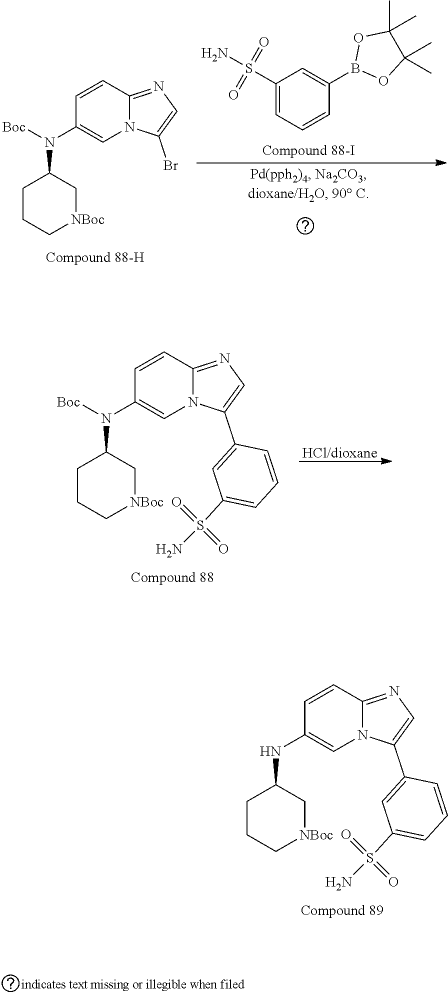

### Step 1: Compound 88-C tert-butyl (R)-3-((6-nitropyridin-3-yl)amino)piperidin-1-carboxylate

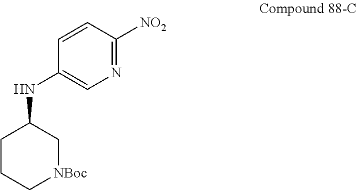 [SMILES: CC(C)(C)OC(=O)N1CCC[C@@H](Nc2ccc([N+](=O)[O-])nc2)C1]

A solution containing compound 88-A (1.0 g, 4.93 mmol) and compound 88-B (986.6 mg, 4.93 mmol) in toluene (20 mL) was added with Pd(OAc)₂ (110.6 mg, 492.62 μmol), XantPhos (285 mg, 492.62 μmol), and cesium carbonate (4.82 g, 14.78 mmol). The reaction solution was stirred under nitrogen at 100° C. for 4 hours of reaction. Compound 88-A (Rf=0.8) was monitored by TLC (PE:EtOAc=1:1) to be completely reacted, and a new spot (Rf=0.3) was generated. The reaction solution was diluted with water (50 mL), and extracted with ethyl acetate (50 mL×2). The combined organic layers were washed with saturated brine (100 mL), dried over anhydrous sodium sulfate, filtered, and concentrated. The obtained residue was separated and purified by silica gel column chromatography (SiO₂, petroleum ether:ethyl acetate=2:1-1:2, Rf=0.3) to obtain yellow solid compound 88-C tert-butyl (R)-3-((6-nitrogen) pyridin-3-yl)amino)piperidin-1-carboxylate (1.37 g).

LC_MS: (ES⁺): m/z 323.0 [M+Na]⁺.

### Step 2: Compound 88-D tert-butyl (R)-3-(((tert-butoxycarbonyl)(6-nitropyridin-3-yl)amino)piperidin-1-formate

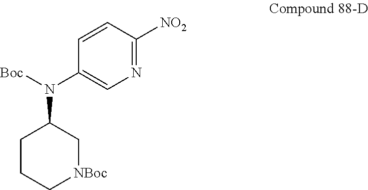 [SMILES: CC(C)(C)OC(=O)N1CCC[C@@H](N(C(=O)OC(C)(C)C)c2ccc([N+](=O)[O-])nc2)C1]

A solution of compound 88-C (1.32 g, 4.09 mmol) and di-tert-butyl dicarbonate (1.07 g, 4.91 mmol) in dichloromethane (20 mL) was added with N,N-diisopropylethylamine (1.59 g, 12.28 mmol) and 4-dimethylaminopyridine (50 mg, 409.47 μmol). The reaction solution was stirred at 40° C. for 12 hours of reaction. TLC (petroleum ether:ethyl acetate=2:1) showed that most of the compound 88-C(Rf=0.2) was completely reacted, and a new spot (Rf=0.6) was generated. The reaction solution was diluted with water (50 mL), and extracted with dichloromethane (50 mL×2). The combined organic layers were washed with saturated brine (100 mL), dried over anhydrous sodium sulfate, filtered, and concentrated. The residue was separated and purified by silica gel column chromatography (SiO₂, petroleum ether:ethyl acetate=4:1-2:1) to obtain yellow solid compound 88-D tert-butyl (R)-3-(((tert-butoxycarbonyl)(6-nitropyridin-3-yl)amino)piperidin-1-formate (1.4 g).

LC_MS: (ES⁺): m/z 445.0 [M+Na]⁺.

¹H NMR (400 MHz, MeOD) δ 8.43 (d, J=2.3 Hz, 1H), 8.34 (dd, J=8.6, 0.4 Hz, 1H), 8.01 (dd, J=8.6, 2.5 Hz, 1H), 4.36-4.23 (m, 1H), 4.04 (t, J=11.1 Hz, 1H), 3.94 (d, J=13.1 Hz, 1H), 2.81 (s, 1H), 2.56 (s, 1H), 2.06-1.85 (m, 1H), 1.81-1.62 (m, 1H), 1.53 (ddd, J=16.8, 8.5, 4.0 Hz, 1H), 1.46 (s, 8H), 1.41 (s, 9H).

### Step 3: Compound 88-E tert-butyl (R)-3-((6-aminopyridin-3-yl)(tert-butoxycarbonyl)amino)piperidin-1-formate

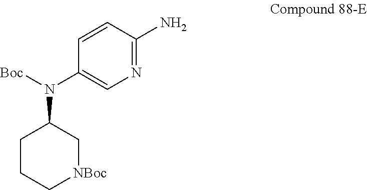 [SMILES: CC(C)(C)OC(=O)N1CCC[C@@H](N(C(=O)OC(C)(C)C)c2ccc(N)nc2)C1]

A solution of compound 88-D (1.3 g, 3.08 mmol) in tetrahydrofuran (20 mL) was added with Pd/C (130 mg). The reaction solution was stirred under a hydrogen balloon at 20° C. for 12 hours of reaction. The reaction was monitored by LCMS to be completed. The reaction solution was filtered through diatomite. The filtrate was concentrated. The residue was used directly in the next reaction without purification. A brown solid compound 88-E tert-butyl (R)-3-((6-aminopyridin-3-yl)(tert-butoxycarbonyl)amino)piperidin-1-formate (1.2 g) was obtained.

LC_MS: (ES⁺): m/z 393.1 [M+H]⁺.

### Step 4: Compound 88-G tert-butyl (R)-3-(((tert-butoxycarbonyl)(imidazo[1,2-a]pyridin-6-yl)amino)amino)piperidin-1-carboxylate

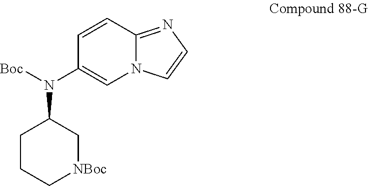 [SMILES: CC(C)(C)OC(=O)N1CCC[C@@H](N(C(=O)OC(C)(C)C)c2ccc3nccn3c2)C1]

A solution containing compound 88-E (300 mg, 764.33 μmol) and compound 88-F (300 mg, 1.53 mmol, 40% wt aqueous solution) in ethanol (10 mL) was stirred at 80° C. for 15 hours of reaction. TLC (petroleum ether:ethyl acetate=1:1) monitoring showed that compound 88-E (Rf=0.6) was reacted completely, and a new spot (Rf=0.3) was generated. The reaction solution was concentrated, then diluted with ethyl acetate (30 mL), and adjusted to pH=9 with sodium bicarbonate solution. The organic layer was collected, washed with saturated brine (50 mL), dried over anhydrous sodium sulfate, filtered, and concentrated. The residue was separated and purified by silica gel column chromatography (SiO₂, petroleum ether:ethyl acetate=1:1-0:1, Rf=0.3) to obtain a brown oil compound 88-G tert-butyl (R)-3-((tert-butoxycarbonyl)(imidazo[1,2-a]pyridin-6-yl)amino)amino)piperidin-1-carboxylate (230 mg).

LC_MS: (ES): m/z 417.1 [M+H]⁺.

### Step 5: Compound 88-H tert-butyl(R)-3-((3-bromoimidazo[1,2-a]pyridin-6-yl)(tert-butoxycarbonyl)amino)piperidin-1-formate

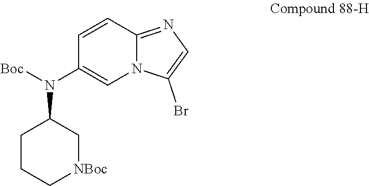 [SMILES: CC(C)(C)OC(=O)N1CCC[C@@H](N(C(=O)OC(C)(C)C)c2ccc3ncc(Br)n3c2)C1]

A solution of compound 88-G tert-butyl (R)-3-((tert-butoxycarbonyl)(imidazo[1,2-a]pyridin-6-yl)amino)amino)piperidin-1-carboxylate (200 mg, 0.48 mmol) in dichloromethane (6 ml) was added with N-bromosuccinimide (85.57 mg, 0.48 mmol). The reaction solution was stirred at room temperature for 1 hour of reaction. The reaction was monitored by TLC to be completed. The reaction mixture was concentrated under reduced pressure, and the resulting residue was subjected to silica gel column chromatography (eluted with petroleum ether containing 0%-50% ethyl acetate) to obtain yellow solid compound 88-H tert-butyl(R)-3-((3-bromoimidazo[1,2-a]pyridin-6-yl) (tert-butoxycarbonyl)amino)piperidin-1-formate (200 mg).

LC_MS: (ES⁺): m/z 495.0 [M+H]⁺.

### Step 6: Compound 88 tert-butyl (R)-3-((tert-butoxycarbonyl)(3-(3-sulfamoylphenyl)imidazo[1,2-a]pyridin-6-yl)amino)piperidin-1-formate

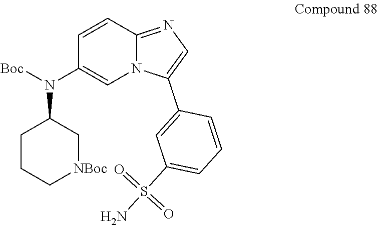 [SMILES: CC(C)(C)OC(=O)N1CCC[C@@H](N(C(=O)OC(C)(C)C)c2ccc3ncc(-c4cccc(S(N)(=O)=O)c4)n3c2)C1]

A solution containing compound 88-H (100 mg, 0.202 mmol), compound 88-I (142.88 mg, 0.504 mmol) and sodium carbonate (64.23 mg, 0.606 mmol) in 1,4-dioxane (3 ml) and water (1 ml) was added with Pd(PPh₃)₄ (16.34 mg, 0.0141 mmol) under nitrogen at room temperature. The reaction mixture was replaced with nitrogen three times, and stirred at 90° C. for 6 hours of reaction. The reaction was monitored by TLC to be completed. The reaction mixture was extracted with water and ethyl acetate. The combined organic layers were dried over anhydrous sodium sulfate and concentrated under reduced pressure. The resulting crude product was separated and purified by preparative TLC (eluted with dichloromethane containing 4.76% methanol) to obtain yellow solid compound 88 tert-butyl (R)-3-((tert-butoxycarbonyl)(3-(3-sulfamoylphenyl)imidazo[1,2-a]pyridin-6-yl)amino)piperidin-1-formate (51 mg).

LC_MS: (ES⁺): m/z 572.1 [M+H]⁺.

### Step 7: Compound 89 (R)-3-(6-(piperidin-3-ylamino)imidazo[1,2-a]pyridin-3-yl)benzenesulfonamide

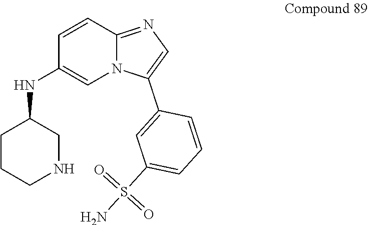 [SMILES: NS(=O)(=O)c1cccc(-c2cnc3ccc(N[C@@H]4CCCNC4)cn23)c1]

A solution of compound 88 (50 mg, 0.0848 mmol) in methanol (2 ml) was added with a hydrochloric acid/dioxane (2 ml, 4.0 mol/L) solution. The reaction mixture was stirred at room temperature for 6 hours. The reaction was monitored by TLC to be completed. The reaction solution was concentrated under reduced pressure to obtain yellow solid compound 89 (R)-3-(6-(piperidin-3-ylamino)imidazo[1,2-a]pyridin-3-yl)benzenesulfonamide (39.5 mg).

LC_MS: (ES⁺): m/z 372.0 [M+H]⁺.

¹H NMR (400 MHz, DMSO) δ 8.98 (s, 2H), 8.29 (s, 1H), 8.13 (s, 1H), 7.97 (dd, J=17.8, 7.9 Hz, 2H), 7.83 (dd, J=15.6, 8.5 Hz, 2H), 7.74 (s, 1H), 7.58 (d, J=5.0 Hz, 3H), 6.62 (s, 1H), 3.66 (dd, J=13.7, 5.3 Hz, 2H), 3.14 (s, 1H), 2.87 (s, 1H), 2.69 (d, J=9.8 Hz, 1H), 1.94-1.81 (m, 2H), 1.72 (d, J=10.4 Hz, 1H), 1.52 (d, J=10.1 Hz, 1H).

The following compounds were synthesized referring to the synthesis of compound 89:

### Synthetic Route of Compound 90

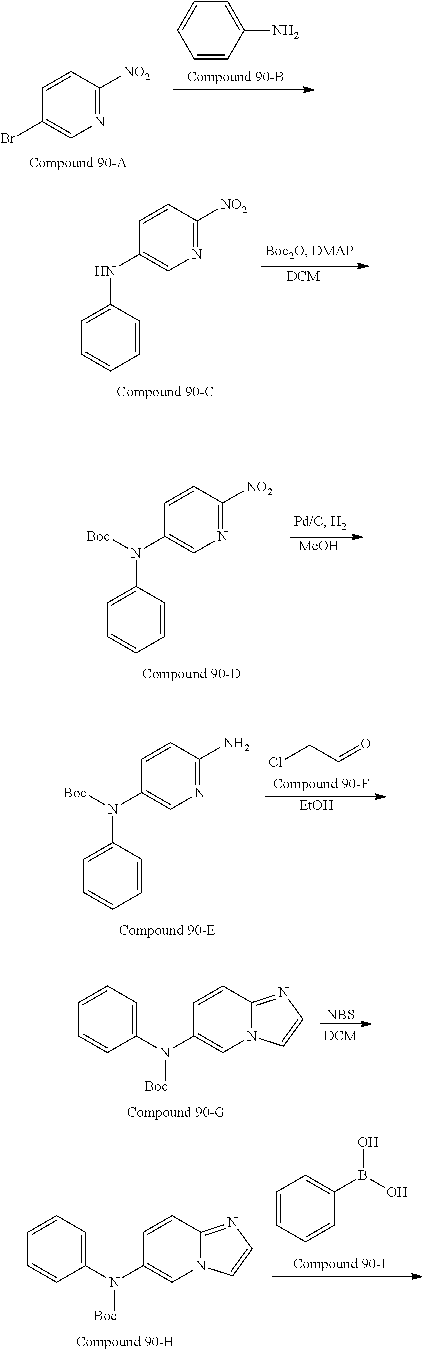

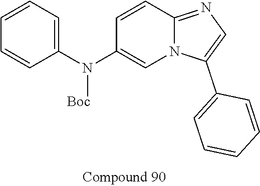 [SMILES: CC(C)(C)OC(=O)N(c1ccccc1)c1ccc2ncc(-c3ccccc3)n2c1]

Step 1: The synthesis of compound 90-C was performed referring to the first step of the synthesis method of compound 89.

Step 2: The synthesis of compound 90-D was performed referring to the second step of the synthesis method of compound 89.

Step 3: The synthesis of compound 90-E was performed referring to the third step of the synthesis method of compound 89.

Step 4: The synthesis of compound 90-G was performed referring to the fourth step of the synthesis method of compound 89.

Step 5: The synthesis of compound 90-H was performed referring to the fifth step of the synthesis method of compound 89.

Step 6: The synthesis of compound 90 was performed referring to the sixth step of the synthesis method of compound 89.

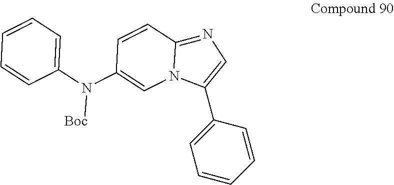 [SMILES: CC(C)(C)OC(=O)N(c1ccccc1)c1ccc2ncc(-c3ccccc3)n2c1]

LC_MS: (ES⁺): m/z 386.2 [M+H]⁺.

### Example 12: Synthesis of Compound 91

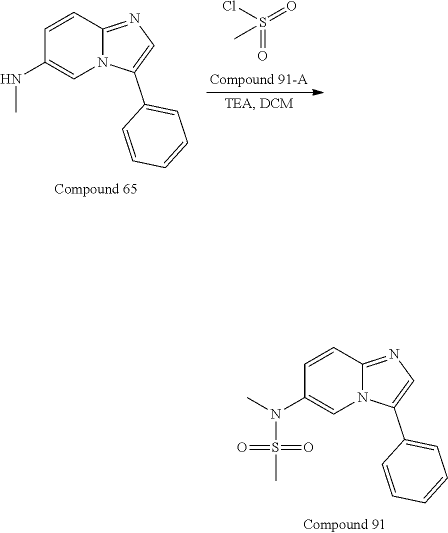

### Step 1: Compound 91 N-methyl-N-(3-phenylimidazo[1,2-a]pyridin-6-yl)methanesulfonamide

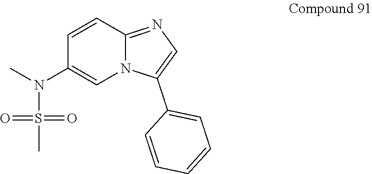 [SMILES: CN(c1ccc2ncc(-c3ccccc3)n2c1)S(C)(=O)=O]

A solution containing compound 65 N-methyl-3-phenylimidazo[1,2-a]pyridin-6-amine (65 mg, 0.291 mmol) and triethylamine (32.32 mg, 0.32 mmol) in dichloromethane (4 ml) was added with compound 91-A methylsulfonyl chloride (36.65 mg, 0.32 mmol) under nitrogen at 0° C. The reaction mixture was warmed from 0° C. to room temperature and stirred for 14 hours of reaction. The reaction was monitored by TLC to be completed. The reaction solution was extracted with water and dichloromethane. The combined organic layers were dried over anhydrous sodium sulfate and concentrated under reduced pressure. The resulting crude product was separated and purified by preparative TLC to obtain white solid compound 91 N-methyl-N-(3-phenylimidazo[1,2-a]pyridin-6-yl)methanesulfonamide (15 mg).

LC_MS: (ES⁺): m/z 302.0 [M+H]⁺.

¹H NMR (500 MHz, DMSO) δ 8.62 (s, 1H), 7.94 (s, 1H), 7.78 (s, 3H), 7.68 (s, 2H), 7.56 (s, 2H), 3.38 (s, 3H), 3.15 (s, 3H).

### Example 13: Synthesis of Compound 92

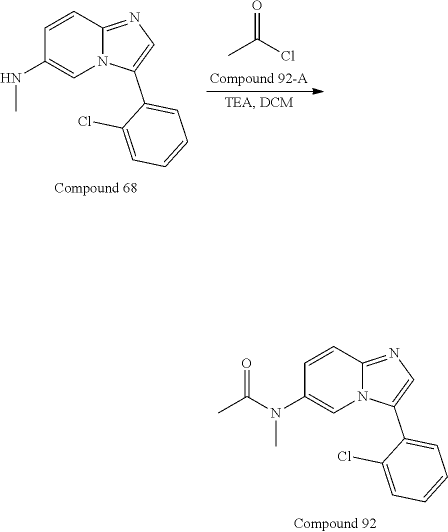

### Step 1: Compound 92 N-(3-(2-chlorophenyl)imidazo[1,2-a]pyridin-6-yl)-N-methylacetamide

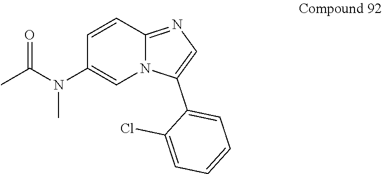 [SMILES: CC(=O)N(C)c1ccc2ncc(-c3ccccc3Cl)n2c1]

A solution containing compound 68 3-(2-chlorophenyl)-N-methylimidazo[1,2-a]pyridin-6-amine (70 mg, 0.271 mmol) and triethylamine (30.3 mg, 0.3 mmol) in dichloromethane (4 ml) was added with compound 92-A acetyl chloride (24.45 mg, 0.3 mmol) under nitrogen at 0° C. The reaction mixture was warmed from 0° C. to room temperature and stirred for 4 hours of reaction. The reaction was monitored by TLC to be completed. The reaction solution was extracted with water and dichloromethane. The combined organic layers were dried over anhydrous sodium sulfate and concentrated under reduced pressure. The resulting crude product was separated and purified by preparative TLC to obtain brown solid compound 92 N-(3-(2-chlorophenyl)imidazo[1,2-a]pyridin-6-yl)-N-methylacetamide (30 mg).

LC_MS: (ES⁺): m/z 300.0 [M+H]⁺.

¹H NMR (500 MHz, DMSO) δ 8.24 (s, 1H), 7.79-7.74 (m, 2H), 7.69 (dd, J=7.9, 1.2 Hz, 1H), 7.61 (d, J=7.3 Hz, 1H), 7.57 (dd, J=7.5, 1.9 Hz, 1H), 7.54 (dd, J=5.3, 1.8 Hz, 1H), 7.51 (dd, J=7.4, 1.4 Hz, 1H), 7.38 (d, J=9.7 Hz, 1H), 3.10 (s, 3H), 1.81 (s, 3H).

The following compounds were synthesized referring to the synthesis of compound 92:

### Synthesis of Compound 93

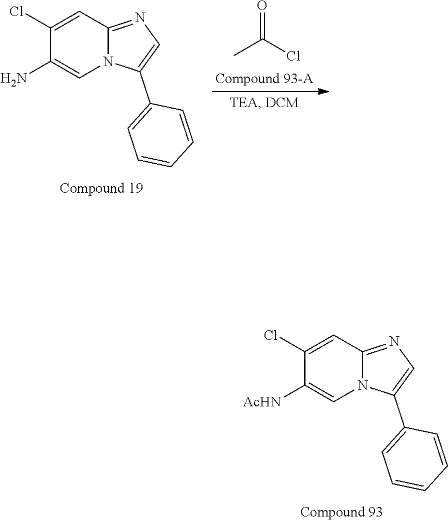

Step 1: The synthesis of compound 93 was performed referring to the first step of the synthesis method of compound 92.

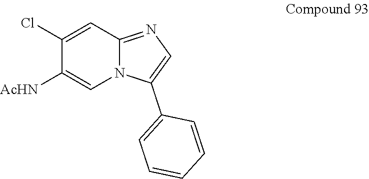 [SMILES: CC(=O)Nc1cn2c(-c3ccccc3)cnc2cc1Cl]

LC_MS: (ES⁺): m/z 286.0 [M+H]⁺.

### Example 14: Synthesis of Compound 94

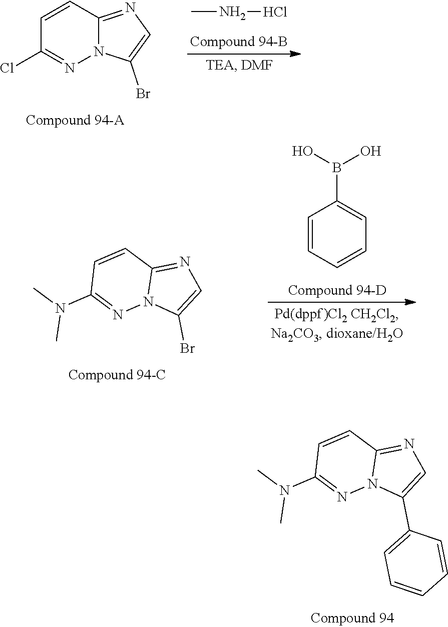

### Step 1: Compound 94-C 3-bromo-N,N-dimethylimidazo[1,2-b]pyridazin-6-amine

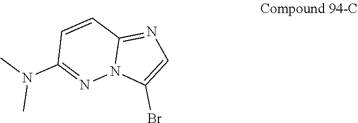

A solution containing compound 94-A (40 mg, 0.172 mmol), compound 94-B methylamine hydrochloride (17.42 mg, 0.258 mmol) and triethylamine (52.21 mg, 0.516 mmol) in N,N-dimethylformamide (2 ml) was stirred at 110° C. for 48 hours of reaction. The reaction was monitored by TLC to be completed. The reaction solution was extracted with water and ethyl acetate. The combined organic layers were washed with saturated brine, dried over anhydrous sodium sulfate, and concentrated under reduced pressure. The resulting crude product was separated and purified by preparative TLC (eluted with dichloromethane containing 3.22% methanol) to obtain brown solid compound 94-C 3-bromo-N,N-dimethylimidazo[1,2-b]pyridazin-6-amine (14 mg).

LC_MS: (ES⁺): m/z 240.9 [M+H]⁺.

### Step 2: Compound 94 N,N-dimethyl-3-phenylimidazo[1,2-b]pyridazin-6-amine

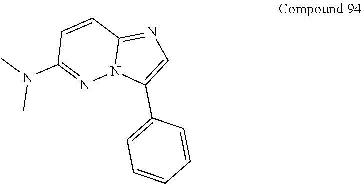 [SMILES: CN(C)c1ccc2ncc(-c3ccccc3)n2n1]

A solution containing compound 94-C 3-bromo-N,N-dimethylimidazo[1,2-b]pyridazin-6-amine (14 mg, 0.058 mmol), compound 94-D phenylboronic acid (7.79 mg, 0.069 mmol), and saturated sodium carbonate solution (0.5 ml) in 1,4-dioxane (1 ml) was added with Pd(dppf)Cl₂—CH₂Cl₂ (4.74 mg, 0.0058 mmol) under a nitrogen atmosphere at room temperature. The reaction mixture was replaced with nitrogen three times, and stirred at 80° C. for 14 hours of reaction. The reaction was monitored by TLC to be completed. The reaction mixture was cooled to room temperature, and extracted with water and ethyl acetate. The combined organic layers were dried over anhydrous sodium sulfate and concentrated under reduced pressure. The resulting crude product was separated and purified by preparative TLC (eluted with dichloromethane containing 4.76% methanol) to obtain white solid compound 94 N,N-dimethyl-3-phenylimidazo[1,2-b]pyridazin-6-amine (6 mg).

LC_MS: (ES⁺): m/z 239.1 [M+H]⁺.

¹H NMR (400 MHz, DMSO) δ 8.18 (dd, J=8.4, 1.1 Hz, 2H), 7.94 (s, 1H), 7.89 (d, J=10.0 Hz, 1H), 7.46 (dd, J=10.7, 4.8 Hz, 2H), 7.31 (d, J=7.4 Hz, 1H), 7.11 (d, J=10.0 Hz, 1H), 3.09 (s, 6H).

### Example 15: Synthesis of Compound 95

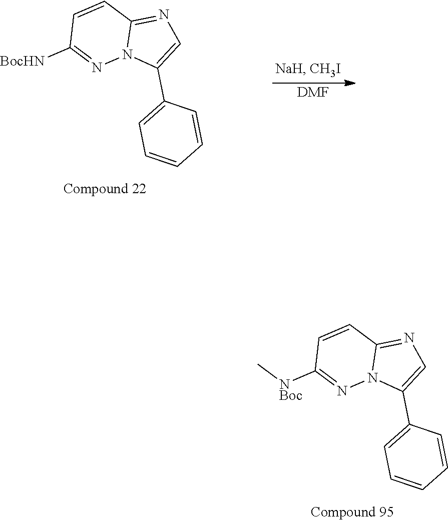

### Step 1: Compound 95 tert-butyl methyl(3-phenylimidazo[1,2-b]pyridazin-6-yl) carbamate

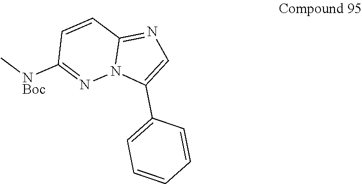 [SMILES: CN(C(=O)OC(C)(C)C)c1ccc2ncc(-c3ccccc3)n2n1]

A solution of compound 22 (18.5 mg, 0.0597 mmol) in N,N-dimethylformamide (1 ml) was added with sodium hydride (2.39 mg, 0.0597 mmol, 60% dispersed in mineral oil) and methyl iodide (16.89 mg, 0.119 mmol) at 0° C. under nitrogen. The mixed reactants were heated from 0° C. to room temperature and stirred for 14 hours of reaction. The reaction was monitored by TLC to be completed. The reaction solution was extracted with water and ethyl acetate. The combined organic layers were washed with saturated brine, dried over anhydrous sodium sulfate, and concentrated under reduced pressure. The resulting crude product was separated and purified by preparative TLC (eluted with dichloromethane containing 4.76% methanol) to obtain light yellow solid compound 95 tert-butyl methyl (3-phenylimidazo[1,2-b]pyridazin-6-yl) carbamate (7 mg).

LC_MS: (ES⁺): m/z 325.1 [M+H]⁺.

### Example 16: Synthesis of Compound 96

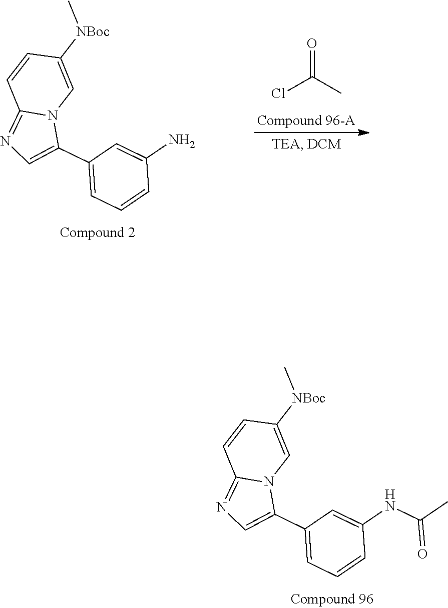

### Step 1: Compound 96 tert-butyl (3-(3-acetylaminophenyl)imidazo[1,2-a]pyridin-6-yl)(methyl)carbamate

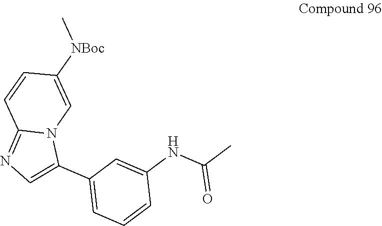 [SMILES: CC(=O)Nc1cccc(-c2cnc3ccc(N(C)C(=O)OC(C)(C)C)cn23)c1]

A solution containing compound 2 (20 mg, 0.0591 mmol) and pyridine (9.35 mg, 0.118 mmol) in dichloromethane (4 ml) was added with compound 96-A acetyl chloride (4.64 mg, 0.0591 mmol) under nitrogen at 0° C. The reaction mixture was heated from 0° C. to room temperature and stirred for 4 hours of reaction. The reaction was monitored by TLC to be completed. The reaction solution was extracted with water and dichloromethane. The combined organic layers were dried over anhydrous sodium sulfate and concentrated under reduced pressure. The resulting residue was separated and purified by preparative TLC (eluted with dichloromethane containing 4.76% methanol) to obtain compound 96 tert-butyl 3-(3-acetylaminophenyl)imidazo[1,2-a]pyridin-6-yl)(methyl)carbamate (19 mg) as a yellow solid.

LC_MS: (ES⁺): m/z 339.2 [M+H]⁺.

¹H NMR (400 MHz, DMSO) δ 10.11 (s, 1H), 8.48 (s, 1H), 7.88 (s, 1H), 7.75 (s, 1H), 7.68 (d, J=9.2 Hz, 1H), 7.57 (d, J=7.8 Hz, 1H), 7.46 (t, J=7.8 Hz, 1H), 7.36 (d, J=9.5 Hz, 1H), 7.30 (d, J=7.5 Hz, 1H), 3.19 (s, 3H), 2.05 (s, 3H), 1.35 (s, 9H).

### Example 17: Synthesis of Compound 97

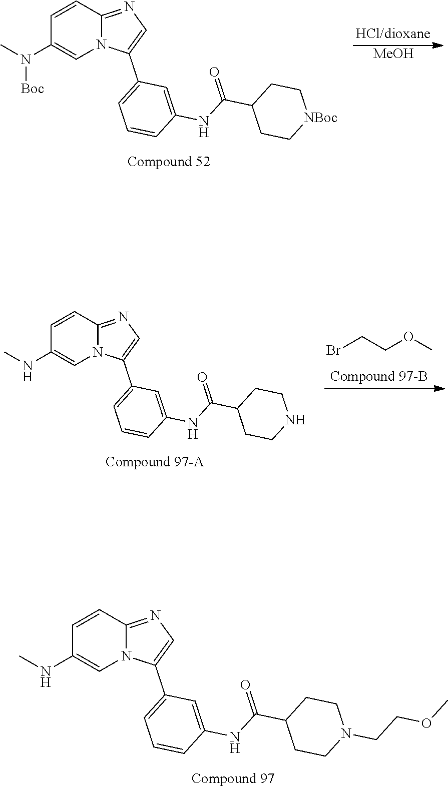

### Step 1: Compound 97-A N-(3-(6-(methylamino)imidazo[1,2-a]pyridin-3-yl)phenyl)piperidin-4-carboxamide

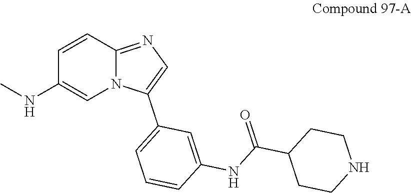 [SMILES: CNc1ccc2ncc(-c3cccc(NC(=O)C4CCNCC4)c3)n2c1]

Compound 52 (70 mg, 0.127 mmol) and hydrochloric acid/dioxane (1 ml) were added to a methanol (1 ml) solution. The reaction solution was stirred at room temperature for 6 hours of reaction. The reaction was monitored by TLC to be completed. The reaction solution was concentrated under reduced pressure to obtain a brown solid crude compound 97-A N-(3-(6-(methylamino)imidazo[1,2-a]pyridin-3-yl)phenyl)piperidin-4-carboxamide (50 mg).

LC_MS: (ES⁺): m/z 350.2 [M+H]⁺.

### Step 2: Compound 97 1-(2-methoxyethyl)-N-(3-(6-(methylamino)imidazo[1,2-a]pyridin-3-yl)phenyl)piperidin-4-carboxamide

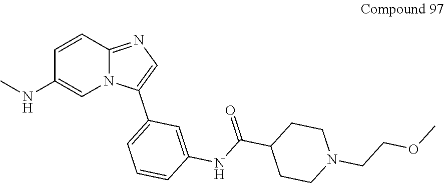

A solution containing compound 97-A (20 mg, 0.0573 mmol), compound 97-B (7.96 mg, 0.0573 mmol) and potassium carbonate (31.63 mg, 0.23 mmol) in N,N-dimethylformamide (1 ml) was stirred at 70° C. for 16 hours of reaction. The reaction was monitored by TLC to be completed. The reaction mixture was extracted with ethyl acetate. The combined organic layers were washed with saturated brine, dried over anhydrous sodium sulfate, and concentrated under reduced pressure. The obtained crude product was separated and purified by preparative TLC (eluted with dichloromethane containing 11.1% methanol) to obtain a brown gum compound 97 1-(2-methoxyethyl)-N-(3-(6-(methylamino)imidazo[1,2-a]pyridin-3-yl)phenyl)piperidin-4-carboxamide (3.2 mg).

LC_MS: (ES⁺): m/z 408.2 [M+H]⁺.

### Example 18: Synthesis of Compound 98

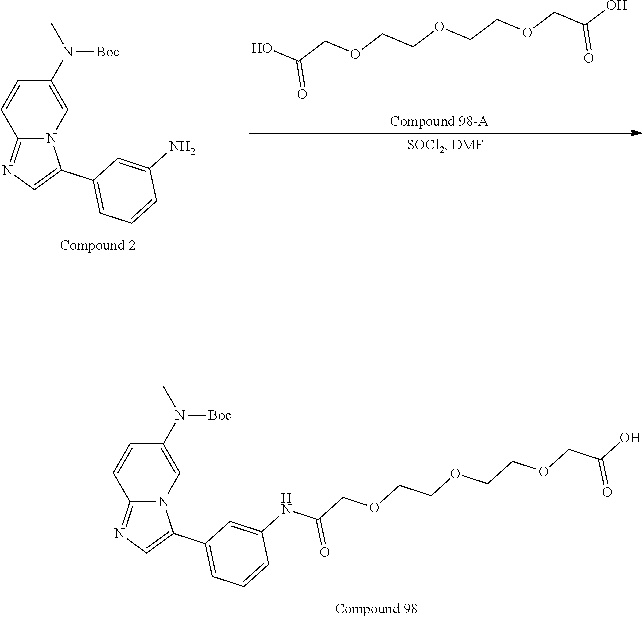 [SMILES: CN(C(=O)OC(C)(C)C)c1ccc2ncc(-c3cccc(N)c3)n2c1 ; O=C(O)COCCOCCOCC(=O)O ; CN(C(=O)OC(C)(C)C)c1ccc2ncc(-c3cccc(NC(=O)COCCOCCOCC(=O)O)c3)n2c1]

### Step 1: Compound 98 2-(2-(2-(2-((3-(6-((tert-butoxycarbonyl))(methyl)amino)imidazo[1,2-a]pyridin-3-yl)phenyl)amino)-2-oxoethoxy)ethoxy)ethoxy)acetic acid

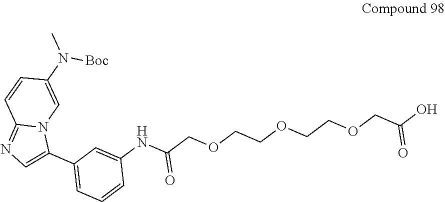 [SMILES: CN(C(=O)OC(C)(C)C)c1ccc2ncc(-c3cccc(NC(=O)COCCOCCOCC(=O)O)c3)n2c1]

A solution of compound 98-A (100 mg, 0.45 mmol) in thionyl chloride (3 ml) was stirred at 60° C. for 3.5 hours of reaction. The reaction solution was concentrated under reduced pressure, and the residue was dissolved in dichloromethane (2 mL). The above reaction solution was added with a dichloromethane (1 mL) solution containing compound 2 (50.76 mg, 0.15 mmol) and triethylamine (75.89 mg, 0.75 mmol) at 0° C., stirred at 0° C. for 3 hours of reaction. The reaction was monitored by TLC to be completed. The reaction solution was extracted with water (10 ml) and dichloromethane (10 ml×3). The organic layer was dried over anhydrous sodium sulfate and concentrated under reduced pressure. The crude product obtained was subjected to Prep-TLC (eluted with dichloromethane containing 12.5% methanol) to obtain yellow solid compound 98 2-(2-(2-(2-((3-(6-((tert-butoxycarbonyl))(methyl)amino)imidazo[1,2-a]pyridin-3-yl)phenyl)amino)-2-oxoethoxy)ethoxy)ethoxy)acetic acid (60 mg).

LC_MS: (ES⁺): m/z 543.3 [M+H]⁺.

¹H NMR (400 MHz, DMSO) δ 10.21 (s, 1H), 8.49 (s, 1H), 7.95 (s, 1H), 7.79-7.70 (m, 2H), 7.63 (d, J=9.4 Hz, 1H), 7.47 (t, J=7.9 Hz, 1H), 7.31 (dd, J=17.5, 7.8 Hz, 2H), 4.15 (s, 2H), 3.65 (d, J=6.2 Hz, 4H), 3.62-3.57 (m, 2H), 3.53 (s, 4H), 3.19 (s, 3H), 1.34 (s, 9H).

### Example 19: Synthesis of Compound 99

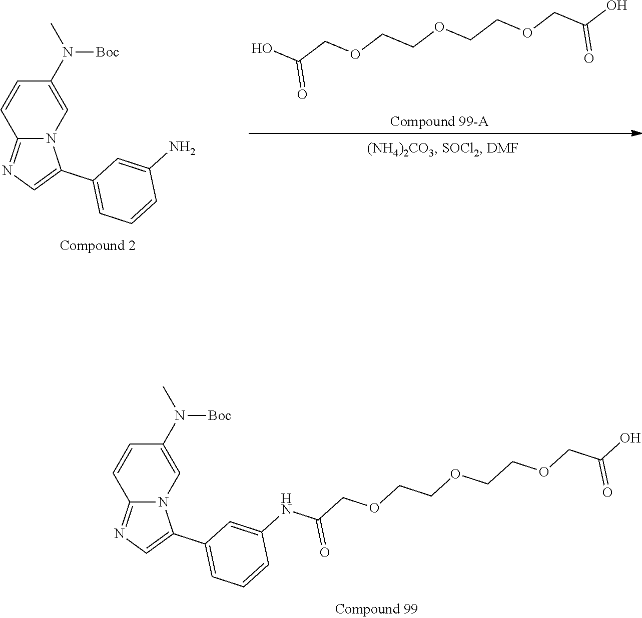 [SMILES: CN(C(=O)OC(C)(C)C)c1ccc2ncc(-c3cccc(N)c3)n2c1 ; O=C(O)COCCOCCOCC(=O)O ; CN(C(=O)OC(C)(C)C)c1ccc2ncc(-c3cccc(NC(=O)COCCOCCOCC(=O)O)c3)n2c1]

### Step 1: Compound 99 tert-butyl (3-(3-(2-(2-(2-(2-amino-2-oxoethoxy)ethoxy)ethoxy) acetamido)phenyl)imidazolin[1,2-a]pyridin-6-yl)(methyl)carbamate

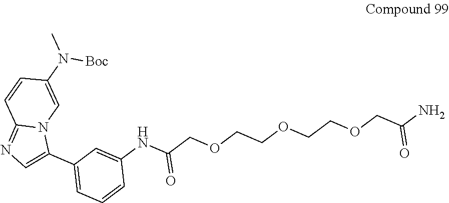 [SMILES: CN(C(=O)OC(C)(C)C)c1ccc2ncc(-c3cccc(NC(=O)COCCOCCOCC(N)=O)c3)n2c1]

A solution of compound 99-A (49.28 mg, 0.222 mmol) in thionyl chloride (1 ml) was stirred at 60° C. for 5 hours of reaction. The reaction solution was concentrated under reduced pressure, and the residue was dissolved in dichloromethane (2 mL). The above reaction solution was added with a dichloromethane (1 mL) solution containing compound 2 (25 mg, 0.074 mmol) and triethylamine (37.38 mg, 0.369 mmol) at 0° C., and stirred at 0° C. for 3 hours of reaction. Finally, the reaction mixture was added with ammonium carbonate (14.22 mg, 0.148 mmol), and stirred at room temperature for 14 hours of reaction. The reaction was monitored by TLC to be completed. The reaction solution was extracted with water (10 ml) and dichloromethane (10 ml×3). The organic layer was dried over anhydrous sodium sulfate and concentrated under reduced pressure. The crude product obtained was subjected to Prep-TLC (eluted with dichloromethane containing 9.1% methanol) to obtain yellow solid compound 99 tert-butyl (3-(3-(2-(2-(2-(2-amino-2-oxoethoxy)ethoxy)ethoxy)ethoxy)acetamido)phenyl)imidazolin[1,2-a]pyridin-6-yl)(methyl)carbamate (9 mg).

LC_MS: (ES⁺): m/z 542.3 [M+H]⁺.

### Example 20: Synthesis of Compound 100, Compound 101 and Compound 102

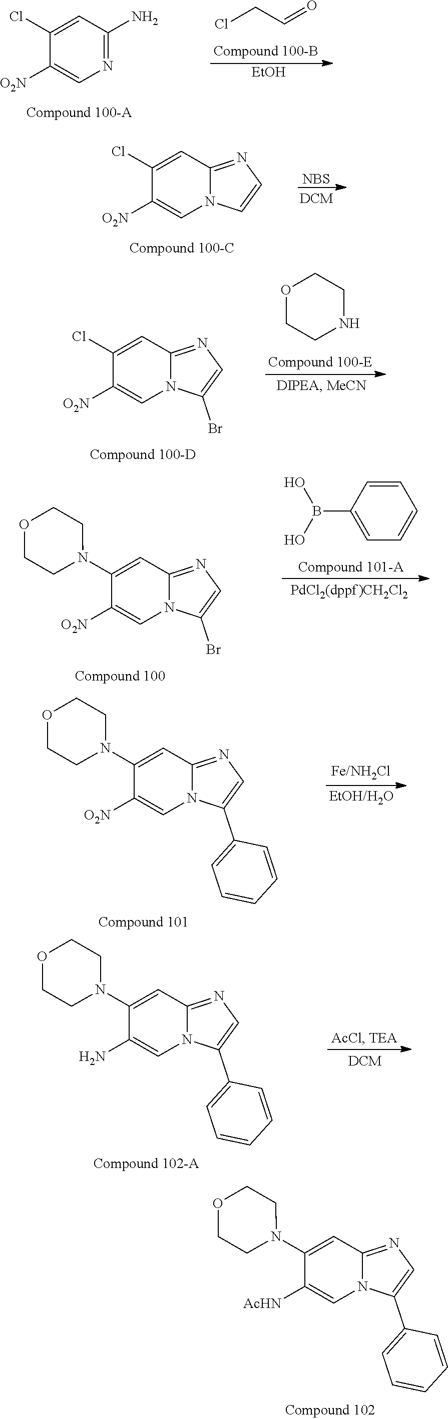

Step 1: The synthesis of compound 100-C was performed referring to the third step of the synthesis process of intermediate BB5.

Step 2: The synthesis of compound 100-D was performed referring to the fourth step of the synthesis process of intermediate BB5.

Step 3: Compound 100 4-(3-bromo-6-nitroimidazo[1,2-a]pyridin-7-yl)morpholine

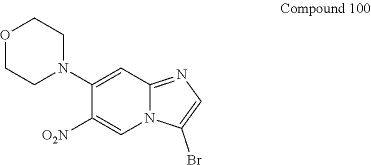 [SMILES: O=[N+]([O-])c1cn2c(Br)cnc2cc1N1CCOCC1]

A solution containing compound 100-D (150 mg, 542.55 μmol) and compound 100-E (70.9 mg) in acetonitrile (3 mL) was added with DIPEA (140.2 mg). The reaction solution was stirred at 20° C. for 12 hours of reaction. The product Ms was detected by LCMS. The reaction solution was diluted with water (20 mL), and extracted with ethyl acetate (20 mL×2). The organic layers were combined, washed with saturated brine (20 mL), dried over anhydrous sodium sulfate, filtered and concentrated. The residue was separated and purified by silica gel column chromatography (eluted with petroleum ether solution containing 50% ethyl acetate) to obtain yellow solid compound 100 4-(3-bromo-6-nitroimidazo[1,2-a]pyridin-7-yl)morpholine (45 mg).

LC_MS: (ES⁺): m/z 327.0 [M+H]⁺.

Step 4: The synthesis of compound 101 was performed referring to the first step of the synthesis process of compound 2.

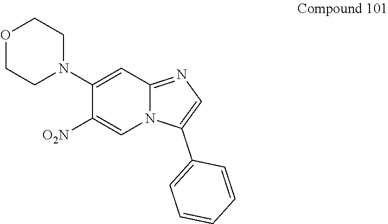 [SMILES: O=[N+]([O-])c1cn2c(-c3ccccc3)cnc2cc1N1CCOCC1]

LC_MS: (ES⁺): m/z 325.1 [M+H]⁺.

### Step 5: Compound 102-A 7-morpholin-3-phenylimidazo[1,2-a]pyridin-6-amine

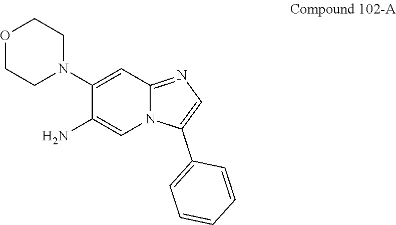 [SMILES: Nc1cn2c(-c3ccccc3)cnc2cc1N1CCOCC1]

A mixed solution of compound 101 (14 mg, 43.16 μmol) in ethanol (2 mL) and water (0.2 mL) was added with NH₄Cl (11.5 mg, 215.82 μmol) and iron powder (12.1 mg, 215.82 mol). The reaction solution was stirred at 20° C. for 12 hours of reaction. The reaction was monitored by LCMS to be completed. The reaction solution was filtered through diatomite, and the mother liquor was concentrated. The residue was diluted with water (10 mL) and extracted with ethyl acetate (10 mL×2). The organic layers were combined, washed with saturated brine (20 mL), dried over anhydrous sodium sulfate, filtered and concentrated. The residue was used directly in the next step without further purification. A yellow oil compound 102-A 7-morpholin-3-phenylimidazo[1,2-a]pyridin-6-amine (12.7 mg) was obtained.

LC_MS: (ES⁺): m/z 295.1 [M+H]⁺.

### Step 6: Compound 102 N-(7-morpholin-3-phenylimidazol[1,2-a]pyridin-6-yl)acetamide

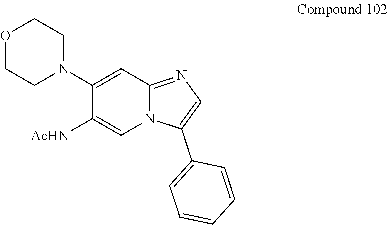 [SMILES: CC(=O)Nc1cn2c(-c3ccccc3)cnc2cc1N1CCOCC1]

A solution containing compound 102-A (12.7 mg, 43.14 μmol) and triethylamine (129.43 μmol) in dichloromethane (1 mL) was added with acetyl chloride (4.1 mg, 51.77 μmol). The reaction solution was stirred at 20° C. for 12 hours of reaction. The product Ms was detected by LCMS. The reaction solution was concentrated. The residue was separated and purified by Prep-TLC (eluted with a dichloromethane solution containing 10% methanol) to obtain a yellow gum compound 102 N-(7-morpholin-3-phenylimidazo[1,2-a]pyridin-6-yl)acetamide (0.6 mg).

LC_MS: (ES⁺): m/z 337.1 [M+H]⁺.

### Example 21: Synthesis of Compound 103, Compound 104 and Compound 105

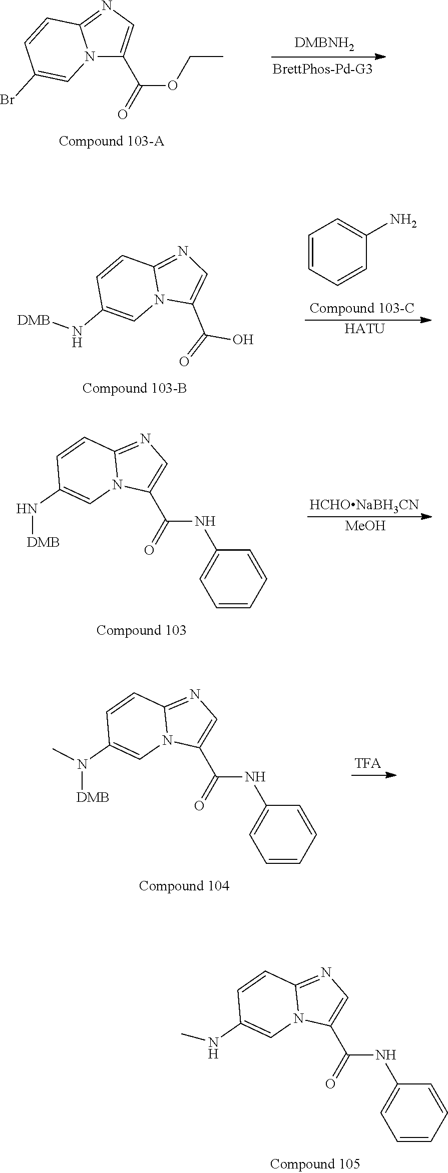

### Step 1: Compound 103-B 6-((3,4-dimethylbenzyl)amino)imidazo[1,2-a]pyridin-3-carboxylic acid

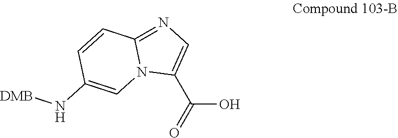

A solution containing compound 103-A (500 mg, 1.86 mmol) and 2,4-dimethoxybenzylamine (372.8 mg, 2.23 mmol) in dioxane (20 mL) was added sodium tert-butoxide (357.5 mg, 3.72 mmol) and BrettPhos-Pd-G3 (84.3 mg, 0.093 mmol). The reaction solution was stirred at 100° C. under nitrogen for 12 hours of reaction. The reaction was monitored by LCMS to be completed. The reaction solution was diluted with water (20 mL), and extracted with ethyl acetate (20 mL×2). The organic layer was adjusted to pH=7 with 2N HCL. A large amount of solids were generated. The mixture was filtered. The filter cake was collected and dried under vacuum. The residue was used directly in the next step without further purification. A yellow solid compound 103-B 6-((3,4-dimethylbenzyl)amino)imidazo[1,2-a]pyridin-3-carboxylic acid (230 mg) was obtained.

LC_MS: (ES⁺): m/z 337.1 [M+H]⁺.

### Step 2: Compound 103 6-((3,4-dimethylbenzyl)amino)-N-phenylimidazo[1,2-a]pyridin-3-carboxamide

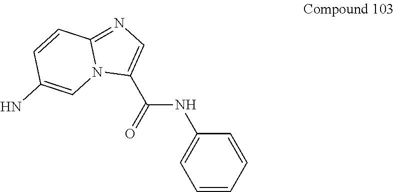 [SMILES: Nc1ccc2ncc(C(=O)Nc3ccccc3)n2c1]

A solution containing compound 103-B (100 mg, 305.49 μmol) and compound 103-C (56.9 mg, 610.99 μmol) in DMF (2 mL) was added with DIPEA (118.5 mg, 916.48 μmol) and HATU (139.4 mg, 366.59 μmol). The reaction solution was stirred at 20° C. for 3 hours of reaction. TLC (DCM:MeOH=10:1) monitoring showed that compound 103-B (Rf=0.05) was completely consumed and a new spot (Rf=0.5) was generated. The reaction solution was diluted with water (10 mL), and extracted with ethyl acetate (10 mL×2). The organic layers were combined, washed with saturated brine (20 mL), dried over anhydrous sodium sulfate, filtered and concentrated. The residue was subjected to Prep-TLC (eluted with dichloromethane containing 10% methanol) to obtain compound 103 6-((3,4-dimethylbenzyl)amino)-N-phenylimidazo[1,2-a]pyridin-3-carboxamide (95 mg).

LC_MS: (ES⁺): m/z 403.2 [M+H]⁺.

### Step 3: Compound 104 6-((3,4-dimethylbenzyl)(methyl)amino)-N-phenylimidazo[1,2-a]pyridin-3-carboxamide

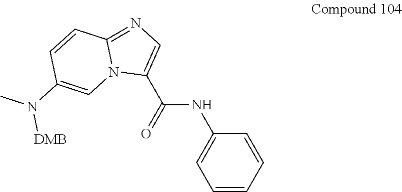

A solution containing compound 103 (95 mg, 236.05 μmol) and formaldehyde (98.4 mg, 1.18 mmol, 36% wt in water) in methanol (1 mL) was added with sodium cyanoborohydride (29.7 mg, 472.10 μmol) and one drop of acetic acid. The reaction solution was stirred at 20° C. for 12 hours of reaction. TLC (DCM:MeOH=10:1) monitoring showed that most of the compound 103 (Rf=0.55) was consumed and a new spot (Rf=0.6) was generated. The reaction solution was diluted with water (10 mL), and extracted with dichloromethane (10 mL×2). The organic layers were combined, washed with saturated brine (10 mL), dried over anhydrous sodium sulfate, filtered and concentrated. The residue was subjected to Prep-TLC (eluted with dichloromethane containing 5% methanol) to obtain compound 104 6-((3,4-dimethylbenzyl)(methyl)amino)-N-phenylimidazo[1,2-a]pyridin-3-carboxamide as a yellow solid (42 mg).

LC_MS: (ES⁺): m/z 417.2 [M+H]⁺.

### Step 4: Compound 105 6-(methylamino)-N-phenylimidazo[1,2-a]pyridin-3-carboxamide

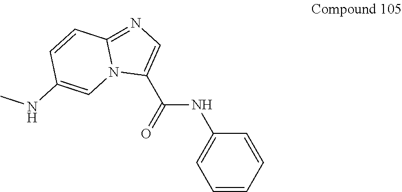 [SMILES: CNc1ccc2ncc(C(=O)Nc3ccccc3)n2c1]

A solution of compound 104 (37.0 mg, 88.84 μmol) in dichloromethane (3 mL) was added with trifluoroacetic acid (1 mL). The reaction solution was stirred at 45° C. for 3 hours of reaction. The reaction was monitored by TLC (DCM:MeOH=10:1) to be completed. The reaction solution was concentrated, diluted with saturated sodium bicarbonate solution (10 mL), and extracted with dichloromethane (10 mL×2). The organic layers were combined, washed with saturated brine (20 mL), dried over anhydrous sodium sulfate, filtered and concentrated. The residue was subjected to Prep-TLC (eluted with dichloromethane containing 10% methanol) to obtain compound 105 6-(methylamino)-N-phenylimidazo[1,2-a]pyridin-3-carboxamide as a white solid (12.1 mg).

LC_MS: (ES) m/z 267.1 [M+H]⁺.

### Example 22: Synthesis of Compound 106

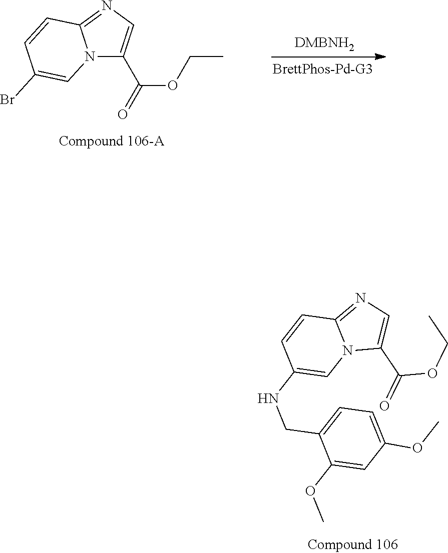 [SMILES: CCOC(=O)c1cnc2ccc(Br)cn12 ; CCOC(=O)c1cnc2ccc(NCc3ccc(OC)cc3OC)cn12]

### Step 1: Compound 106 ethyl 6-((2,4-dimethoxybenzyl)amino)imidazo[1,2-a]pyridin-3-carboxylate

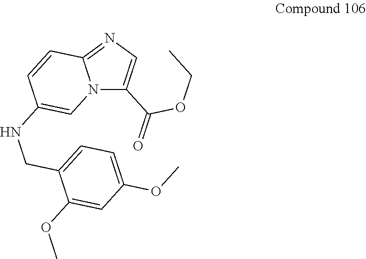 [SMILES: CCOC(=O)c1cnc2ccc(NCc3ccc(OC)cc3OC)cn12]

A solution containing compound 106-A (20 mg, 74.32 μmol) and 2,4-dimethoxybenzylamine (14.9 mg, 89.19 μmol) in dioxane (20 mL) was added with cesium carbonate (72.65 mg, 222.97 μmol) and BrettPhos-Pd-G3 (3.3 mg, 3.716 μmol). The reaction solution was stirred at 100° C. under nitrogen for 12 hours of reaction. The reaction was monitored by LCMS to be completed. The reaction solution was diluted with water (10 mL), and extracted with ethyl acetate (10 mL×2). The organic layers were combined, washed with saturated brine (20 mL), dried over anhydrous sodium sulfate, filtered, and concentrated. The residue was subjected to Prep-TLC (eluted with a petroleum ether solution containing 50% ethyl acetate) to obtain a green gum compound 106 ethyl 6-((2,4-dimethoxybenzyl)amino)imidazo[1,2-a]pyridin-3-carboxylate (4.1 mg).

LC_MS: (ES⁺): m/z 356.1 [M+H]⁺.

### Example 23: Synthesis of Compound 107

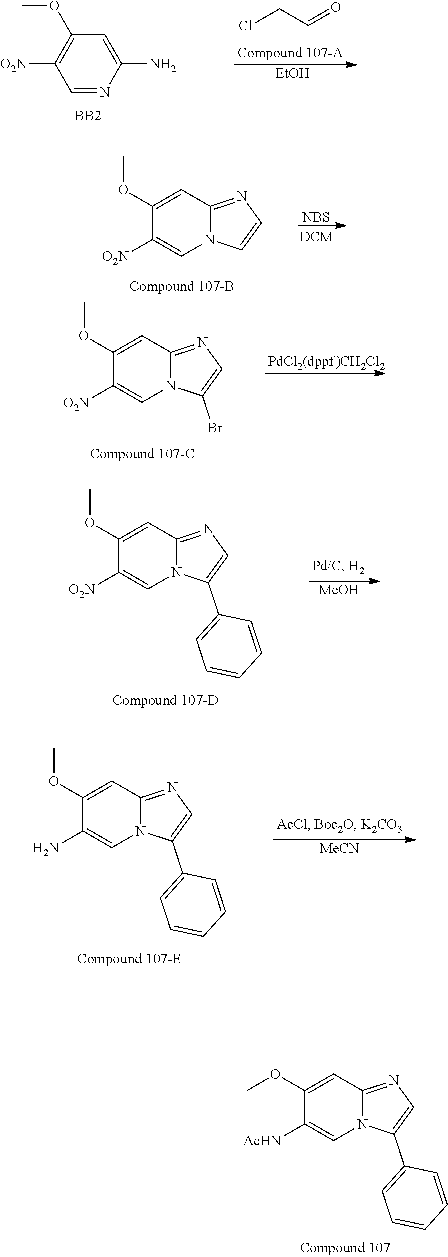

Step 1: The synthesis of compound 107-B was performed referring to the third step of the synthesis process of intermediate BB5.

Step 2: The synthesis of compound 107-C was performed referring to the fourth step of the synthesis process of intermediate BB5.

Step 3: The synthesis of compound 107-D was performed referring to the first step of the synthesis process of compound 2.

Step 4: Compound 107-E 7-methoxy-3-phenylimidazo[1,2-a]pyridin-6-amine

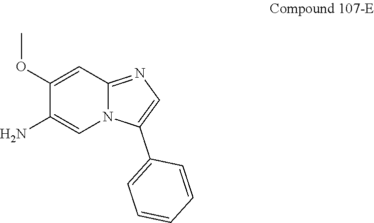 [SMILES: COc1cc2ncc(-c3ccccc3)n2cc1N]

A solution of compound 107-D (80 mg, 297.11 μmol) in methanol (5 mL) was added with palladium/carbon (10 mg). The reaction solution was stirred under a hydrogen balloon atmosphere at 20° C. for 12 hours of reaction. TLC (DCM:MeOH=10:1) monitoring showed that compound 107-D (Rf=0.7) was reacted completely, and a new spot (Rf=0.6) was generated. The reaction solution was filtered through diatomite, the mother liquor was concentrated, and the residue was used directly in the next step without further purification. A brown oil crude compound 107-E 7-methoxy-3-phenylimidazo[1,2-a]pyridin-6-amine (71 mg) was obtained.

LC_MS: (ES⁺): m/z 240.1 [M+H]⁺.

### Step 5: Compound 107 N-(7-methoxy-3-phenylimidazo[1,2-a]pyridin-6-yl)acetamide

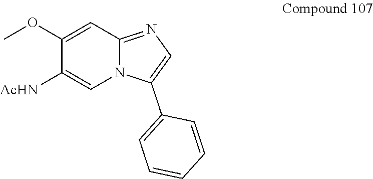 [SMILES: COc1cc2ncc(-c3ccccc3)n2cc1NC(C)=O]

A solution containing compound 107-E (33 mg, 137.91 μmol) in acetonitrile (2 mL) was added with potassium carbonate (38.1 mg, 275.83 μmol), acetyl chloride (23.8 mg, 303.42 μmol) and di-tert-butyl dicarbonate (60.2 mg, 275.82 μmol). The reaction solution was stirred at 60° C. for 12 hours of reaction. The reaction was monitored by TLC (DCM:MeOH=10:1) to be completed. The reaction solution was diluted with water (10 mL), and extracted with dichloromethane (10 mL×2). The organic layers were combined, washed with saturated brine (10 mL), dried over anhydrous sodium sulfate, filtered and concentrated. The residue was separated and purified by Prep-TLC (eluted with petroleum ether containing 6.7% dichloromethane and 75% ethyl acetate) to obtain yellow solid compound 107 N-(7-methoxy-3-phenylimidazo[1,2-a]pyridin-6-yl)acetamide (3.6 mg).

LC_MS: (ES⁺): m/z 282.1 [M+H]⁺.

### Example 24: Synthesis of Compound 108 and Compound 109

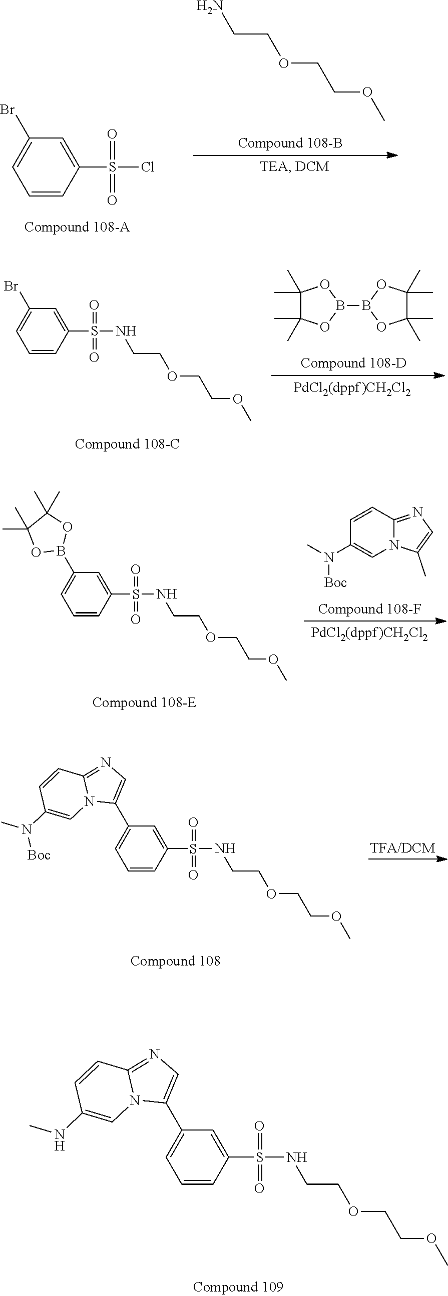

### Step 1: Compound 108-C 3-bromo-N-(2-(2-methoxyethoxy)ethyl)benzenesulfonamide

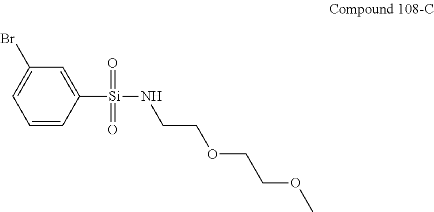

A solution containing compound 108-A 3-bromobenzenesulfonyl chloride (800 mg, 3.13 mmol) and triethylamine (633.7 mg, 6.26 mmol) in dichloromethane was added with compound 108-B 2-(2-methoxyethoxy)ethan-1-amine (391.8 mg, 3.29 mmol). The reaction solution was stirred at 0° C. for 12 hours of reaction. The reaction was monitored by TLC (petroleum ether:ethyl acetate=1:1) to be completed. The reaction solution was diluted with water (20 mL), and extracted with dichloromethane (20 mL×2). The organic layers were combined, washed with saturated brine (20 mL), dried over anhydrous sodium sulfate, filtered and concentrated. The residue was separated and purified by silica gel column chromatography (petroleum ether:ethyl acetate=1:1) to obtain compound 108-C 3-bromo-N-(2-(2-methoxyethoxy)ethyl)benzenesulfonamide (840 mg) as an oil.

LC_MS: (ES⁺): m/z 337.9 [M+H]⁺.

### Step 2: Compound 108-E N-(2-(2-methoxyethoxy)ethyl)-3-(4,4,5,5-tetramethyl-1,3,2-dioxaborolan-2-yl)benzenesulfonamide

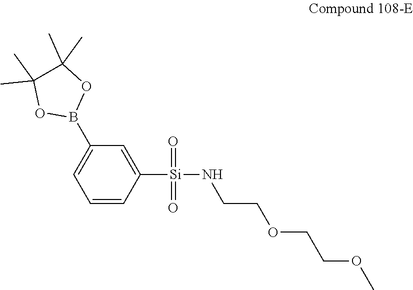

A solution containing compound 108-C 3-bromo-N-(2-(2-methoxyethoxy)ethyl)benzenesulfonamide (840 mg, 2.48 mmol) and compound 108-D bis(pinacolato)borate (946.0 mg, 3.73 mmol) in 1,4-dioxane was added with potassium acetate (731.2 mg, 7.45 mmol) and [1,1′-bis(diphenylphosphino)ferrocene]palladium dichloride dichloromethane complex (202.5 mg, 0.248 mmol). The reaction solution was stirred under a nitrogen atmosphere at 85° C. for 12 hours of reaction. The reaction was monitored by TLC (petroleum ether:ethyl acetate=1:1) to be completed. The reaction solution was filtered through diatomite, and the mother liquor was diluted with water (20 mL), and extracted with ethyl acetate (20 mL×2). The organic layers were combined, washed with saturated brine (20 mL), dried over anhydrous sodium sulfate, filtered and concentrated. The residue was separated and purified by silica gel column chromatography (petroleum ether:ethyl acetate=1:1) to obtain crude compound 108-E N-(2-(2-methoxyethoxy)ethyl)-3-(4,4,5,5-tetramethyl-1,3,2-dioxaborolan-2-yl)benzenesulfonamide as a yellow oil (806 mg).

LC_MS: (ES⁺): m/z 385.6 [M+H]⁺.

### Step 3: Compound 108 tert-butyl (3-(3-(N-(2-(2-methoxyethoxy)ethyl)sulfamoyl)phenyl)imidazo[1,2-a]pyridin-6-yl)(methyl)carbamate

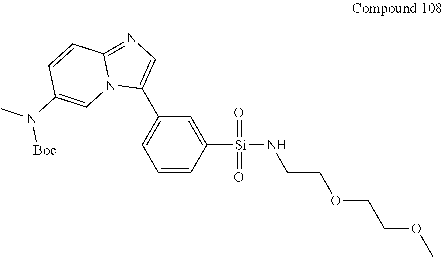

The synthesis of this step was performed referring to the first step of the synthesis process of compound 2.

LC_MS: (ES⁺): m/z 505.1 [M+H]⁺.

### Step 4: Compound 109 N-(2-(2-methoxyethoxy)ethyl)-3-(6-(methylamino)imidazo[1,2-a]pyridin-3-yl)benzenesulfonamide

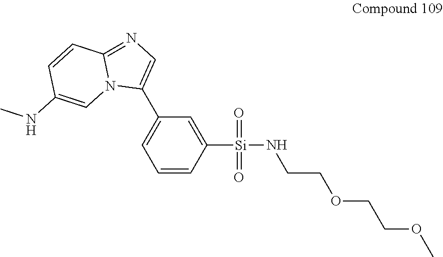

The synthesis of this step was performed referring to the first step of the synthesis process of compound 63.

LC_MS: (ES⁺): m/z 405.0 [M+H]⁺.

The following compounds were synthesized referring to the synthesis of compound 108 and compound 109.

### Synthesis of Compound 110

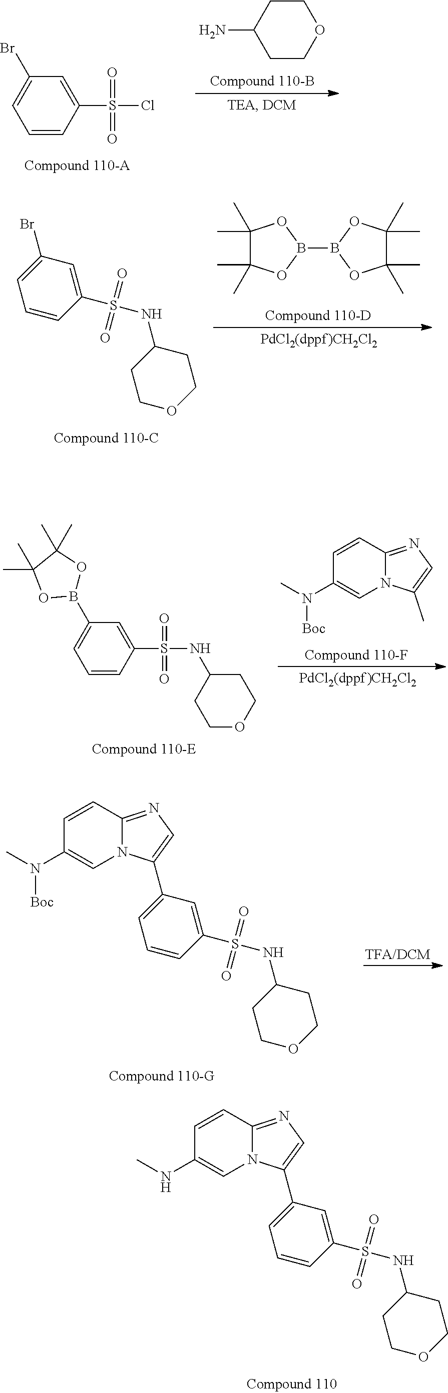

Step 1: The synthesis of compound 110-C was performed referring to the first step of the synthesis process of compound 109.

Step 2: The synthesis of compound 110-E was performed referring to the second step of the synthesis process of compound 109.

Step 3: The synthesis of compound 110-G was performed referring to the third step of the synthesis process of compound 109.

### Step 4: Compound 110 3-(6-(methylamino)imidazo[1,2-a]pyridin-3-yl)-N-(tetrahydro-2H-pyran-4-yl)benzenesulfonamide

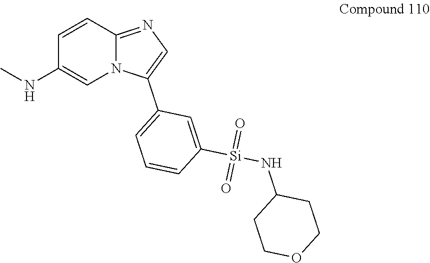

The synthesis of this step was performed referring to the fourth step of the synthesis process of compound 109.

LC_MS: (ES⁺): m/z 387.0 [M+H]⁺.

### Example 25: Synthesis of Compound 111 and Compound 112

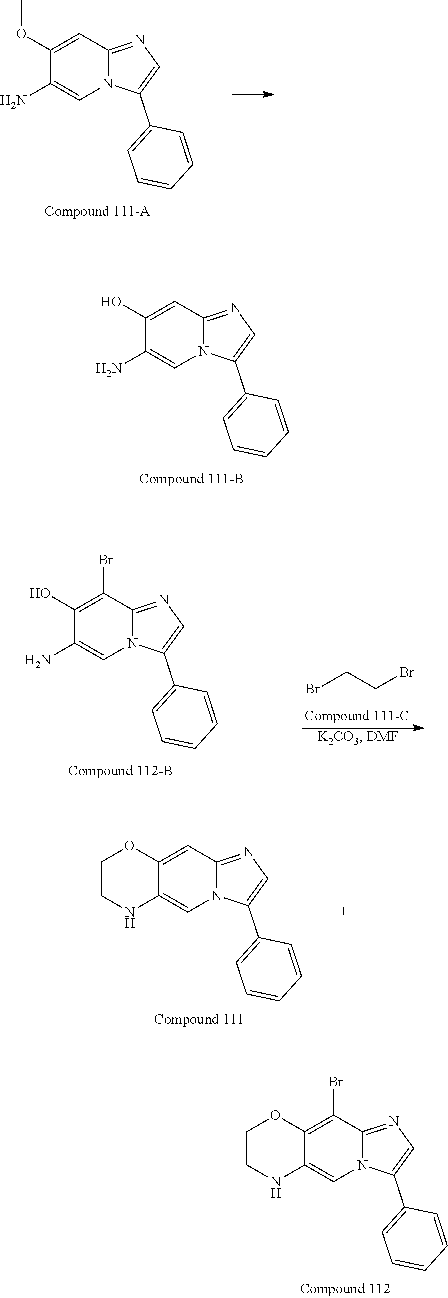

### Step 1: Compound 111-B and Compound 112-B: 6-amino-3-phenylimidazo[1,2-a]pyridin-7-ol and 6-amino-8-bromo-3-phenylimidazo[1,2-a]pyridin-7-ol

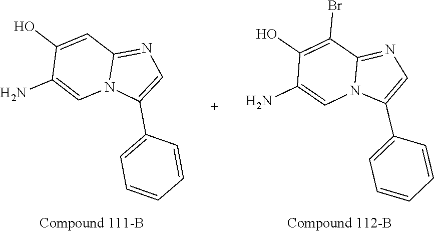 [SMILES: Nc1cn2c(-c3ccccc3)cnc2cc1O ; Nc1cn2c(-c3ccccc3)cnc2c(Br)c1O]

A solution of compound 111-A (20 mg, 83.58 μmol) in dichloromethane (5 mL) was added with boron tribromide (41.9 mg, 167.17 μmol) at 0° C. The reaction solution was stirred for 12 hours of reaction. LCMS detected the MS of compound 111-B and compound 112-B. The reaction solution was concentrated. The residue was used directly in the next step without further purification. Thus, yellow solid crude product compounds 111-B and 112-B: 6-amino-3-phenylimidazo[1,2-a]pyridin-7-ol, and 6-amino-8-bromo-3-phenylimidazo[1,2-a]pyridin-7-ol were obtained (18.8 mg).

LC_MS: (ES⁺): m/z 226.1 [M+H]⁺.

### Step 2: Compound 111 7-phenyl-3,4-dihydro-2H-imidazo[1′,2′:1,6]pyrido[4,3-b][1,4]oxazine and Compound 112 10-bromo-7-phenyl-3,4-dihydro-2H-imidazol[1′,2′:1,6]pyrido[4,3-b][1,4]oxazine

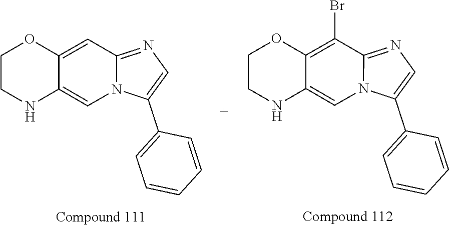 [SMILES: c1ccc(-c2cnc3cc4c(cn23)NCCO4)cc1 ; Brc1c2c(cn3c(-c4ccccc4)cnc13)NCCO2]

A solution containing compound 111-B and compound 112-B (18.8 mg, 83.46 μmol) in DMF (2 mL) was added with 1,2-dibromoethane (18.8 mg, 100.15 μmol) and potassium carbonate (34.6 mg, 250.39 μmol). The reaction was stirred at 100° C. for 12 hours of reaction. The target product and by-products were detected by LCMS. The reaction solution was diluted with water (10 mL), and extracted with ethyl acetate (10 mL×2). The organic layers were combined, washed with saturated brine (10 mL), dried over anhydrous sodium sulfate, filtered and concentrated. The residue was subjected to silica gel column chromatography (eluted with dichloromethane containing 10% methanol) to obtain yellow oil compound 111 7-phenyl-3,4-dihydro-2H-imidazo[1′,2′:1,6]pyrido[4,3-b][1,4]oxazine (3.0 mg) and yellow oil compound 112 10-bromo-7-phenyl-3,4-dihydro-2H-imidazol[1′,2′:1,6]pyrido [4,3-b][1,4]oxazine (3.4 mg).

LC_MS: (ES): m/z 252.1 [M+H]⁺.

LC_MS: (ES): m/z 330.0 [M+H]⁺.

### Example 26: Synthesis of Compound 113

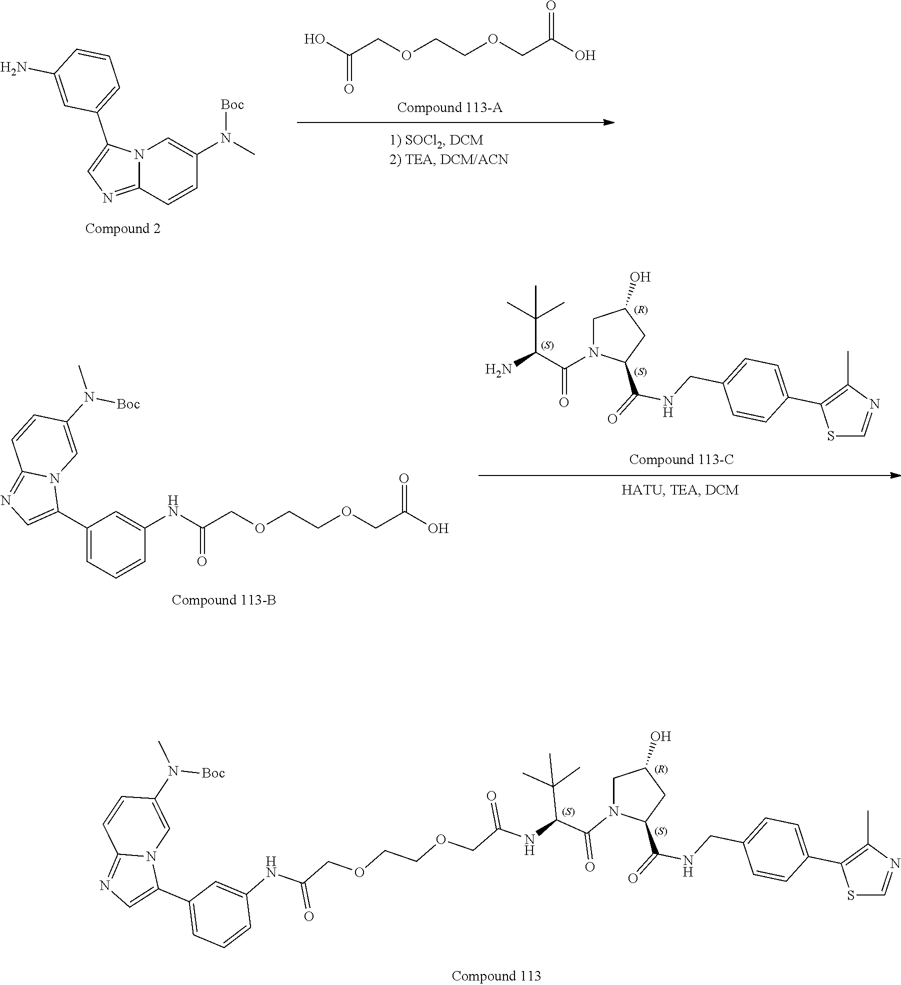

### Step 1: Compound 113-B 2-(2-(2-((3-(6-((tert-butoxycarbonyl)(methyl)amino)imidazo[1,2-a]pyridin-3-yl)phenyl)amino)-2-oxyethoxy)ethoxy)acetic acid

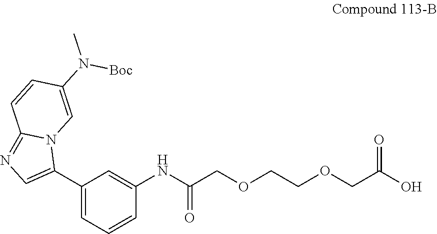 [SMILES: CN(C(=O)OC(C)(C)C)c1ccc2ncc(-c3cccc(NC(=O)COCCOCC(=O)O)c3)n2c1]

Compound 113-A (40 mg, 220 μmol) was added to thionyl chloride (4 mL). The reaction solution was stirred at 60° C. for 2 hours until the solution was clear. The reaction solution was concentrated under reduced pressure, and dried. The residue was dissolved with dichloromethane (30 mL), and then added with compound 2 (25 mg, 74 μmol) and triethylamine (52 μL, 370 μmol) at 0° C. The reaction solution was stirred at 0° C. for 3 hours of reaction. After the reaction was completed, the reaction solution was added with aqueous hydrochloric acid solution (30 mL, 0.1 N), and extracted with ethyl acetate (30 mL×2). The combined organic layers were washed with saturated brine (30 mL), dried over anhydrous Na₂SO₄, filtered and concentrated. The residue was separated and purified by preparative thin-layer chromatography (eluent: dichloromethane solution containing 15% methanol) to obtain compound 113-B (20 mg) as a yellow solid.

LC_MS: (ES⁺): m/z 499.2 [M+H]⁺.

### Step 2: Compound 113 tert-butyl (3-(3-(2-(2-(2-((((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)acetamido)phenyl)imidazo[1,2-a]pyridin-6-yl)(methyl)carbamate

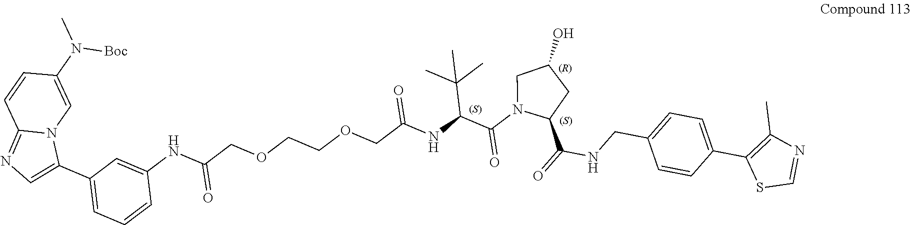

A solution containing compound 113-B (20 mg, 40 μmol), compound 113-C (18 mg, 40 mol) and triethylamine (12 μL, 80 μmol) in dichloromethane was added with HATU (23 mg, 60 mol). The reaction mixture was stirred at room temperature for 9 hours of reaction. After the reaction was completed, the reaction solution was diluted with water (20 mL) and extracted with dichloromethane (20 mL×2). The combined organic layers were washed with saturated brine (20 mL), dried over anhydrous Na₂SO₄, filtered, and concentrated. The residue was separated and purified by preparative thin-layer chromatography (eluent: dichloromethane solution containing 8% methanol) to obtain compound 113 (15 mg) as a white solid.

LC_MS: (ES⁺): m/z 911.4 [M+H]⁺.

### Example 27: Synthesis of Compound 114

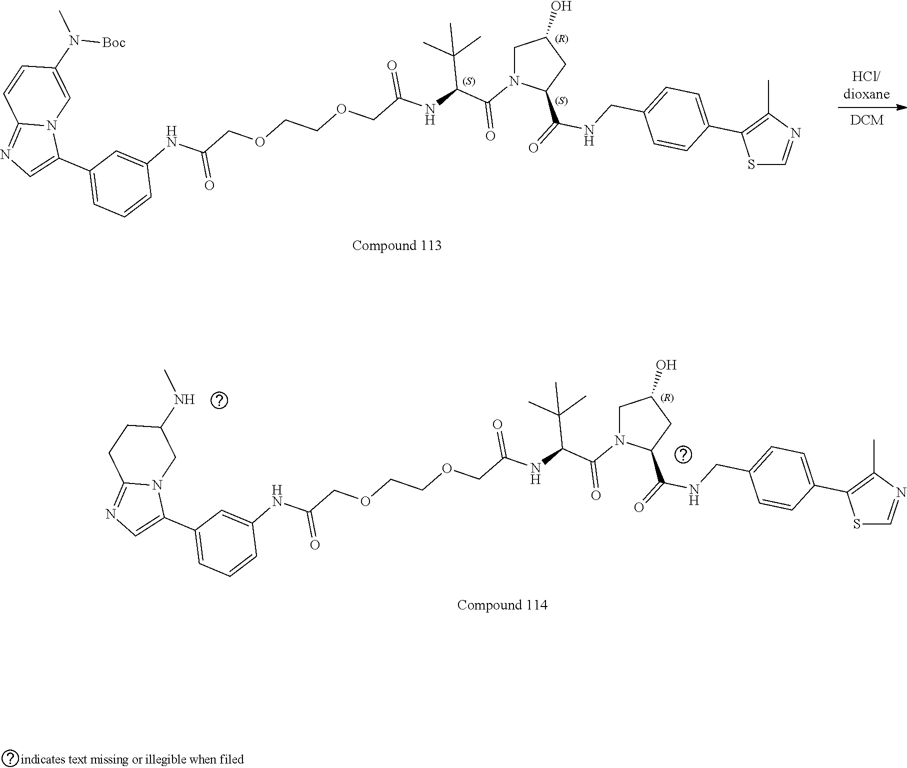

### Step 1: Compound 114 (2S,4R)-1-((S)-3,3-dimethyl-2-(2-(2-(2-((3-(6-(methylamino)imidazo[1,2-a]pyridin-3-yl)phenyl)amino)-2-oxoethoxy)ethoxy)acetamido)butyryl)-4-hydroxy-N-(4-(4-methylthiazol-5-yl)benzyl)pyrrolidin-2-carboxamide hydrochloride

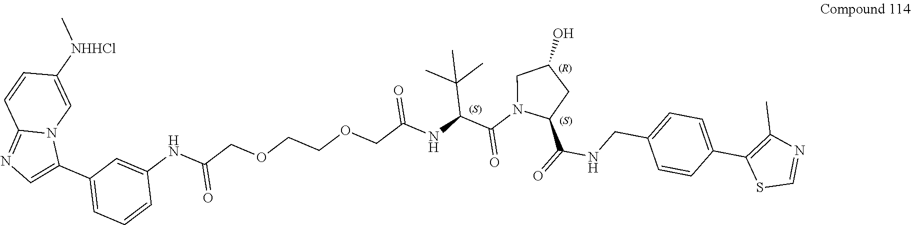

Compound 113 (5 mg, 5.5 μmol) was dissolved in dichloromethane (3 mL). The reaction solution was then added with a solution of hydrogen chloride in dioxane (1 mL, 4 M), and stirred at room temperature for 3 hours of reaction. After the reaction was completed, the reaction solution was concentrated under reduced pressure, and dried to obtain a yellow solid compound 114 (4 mg).

LC_MS: (ES⁺): m/z 811.3 [M+H]⁺.

### Example 28: Synthesis of Compound 115

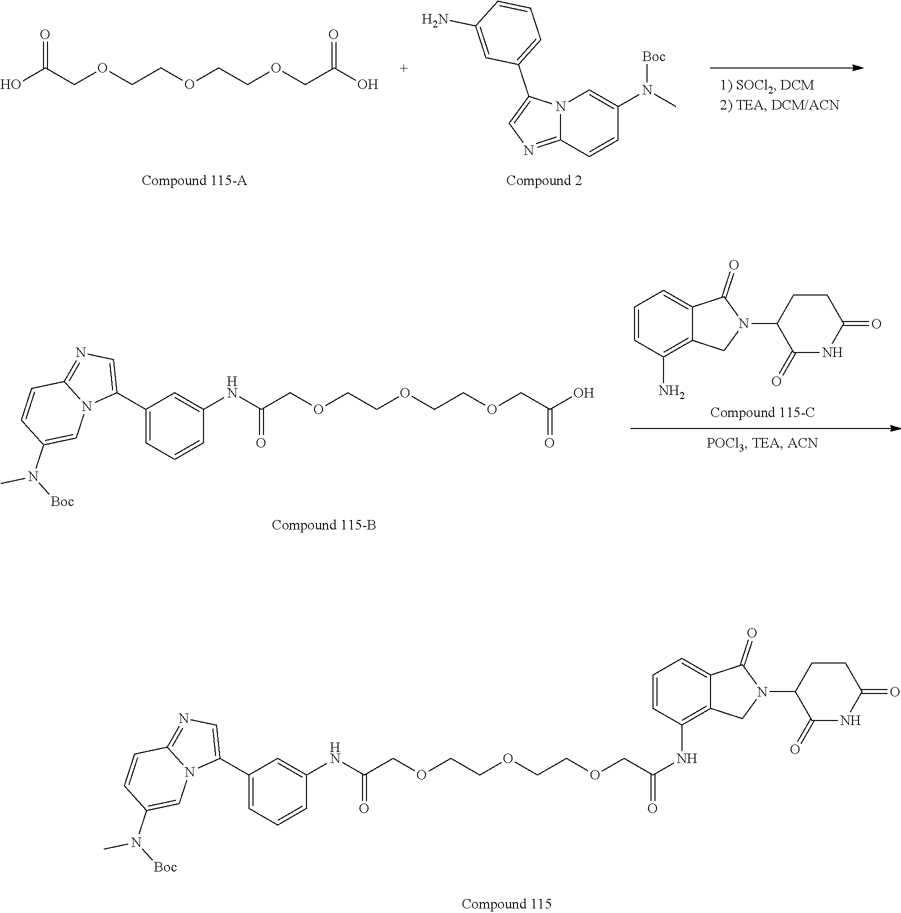 [SMILES: O=C(O)COCCOCCOCC(=O)O ; CN(C(=O)OC(C)(C)C)c1ccc2ncc(-c3cccc(N)c3)n2c1 ; CN(C(=O)OC(C)(C)C)c1ccc2ncc(-c3cccc(NC(=O)COCCOCCOCC(=O)O)c3)n2c1 ; Nc1cccc2c1CN(C1CCC(=O)NC1=O)C2=O ; CN(C(=O)OC(C)(C)C)c1ccc2ncc(-c3cccc(NC(=O)COCCOCCOCC(=O)Nc4cccc5c4CN(C4CCC(=O)NC4=O)C5=O)c3)n2c1]

### Step 1: Compound 115-B 2-(2-(2-(2-((3-(6-((tert-butoxycarbonyl)(methyl)amino)imidazo[1,2-a]pyridin-3-yl)phenyl)amino)-2-oxoethoxy)ethoxy)ethoxy)acetic acid

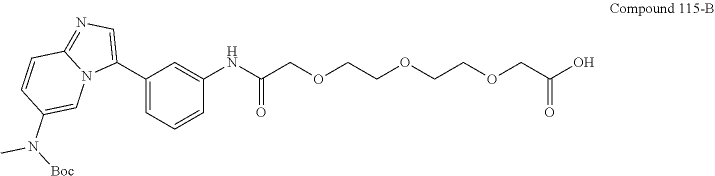 [SMILES: CN(C(=O)OC(C)(C)C)c1ccc2ncc(-c3cccc(NC(=O)COCCOCCOCC(=O)O)c3)n2c1]

The synthesis of this step was performed referring to the first step of the synthesis process of compound 113.

### Step 2: Compound 115 tert-butyl (3-(3-(2-(2-(2-(2-((2-(2,6-dioxopiperidin-3-yl))-1-oxoisoindolin-4-yl)amino)-2-oxoethoxy)ethoxy)ethoxy)acetamido)phenyl)imidazo[1,2-a]pyridin-6-yl)(methyl) carbamate

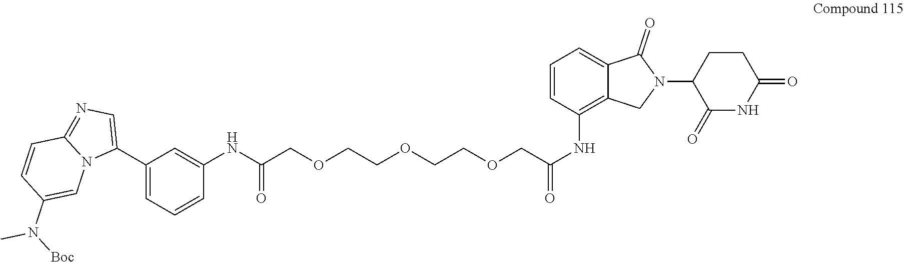 [SMILES: CN(C(=O)OC(C)(C)C)c1ccc2ncc(-c3cccc(NC(=O)COCCOCCOCC(=O)Nc4cccc5c4CN(C4CCC(=O)NC4=O)C5=O)c3)n2c1]

A solution containing compound 115-B (20 mg, 40 μmol), compound 115-C (14 mg, 55 mol) and triethylamine (15 μL, 111 μmol) in acetonitrile (3 mL) was added with phosphorus oxychloride (7 μL, 74 μmol). The reaction mixture was stirred at room temperature for 4 hours of reaction. After the reaction was completed, the reaction solution was diluted with water (20 mL) and extracted with dichloromethane (20 mL×2). The combined organic layers were washed with saturated brine (20 mL), dried over anhydrous Na₂SO₄, filtered, and concentrated. The residue was separated and purified by preparative thin-layer chromatography (eluent: dichloromethane solution containing 10% methanol) to obtain compound 115 (5.0 mg) as a white solid.

LC_MS: (ES⁺): m/z 784.3 [M+H]⁺.

### Example 29: Synthesis of Compound 116

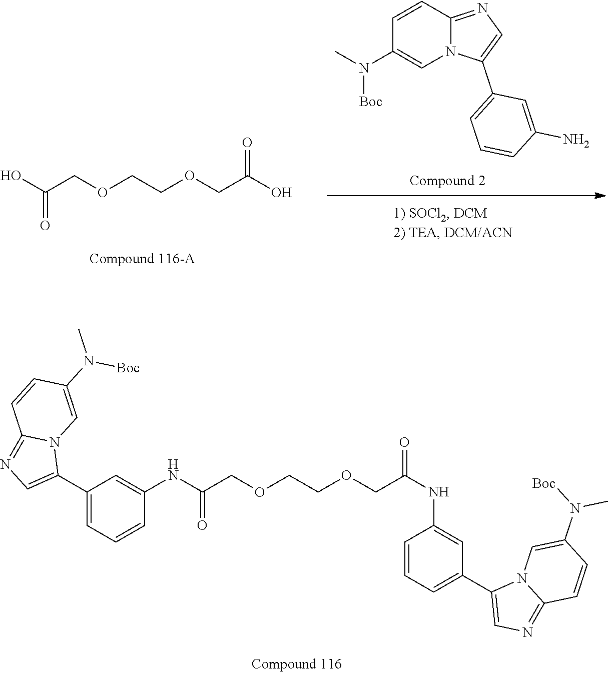 [SMILES: O=C(O)COCCOCC(=O)O ; CN(C(=O)OC(C)(C)C)c1ccc2ncc(-c3cccc(N)c3)n2c1 ; CN(C(=O)OC(C)(C)C)c1ccc2ncc(-c3cccc(NC(=O)COCCOCC(=O)Nc4cccc(-c5cnc6ccc(N(C)C(=O)OC(C)(C)C)cn56)c4)c3)n2c1]

### Step 1: Compound 116 ((((((2,2′-(ethane-1,2-diylbis(oxy))bis(acetyl))bis(azadialkyl))bis(31-phenylene))bis(imidazo[1,2-a]pyridin-3,6-diyl))bis(tert butylmethylcarbamate)

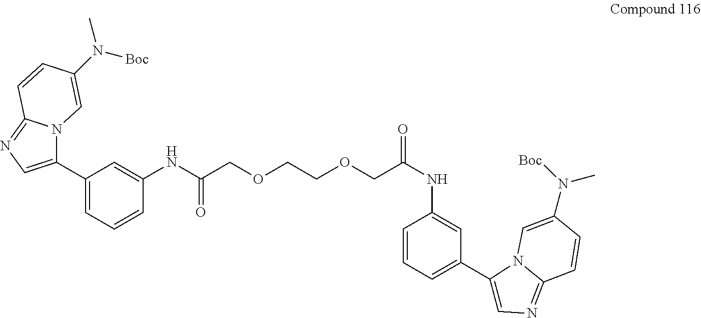 [SMILES: CN(C(=O)OC(C)(C)C)c1ccc2ncc(-c3cccc(NC(=O)COCCOCC(=O)Nc4cccc(-c5cnc6ccc(N(C)C(=O)OC(C)(C)C)cn56)c4)c3)n2c1]

Compound 116-A (40 mg, 220 μmol) was added to thionyl chloride (4 mL). The reaction solution was stirred at 60° C. for 2 hours until the solution was clear. The reaction solution was concentrated under reduced pressure, and dried. The residue was dissolved with dichloromethane (30 mL), and then added with compound 2 (25 mg, 74 μmol) and triethylamine (52 μL, 370 μmol) at 0° C. The reaction solution was stirred at 0° C. for 3 h of reaction. After the reaction was completed, the reaction solution was added with aqueous hydrochloric acid solution (30 mL, 0.1 N), and extracted with ethyl acetate (30 mL×2). The combined organic layers were washed with saturated brine (30 mL), dried over anhydrous Na₂SO₄, filtered, and concentrated. The residue was separated and purified by preparative thin-layer chromatography (eluent: dichloromethane solution containing 8% methanol) to obtain compound 116 (10 mg) as a white solid.

LC_MS: (ES⁺): m/z 819.4 [M+H]⁺.

¹H NMR (400 MHz, DMSO) δ 8.91 (s, 2H), 8.23 (d, J=82.5 Hz, 4H), 7.77 (d, J=19.8 Hz, 6H), 7.42 (t, J=7.9 Hz, 2H), 7.32 (d, J=9.4 Hz, 2H), 7.22 (s, 2H), 4.27 (s, 4H), 4.01 (s, 4H), 3.28 (d, J=6.1 Hz, 6H), 1.47 (s, 18H).

### Synthesis of Compound 117

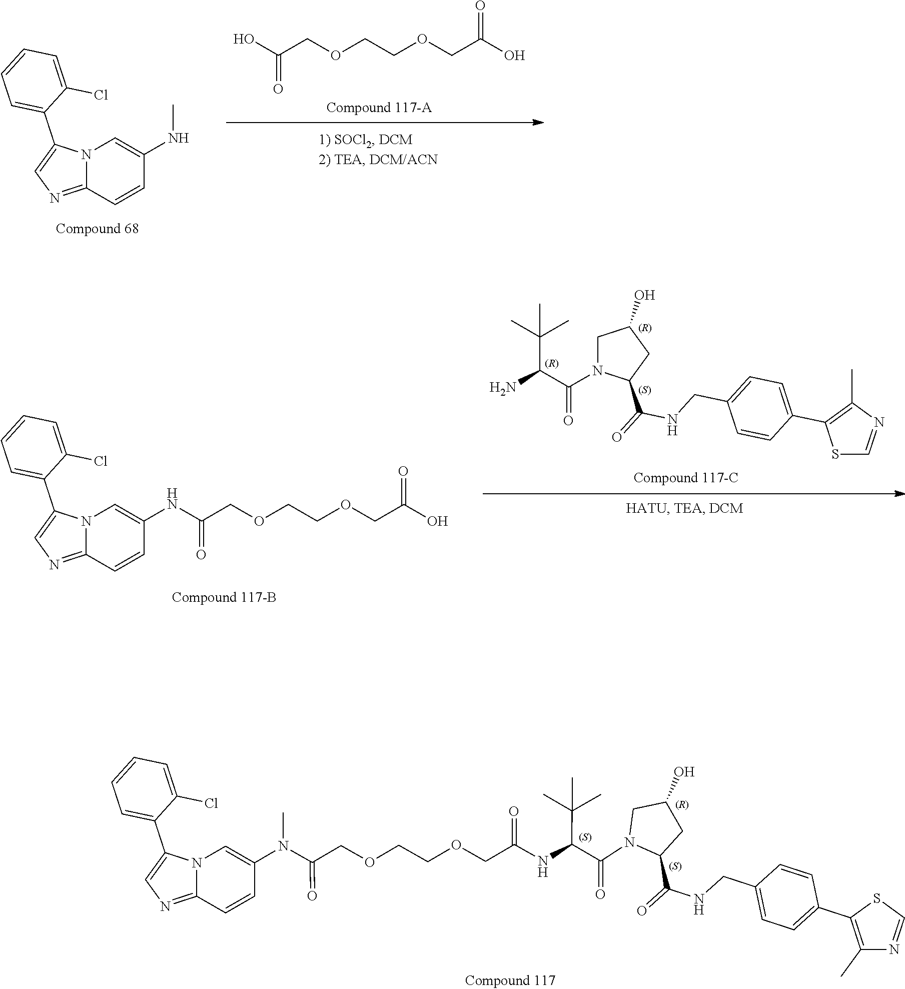

Step 1: The synthesis of compound 117-B was performed referring to the first step of the synthesis process of compound 113.

Step 2: The synthesis of compound 117 was performed referring to the second step of the synthesis process of compound 113.

### (2S,4R)-1-((S)-12-(tert-butyl)-2-(3-(2-chlorophenyl)imidazo[1,2-a]pyridin-6-yl)-3,10-dioxo-5,8-dioxa-2,11-diazahexadecane-13-acyl)-4-hydroxy-N-(4-(4-methylthiazol-5-yl)benzyl)pyrrolidin-2-carboxamide

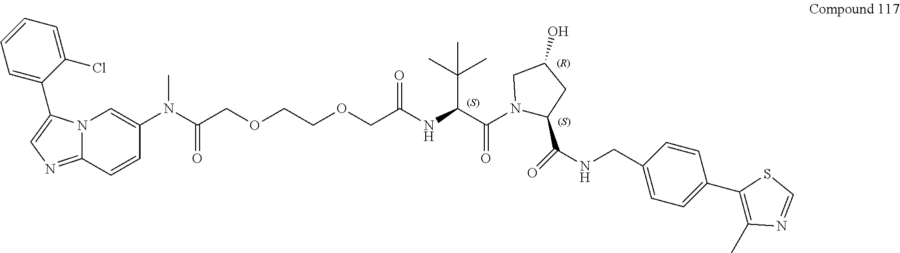

LC_MS: (ES⁺): m/z 830.3 [M+H]⁺.

### Synthesis of Compound 118

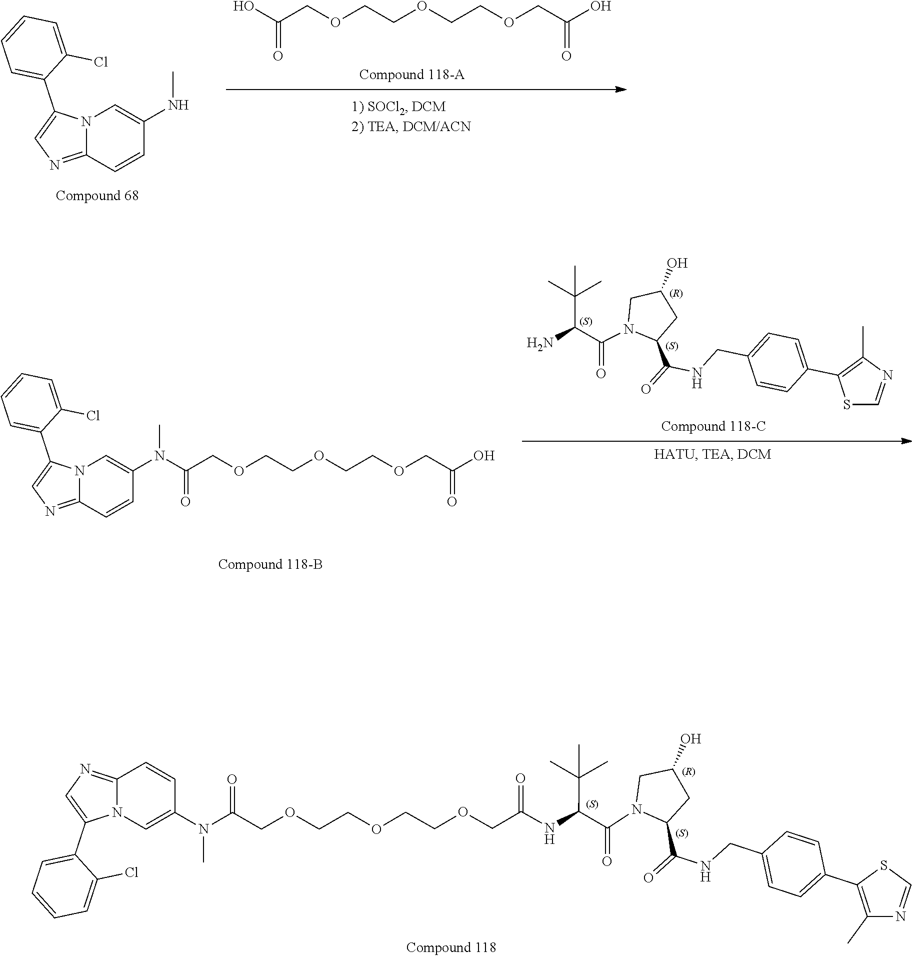

Step 1: The synthesis of compound 118-B was performed referring to the first step of the synthesis process of compound 113.

Step 2: The synthesis of compound 118 was performed referring to the second step of the synthesis process of compound 113.

### (2S,4R)-1-((S)-15-(tert-butyl)-2-(3-(2-chlorophenyl)imidazo[1,2-a]pyridin-6-yl)-3,13-di oxo-5,8,11-trioxa-2,14-diazahexadecane-16-acyl)-4-hydroxy-N-(4-(4-methylthiazol-5-yl)benzyl) pyrrolidin-2-carboxamide

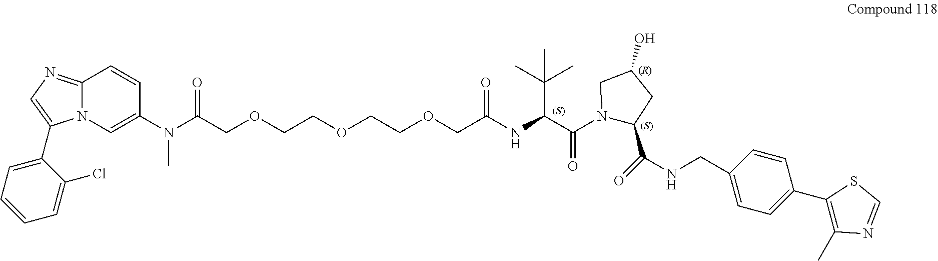

LC_MS: (ES⁺): m/z 874.3 [M+H]⁺.

### Synthesis of Compound 119

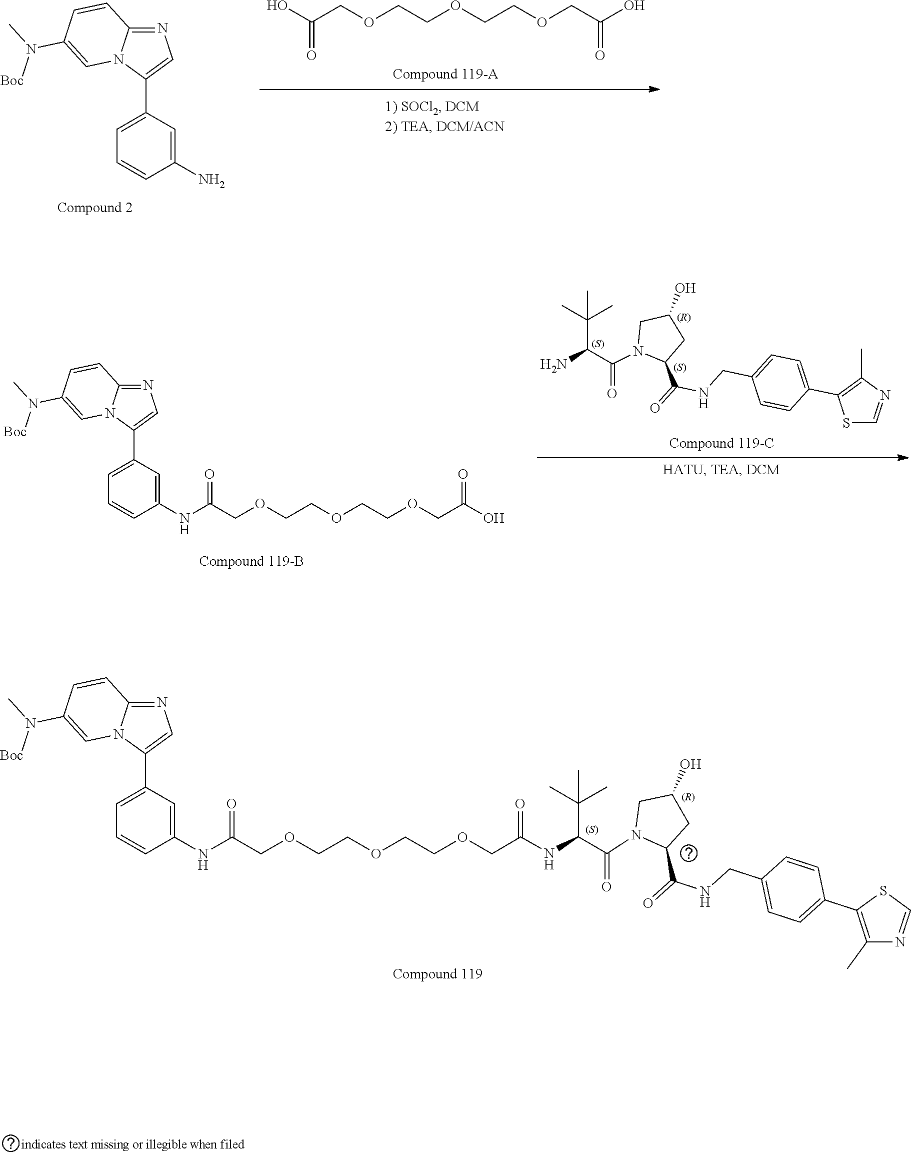

Step 1: The synthesis of compound 119-B was performed referring to the first step of the synthesis process of compound 113.

Step 2: The synthesis of compound 119 was performed referring to the second step of the synthesis process of compound 113.

### Tert-butyl (3-(3-((S)-13-((2S,4R)-4-hydroxy-2-((4-(4-methylthiazol-5-yl)benzyl)carbamoyl)pyrrolidin-1-carbonyl)-14,14-dimethyl-11-oxo-3,6,9-trioxa-12-azaglutaramide)phenyl)imidazo[1,2-a]pyridin-6-yl)(methyl)carbamate

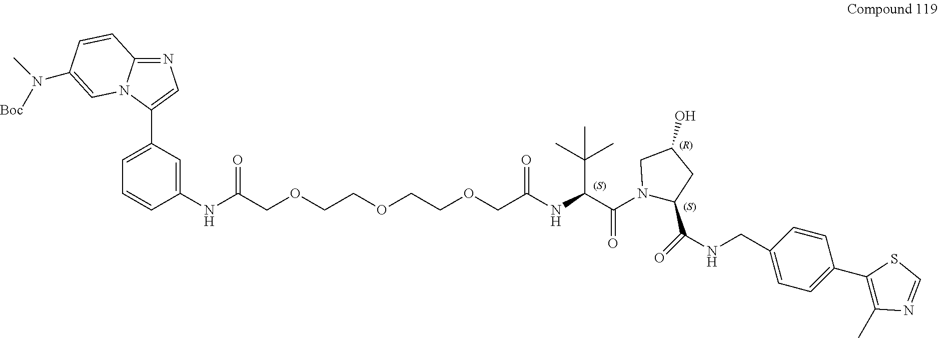

LC_MS: (ES⁺): m/z 955.4 [M+H]⁺.

### Synthesis of Compound 120

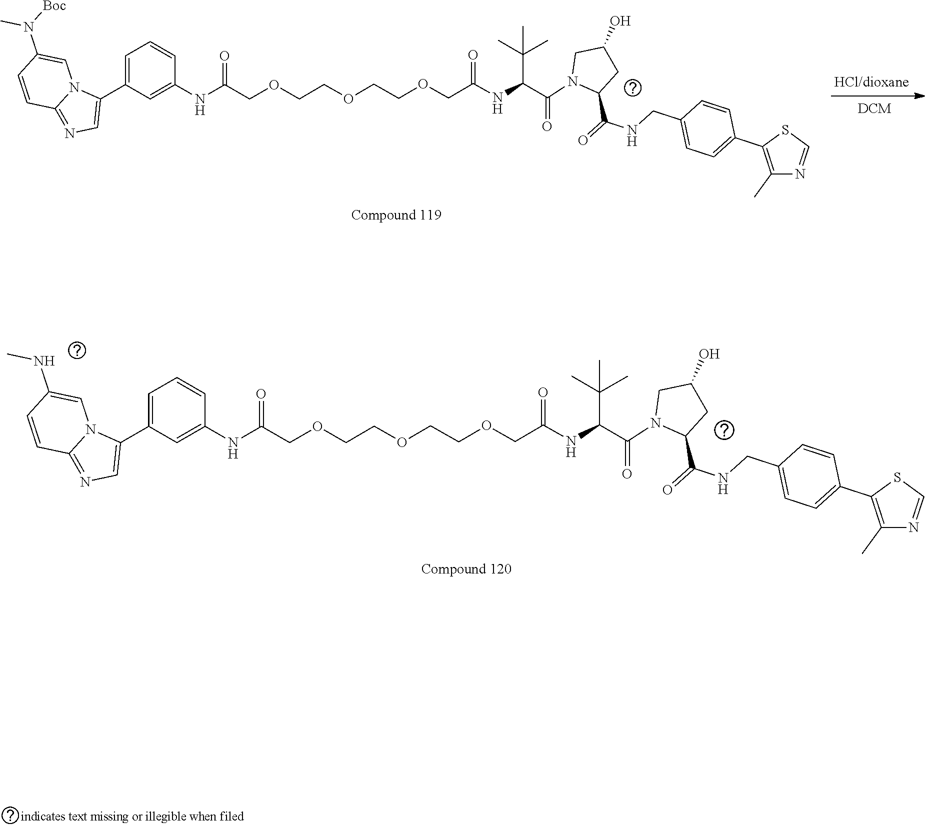 [SMILES: Cc1ncsc1-c1ccc(CNC(=O)[C@@H]2C[C@@H](O)CN2C(=O)[C@@H](NC(=O)COCCOCCOCC(=O)Nc2cccc(-c3cnc4ccc(N(C)C(=O)OC(C)(C)C)cn34)c2)C(C)(C)C)cc1 ; CNc1ccc2ncc(-c3cccc(NC(=O)COCCOCCOCC(=O)N[C@H](C(=O)N4C[C@H](O)C[C@H]4C(=O)NCc4ccc(-c5scnc5C)cc4)C(C)(C)C)c3)n2c1]

Step 1: The synthesis of compound 120 was performed referring to the first step of the synthesis process of compound 114.

### (2S,4R)-1-((S)-2-(tert-butyl)-14-((3-(6-(methylamino)imidazo[1,2-a]pyridin-3-yl)phenyl)amino)-4,14-dioxo-6,9,12-trioxa-3-azatetradecanoyl)-4-hydroxy-N-(4-(4-methylthiazol-5-yl)benzyl)pyrrolidin-2-carboxamide hydrochloride

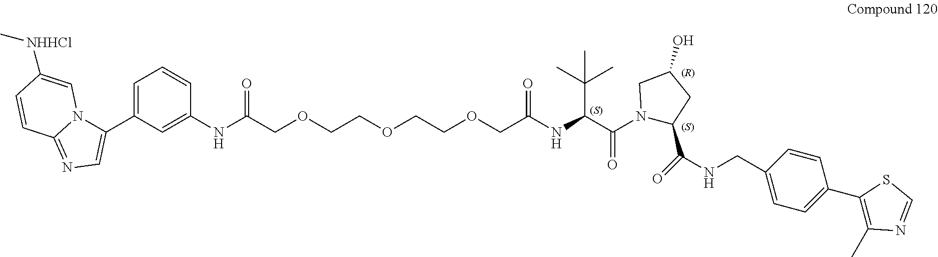

LC_MS: (ES⁺): m/z 855.3 [M+H]⁺.

### Synthesis of Compound 121

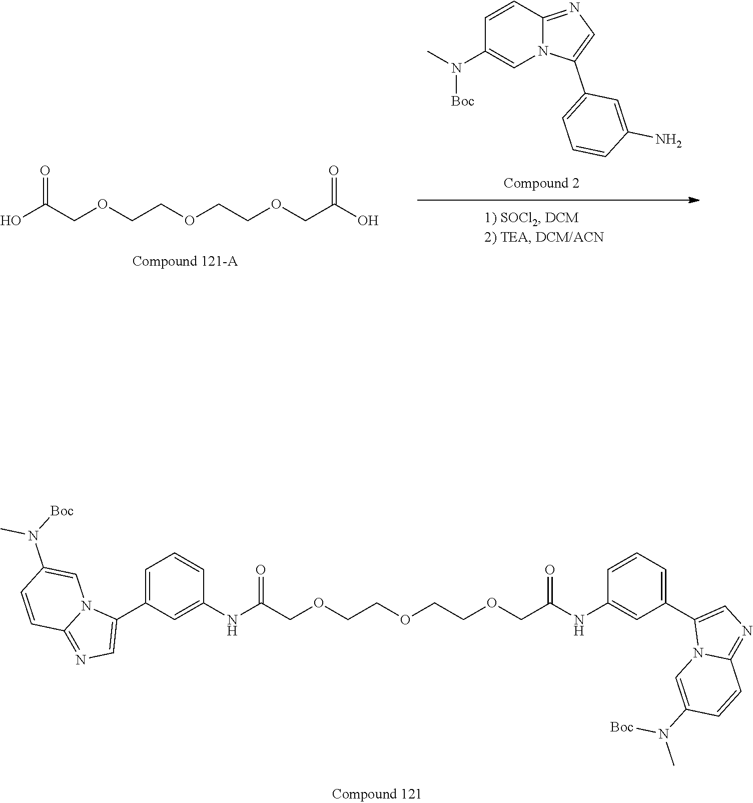 [SMILES: CN(C(=O)OC(C)(C)C)c1ccc2ncc(-c3cccc(N)c3)n2c1 ; O=C(O)COCCOCCOCC(=O)O ; CN(C(=O)OC(C)(C)C)c1ccc2ncc(-c3cccc(NC(=O)COCCOCCOCC(=O)Nc4cccc(-c5cnc6ccc(N(C)C(=O)OC(C)(C)C)cn56)c4)c3)n2c1]

Step 1: The synthesis of compound 121 was performed referring to the first step of the synthesis process of compound 116.

### ((((2,2′-((oxybis(ethane-2,1-diyl))bis(oxy))bis(acetyl))bis(azadiyl))bis(3,1-phylene))bis(imidazo[1,2-a]pyridin-3,6-diyl))bis(tert-butyl (methyl)carbamate)

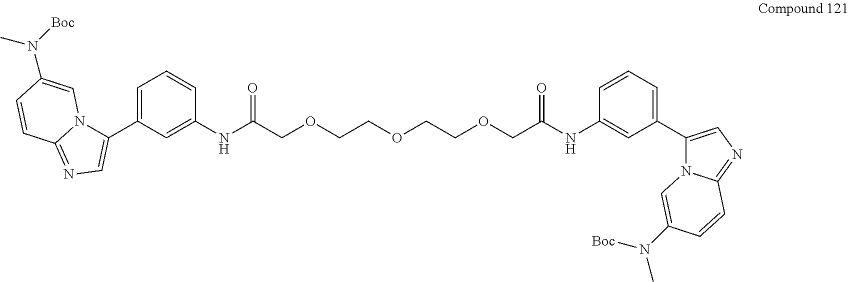 [SMILES: CN(C(=O)OC(C)(C)C)c1ccc2ncc(-c3cccc(NC(=O)COCCOCCOCC(=O)Nc4cccc(-c5cnc6ccc(N(C)C(=O)OC(C)(C)C)cn56)c4)c3)n2c1]

LC_MS: (ES⁺): m/z 863.4 [M+H]⁺.

¹H NMR (400 MHz, DMSO) δ 8.89 (s, 2H), 8.32 (s, 5H), 7.74 (s, 5H), 7.45 (t, J=23.9 Hz, 4H), 7.26-7.21 (m, 2H), 4.12 (s, 4H), 3.82 (s, 8H), 3.26 (s, 6H), 1.43 (s, 18H).

### Synthesis of Compound 122

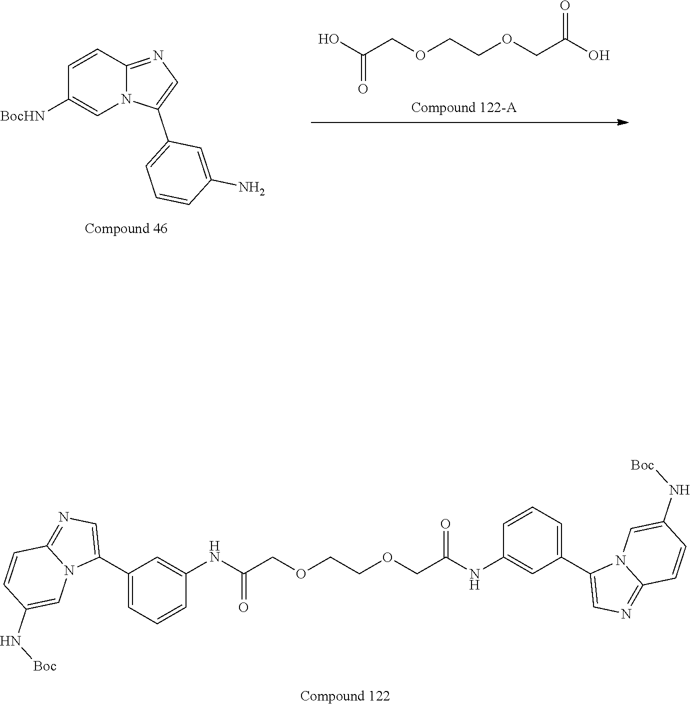 [SMILES: CC(C)(C)OC(=O)Nc1ccc2ncc(-c3cccc(N)c3)n2c1 ; O=C(O)COCCOCC(=O)O ; CC(C)(C)OC(=O)Nc1ccc2ncc(-c3cccc(NC(=O)COCCOCC(=O)Nc4cccc(-c5cnc6ccc(NC(=O)OC(C)(C)C)cn56)c4)c3)n2c1]

Step 1: The synthesis of compound 122 was performed referring to the first step of the synthesis process of compound 116.

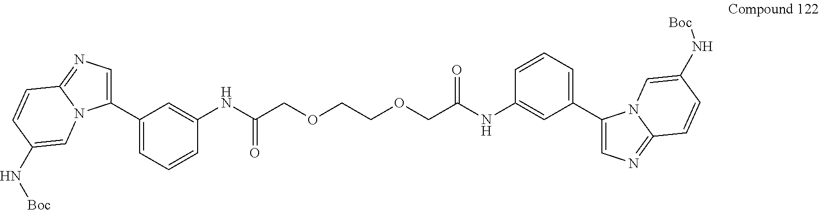 [SMILES: CC(C)(C)OC(=O)Nc1ccc2ncc(-c3cccc(NC(=O)COCCOCC(=O)Nc4cccc(-c5cnc6ccc(N)cn56)c4)c3)n2c1 ; "Boc"]

LC_MS: (ES⁺): m/z 791.1 [M+H]⁺.

### Synthesis of Compound 123

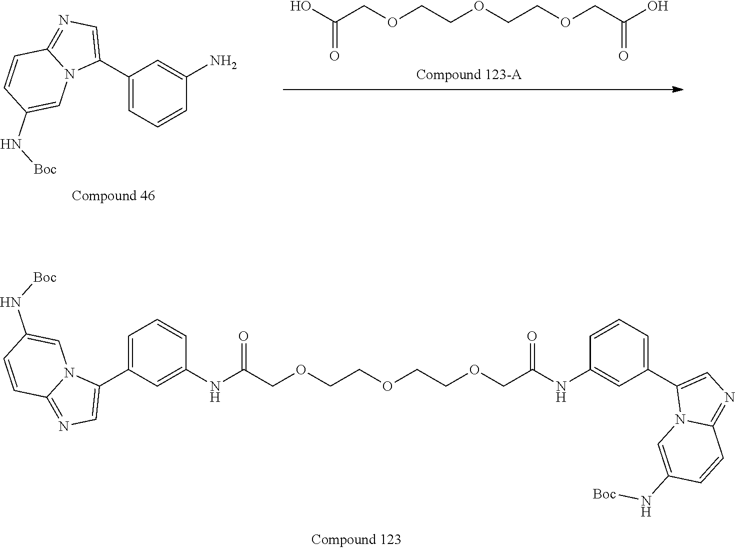 [SMILES: CC(C)(C)OC(=O)Nc1ccc2ncc(-c3cccc(N)c3)n2c1 ; O=C(O)COCCOCCOCC(=O)O ; CC(C)(C)OC(=O)Nc1ccc2ncc(-c3cccc(NC(=O)COCCOCCOCC(=O)Nc4cccc(-c5cnc6ccc(NC(=O)OC(C)(C)C)cn56)c4)c3)n2c1]

Step 1: The synthesis of compound 123 was performed referring to the first step of the synthesis process of compound 116.

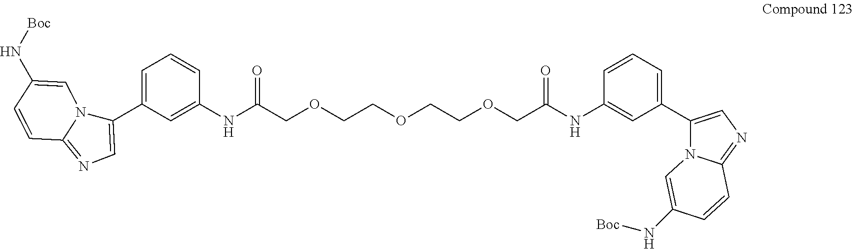 [SMILES: CC(C)(C)OC(=O)Nc1ccc2ncc(-c3cccc(NC(=O)COCCOCCOCC(=O)Nc4cccc(-c5cnc6ccc(NC(=O)OC(C)(C)C)cn56)c4)c3)n2c1]

LC_MS: (ES⁺): m/z 835.1 [M+H]⁺.

### Example 30: Synthesis of Compound 124 and Compound 125

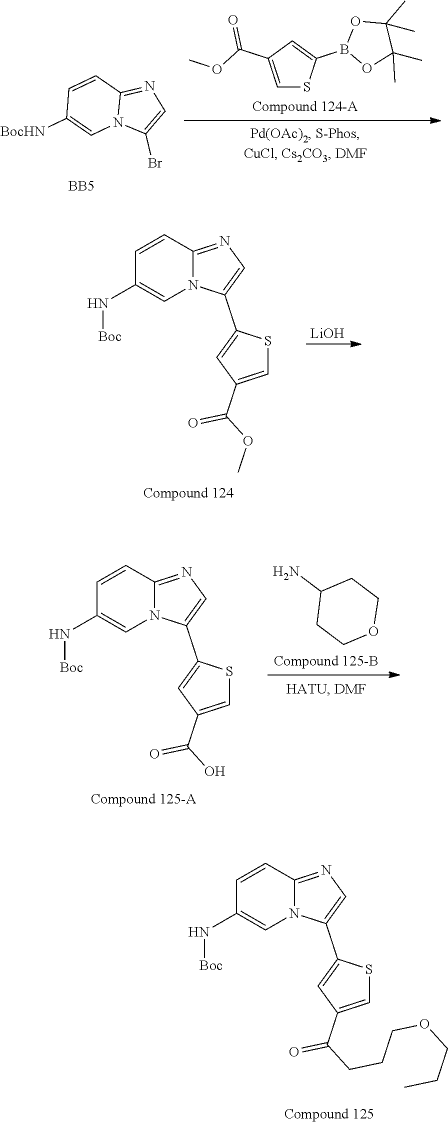

### Step 1: Compound 124 methyl 5-(6-((tert-butoxycarbonyl)amino)imidazo[1,2-a]pyridin-3-yl)thiophene-3-carboxylate

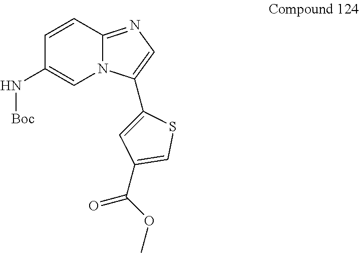 [SMILES: COC(=O)c1csc(-c2cnc3ccc(NC(=O)OC(C)(C)C)cn23)c1]

Under nitrogen protection, a solution containing intermediate BB5 tert-butyl (3-bromoimidazo[1,2-a]pyridin-6-yl)carbamate (30 mg, 0.096 mmol), compound 124-A methyl 5-(4,4,5,5-tetramethyl-1,3,2-dioxaboran-2-yl)thiophene-3-carboxylate (38.65 mg, 0.144 mmol), cesium carbonate (125.11 mg, 0.384 mmol), S-Phos (7.9 mg, 0.0192 mmol) and cuprous chloride (9.5 mg, 0.096 mmol) in N,N-dimethylformamide was added with palladium acetate (1.08 mg, 0.0048 mmol). The reaction system was replaced with nitrogen 3 times, heated to 100° C., and stirred for 18 hours of reaction. The reaction was monitored by TLC to be completed. The reaction solution was extracted with water (15 ml) and ethyl acetate (15 ml×3). The combined organic layers were washed with saturated brine, dried over anhydrous sodium sulfate, and concentrated under reduced pressure. The resulting crude product was separated and purified by preparative TLC (eluted with dichloromethane containing 4.76% methanol) to obtain a yellow solid compound 124 methyl 5-(6-((tert-butoxycarbonyl)amino)imidazo[1,2-a]pyridin-3-yl)thiophene-3-carboxylate (12 mg).

LC_MS: (ES⁺): m/z 374.0 [M+H]⁺.

### Step 2: Compound 125-A 5-(6-((tert-butoxycarbonyl)amino)imidazo[1,2-a]pyridin-3-yl)thiophene-3-carboxylic acid

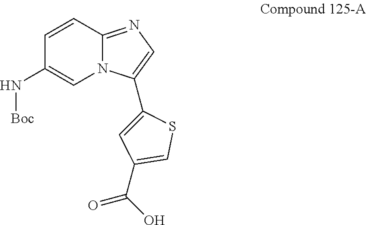 [SMILES: CC(C)(C)OC(=O)Nc1ccc2ncc(-c3cc(C(=O)O)cs3)n2c1]

A solution of compound 124 methyl 5-(6-((tert-butoxycarbonyl)amino)imidazo[1,2-a]pyridin-3-yl)thiophene-3-carboxylate (12 mg, 0.032 mmol) in methanol/water/tetrahydrofuran (1.5 ml, 1:1:1) was added with lithium hydroxide hydrate (6.71 mg, 0.16 mmol). The reaction solution was stirred at room temperature for 5 hours of reaction. The reaction was monitored by TLC to be completed. The reaction mixture was acidified with dilute hydrochloric acid and concentrated under reduced pressure to obtain yellow solid compound 125-A 5-(6-((tert-butoxycarbonyl)amino)imidazo[1,2-a]pyridin-3-yl)thiophene-3-carboxylic acid (10 mg, crude product). The crude product was directly used in the next reaction.

### Step 3: Compound 125 tert-butyl (3-(4-((tetrahydro-2H-pyran-4-yl)carbamoyl)thiophen-2-yl)imidazo[1,2-a]pyridin-6-yl) carbamate

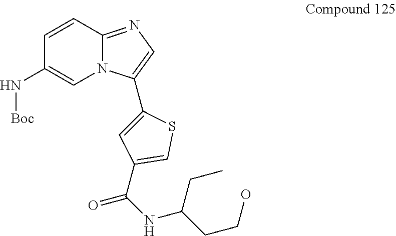 [SMILES: CCC(CCO)NC(=O)c1csc(-c2cnc3ccc(NC(=O)OC(C)(C)C)cn23)c1]

A solution containing compound 125-A 5-(6-((tert-butoxycarbonyl)amino)imidazo[1,2-a]pyridin-3-yl)thiophene-3-carboxylic acid (10 mg, 0.0278 mmol), compound 125-B tetrahydro-2H-pyran-4-amine (2.81 mg, 0.0278 mmol), HATU (10.56 mg, 0.0278 mmol) and N,N-diisopropylethylamine (10.76 mg, 0.0834 mmol) in N,N-dimethylformamide (1 ml) was stirred at room temperature for 18 hours of reaction. The reaction was monitored by TLC to be completed. The reaction solution was extracted with water and ethyl acetate. The combined organic layers were washed with saturated brine, dried over anhydrous sodium sulfate, and concentrated under reduced pressure. The resulting crude product was separated and purified by preparative TLC (eluted with dichloromethane containing 4.76% methanol) to obtain yellow solid compound 125 tert-butyl (3-(4-((tetrahydro-2H-pyran-4-yl)carbamoyl)thiophen-2-yl)imidazo[1,2-a]pyridin-6-yl) carbamate (2.3 mg).

LC_MS: (ES⁺): m/z 443.1 [M+H]⁺.

The following compounds were synthesized referring to the synthesis of compound 124 and compound 125.

### Synthesis of Compound 126 and Compound 127

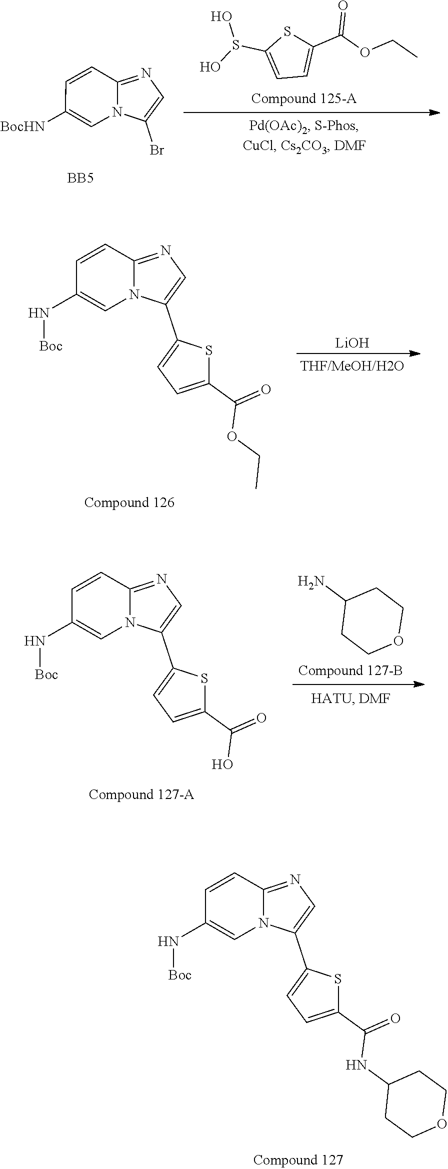

### Step 1: Compound 126 ethyl 5-(6-((tert-butoxycarbonyl)amino)imidazo[1,2-a]pyridin-3-yl)thiophen-2-carboxylate

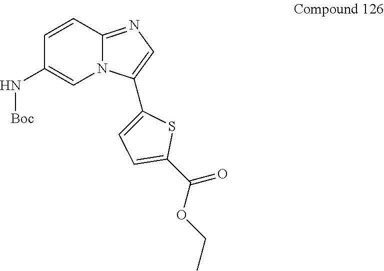 [SMILES: CCOC(=O)c1ccc(-c2cnc3ccc(NC(=O)OC(C)(C)C)cn23)s1]

This step was performed referring to the first step of the synthesis process of compound 125.

LC_MS: (ES⁺): m/z 2.636 [M+H]⁺.

Step 2: Compound 127-A was synthesized referring to the second step of the synthesis process of compound 125.

### Step 3: Compound 127 tert-butyl (3-(5-((tetrahydro-2H-pyran-4-yl)carbamoyl)thiophen-2-yl)imidazo[1,2-a]pyridin-6-yl)carbamate

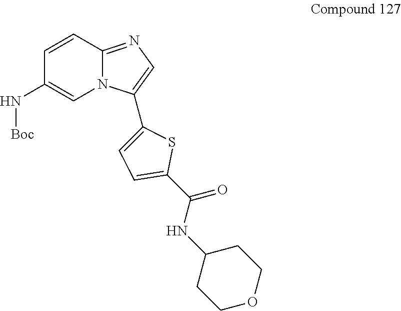 [SMILES: CC(C)(C)OC(=O)Nc1ccc2ncc(-c3ccc(C(=O)NC4CCOCC4)s3)n2c1]

This step was performed referring to the third step of the synthesis process of compound 125.

LC MS: (ES⁺): m/z 443.1 [M+H]⁺.

### Example 31: Synthesis of Compound 128 and Compound 129

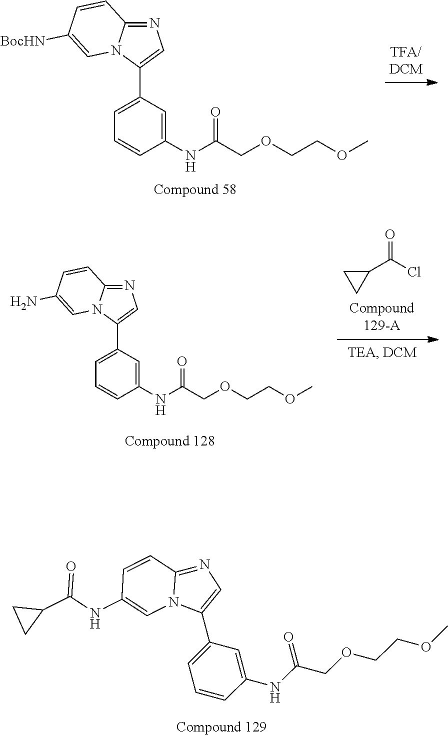 [SMILES: COCCOCC(=O)Nc1cccc(-c2cnc3ccc(NC(=O)OC(C)(C)C)cn23)c1 ; COCCOCC(=O)Nc1cccc(-c2cnc3ccc(N)cn23)c1 ; O=C(Cl)C1CC1 ; COCCOCC(=O)Nc1cccc(-c2cnc3ccc(NC(=O)C4CC4)cn23)c1]

### Step 1: Compound 128 N-(3-(6-aminoimidazo[1,2-a]pyridin-3-yl)phenyl)-2-(2-methoxyethoxy)acetamide

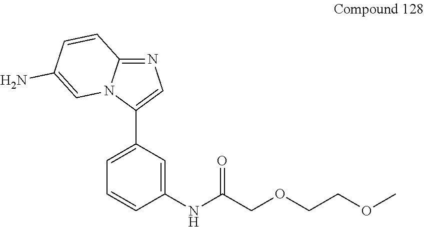 [SMILES: COCCOCC(=O)Nc1cccc(-c2cnc3ccc(N)cn23)c1]

A solution containing compound 58 tert-butyl (3-(3-(2-(2-methoxyethoxy)acetamido)phenyl)imidazo[1,2-a]pyridin-6-yl)carbamate (200 mg, 0.454 mmol) in dichloromethane (4 ml) and trifluoroacetic acid (800 l) was stirred at room temperature for 5 hours of reaction. The reaction was monitored by TLC to be completed. The reaction mixture was concentrated under reduced pressure. The obtained crude product was separated and purified by silica gel column chromatography (eluted with dichloromethane containing 4.76%-9.0% methanol) to obtain brown solid compound 128 N-(3-(6-aminoimidazo[1,2-a]pyridin-3-yl)phenyl)-2-(2-methoxyethoxy)acetamide (150 mg).

LC_MS: (ES⁺): m/z 341.1 [M+H]⁺.

### Step 2: Compound 129 N-(3-(3-(2-(2-methoxyethoxy)acetamido)phenyl)imidazo[1,2-a]pyridin-6-yl)cyclopropanecarboxamide

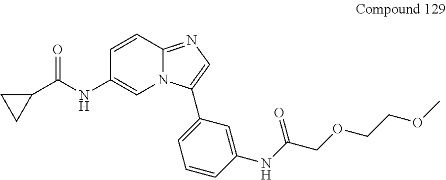 [SMILES: COCCOCC(=O)Nc1cccc(-c2cnc3ccc(NC(=O)C4CC4)cn23)c1]

A solution containing compound 128 N-(3-(6-aminoimidazo[1,2-a]pyridin-3-yl)phenyl)-2-(2-methoxyethoxy)acetamide (30 mg, 0.0881 mmol) and triethylamine (26.74 mg, 0.264 mmol) in dichloromethane (2 ml) was added with cyclopropaneformyl chloride (9.67 mg, 0.0925 mmol). The reaction solution was heated from 0° C. to room temperature and stirred for 18 hours of reaction. The reaction was monitored by TLC to be completed. The reaction solution was extracted with water and dichloromethane. The combined organic layers were dried over anhydrous sodium sulfate and concentrated under reduced pressure. The crude product obtained was separated and purified by preparative TLC (eluted with dichloromethane containing 4.76% methanol) to obtain yellow solid compound 129 N-(3-(3-(2-(2-methoxyethoxy)acetamido)phenyl)imidazo[1,2-a]pyridin-6-yl)cyclopropanecarboxamide (16 mg).

LC_MS: (ES⁺): m/z 409.0 [M+H]⁺.

¹H NMR (400 MHz, CDCl₃) δ 9.29 (d, J=20.4 Hz, 2H), 9.15 (s, 1H), 7.90-7.75 (m, 2H), 7.71 (s, 1H), 7.61 (d, J=8.2 Hz, 1H), 7.47 (dd, J=13.6, 5.7 Hz, 2H), 7.31 (s, 1H), 4.15 (s, 2H), 3.82-3.78 (m, 2H), 3.66-3.62 (m, 2H), 3.46 (s, 3H), 1.82 (dd, J=8.0, 3.7 Hz, 1H), 1.03-0.97 (m, 2H), 0.84 (dd, J=7.6, 3.1 Hz, 2H).

The following compounds were synthesized referring to the synthesis of compound 129

### Synthesis of Compound 130

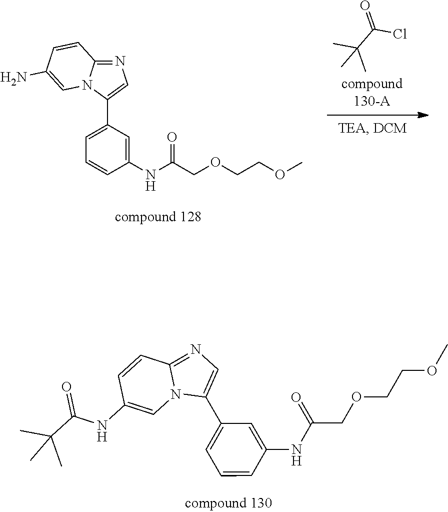

Step 1: The synthesis of compound 130 was performed referring to the second step of the synthesis process of compound 129.

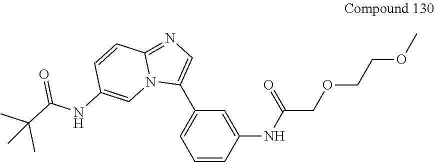 [SMILES: COCCOCC(=O)Nc1cccc(-c2cnc3ccc(NC(=O)C(C)(C)C)cn23)c1]

LC_MS: (ES⁺): m/z 425.1 [M+H]⁺.

¹H NMR (400 MHz, DMSO) δ 9.85 (s, 1H), 9.41 (s, 1H), 9.26 (s, 1H), 7.87 (t, J=1.7 Hz, 1H), 7.79 (d, J=8.2 Hz, 1H), 7.68 (d, J=8.5 Hz, 2H), 7.56 (dd, J=14.0, 6.2 Hz, 2H), 7.35 (d, J=7.8 Hz, 1H), 4.12 (s, 2H), 3.72-3.68 (m, 2H), 3.56-3.53 (m, 2H), 3.30 (s, 3H), 1.23 (s, 9H).

### Synthesis of Compound 131

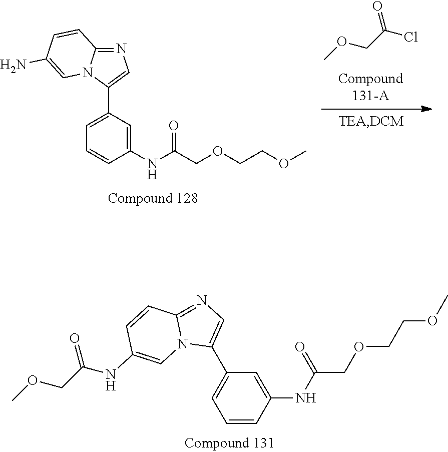

Step 1: The synthesis of compound 131 was performed referring to the second step of the synthesis process of compound 129.

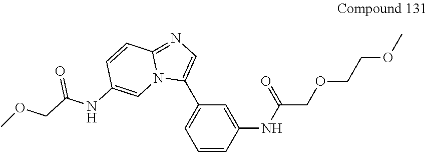

LC_MS: (ES⁺): m/z 413.1 [M+H].

¹H NMR (400 MHz, DMSO) δ 10.04 (s, 1H), 9.84 (s, 1H), 9.30 (s, 1H), 7.89 (t, J=1.7 Hz, 1H), 7.80-7.76 (m, 1H), 7.71 (d, J=8.1 Hz, 2H), 7.60-7.51 (m, 2H), 7.36 (d, J=7.9 Hz, 1H), 4.12 (s, 2H), 4.03 (s, 2H), 3.71-3.68 (m, 2H), 3.56-3.53 (m, 2H), 3.39 (s, 3H), 3.30 (s, 3H).

### Example 32: Synthesis of Compound 132, Compound 133 and Compound 134

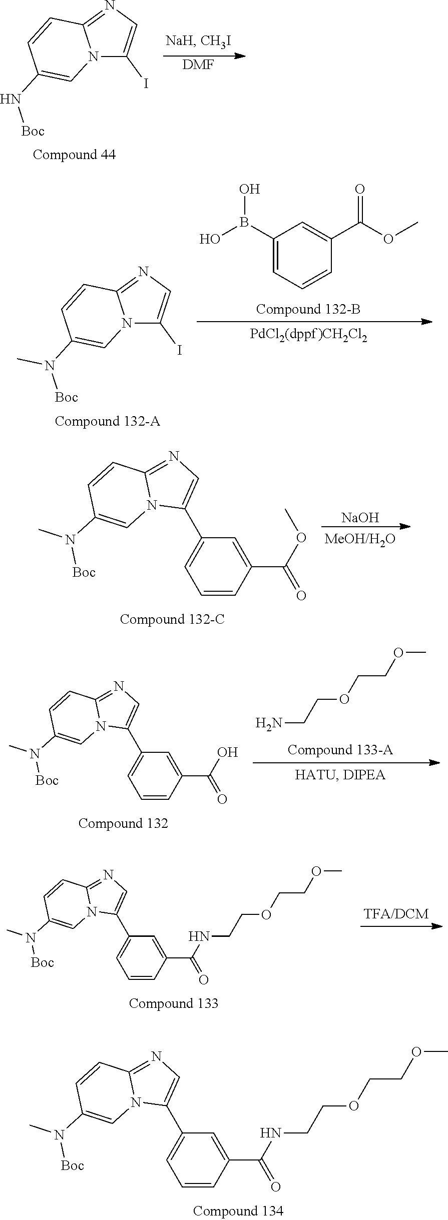

Step 1: The synthesis of compound 132-A was performed referring to the fifth step of the synthesis process of intermediate BB-6.

Step 2: The synthesis of compound 132-C was performed referring to the first step of the synthesis process of compound 2.

Step 3: Compound 132 3-(6-((tert-butoxycarbonyl)(methyl)amino)imidazo[1,2-a]pyridin-3-yl)benzoic acid

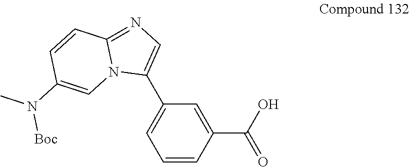 [SMILES: CN(C(=O)OC(C)(C)C)c1ccc2ncc(-c3cccc(C(=O)O)c3)n2c1]

A solution containing compound 132-C methyl 3-(6-((tert-butoxycarbonyl)(methyl)amino)imidazo[1,2-a]pyridin-3-yl)benzoate (148 mg, 0.388 mmol) in methanol (3 ml) was added with a solution of sodium hydroxide (46.56 mg, 1.164 mmol) in water (1 ml). The reaction mixture was stirred at room temperature for 4 hours of reaction. The reaction was monitored by TLC to be completed. The pH of the reaction solution was adjusted to 5-6 with dilute hydrochloric acid, and the reaction mixture was concentrated under reduced pressure to obtain brown solid compound 132 3-(6-((tert-butoxycarbonyl)(methyl)amino)imidazo[1,2-a]pyridin-3-yl)benzoic acid (138 mg, crude product), which was directly used in the next reaction.

LC_MS: (ES⁺): m/z 368.0 [M+H]⁺.

### Step 4: Compound 133 tert-butyl (3-(3-((2-(2-methoxyethoxy)ethyl)carbamoyl)phenyl)imidazo[1,2-a]pyridin-6-yl)(methyl)carbamate

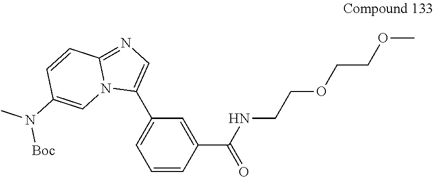

A solution containing compound 132 3-(6-((tert-butoxycarbonyl)(methyl)amino)imidazo[1,2-a]pyridin-3-yl)benzoic acid (50 mg, 0.136 mmol), compound 133-A 2-(2-methoxyethoxy)eth-1-amine (16.2 mg, 0.136 mmol) and 2-(7-azobenzotriazole)-N,N,N′,N′-tetramethylurea hexafluorophosphate (51.68 mg, 0.136 mmol) in N,N-dimethylformamide (1 ml) was added with N,N-diisopropylethylamine (52.63 mg, 0.408 mmol). The reaction mixture was stirred at room temperature for 4 hours of reaction. The reaction was monitored by TLC to be completed. The reaction solution was extracted with water and ethyl acetate. The combined organic layers were washed with saturated brine, dried over anhydrous sodium sulfate, and concentrated under reduced pressure. The resulting crude product was separated and purified by preparative TLC (eluted with dichloromethane containing 8.33% methanol) to obtain yellow gel compound 133 tert-butyl (3-(3-((2-(2-methoxyethoxy)ethyl)carbamoyl)phenyl)imidazo[1,2-a]pyridin-6-yl) (methyl)carbamate (44 mg).

LC_MS: (ES⁺): m/z 469.1 [M+H]⁺.

Step 5: The synthesis of compound 134 was performed referring to the synthesis process of compound 63.

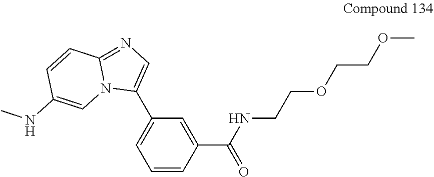

LC_MS: (ES) m/z 369.1 [M+H]⁺.

The following compounds were synthesized referring to the synthesis of compound 134

### Synthesis of Compound 135 and Compound 136

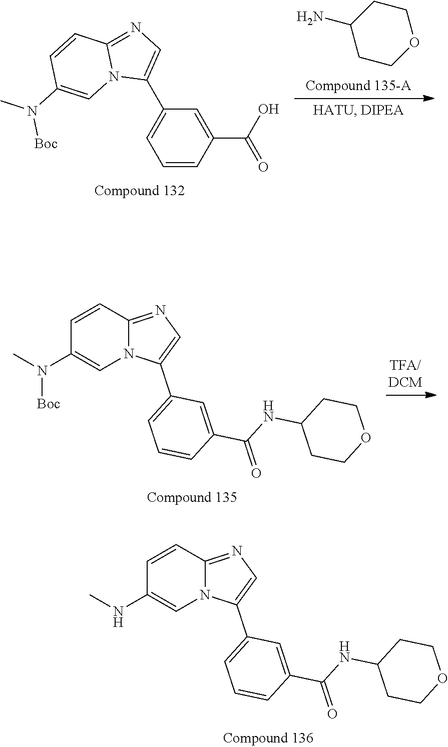 [SMILES: CN(C(=O)OC(C)(C)C)c1ccc2ncc(-c3cccc(C(=O)O)c3)n2c1 ; NC1CCOCC1 ; CN(C(=O)OC(C)(C)C)c1ccc2ncc(-c3cccc(C(=O)NC4CCOCC4)c3)n2c1 ; CNc1ccc2ncc(-c3cccc(C(=O)NC4CCOCC4)c3)n2c1]

Step 1: The synthesis of compound 135 was performed referring to the fourth step of the synthesis process of compound 134.

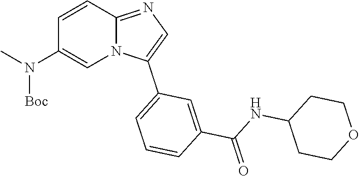 [SMILES: CN(C(=O)OC(C)(C)C)c1ccc2ncc(-c3cccc(C(=O)NC4CCOCC4)c3)n2c1]

LC_MS: (ES⁺): m/z 451.1 [M+H]⁺.

Step 2: The synthesis of compound 136 was performed referring to the fifth step of the synthesis process of compound 134.

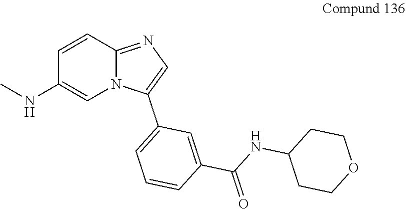 [SMILES: CNc1ccc2ncc(-c3cccc(C(=O)NC4CCOCC4)c3)n2c1]

LC_MS: (ES⁺): m/z 351.0 [M+H]⁺.

¹H NMR (400 MHz, CDCl₃) δ 7.98 (s, 2H), 7.86 (d, J=7.1 Hz, 1H), 7.66 (s, 2H), 7.58 (t, J=7.7 Hz, 1H), 7.35 (s, 1H), 6.95 (s, 1H), 4.28-4.17 (m, 1H), 4.02 (d, J=10.6 Hz, 2H), 3.58-3.49 (m, 2H), 2.75 (s, 3H), 2.07-1.96 (m, 2H), 1.68-1.60 (m, 2H).

### Example 33: Synthesis of Compound 137

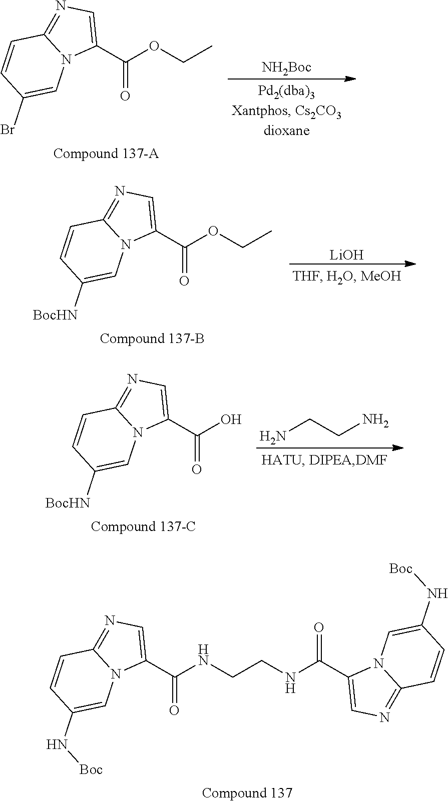

### Step 1: Compound 137-B ethyl 6-((tert-butoxycarbonyl)amino)imidazo[1,2-a]pyridin-3-carboxylate

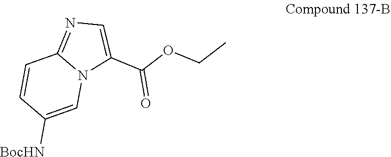 [SMILES: CCOC(=O)c1cnc2ccc(NC(=O)OC(C)(C)C)cn12]

A suspension containing compound 137-A ethyl 6-bromoimidazo[1,2-a]pyridin-3-carboxylate (1 g, 3.37 mmol), tert-butyl carbamate (592 mg, 5.06 mmol), 9,9-dimethyl-4,5-bis(diphenylphosphino)xanthene (390 mg, 0.67 mmol) and cesium carbonate (3.3 g, 10.11 mmol) in 1,4-dioxane (1 ml) was added with tris(dibenzylideneacetone)dipalladium (309 mg, 0.34 mmol) under a nitrogen atmosphere at room temperature. The reaction solution was replaced with nitrogen three times, and stirred at 110° C. for 16 hours of reaction. The reaction was monitored by TLC to be completed. The reaction solution was cooled to room temperature and partitioned between water (20 ml) and ethyl acetate (20 ml). The organic layer was collected and the aqueous layer was extracted with dichloromethane containing 10% methanol (10 ml×3). The organic layers were combined, washed with saturated brine (30 ml), dried over anhydrous sodium sulfate, and concentrated under reduced pressure. The resulting crude product was separated and purified by Prep-TLC (eluted with dichloromethane containing 0.8% methanol) to obtain a light yellow solid compound 137-B ethyl 6-((tert-butoxycarbonyl)amino)imidazo[1,2-a]pyridin-3-carboxylate (600 mg, 52%).

LC_MS: (ES⁺): m/z 306.6 [M+H]⁺.

¹H NMR (400 MHz, CDCl₃): δ 1.42 (t, J=7.2 Hz, 3H), 1.54 (s, 9H), 4.39-4.44 (m, 2H), 6.60 (s, 1H), 7.36-7.38 (m, 1H), 7.66 (d, J=9.6 Hz, 1H), 8.26 (s, 1H), 9.67 (s, 1H).

### Step 2: Compound 137-C 6-((tert-butoxycarbonyl)amino)imidazo[1,2-a]pyridin-3-carboxylic acid

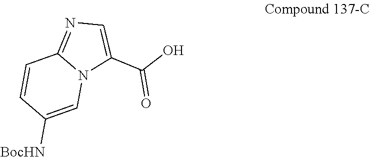 [SMILES: CC(C)(C)OC(=O)Nc1ccc2ncc(C(=O)O)n2c1]

A mixed solution containing compound 137-B ethyl 6-((tert-butoxycarbonyl)amino)imidazo[1,2-a]pyridin-3-carboxylate (600 mg, 1.97 mmol) and lithium hydroxide monohydrate (248 mg, 5.90 mmol) in tetrahydrofuran (4 ml)-water (1 ml)-methanol (1 ml) was stirred at room temperature for 4 hours of reaction. The reaction was monitored by TLC to be completed. The reaction solution was acidified to pH 5-6 with 1N hydrochloric acid, and extracted with dichloromethane containing 10% methanol (20 ml×5). The organic layers were combined, washed with saturated brine (30 ml), dried over anhydrous sodium sulfate, and concentrated under reduced pressure to obtain crude white solid compound 137-C 6-((tert-butoxycarbonyl)amino)imidazo[1,2-a]pyridin-3-carboxylic acid (300 mg, 55%), which was used in the next step without further purification.

LC_MS: (ES⁺): m/z 278.4 [M+H]⁺.

¹H NMR (400 MHz, DMSO-d6): δ 1.50 (s, 9H), 7.38-7.41 (m, 1H), 7.62 (d, J=9.2 Hz, 1H), 8.02 (s, 1H), 9.55 (s, 1H), 9.86 (s, 1H).

### Step 3: Compound 137 di-tert-butyl (3,3′-((ethane-1,2-diylbis(azadiyl))bis(carbonyl)) bis(imidazo[1,2-a]pyridin-6,3-diyl))dicarbamate

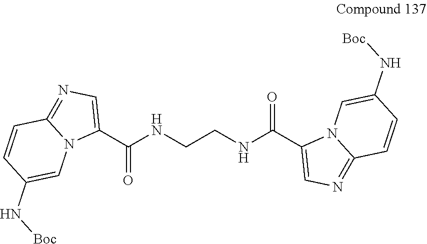 [SMILES: CC(C)(C)OC(=O)Nc1ccc2ncc(C(=O)NCCNC(=O)c3cnc4ccc(NC(=O)OC(C)(C)C)cn34)n2c1]

A solution containing compound 137-C 6-((tert-butoxycarbonyl)amino)imidazo[1,2-a]pyridin-3-carboxylic acid (20 mg, 0.072 mmol), ethane-1,2-diamine (2.17 mg, 0.036 mmol) and N-ethyl-N-isopropylpropan-2-amine (14 mg, 0.108 mmol) in DMF (1 mL) was added with HATU (42 mg, 0.11 mmol). The reaction solution was warmed to room temperature, and stirred for 30 minutes of reaction. The reaction was monitored by TLC to be completed. The mixture was partitioned between ethyl acetate (10 ml) and water (10 ml). The organic layer was collected, washed with saturated brine (10 ml), dried over anhydrous sodium sulfate, and concentrated under reduced pressure. The resulting crude product was separated and purified to preparative TLC (eluted with dichloromethane (50%)-ethyl acetate (50%) containing 10% ammonia methanol) was separated and purified to obtain white solid compound 137 di-tert-butyl (3,3′-((ethane-1,2-diylbis(azadiyl))bis(carbonyl))bis(imidazo[1,2-a]pyridin-6,3-diyl))dicarbamate (8.7 mg, 42%).

LC_MS: (ES⁺): m/z 579.3 [M+H]⁺.

¹H NMR (400 MHz, DMSO-d6): δ 1.50 (s, 18H), 3.47-3.48 (m, 4H), 7.38-7.40 (m, 2H), 7.61-7.64 (m, 2H), 8.26 (s, 2H), 8.58-8.60 (m, 2H), 9.55 (s, 2H), 9.98 (s, 2H)

Using the corresponding amine compound, the following compounds were synthesized referring to the synthesis of compound 137

### Synthesis of Compound 138

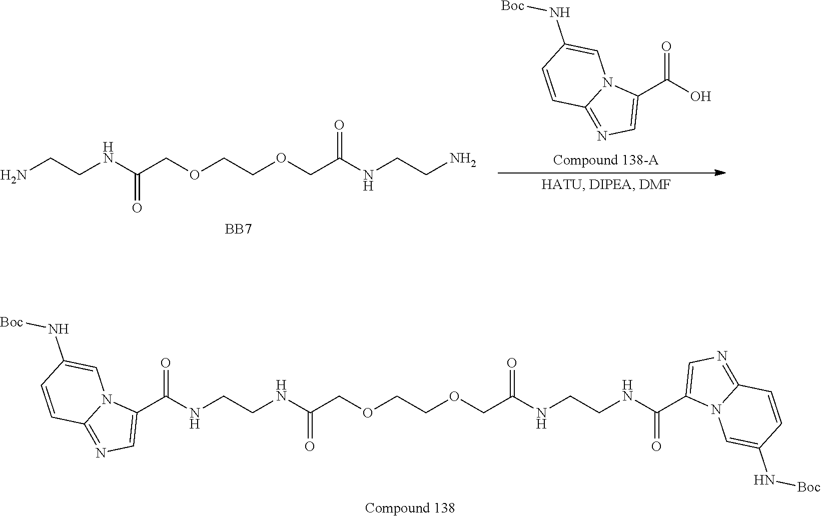

Step 1: The synthesis of compound 138 was performed referring to the third step of the synthesis process of compound 137.

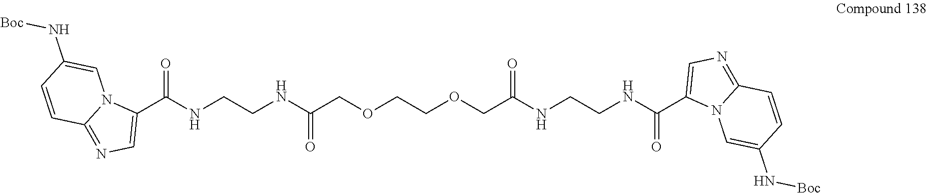 [SMILES: CC(C)(C)OC(=O)Nc1ccc2ncc(C(=O)NCCNC(=O)COCCOCC(=O)NCCNC(=O)c3cnc4ccc(NC(=O)OC(C)(C)C)cn34)n2c1]

LC_MS: (ES⁺): m/z 781.6 [M+H]⁺.

¹H NMR (400 MHz, DMSO-d6): δ 1.49 (s, 18H), 3.29 (s, 4H), 3.34-3.36 (m, 4H), 3.60 (s, 4H), 3.88 (s, 4H), 7.36-7.38 (m, 2H), 7.60-7.62 (d, J=9.6 Hz, 2H), 7.91 (t, J=5.6 Hz, 2H), 8.21 (s, 2H), 8.45 (t, J=5.2 Hz, 2H), 9.53 (s, 2H), 9.95 (s, 2H).

### Synthesis of Compound 139

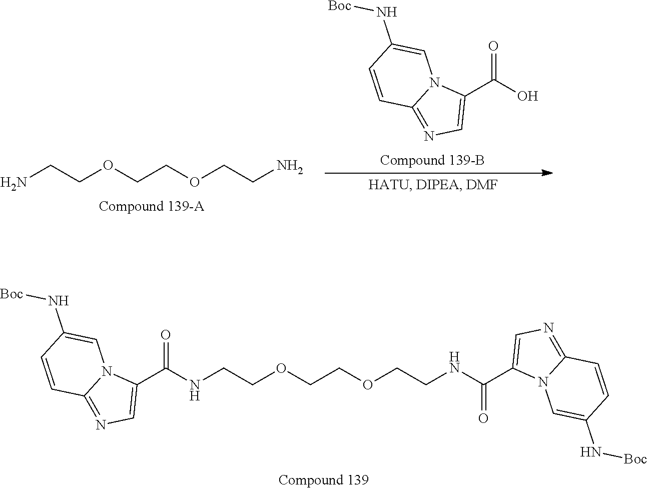

Step 1: The synthesis of compound 139 was performed referring to the third step of the synthesis process of compound 137.

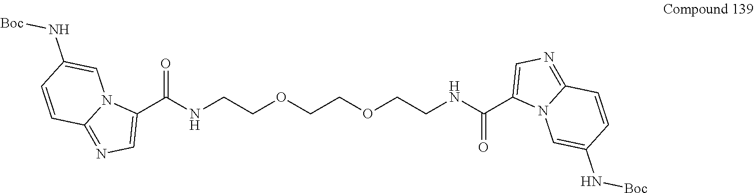 [SMILES: CC(C)(C)OC(=O)Nc1ccc2ncc(C(=O)NCCOCCOCCNC(=O)c3cnc4ccc(NC(=O)OC(C)(C)C)cn34)n2c1]

LC_MS: (ES⁺): m/z 667.2 [M+H]⁺.

¹HNMR (400 MHz, DMSO-d6): δ 1.49 (s, 18H), 3.40-3.44 (m, 4H), 3.54-3.58 (m, 8H), 7.35-7.38 (m, 2H), 7.61 (d, J=9.6 Hz, 2H), 8.26 (s, 2H), 8.47 (t, J=5.2 Hz, 2H), 9.54 (s, 2H), 9.97 (s, 2H).

### Synthesis of Compound 140

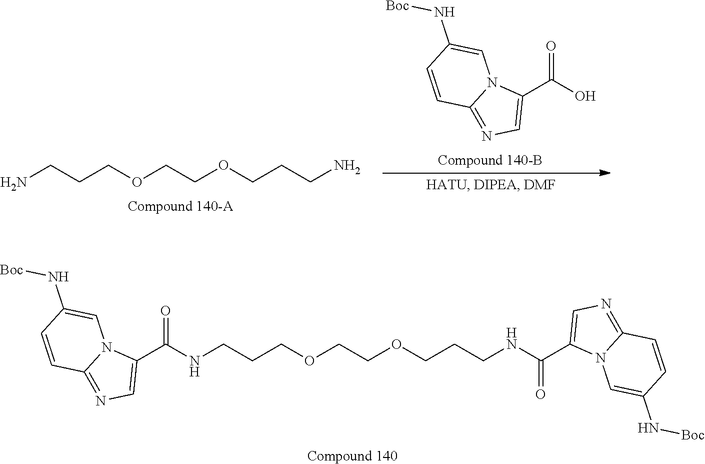

Step 1: The synthesis of compound 140 was performed referring to the third step of the synthesis process of compound 137.

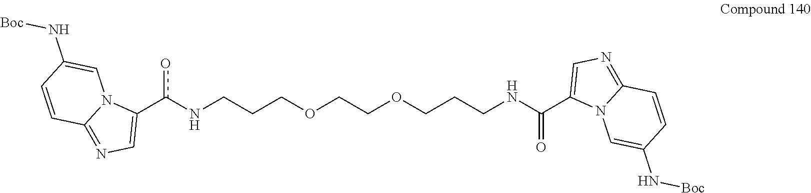

LC_MS: (ES⁺): m/z 695.2 [M+H]⁺.

¹H NMR (400 MHz, DMSO-d6): δ 1.49 (s, 18H), 1.74-1.81 (m, 4H), 3.29 (s, 2H), 3.34 (brs, 2H), 3.47-3.53 (m, 8H), 7.36 (d, J=9.6 Hz, 2H), 7.61 (d, J=9.6 Hz, 2H), 8.22 (s, 2H), 8.36 (t, J=5.6 Hz, 2H), 9.52 (s, 2H), 9.56 (s, 2H).

### Synthesis of Compound 141

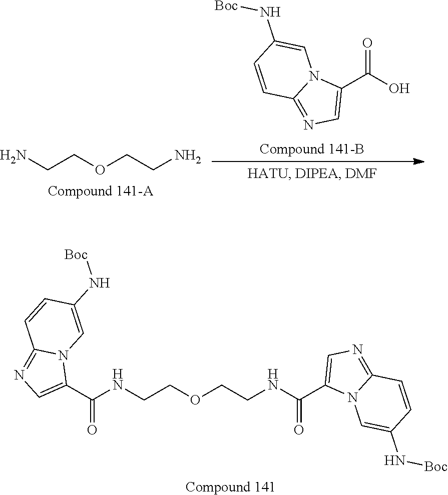

Step 1: The synthesis of compound 141 was performed referring to the third step of the synthesis process of compound 137.

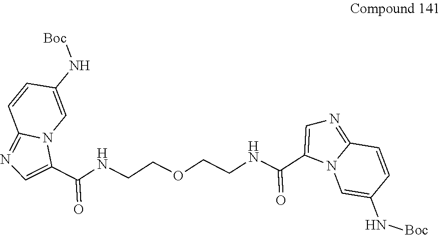 [SMILES: CC(C)(C)OC(=O)Nc1ccc2ncc(C(=O)NCCOCCNC(=O)c3cnc4ccc(NC(=O)OC(C)(C)C)cn34)n2c1]

LC_MS: (ES⁺): m/z 623.6 [M+H]⁺.

¹H NMR (400 MHz, DMSO-d6): δ 1.49 (s, 18H), 3.45-3.49 (m, 4H), 3.61 (t, J=5.8 Hz, 4H), 7.37-7.40 (m, 2H), 7.61 (d, J=9.6 Hz, 2H), 8.26 (s, 2H), 8.46 (t, J=5.6 Hz, 2H), 9.54 (s, 2H), 9.96 (s, 2H).

### Example 34: Synthesis of Compound 142

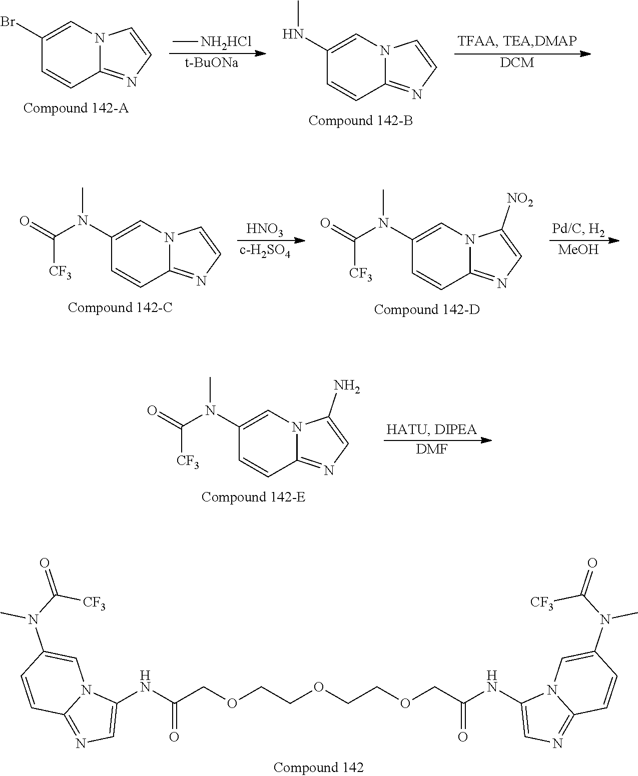

### Step 1: Compound 142-B N-methylimidazo[1,2-a]pyridin-6-amine

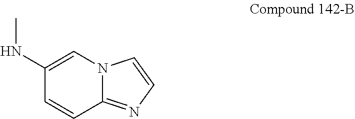

A solution containing compound 142-A 6-bromoimidazo[1,2-a]pyridine (50 mg, 0.25 mmol), (R)-1-[(S)-2-(dicyclohexylphosphino)ferrocenyl]ethyl tert-butylphosphine (cas:158923-11-6)(14 mg, 0.025 mmol), bis[tris(2-methylphenyl)phosphine]palladium (cas:69861-71-8)(18 mg, 0.025 mmol) and sodium tert-butoxide (328 mg, 3.3 mmol) in 1,4-dioxane was replaced with nitrogen three times, and then added with methylamine hydrochloride (171 mg, 2.5 mmol) rapidly. The reaction solution was sealed and stirred at 100° C. for 24 hours of reaction. The reaction was monitored by TLC to be completed. The reaction solution was partitioned between ethyl acetate (20 ml) and water (20 ml). The organic layer was collected, washed with saturated brine (10 ml), dried over anhydrous sodium sulfate, and concentrated under reduced pressure. The obtained crude product was separated and purified by silica gel column chromatography (eluted with dichloromethane containing 5% methanol) to obtain a gray oil compound 142-B N-methylimidazo[1,2-a]pyridin-6-amine (17 mg, 45%).

LC_MS: (ES⁺): m/z 148.1 [M+H]⁺.

### Step 2: Compound 142-C 2,2,2-trifluoro-N-(imidazo[1,2-a]pyridin-6-yl)-N-methylacetamide

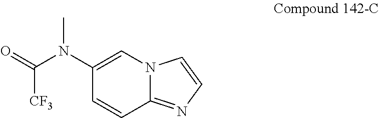 [SMILES: CN(C(=O)C(F)(F)F)c1ccc2nccn2c1]

A solution containing compound 142-B N-methylimidazo[1,2-a]pyridin-6-amine (50 mg, 0.34 mmol), triethylamine (114 mg, 1.12 mmol) and N,N-dimethylpyridin-4-amine (4 mg, 0.034 mmol) in dichloromethane (1 ml) was added with trifluoroacetic anhydride (86 mg, 0.41 mmol) dropwise at 0° C. The reaction solution was warmed to room temperature and stirred for 15 hours of reaction. The reaction was monitored by TLC to be completed. The reaction solution was partitioned between dichloromethane (10 ml) and water (10 ml). The organic layer was collected, washed with saturated brine (10 ml), dried over anhydrous sodium sulfate, and concentrated under reduced pressure. The obtained crude product was separated and purified by silica gel column chromatography (eluted with dichloromethane containing 3% methanol) to obtain a yellow oil compound 142-C 2,2,2-trifluoro-N-(imidazo[1,2-a]pyridin-6-yl)-N-methylacetamide (33 mg, 40%).

LC_MS: (ES⁺): m/z 244.10 [M+H]⁺.

### Step 3: Compound 142-D 2,2,2-trifluoro-N-methyl-N-(3-nitroimidazo[1,2-a]pyridin-6-yl)acetamide

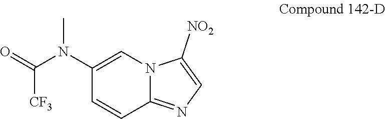 [SMILES: CN(C(=O)C(F)(F)F)c1ccc2ncc([N+](=O)[O-])n2c1]

A solution containing compound 142-C 2,2,2-trifluoro-N-(imidazo[1,2-a]pyridin-6-yl)-N-methylacetamide (33 mg, 0.14 mmol) in concentrated sulfuric acid (1 ml) was added with concentrated nitric acid (9 mg, 0.14 mmol) under stirring at 0° C. The reaction mixture was warmed to room temperature and stirred for 1 hour of reaction. The reaction was monitored by TLC to be completed. The reaction solution was basified to pH=7 with saturated aqueous sodium carbonate solution, and then partitioned between ethyl acetate (10 ml) and water (10 ml). The organic layer was collected, washed with saturated brine (10 ml), dried over anhydrous sodium sulfate, and concentrated under reduced pressure to obtain a yellow solid crude compound 142-D 2,2,2-trifluoro-N-methyl-N-(3-nitroimidazo[1,2-a]pyridin-6-yl)acetamide (30 mg, 76%), which was used directly in the next step without further purification.

LC_MS: (ES⁺): m/z 288.95 [M+H]⁺.

### Step 4: Compound 142-E N-(3-aminoimidazo[1,2-a]pyridin-6-yl)-2,2,2-trifluoro-N-methylacetamide

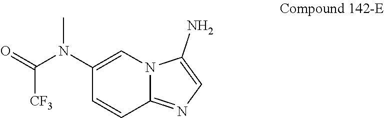 [SMILES: CN(C(=O)C(F)(F)F)c1ccc2ncc(N)n2c1]

A solution containing palladium carbon (20%, 6 mg) and compound 142-D 2,2,2-trifluoro-N-methyl-N-(3-nitroimidazo[1,2-a]pyridin-6-yl)acetamide (30 mg, 0.1 mmol) in methanol (15 ml) was stirred under a hydrogen atmosphere (hydrogen balloon) at room temperature for reaction overnight. The reaction was monitored by TLC to be completed. The reaction mixture was filtered to remove palladium carbon, and rinsed with ethanol (5 ml×2). The filtrates were combined and concentrated under reduced pressure. The obtained crude product was separated and purified by preparative TLC (eluted with dichloromethane containing 5% methanol) to obtain a brown oil compound 142-E N-(3-aminoimidazo[1,2-a]pyridin-6-yl)-2,2,2-trifluoro-N-methylacetamide (8 mg, 30%).

LC_MS: (ES⁺): m/z 259.10 [M+H]⁺.

### Step 5: Compound 142 N,N′-(3,3′-((2,2′-((oxybis(ethane-2,1-diyl))bis(oxy))bis (acetyl))bis(azadialkyl))bis(imidazo[1,2-a]pyridin-6,3-diyl))bis(2,2,2-trifluoro-N-methylacetamide)

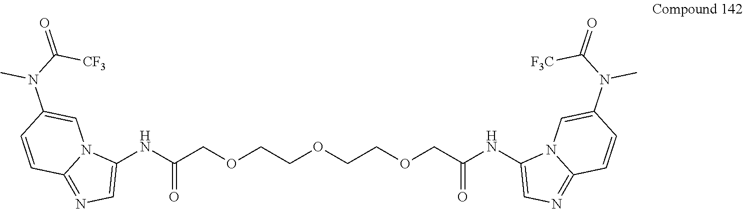

The synthesis of this step was performed referring to Example 29, the synthesis procedure of compound 123.

LC_MS: (ES⁺): m/z 703.50 [M+H]⁺.

¹H NMR (400 MHz, CDCl₃): δ 3.37 (s, 6H), 3.79-3.80 (m, 8H), 4.04-4.18 (m, 4H), 7.10-7.13 (m, 2H), 7.57-7.69 (m, 4H), 7.93-8.01 (m, 2H), 8.88-9.07 (m, 2H).

The following compounds were synthesized referring to the corresponding routes described in compound 2, intermediate BB5 or BB6:

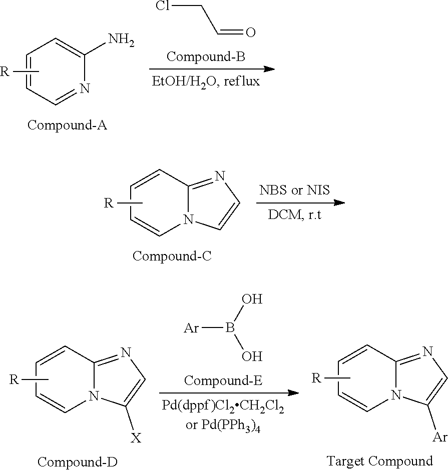

Compound 143

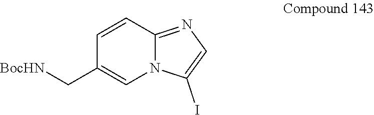 [SMILES: CC(C)(C)OC(=O)NCc1ccc2ncc(I)n2c1]

LC_MS: (ES⁺): m/z 373.9 [M+H]⁺.

¹H NMR (400 MHz, DMSO) δ 8.14 (s, 1H), 7.70 (s, 1H), 7.59 (d, J=9.2 Hz, 1H), 7.52 (t, J=5.9 Hz, 1H), 7.24 (dd, J=9.2, 1.5 Hz, 1H), 4.22 (d, J=6.0 Hz, 2H), 3.17 (d, J=4.9 Hz, 1H), 1.41 (s, 9H).

Compound 144

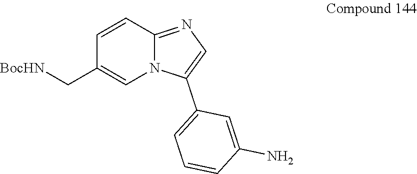 [SMILES: CC(C)(C)OC(=O)NCc1ccc2ncc(-c3cccc(N)c3)n2c1]

LC_MS: (ES⁺): m/z 339.1 [M+H]⁺.

Compound 145

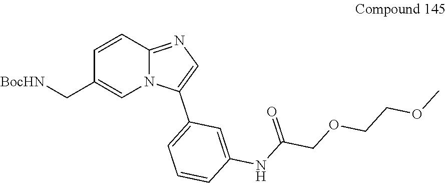 [SMILES: COCCOCC(=O)Nc1cccc(-c2cnc3ccc(CNC(=O)OC(C)(C)C)cn23)c1]

LC_MS: (ES⁺): m/z 455.1 [M+H]⁺.

Compound 146

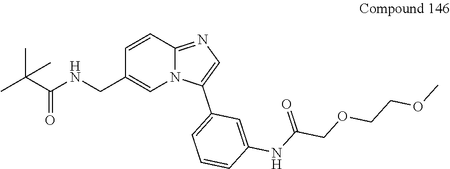 [SMILES: COCCOCC(=O)Nc1cccc(-c2cnc3ccc(CNC(=O)C(C)(C)C)cn23)c1]

LC_MS: (ES⁺): m/z 439.1 [M+H]⁺.

Compound 147

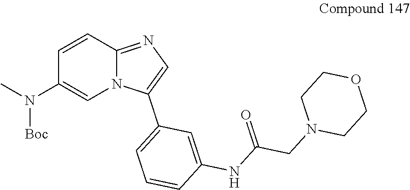 [SMILES: CN(C(=O)OC(C)(C)C)c1ccc2ncc(-c3cccc(NC(=O)CN4CCOCC4)c3)n2c1]

LC_MS: (ES⁺): m/z 466.1 [M+H]⁺.

¹H NMR (400 MHz, DMSO) δ 9.92 (s, 1H), 8.52 (d, J=1.2 Hz, 1H), 7.94 (t, J=1.7 Hz, 1H), 7.78 (s, 1H), 7.73-7.64 (m, 2H), 7.50 (t, J=7.9 Hz, 1H), 7.34 (ddd, J=11.4, 8.8, 4.2 Hz, 2H), 3.67-3.61 (m, 4H), 3.22 (s, 3H), 3.17 (s, 2H), 2.55-2.51 (m, 4H), 1.37 (s, 9H).

Compound 148

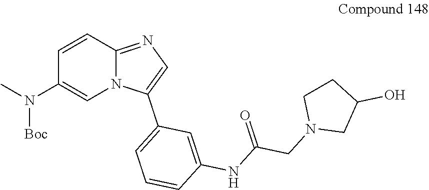 [SMILES: CN(C(=O)OC(C)(C)C)c1ccc2ncc(-c3cccc(NC(=O)CN4CCC(O)C4)c3)n2c1]

LC_MS: (ES⁺): m/z 466.2 [M+H]⁺.

Compound 149

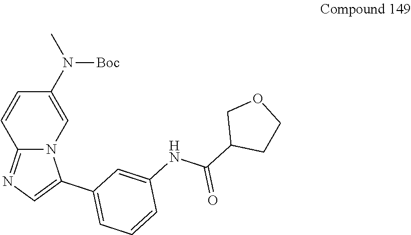 [SMILES: CN(C(=O)OC(C)(C)C)c1ccc2ncc(-c3cccc(NC(=O)C4CCOC4)c3)n2c1]

LC_MS: (ES⁺): m/z 437.2 [M+H]⁺.

¹H NMR (400 MHz, DMSO) δ 10.22 (s, 1H), 8.50 (s, 1H), 7.92 (s, 1H), 7.77 (s, 1H), 7.65 (dd, J=17.5, 8.6 Hz, 2H), 7.49 (t, J=7.9 Hz, 1H), 7.34 (d, J=8.0 Hz, 2H), 3.96 (t, J=8.1 Hz, 1H), 3.74 (dq, J=15.3, 7.5 Hz, 3H), 3.22 (s, 3H), 3.17 (d, J=7.2 Hz, 1H), 2.10 (dd, J=14.2, 7.0 Hz, 2H), 1.37 (s, 9H).

Compound 150

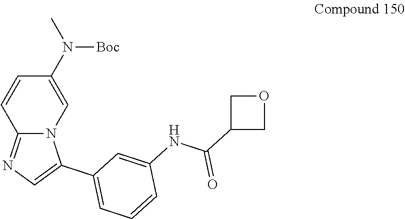 [SMILES: CN(C(=O)OC(C)(C)C)c1ccc2ncc(-c3cccc(NC(=O)C4COC4)c3)n2c1]

LC_MS: (ES⁺): m/z 423.1 [M+H]⁺.

¹H NMR (400 MHz, DMSO) δ 10.13 (s, 1H), 8.51 (s, 1H), 7.94 (s, 1H), 7.78 (s, 1H), 7.65 (dd, J=12.4, 9.2 Hz, 2H), 7.51 (t, J=7.9 Hz, 1H), 7.35 (t, J=7.6 Hz, 2H), 4.75-4.69 (m, 4H), 4.03-3.95 (m, 1H), 3.22 (s, 3H), 1.38 (s, 9H).

Compound 151

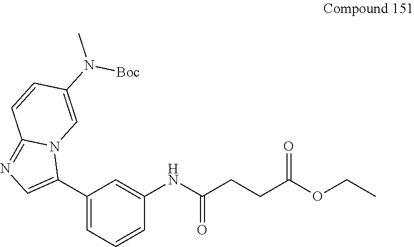 [SMILES: CCOC(=O)CCC(=O)Nc1cccc(-c2cnc3ccc(N(C)C(=O)OC(C)(C)C)cn23)c1]

LC_MS: (ES⁺): m/z 467.2 [M+H]⁺.

### Example 35: Synthesis of Compound 152

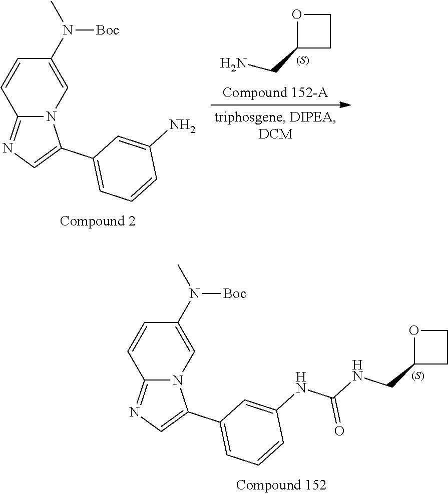

A solution containing compound 2 (30 mg, 88.65 μmol) and N,N-diisopropylethylamine (22.9 mg, 177.3 μmol) in dichloromethane (3 mL) was added with triphosgene (8.7 mg, 29.25 mol) at 0° C. After stirring for 30 minutes, the reaction solution was added with a solution containing compound 152-A (23.2 mg, 265.95 μmol) and N,N-diisopropylethylamine (34.4 mg, 265.95 μmol) in dichloromethane (2 mL), and heated to room temperature for 12 hours of reaction. The reaction was monitored by LCMS to be completed. The reaction solution was diluted with water (10 mL), and extracted with dichloromethane (10 mL×2). The organic layers were combined, washed with saturated brine (10 mL), dried over anhydrous sodium sulfate, filtered and concentrated. The residue was separated and purified by Prep-TLC (eluted with dichloromethane containing 10% methanol) to obtain compound 152 (16.8 mg) as a yellow solid.

LC_MS: (ES⁺): m/z 452.1 [M+H]⁺.

### Example 36: Synthesis of Compound 153

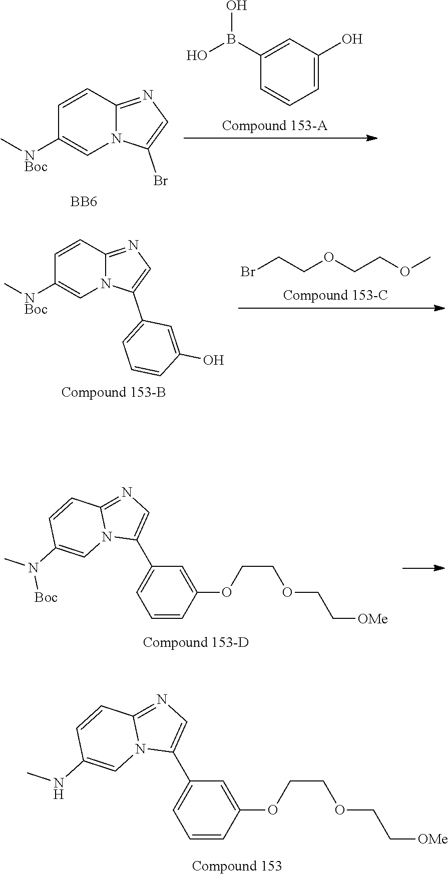

Step 1: The synthesis of compound 153-B was performed referring to the first step of the synthesis process of compound 2.

### Step 2: Compound 153-D tert-butyl(3-(3-(2-(2-methoxyethoxy)ethoxy)phenyl)imidazo[1,2-a]pyridin-6-yl)(methyl)carbamate

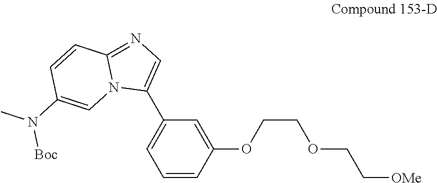 [SMILES: COCCOCCOc1cccc(-c2cnc3ccc(N(C)C(=O)OC(C)(C)C)cn23)c1]

A solution containing compound 153-B (50 mg, 0.15 mmol), compound 153-C (27 mg, 0.15 mmol), potassium carbonate (61 mg, 0.44 mmol) and potassium iodide (3 mg, 0.015 mmol) in anhydrous N,N-dimethylformamide (2 ml) was stirred at 50° C. for 15 hours of reaction. The reaction was monitored by TLC to be completed. The reaction solution was partitioned between ethyl acetate (20 mL) and water (20 mL). The organic layer was collected, washed with saturated brine (10 mL), dried over anhydrous sodium sulfate, and concentrated under reduced pressure. The obtained crude product was separated and purified by Prep-HPLC to obtain a colorless oil compound 153-D tert-butyl(3-(3-(2-(2-methoxyethoxy)ethoxy)phenyl)imidazo[1,2-a]pyridin-6-yl)(methyl)carbamate (36 mg).

LC_MS: (ES⁺): m/z 442.60 [M+H]⁺.

¹H NMR (400 MHz, CDCl₃): δ 1.46 (s, 9H), 3.27 (s, 3H), 3.39 (s, 3H), 3.57-3.60 (m, 2H), 3.72-3.75 (m, 2H), 3.90 (t, J=4.8 Hz, 2H), 4.22 (t, J=4.8 Hz, 2H), 7.03-7.06 (m, 1H), 7.10-7.13 (m, 2H), 7.38 (d, J=9.6 Hz, 1H), 7.47 (t, J=4.0 Hz, 1H), 7.75 (s, 1H), 7.94 (d, J=12.4 Hz, 1H), 8.34 (s, 1H).

Step 3: The synthesis of compound 153 was performed referring to the first step of the synthesis method of compound 63.

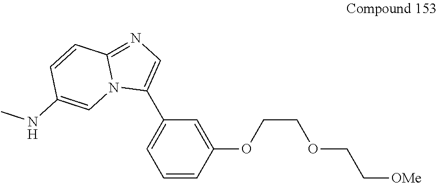 [SMILES: CNc1ccc2ncc(-c3cccc(OCCOCCOC)c3)n2c1]

LC_MS: (ES⁺): m/z 342.30 [M+H]⁺.

¹H NMR (400 MHz, CD₃OD): δ 2.73 (s, 3H), 3.36 (s, 3H), 3.56-3.58 (m, 2H), 3.70-3.72 (m, 2H), 3.86-3.88 (m, 2H), 4.20-4.22 (m, 2H), 7.06-7.12 (m, 2H), 7.20-7.23 (m, 2H), 7.46-7.51 (m, 2H), 7.58 (s, 1H), 7.66 (d, J=9.6 Hz, 1H).

The synthesis of compound 154 was performed referring to the second step (using compound 154-B) and the third step of the synthetic route of compound 153:

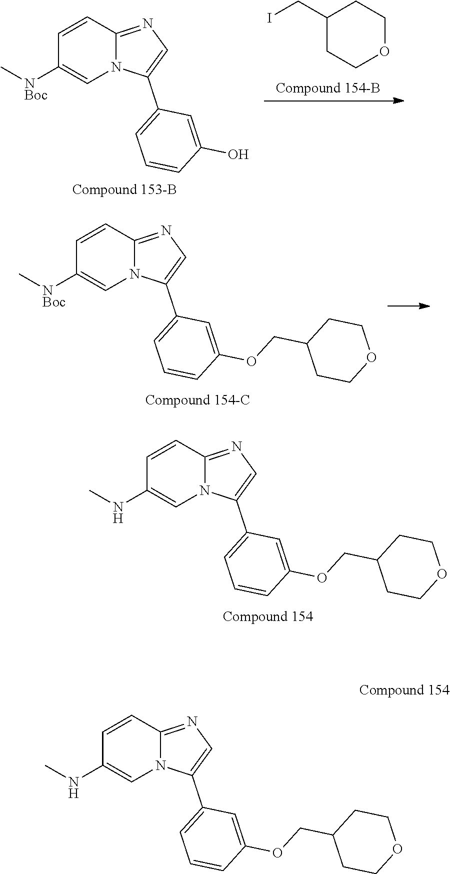

LC_MS: (ES⁺): m/z 338.65 [M+H]⁺.

¹H NMR (400 MHz, CDCl₃): δ 1.42-1.53 (m, 2H), 1.76-1.79 (m, 2H), 2.05-2.14 (m, 1H), 2.76 (s, 3H), 3.42-3.48 (m, 2H), 3.85 (d, J=6.4 Hz, 2H), 4.01-4.04 (m, 2H), 6.92-6.98 (m, 2H), 7.05 (s, 1H), 7.13 (d, J=7.2 Hz, 1H), 7.39-7.42 (m, 2H), 7.64 (s, 1H), 8.12 (brs, 1H).

The following compounds were synthesized referring to the synthetic route of compound 153 in Example 36 using corresponding reagents:

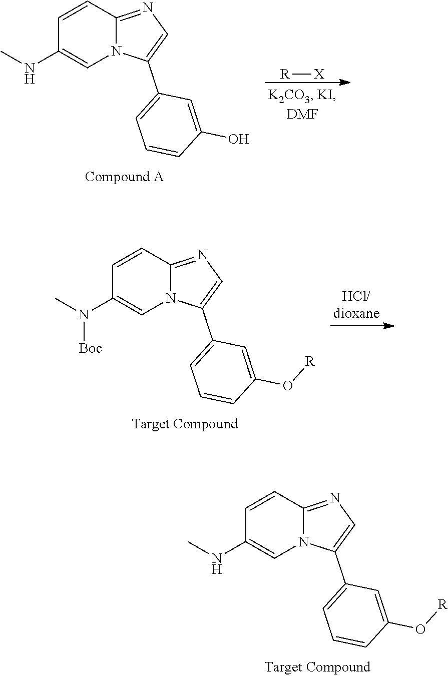

| Number | Structural formula | Mass spectrometry and NMR |
|---|---|---|
| Compound 155 | 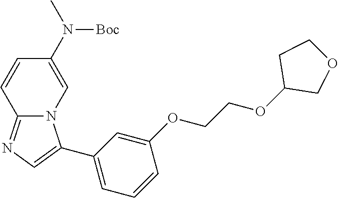 [SMILES: CN(C(=O)OC(C)(C)C)c1ccc2ncc(-c3cccc(OCCOC4CCOC4)c3)n2c1] | LC_MS: (ES+): m/z 454.23 [M + H]+. |
| Compound 156 | 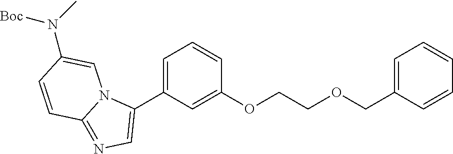 [SMILES: CN(C(=O)OC(C)(C)C)c1ccc2ncc(-c3cccc(OCCOCc4ccccc4)c3)n2c1] | LC_MS: (ES+): m/z 474.23 [M + H]+. |
| Compound 157 | 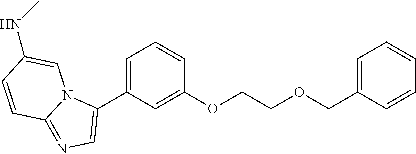 [SMILES: CNc1ccc2ncc(-c3cccc(OCCOCc4ccccc4)c3)n2c1] | LC_MS: (ES+): m/z 374.18 [M + H]+. |

### Example 37: Synthetic Route of Compound 158 and Compound 159

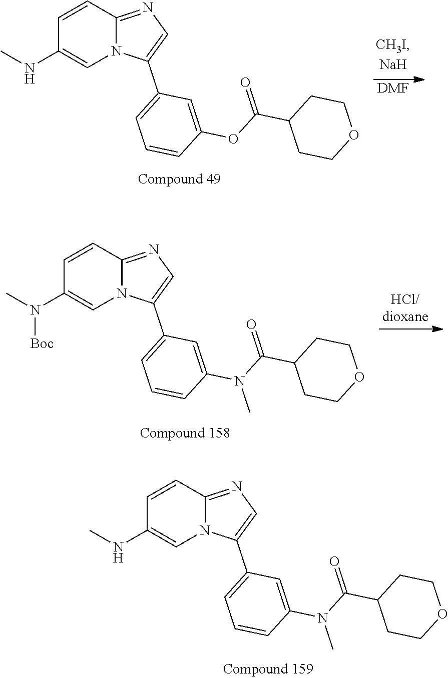

### Step 1: Compound 158 tert-butyl methyl (3-(3-(N-methyltetrahydro-2H-pyran-4-carboxamido)phenyl)imidazo[1,2-a]pyridin-6-yl)carbamate

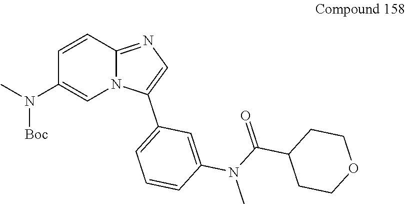 [SMILES: CN(C(=O)OC(C)(C)C)c1ccc2ncc(-c3cccc(N(C)C(=O)C4CCOCC4)c3)n2c1]

A solution of compound 49 (200.00 mg, 443.91 μmol) in DMF (10 ml) was added with sodium hydride (19.53 mg, 488.30 μmol, 60%) and methyl iodide (75.61 mg, 532.70 μmol) under a nitrogen atmosphere at 0° C. The reaction solution was stirred at 0° C. for 1 hour of reaction. The reaction was monitored by TLC to be completed. The reaction solution was poured into saturated ammonium chloride solution (20 mL), and extracted with ethyl acetate (15 mL×2). The organic phases were combined, washed with saturated brine (30 mL), dried over anhydrous sodium sulfate, and concentrated under reduced pressure. The obtained crude product was separated and purified by silica gel column chromatography (eluent: dichloromethane solution containing 0%-5% methanol) to obtain white solid compound 158 tert-butyl methyl (3-(3-(N-methyltetrahydro-2H-pyran-4-carboxamido)phenyl)imidazo[1,2-a]pyridin-6-yl)carbamate (150 mg).

LC_MS: (ES): m/z 465.24 [M+H]⁺.

Step 2: The synthesis of compound 159 was performed referring to the first step of the synthesis process of Example 6.

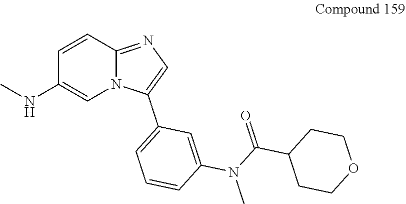 [SMILES: CNc1ccc2ncc(-c3cccc(N(C)C(=O)C4CCOCC4)c3)n2c1]

LC_MS: (ES⁺): m/z 365.19 [M+H]⁺.

Compound 160 was synthesized referring to the synthetic route of compound 158 in Example 37, using corresponding reagents:

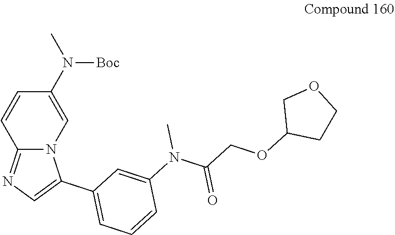 [SMILES: CN(C(=O)COC1CCOC1)c1cccc(-c2cnc3ccc(N(C)C(=O)OC(C)(C)C)cn23)c1]

LC_MS: (ES⁺): m/z 481.24 [M+H]⁺.

### Example 38: Synthetic Routes of Compound 161 and Compound 162

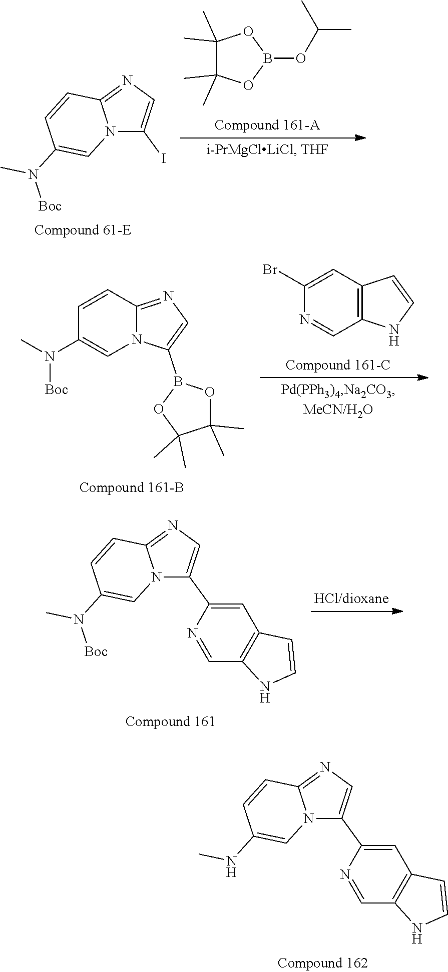

### Step 1: Compound 161-B tert-butyl (3-(4,4,5,5-tetramethyl-1,3,2-dioxaborolan-2-yl)imidazo[1,2-a]pyridin-6-yl)carbamate

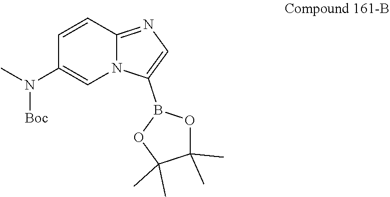

A solution containing compound 61-E tert-butyl (3-iodoimidazo[1,2-a]pyridin-6-yl)(methyl)carbamate (300 mg, 0.8 mmol) and compound 161-A 2-isopropoxy-4,4,5,5-tetramethyl-1,3,2-dioxaborane (225 mg, 1.2 mmol) in anhydrous tetrahydrofuran (10 ml) was slowly added with isopropyl magnesium chloride lithium chloride complex (0.75 ml, 0.96 mmol, 1.3 N in tetrahydrofuran) dropwise. The reaction mixture was stirred at 0° C. for 2 hours of reaction. The reaction was monitored by TLC to be completed. The reaction was quenched with saturated ammonium chloride solution (30 ml). The reaction solution was extracted with ethyl acetate (30 ml). The organic layer was collected, washed with saturated brine (20 ml), dried over anhydrous sodium sulfate, and concentrated under reduced pressure to obtain light red solid compound 161-B tert-butyl (3-(4,4,5,5-tetramethyl-1,3,2-dioxaborolan-2-yl)imidazo[1,2-a]pyridin-6-yl)carbamate (285 mg, crude product).

LC_MS: (ES⁺): m/z 291.8 [M+H]⁺.

### Step 2: Compound 161 tert-butyl (3-(1H-pyrrolo[2,3-c]pyridin-5-yl)imidazo[1,2-a]pyridin-6-yl)(methyl)carbamate

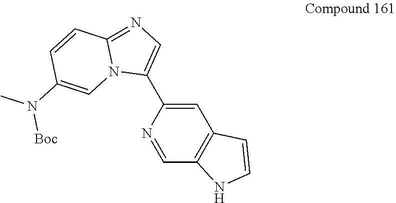 [SMILES: CN(C(=O)OC(C)(C)C)c1ccc2ncc(-c3cc4cc[nH]c4cn3)n2c1]

A suspension containing compound 161-C 5-bromo-1H-pyrrolo[2,3-c]pyridine (100 mg, 0.508 mmol), compound 161-B tert-butyl (3-(4,4,5,5-tetramethyl-1,3,2-dioxaborolan-2-yl)imidazo[1,2-a]pyridin-6-yl)carbamate (284.2 mg, 0.761 mmol, crude product) and sodium carbonate (1N, 1.52 ml, 1.52 mmol) in acetonitrile (5 ml) and water (1 ml) was added with tetrakis(triphenylphosphine)palladium (58.7 mg, 50.75 μmol). The reaction mixture was replaced with nitrogen three times, and stirred at 90° C. for 16 hours of reaction. The reaction was monitored by TLC to be completed. The reaction mixture was partitioned between ethyl acetate (20 ml) and water (10 ml). The organic layer was collected, washed with saturated brine (20 ml), dried over anhydrous sodium sulfate, and concentrated under reduced pressure. The obtained crude product was separated and purified by silica gel column chromatography (eluted with dichloromethane containing 1% methanol) to obtain compound 161 tert-butyl (3-(1H-pyrrolo[2,3-c]pyridin-5-yl)imidazo[1,2-a]pyridin-6-yl)(methyl)carbamate (50 mg).

LC_MS: (ES⁺): m/z 364.2 [M+H]⁺

### Step 3: Compound 162 N-methyl-3-(1H-pyrrolo[2,3-c]pyridin-5-yl)imidazo[1,2-a]pyridin-6-amine

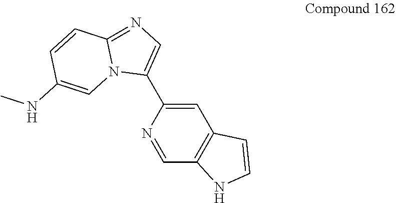 [SMILES: CNc1ccc2ncc(-c3cc4cc[nH]c4cn3)n2c1]

The synthesis of this step was performed referring to the first step of the synthesis process of Example 6.

LC_MS: (ES⁺): m/z 264.1 [M+H]⁺

The following compounds were synthesized referring to the synthetic procedure of compound 161 and compound 162 in Example 38, using corresponding reagents:

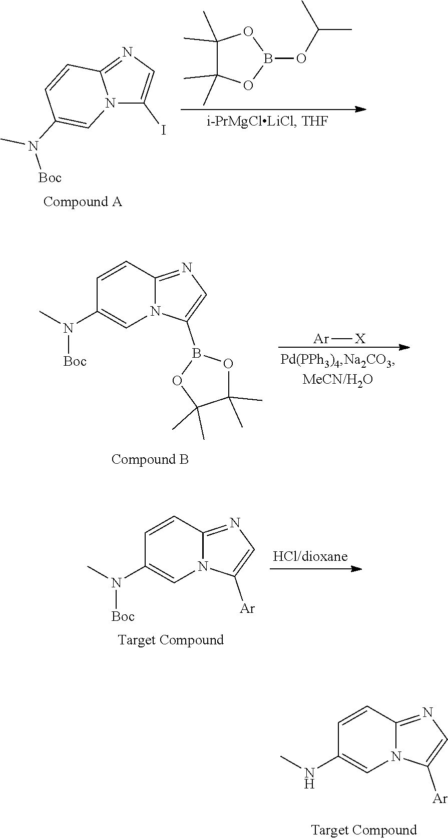

| Number | Structural formula | Mass spectrometry and NMR |
|---|---|---|
| Compound 163 | 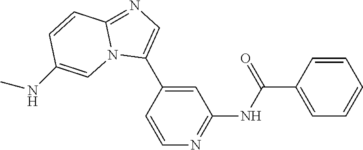 | LC_MS: (ES+): m/z 299.10 [M + H]+. ¹H NMR (400 MHz, DMSO-d6): δ 11.37 (s, 1H), 8.66- 8.64 (m, 2H), 8.54 (s, 1H), 8.10 (d, J = 7.6 Hz, 2H), 7.86 (d, J = 9.2 Hz, 1H), 7.81 (s, 1H), 7.64-7.53 (m, 5H), 2.78 (s, 3H). |
| Compound 164 | 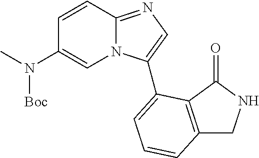 [SMILES: CN(C(=O)OC(C)(C)C)c1ccc2ncc(-c3cccc4c3C(=O)NC4)n2c1] | LC_MS: (ES+): m/z 379.17 [M + H]+. |
| Compound 165 | 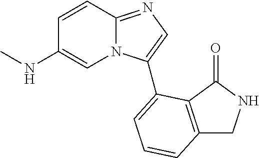 [SMILES: CNc1ccc2ncc(-c3cccc4c3C(=O)NC4)n2c1] | LC_MS: (ES+): m/z 279.12 [M + H]+. |
| Compound 166 | 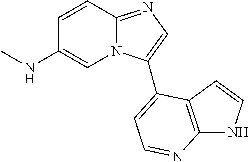 [SMILES: CNc1ccc2ncc(-c3ccnc4[nH]ccc34)n2c1] | LC_MS: (ES+): m/z 264.12 [M + H]+. |
| Compound 167 | 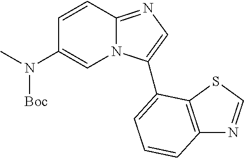 | LC_MS: (ES+): m/z 391.13 [M + H]+. ¹H NMR (400 MHz, CDCl3): δ 9.49 (s, 1H), 8.35 (d, J = 1.2 Hz, 1H), 8.21 (dd, J = 7.6, 1.6 Hz, 1H), 8.00 (s, 1H), 7.80-7.70 (m, 3H), 7.39 (dd, J = 9.6, 1.8 Hz, 1H), 3.17 (s, 3H), 1.36 (s, 9H). |
| Compound 168 | 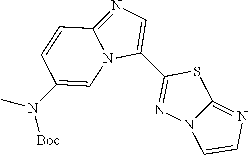 [SMILES: CN(C(=O)OC(C)(C)C)c1ccc2ncc(-c3nn4ccnc4s3)n2c1] | LC_MS: (ES+): m/z 371.12 [M + H]+. ¹H NMR (400 MHz, CDCl3): δ 9.28 (s, 1H), 8.50 (s, 1H), 8.24 (s, 1H), 7.85 (d, J = 9.5 Hz, 1H), 7.62 (dd, J = 9.5, 1.9 Hz, 1H), 7.35 (s, 1H), 3.28 (s, 3H), 1.42 (s, 9H). |
| Compound 169 | 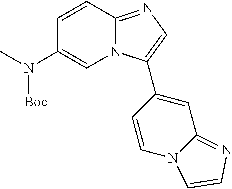 [SMILES: CN(C(=O)OC(C)(C)C)c1ccc2ncc(-c3ccn4ccnc4c3)n2c1] | LC_MS: (ES+): m/z 364.17 [M + H]+. |
| Compound 170 | 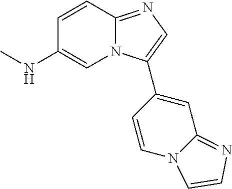 | LC_MS: (ES+): m/z 300.09 [M + H]+. |
| Compound 171 | 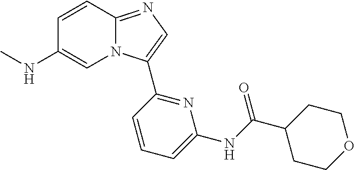 [SMILES: CNc1ccc2ncc(-c3cccc(NC(=O)C4CCOCC4)n3)n2c1] | LC_MS: (ES+): m/z 352.17 [M + H]+. |
| Compound 172 | 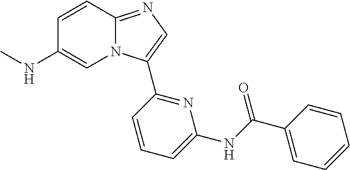 [SMILES: CNc1ccc2ncc(-c3cccc(NC(=O)c4ccccc4)n3)n2c1] | LC_MS: (ES+): m/z 344.14 [M + H]+. ¹H NMR (400 MHz, CDCl3): δ 10.72 (s, 1H), 9.22 (s, 1H), 8.21 (s, 1H), 8.04 (d, J = 7.6 Hz, 2H), 7.90 (t, J = 7.9 Hz, 1H), 7.82 (d, J = 8.0 Hz, 1H), 7.71 (d, J = 7.7 Hz, 1H), 7.63 (t, J = 7.2 Hz, 1H), 7.59-7.47 (m, 3H), 7.05 (d, J = 9.5 Hz, 1H), 5.61 (s, 1H), 2.82 (s, 3H). |
| Compound 173 | 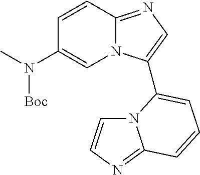 [SMILES: CN(C(=O)OC(C)(C)C)c1ccc2ncc(-c3cccc4nccn34)n2c1] | LC_MS: (ES+): m/z 364.17 [M + H]+. |
| Compound 174 | 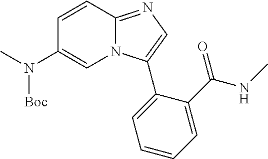 | LC_MS: (ES+): m/z 381.18 [M + H]+. |
| Compound 175 | 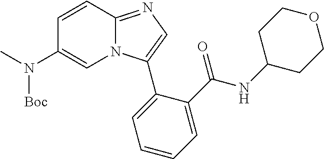 [SMILES: CN(C(=O)OC(C)(C)C)c1ccc2ncc(-c3ccccc3C(=O)NC3CCOCC3)n2c1] | LC_MS: (ES+): m/z 451.23 [M + H]+. |
| Compound 176 | 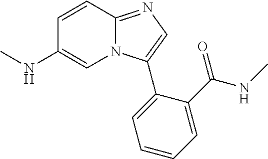 | LC_MS: (ES+): m/z 281.13 [M + H]+. |
| Compound 177 | 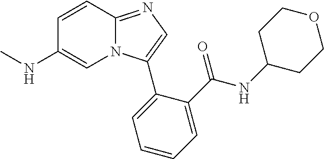 [SMILES: CNc1ccc2ncc(-c3ccccc3C(=O)NC3CCOCC3)n2c1] | LC_MS: (ES+): m/z 351.17 [M + H]+. |
| Compound 178 | 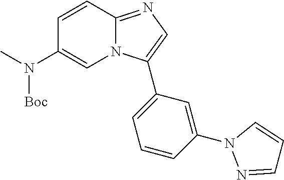 [SMILES: CN(C(=O)OC(C)(C)C)c1ccc2ncc(-c3cccc(-n4cccn4)c3)n2c1] | LC_MS: (ES+): m/z 390.19 [M + H]+. |
| Compound 179 | 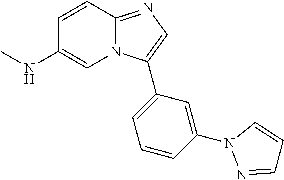 [SMILES: CNc1ccc2ncc(-c3cccc(-n4cccn4)c3)n2c1] | LC_MS: (ES+): m/z 290.13 [M + H]+. |
| Compound 180 | 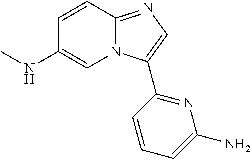 [SMILES: CNc1ccc2ncc(-c3cccc(N)n3)n2c1] | LC_MS: (ES+): m/z 240.12 [M + H]+. |
| Compound 181 | 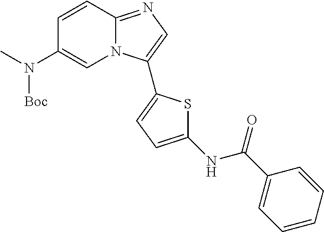 [SMILES: CN(C(=O)OC(C)(C)C)c1ccc2ncc(-c3ccc(NC(=O)c4ccccc4)s3)n2c1] | LC_MS: (ES+): m/z 449.16 [M + H]+. ¹H NMR (400 MHz, CDCl3): δ 9.38 (s, 1H), 8.37 (s, 1H), 7.97-7.95 (m, 2H), 7.72 (s, 1H), 7.60-7.56 (m, 2H), 7.50 (d, J = 7.2 Hz, 2H), 7.18 (d, J = 9.2 Hz, 1H), 7.06 (d, J = 4.0 Hz, 1H), 6.09 (d, J = 4.0 Hz, 1H), 3.27 (s, 3H), 1.46 (s, 9H). |
| Compound 182 | 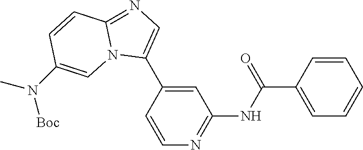 [SMILES: CN(C(=O)OC(C)(C)C)c1ccc2ncc(-c3ccnc(NC(=O)c4ccccc4)c3)n2c1] | LC_MS: (ES+): m/z 444.20 [M + H]+. ¹H NMR (400 MHz, CDCl3): δ 8.74 (s, 1H), 8.69 (d, J = 0.8 Hz, 1H), 8.60 (s, 1H), 8.42 (d, J = 5.2 Hz, 1H), 7.97-7.94 (m, 3H), 7.75 (s, 1H), 7.63-7.58 (m, 1H), 7.55-7.51 (m, 2H), 7.35-7.32 (m, 1H), 7.29-7.27 (m, 1H), 3.38 (s, 3H), 1.47 (s, 9H). |
| Compound 183 | 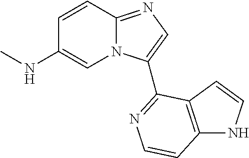 [SMILES: CNc1ccc2ncc(-c3nccc4[nH]ccc34)n2c1] | LC_MS: (ES+): m/z 264.12 [M + H]+. |
| Compound 184 | 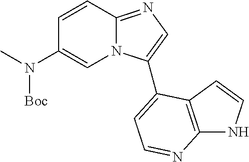 [SMILES: CN(C(=O)OC(C)(C)C)c1ccc2ncc(-c3ccnc4[nH]ccc34)n2c1] | LC_MS: (ES+): m/z 364.17 [M + H]+. ¹H NMR (400 MHz, DMSO) δ 11.94 (s, 1H), 8.49 (s, 1H), 8.37 (d, J = 4.9 Hz, 1H), 8.02 (s, 1H), 7.76 (d, J = 9.6 Hz, 1H), 7.64-7.58 (m, 1H), 7.42 (dd, J = 9.6, 1.5 Hz, 1H), 7.36 (d, J = 4.9 Hz, 1H), 6.43 (d, J = 1.5 Hz, 1H), 3.19 (s, 3H), 1.37 (s, 9H). |
| Compound 185 | 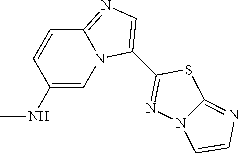 [SMILES: CNc1ccc2ncc(-c3nn4ccnc4s3)n2c1] | LC_MS: (ES+): m/z 271.07 [M + H]+. |
| Compound 186 | 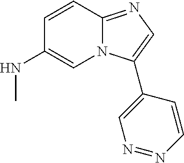 [SMILES: CNc1ccc2ncc(-c3ccnnc3)n2c1] | LC_MS: (ES+): m/z 226.01 [M + H]+. ¹H NMR (400 MHz, CDCl3): δ 9.61 (s, 1H), 9.24 (d, J = 5.6 Hz, 1H), 8.02-8.03 (m, 2H), 7.63 (s, 1H), 7.55 (d, J = 9.6 Hz, 1H), 7.06 (d, J = 9.2 Hz, 1H), 5.86 (d, J = 4.4 Hz, 1H), 2.72 (d, J = 4.4 Hz, 3H). |
| Compound 187 | 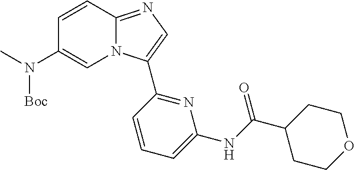 [SMILES: CN(C(=O)OC(C)(C)C)c1ccc2ncc(-c3cccc(NC(=O)C4CCOCC4)n3)n2c1] | LC_MS: (ES+): m/z 452.22 [M + H]+. ¹H NMR (400 MHz, DMSO) δ 10.58 (s, 1H), 10.07 (d, J = 1.4 Hz, 1H), 8.43 (s, 1H), 7.88-7.84 (m, 2H), 7.72 (dd, J = 5.3, 3.3 Hz, 1H), 7.66 (d, J = 9.6 Hz, 1H), 7.42 (dd, J = 9.5, 1.9 Hz, 1H), 3.93 (d, J = 10.9 Hz, 2H), 3.40 (dd, J = 11.4, 3.0 Hz, 2H), 3.33 (s, 3H), 2.85- 2.75 (m, 1H), 1.78-1.67 (m, 4H), 1.39 (s, 9H). |
| Compound 188 | 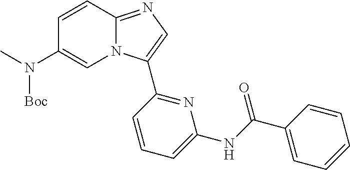 [SMILES: CN(C(=O)OC(C)(C)C)c1ccc2ncc(-c3cccc(NC(=O)c4ccccc4)n3)n2c1] | LC_MS: (ES+): m/z 444.20 [M + H]+. ¹H NMR (400 MHz, DMSO) δ 10.94 (s, 1H), 10.24 (s, J = 1.4 Hz, 1H), 8.47 (s, 1H), 8.06-7.99 (m, 2H), 7.95- 7.89 (m, 2H), 7.85-7.78 (m, 1H), 7.69-7.55 (m, 4H), 7.40 (dd, J = 9.5, 2.0 Hz, 1H), 3.34 (s, 3H), 1.36 (s, 9H). |
| Compound 189 | 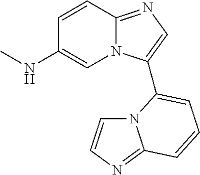 [SMILES: CNc1ccc2ncc(-c3cccc4nccn34)n2c1] | LC_MS: (ES+): m/z 264.12 [M + H]+. |
| Compound 190 | 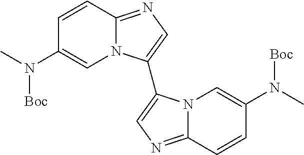 | LC_MS: (ES+): m/z 493.25 [M + H]+. |
| Compound 191 | 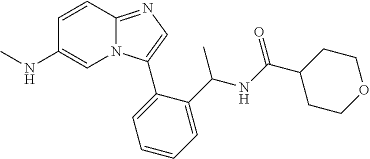 [SMILES: CNc1ccc2ncc(-c3ccccc3C(C)NC(=O)C3CCOCC3)n2c1] | LC_MS: (ES+): m/z 379.21 [M + H]+. |
| Compound 192 | 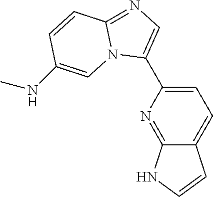 [SMILES: CNc1ccc2ncc(-c3ccc4cc[nH]c4n3)n2c1] | LC_MS: (ES+): m/z 264.12 [M + H]+. |
| Compound 193 | 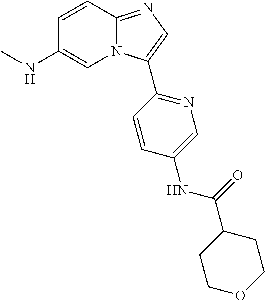 [SMILES: CNc1ccc2ncc(-c3ccc(NC(=O)C4CCOCC4)cn3)n2c1] | LC_MS: (ES+): m/z 352.17 [M + H]+. |
| Compound 194 | 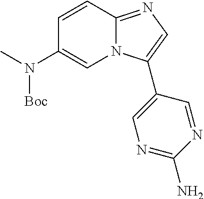 [SMILES: CN(C(=O)OC(C)(C)C)c1ccc2ncc(-c3cnc(N)nc3)n2c1] | LC_MS: (ES+): m/z 341.16 [M + H]+. ¹H NMR (400 MHz, CDCl3) δ 8.48 (s, 1H), 8.09 (s, 1H), 7.78 (d, J = 43.7 Hz, 2H), 7.32 (s, 1H), 5.40 (s, 2H), 3.28 (s, 3H), 1.47 (s, 9H). |
| Compound 195 | 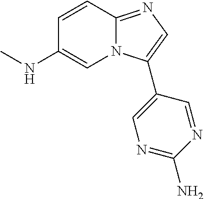 [SMILES: CNc1ccc2ncc(-c3cnc(N)nc3)n2c1] | LC_MS: (ES+): m/z 241.11 [M + H]+. |
| Compound 196 | 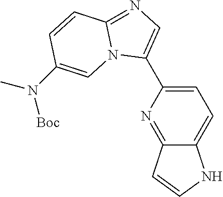 [SMILES: CN(C(=O)OC(C)(C)C)c1ccc2ncc(-c3ccc4[nH]ccc4n3)n2c1] | LC_MS: (ES+): m/z 364.17 [M + H]+. |
| Compound 197 | 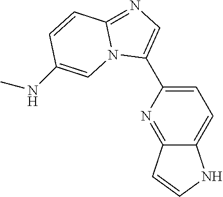 [SMILES: CNc1ccc2ncc(-c3ccc4[nH]ccc4n3)n2c1] | LC_MS: (ES+): m/z 264.12 [M + H]+. |
| Compound 198 | 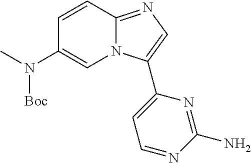 [SMILES: CN(C(=O)OC(C)(C)C)c1ccc2ncc(-c3ccnc(N)n3)n2c1] | LC_MS: (ES+): m/z 341.16 [M + H]+. ¹H NMR (400 MHz, CDCl3): δ 9.96 (s, 1H), 8.35 (s, 1H), 8.22 (d, J = 5.8 Hz, 1H), 7.74 (d, J = 9.4 Hz, 1H), 7.42 (d, J = 9.2 Hz, 1H), 7.06 (d, J = 5.7 Hz, 1H), 5.70 (s, 2H), 3.38 (s, 3H), 1.50 (s, 9H). |
| Compound 199 | 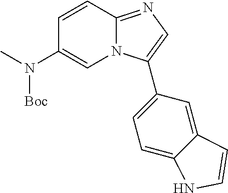 [SMILES: CN(C(=O)OC(C)(C)C)c1ccc2ncc(-c3ccc4[nH]ccc4c3)n2c1] | LC_MS: (ES+): m/z 363.17 [M + H]+. |
| Compound 200 | 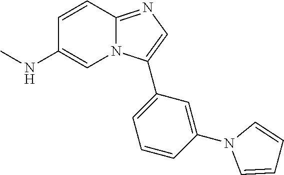 [SMILES: CNc1ccc2ncc(-c3cccc(-n4cccc4)c3)n2c1] | LC_MS: (ES+): m/z 289.14 [M + H]+. ¹H NMR (400 MHz, CDCl3): δ 7.66 (s, 1H), 7.59-7.55 (m, 2H), 7.46-7.43 (m, 3H), 7.26-7.24 (m, 1H), 7.13 (t, J = 2.0 Hz, 2H), 6.94-6.85 (m, 1H), 6.38 (t, J = 2 Hz, 2H), 2.78 (s, 3H). |

### Example 39 Synthetic Route of Compound 201 and Compound 202

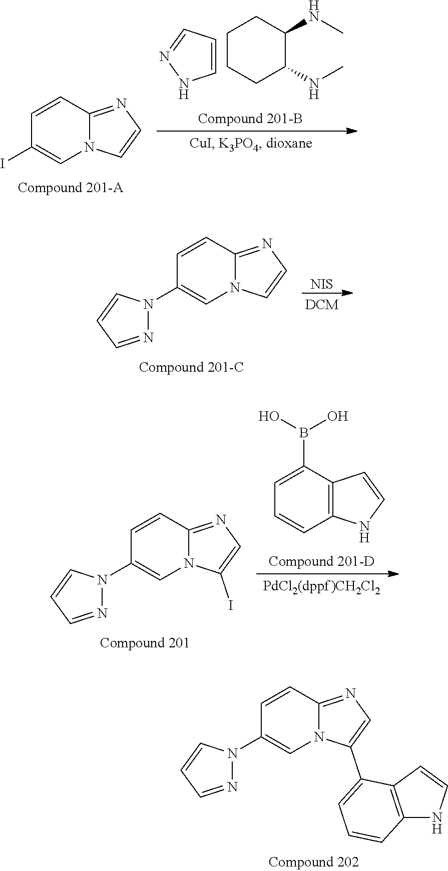

### Step 1: Compound 201-C 6-(1H-pyrazol-1-yl)imidazo[1,2-a]pyridine

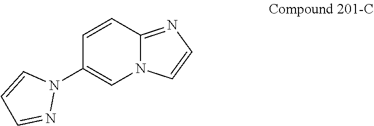 [SMILES: c1cnn(-c2ccc3nccn3c2)c1]

A solution containing compound 201-A 6-iodoimidazo[1,2-a]pyridine (1.0 g, 4.10 mmol) and compound 201-B pyrazole (418.5 mg, 6.15 mmol) in 1,4-dioxane (20 mL) was added with cuprous iodide (78.04 mg, 409.78 μmol), tripotassium phosphate (78.04 mg, 409.78 μmol) and N1,N2-dimethyl transcyclohexane-1,2-diamine (58.29 mg, 409.78 μmol) at room temperature. The reaction mixture was replaced with nitrogen three times, and stirred at 110° C. for 12 h of reaction. The reaction was monitored by TLC to be completed. The reaction solution was filtered through diatomite and concentrated under reduced pressure. The crude product obtained was separated and purified by silica gel column chromatography (eluted with dichloromethane containing 0-10% methanol) to obtain compound 201-C 6-(1H-pyrazol-1-yl)imidazo[1,2-a]pyridine (500 mg).

LC_MS: (ES⁺): m/z 185.07[M+H]⁺

### Step 2: Compound 201 3-iodo-6-(1H-pyrazol-1-yl)imidazo[1,2-a]pyridine

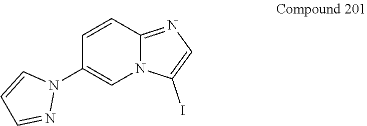 [SMILES: Ic1cnc2ccc(-n3cccn3)cn12]

The synthesis of this step was performed referring to the fourth step of the synthesis process of intermediate BB5.

LC_MS: (ES⁺): m/z 310.97[M+H]⁺

¹H NMR (400 MHz, DMSO) δ 8.71 (d, J=1.4 Hz, 1H), 8.65 (d, J=2.4 Hz, 1H), 7.92 (dd, J=9.7, 2.0 Hz, 1H), 7.85-7.78 (m, 3H), 6.62 (dd, J=5.6, 3.4 Hz, 1H).

### Step 3: Compound 202 3-(1H-indol-4-yl)-6-(1H-pyrazol-1-yl)imidazo[1,2-a]pyridine

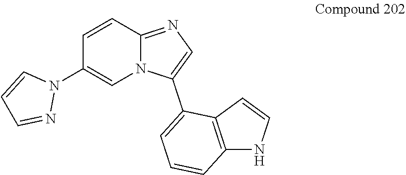 [SMILES: c1cc(-c2cnc3ccc(-n4cccn4)cn23)c2cc[nH]c2c1]

The synthesis of this step was performed referring to the synthesis process of compound 2 in Example 2.

LC_MS: (ES⁺): m/z 300.12[M+H]⁺.

The following compounds were synthesized referring to the synthetic procedure of compound 201 and compound 202 in Example 39, using corresponding reagents:

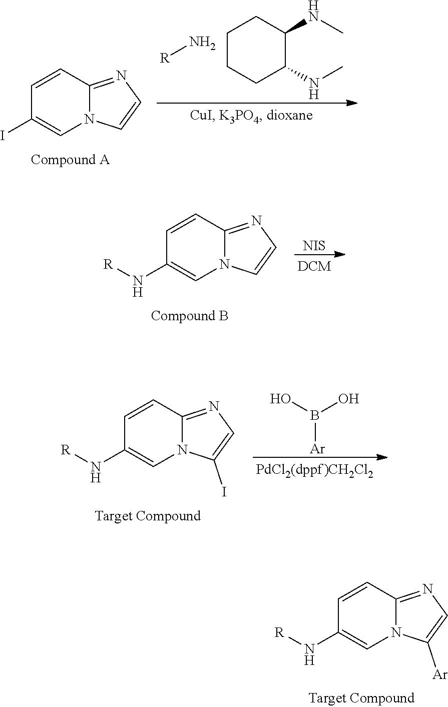

| Number | Structural formula | Mass spectrometry and NMR |
|---|---|---|
| Compound 203 | 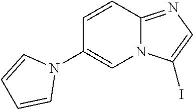 [SMILES: Ic1cnc2ccc(-n3cccc3)cn12] | LC_MS: (ES+): m/z 309.98 [M + H]+. |
| Compound 204 | 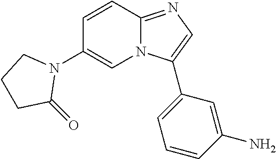 [SMILES: Nc1cccc(-c2cnc3ccc(N4CCCC4=O)cn23)c1] | LC_MS: (ES+): m/z 293.13[M + H]+. |
| Compound 205 | 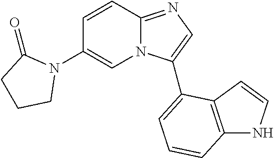 [SMILES: O=C1CCCN1c1ccc2ncc(-c3cccc4[nH]ccc34)n2c1] | LC_MS: (ES+): m/z 317.13[M + H]+. ¹H NMR (400 MHz, DMSO) δ 11.43 (s, 1H), 8.81 (s, 1H), 7.81 (d, J = 12.6 Hz, 2H), 7.68 (d, J = 9.7 Hz, 1H), 7.55 (dd, J = 6.7, 1.8 Hz, 1H), 7.46 (t, J = 2.7 Hz, 1H), 7.27 (dd, J = 9.3, 4.7 Hz, 2H), 6.27 (s, 1H), 3.82 (t, J = 7.0 Hz, 2H), 2.45 (t, J = 8.1 Hz, 2H), 2.12-2.00 (m, 2H). |
| Compound 206 | 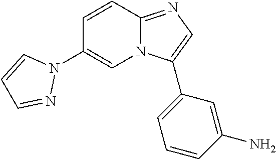 [SMILES: Nc1cccc(-c2cnc3ccc(-n4cccn4)cn23)c1] | LC_MS: (ES+): m/z 276.12[M + H]+. |
| Compound 207 | 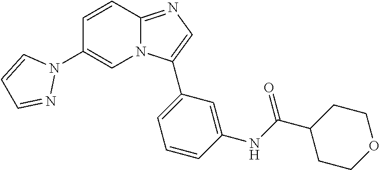 [SMILES: O=C(Nc1cccc(-c2cnc3ccc(-n4cccn4)cn23)c1)C1CCOCC1] | LC_MS: (ES+): m/z 388.17[M + H]+. |

### Example 40: Synthetic Route of Compound 208 and Compound 209

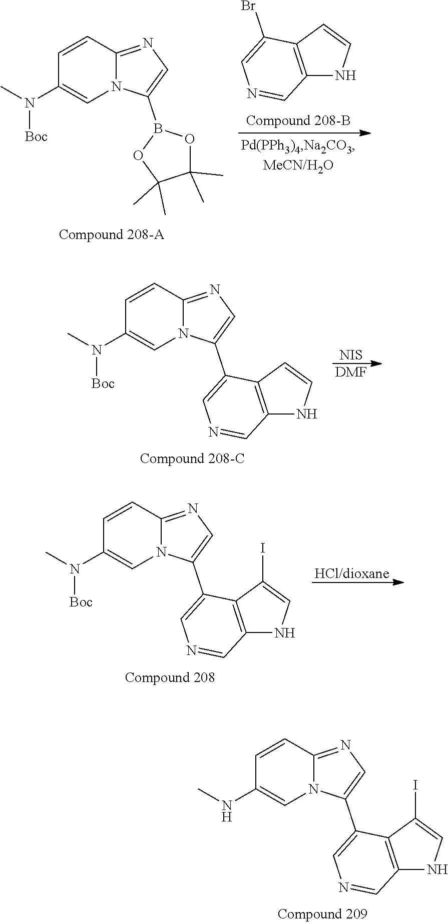

### Step 1: Compound 208-C tert-butyl (3-(1H-pyrrolo[2,3-c]pyridin-4-yl)imidazo[1,2-a]pyridin-6-yl)(methyl)carbamate

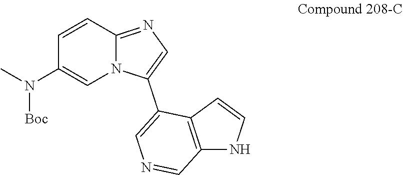 [SMILES: CN(C(=O)OC(C)(C)C)c1ccc2ncc(-c3cncc4[nH]ccc34)n2c1]

The synthesis of this step was performed referring to the second step of the synthesis process of Compound 161 and Compound 162 in Example 38.

LC_MS: (ES⁺): m/z 364.17[M+H]⁺.

### Step 2: Compound 208 tert-butyl (3-(3-iodo-1H-pyrrolo[2,3-c]pyridin-4-yl)imidazo[1,2-a]pyridin-6-yl)(methyl) carbamate

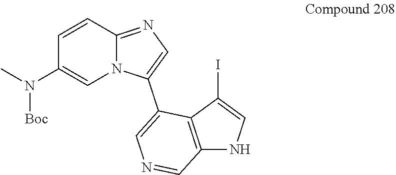 [SMILES: CN(C(=O)OC(C)(C)C)c1ccc2ncc(-c3cncc4[nH]cc(I)c34)n2c1]

The synthesis of this step was performed referring to the fourth step of the synthesis process of intermediate BB5.

LC_MS: (ES⁺): m/z 490.07[M+H]⁺.

### Step 3: Compound 209 (3-(3-iodo-1H-pyrrolo[2,3-c]pyridin-4-yl)-N-methylimidazo[1,2-a]pyridin-6-amine

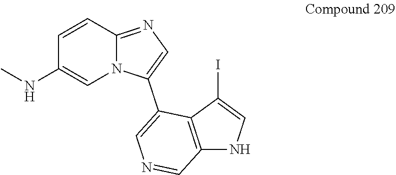 [SMILES: CNc1ccc2ncc(-c3cncc4[nH]cc(I)c34)n2c1]

The synthesis of this step was performed referring to the first step of the synthesis method of Example 6.

LC_MS: (ES⁺): m/z 390.01[M+H]⁺.

Compound 210 3-(3-iodo-1H-indazol-7-yl)-N-methylimidazol[1,2-a]pyridin-6-amine hydrochloride was synthesized referring to the synthesis of compound 209 in Example 40, using corresponding reagents:

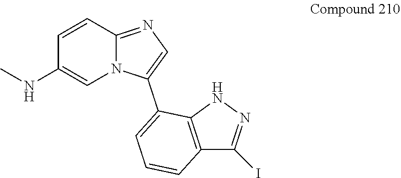 [SMILES: CNc1ccc2ncc(-c3cccc4c(I)n[nH]c34)n2c1]

LC_MS: (ES): m/z 390.01[M+H]⁺.

### Example 41: Synthetic Route of Compound 211 Compound 212, and Compound 213

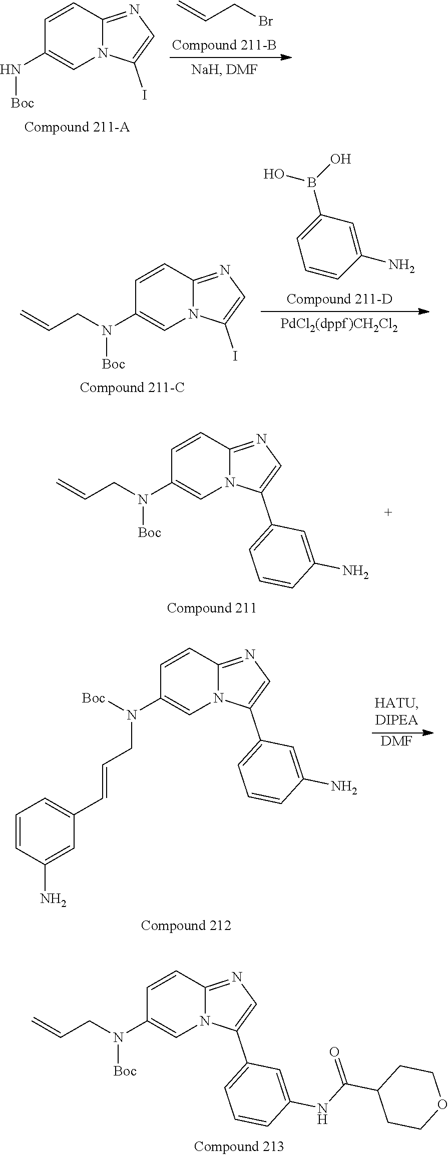

### Step 1: Compound 211-C tert-butyl allyl(3-iodoimidazo[1,2-a]pyridin-6-yl)carbamate

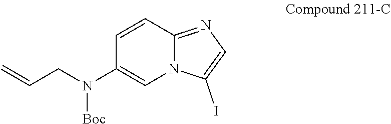 [SMILES: C=CCN(C(=O)OC(C)(C)C)c1ccc2ncc(I)n2c1]

The synthesis of this step was performed referring to the fifth step of the synthesis process of intermediate BB6.

LC_MS: (ES⁺): m/z 400.04[M+H]⁺.

### Step 2: Compound 211 tert-butyl allyl(3-(3-aminophenyl)imidazo[1,2-a]pyridin-6-yl)carbamate

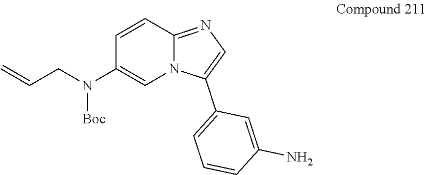 [SMILES: C=CCN(C(=O)OC(C)(C)C)c1ccc2ncc(-c3cccc(N)c3)n2c1]

The synthesis of this step was performed referring to the synthesis process of compound 2 in Example 2.

LC_MS: (ES⁺): m/z 365.19[M+H]⁺.

### Compound 212 tert-butyl (E)-(3-(3-aminophenyl)allyl)(3-(3-aminophenyl)imidazo[1,2-a]pyridin-6-yl)carbamate

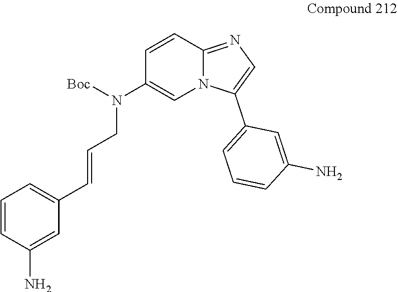 [SMILES: CC(C)(C)OC(=O)N(C/C=C/c1cccc(N)c1)c1ccc2ncc(-c3cccc(N)c3)n2c1]

The synthesis of this step was performed referring to the synthesis process of compound 2 in Example 2.

LC_MS: (ES⁺): m/z 456.23[M+H]⁺.

### Step 3: Compound 213 tert-butyl allyl(3-(3-(tetrahydro-2H-pyran-4-carboxamido)phenyl)imidazo[1,2-a]pyridin-6-yl)carbamate

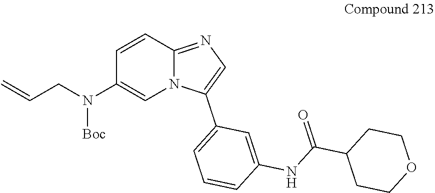 [SMILES: C=CCN(C(=O)OC(C)(C)C)c1ccc2ncc(-c3cccc(NC(=O)C4CCOCC4)c3)n2c1]

The synthesis of this step was performed referring to the synthesis process of compound 47 in Example 3.

LC_MS: (ES) m/z 477.24[M+H]⁺.

The following compounds were synthesized referring to the synthetic procedure of compound 211, compound 212, and compound 213 in Example 41, using corresponding reagents:

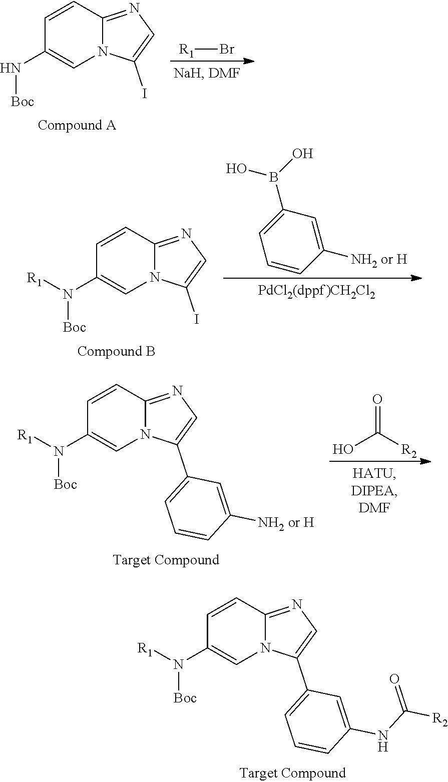

| Number | Structural formula | Mass spectrometry and NMR |
|---|---|---|
| Compound 214 | 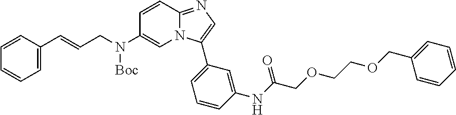 [SMILES: CC(C)(C)OC(=O)N(C/C=C/c1ccccc1)c1ccc2ncc(-c3cccc(NC(=O)COCCOCc4ccccc4)c3)n2c1] | LC_MS: (ES+): m/z 633.30[M + H]+. ¹H NMR (400 MHz, DMSO) δ 9.77 (s, 1H), 8.50 (s, 1H), 7.88 (s, 1H), 7.73 (s, 1H), 7.65 (d, J = 9.5 Hz, 1H), 7.56 (dd, J = 7.4, 3.7 Hz, 1H), 7.39 (d, J = 7.3 Hz, 2H), 7.30 (t, J = 8.7 Hz, 7H), 7.27- 7.22 (m, 4H), 6.51 (d, J = 15.9 Hz, 1H), |
| | | 6.36 (dt, J = 15.9, 6.1 Hz, 1H), 4.52 (s, |
| | | 2H), 4.39 (d, J = 6.0 Hz, 2H), 4.13 (s, |
| | | 2H), 3.74 (dd, J = 5.7, 3.3 Hz, 2H), 3.66 |
| | | (dd, J = 5.8, 3.3 Hz, 2H), 1.39 (s, 9H). |
| Compound 215 | 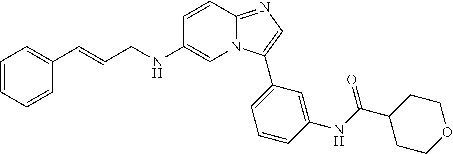 [SMILES: O=C(Nc1cccc(-c2cnc3ccc(NC/C=C/c4ccccc4)cn23)c1)C1CCOCC1] | LC_MS: (ES+): m/z 453.22[M + H]+. |
| Compound 216 | 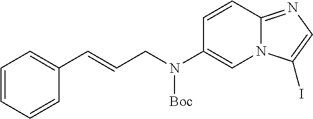 [SMILES: CC(C)(C)OC(=O)N(C/C=C/c1ccccc1)c1ccc2ncc(I)n2c1] | LC_MS: (ES+): m/z 476.08[M + H]+. |
| Compound 217 | 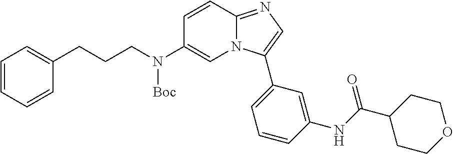 [SMILES: CC(C)(C)OC(=O)N(CCCc1ccccc1)c1ccc2ncc(-c3cccc(NC(=O)C4CCOCC4)c3)n2c1] | LC_MS: (ES+): m/z 555.29[M + H]+. ¹H NMR (400 MHz, DMSO) δ 10.09 (s, 1H), 8.44 (s, 1H), 8.01 (s, 1H), 7.78 (d, J = 2.4 Hz, 1H), 7.68 (d, J = 9.4 Hz, 1H), 7.58 (d, J = 7.9 Hz, 1H), 7.48 (t, J = 7.9 Hz, 1H), 7.29 (t, J = 8.7 Hz, 2H), 7.20 (dd, J = 9.4, 5.2 Hz, 2H), 7.16-7.09 (m, 3H), 3.90 (d, J = 10.9 Hz, 2H), 3.64 (t, J = 7.1 Hz, 2H), 3.35 (s, 2H), 2.58 (dd, J = 15.2, 7.8 Hz, 3H), |
| | | 1.79 (d, J = 6.9 Hz, 2H), 1.73-1.61 |
| | | (m, 4H), 1.35 (s, 9H). |
| Compound 218 | 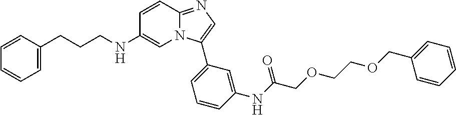 [SMILES: O=C(COCCOCc1ccccc1)Nc1cccc(-c2cnc3ccc(NCCCc4ccccc4)cn23)c1] | LC_MS: (ES+): m/z 535.26[M + H]+. ¹H NMR (400 MHz, DMSO) δ 9.82 (s, 1H), 8.02 (s, 1H), 7.55 (d, J = 8.0 Hz, 4H), 7.45 (t, J = 7.9 Hz, 1H), 7.35-7.28 (m, 5H), 7.26 (dd, J = 10.2, 4.4 Hz, 3H), 7.20 (d, J = 6.8 Hz, 2H), 7.16 (dd, J = 11.4, 4.2 Hz, 1H), 7.06 (d, J = 9.1 Hz, 1H), 5.76 (s, 1H), 4.51 (s, 2H), |
| | | 4.14 (s, 2H), 3.74 (dd, J = 5.6, 3.4 Hz, |
| | | 2H), 3.65 (dd, J = 5.8, 3.2 Hz, 2H), |
| | | 2.96 (d, J = 5.2 Hz, 2H), 2.69 (d, J = |
| | | 3.4 Hz, 2H), 1.90-1.81 (m, 2H). |
| Compound 219 | 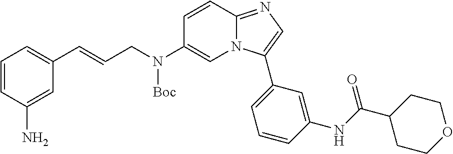 [SMILES: CC(C)(C)OC(=O)N(C/C=C/c1cccc(N)c1)c1ccc2ncc(-c3cccc(NC(=O)C4CCOCC4)c3)n2c1] | LC_MS: (ES+): m/z 568.28[M + H]+. ¹H NMR (400 MHz, CDCl3) δ 8.35 (s, 1H), 7.73 (t, J = 27.0 Hz, 7H), 7.13 (dd, J = 25.3, 17.6 Hz, 4H), 6.74 (d, J = 7.7 Hz, 1H), 6.61 (d, J = 7.1 Hz, 2H), 6.42- 6.34 (m, 1H), 6.29-6.18 (m, 1H), 4.35 (d, J = 6.1 Hz, 2H), 4.06 (d, J = 10.8 Hz, 2H), 3.46 (dd, J = 11.5, 9.7 Hz, 2H), 2.55 (s, 1H), 1.94-1.81 (m, 4H), 1.47 (s, 9H). |
| Compound 220 | 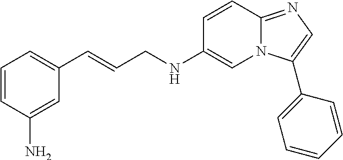 [SMILES: Nc1cccc(/C=C/CNc2ccc3ncc(-c4ccccc4)n3c2)c1] | LC_MS: (ES+): m/z 341.17[M + H]+. |
| Compound 221 | 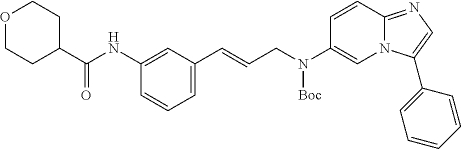 [SMILES: CC(C)(C)OC(=O)N(C/C=C/c1cccc(NC(=O)C2CCOCC2)c1)c1ccc2ncc(-c3ccccc3)n2c1] | LC_MS: (ES+): m/z 553.27[M + H]+. ¹H NMR (400 MHz, CDCl3) δ 8.28 (s, 1H), 7.75 (s, 1H), 7.56 (d, J = 9.7 Hz, 1H), 7.45 (dd, J = 14.6, 7.6 Hz, 4H), 7.39-7.29 (m, 5H), 7.10 (d, J = 7.7 Hz, 1H), 6.42 (d, J = 15.8 Hz, 1H), 6.33- 6.23 (m, 1H), 4.35 (d, J = 6.1 Hz, 2H), 4.07 (d, J = 10.8 Hz, 2H), 3.51-3.42 (m, 2H), 2.53 (ddd, J = 15.4, 11.4, 4.3 |
| | | Hz, 1H), 1.93 (dd, J = 12.4, 3.4 Hz, |
| | | 2H), 1.87 (s, 2H), 1.46 (s, 9H). |

The following compounds were synthesized referring to the synthetic route of intermediates BB5, BB6 and compound 2 in Example 2, using corresponding reagents:

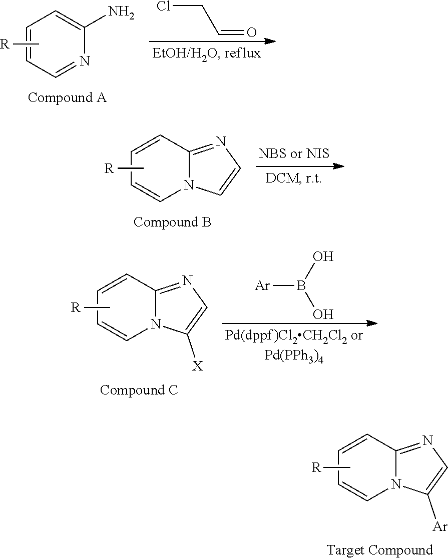

| Number | Structural formula | Mass spectrometry and NMR |
|---|---|---|
| Compound 222 | 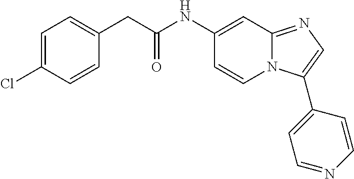 [SMILES: O=C(Cc1ccc(Cl)cc1)Nc1ccn2c(-c3ccncc3)cnc2c1] | LC_MS: (ES+): m/z 363.09 [M + H]+. ¹H NMR (400 MHz, CDCl3): δ 8.74-8.73 (m, 2H), 8.31-8.29 (m, 1H), 7.88-7.80 (m, 2H), 7.52-7.45 (m, 3H), 7.31-7.30 (m, 4H), 3.77 (s, 2H). |
| Compound 223 | 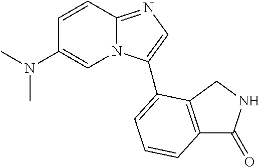 [SMILES: CN(C)c1ccc2ncc(-c3cccc4c3CNC4=O)n2c1] | LC_MS: (ES+): m/z 293.13 [M + H]⁺. ¹H NMR (400 MHz, CDCl₃): δ 8.70 (s, 1H), 7.94 (d, J = 7.4 Hz, 1H), 7.83 (s, 1H), 7.75 (d, J = 7.4 Hz, 1H), 7.69 (t, J = 7.5 Hz, 1H), 7.63 (d, J = 10.1 Hz, 1H), 7.48 (s, 1H), 7.36 (d, J = 9.8 Hz, 1H), 4.45 (s, 2H), 2.82 (s, 6H). |
| Compound 224 | 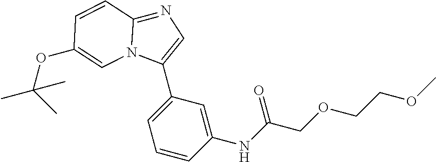 [SMILES: COCCOCC(=O)Nc1cccc(-c2cnc3ccc(OC(C)(C)C)cn23)c1] | LC_MS: (ES+): m/z 398.20 [M + H]+. |
| Compound 225 | 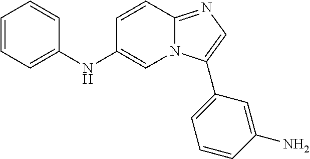 [SMILES: Nc1cccc(-c2cnc3ccc(Nc4ccccc4)cn23)c1] | LC_MS: (ES+): m/z 301.14 [M + H]+. ¹H NMR (400 MHz, CD3OD): δ 8.25 (m, 1H), 7.56-7.52 (m, 2H), 7.29-7.20 (m, 4H), 7.02-7.00 (m, 2H), 6.91-6.83 (m, 3H), 6.78-6.75 (m, 1H). |
| Compound 226 | 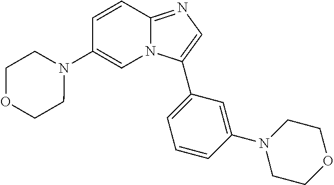 [SMILES: c1cc(-c2cnc3ccc(N4CCOCC4)cn23)cc(N2CCOCC2)c1] | LC_MS: (ES+): m/z 365.19[M + H]+. ¹H NMR (400 MHz, DMSO) δ 7.77 (s, 1H), 7.64 (s, 1H), 7.58 (s, 1H), 7.40 (t, J = 7.9 Hz, 1H), 7.35 (d, J = 8.7 Hz, 1H), 7.15 (s, 1H), 7.11 (d, J = 7.6 Hz, 1H), 7.03 (d, J = 8.3 Hz, 1H), 3.76 (dd, J = 5.6, 3.4 Hz, 8H), 3.22-3.17 (m, 4H), 3.07- 3.00 (m, 4H). |
| Compound 227 | 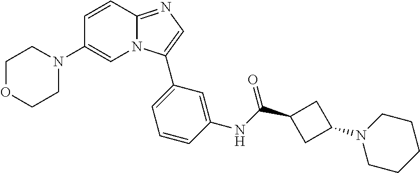 [SMILES: O=C(Nc1cccc(-c2cnc3ccc(N4CCOCC4)cn23)c1)[C@H]1C[C@H](N2CCCCC2)C1] | LC_MS: (ES+): m/z 460.26[M + H]+. ¹H NMR (400 MHz, DMSO) δ 10.21 (s, 1H), 8.05 (s, 1H), 7.89 (s, 1H), 7.65 (s, 1H), 7.58 (d, J = 9.8 Hz, 1H), 7.54 (d, J = 8.4 Hz, 1H), 7.49 (t, J = 7.8 Hz, 1H), 7.41-7.33 (m, 2H), 3.81-3.75 (m, 4H), 3.32 (d, J = 2.3 Hz, 1H), 3.17 (d, J = 5.2 Hz, 1H), 3.14-3.00 (m, 4H), 2.80-2.56 (m, 4H), 2.43 (s, 4H), 1.66 (s, 6H). |
| Compound 228 | 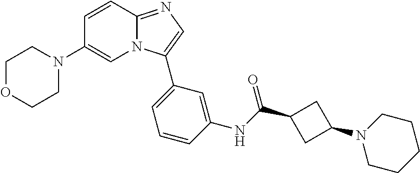 [SMILES: O=C(Nc1cccc(-c2cnc3ccc(N4CCOCC4)cn23)c1)[C@H]1C[C@@H](N2CCCCC2)C1] | LC_MS: (ES+): m/z 460.26[M + H]+. ¹H NMR (400 MHz, DMSO) δ 10.21 (s, 1H), 8.00 (s, 1H), 7.85 (s, 1H), 7.64 (s, 1H), 7.59 (d, J = 8.0 Hz, 2H), 7.49 (t, J = 7.9 Hz, 1H), 7.38 (d, J = 6.7 Hz, 2H), 3.80-3.75 (m, 4H), 3.33-3.31 (m, 1H), 3.09-3.05 (m, 4H), 3.01-2.96 (m, 1H), 2.78-2.60 (m, 4H), 2.39-2.30 (m, 4H), 1.71 (d, J = 63.3 Hz, 6H). |
| Compound 229 | 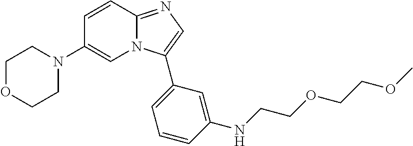 [SMILES: COCCOCCNc1cccc(-c2cnc3ccc(N4CCOCC4)cn23)c1] | LC_MS: (ES+): m/z 397.22[M + H]+. |
| Compound 230 | 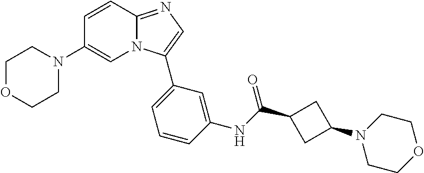 [SMILES: O=C(Nc1cccc(-c2cnc3ccc(N4CCOCC4)cn23)c1)[C@H]1C[C@@H](N2CCOCC2)C1] | LC_MS: (ES+): m/z 462.24[M + H]+. |
| Compound 231 | 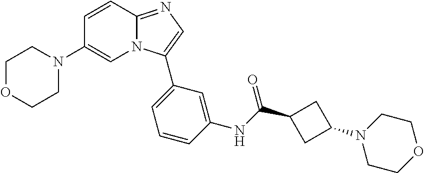 [SMILES: O=C(Nc1cccc(-c2cnc3ccc(N4CCOCC4)cn23)c1)[C@H]1C[C@H](N2CCOCC2)C1] | LC_MS: (ES+): m/z 462.24[M + H]+. |
| Compound 232 | 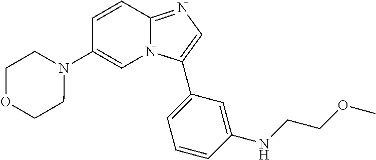 [SMILES: COCCNc1cccc(-c2cnc3ccc(N4CCOCC4)cn23)c1] | LC_MS: (ES+): m/z 353.19[M + H]+. |
| Compound 233 | 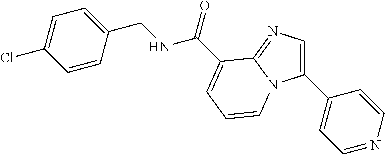 [SMILES: O=C(NCc1ccc(Cl)cc1)c1cccn2c(-c3ccncc3)cnc12] | LC_MS: (ES+): m/z 363.09[M + H]+. |
| Compound 234 | 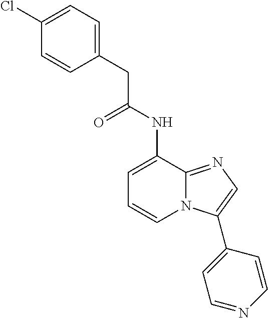 [SMILES: O=C(Cc1ccc(Cl)cc1)Nc1cccn2c(-c3ccncc3)cnc12] | LC_MS: (ES+): m/z 363.09[M + H]+. ¹H NMR (400 MHz, CDCl3): δ 8.98 (brs, 1H), 8.77 (brs, 2H), 8.30-8.28 (d, J = 7.6 Hz, 1H), 8.15-8.13 (d, J = 6.8 Hz, 1H), 7.74 (s, 1H), 7.50 (m, 2H), 7.38- 7.37 (m, 4H) , 6.95-6.92 (t, J = 7.2 Hz, 1H) 3.85 (s, 2H). |
| Compound 235 | 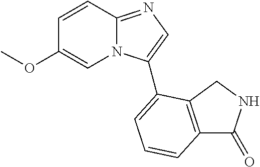 [SMILES: COc1ccc2ncc(-c3cccc4c3CNC4=O)n2c1] | LC_MS: (ES+): m/z 280.10[M + H]+. |

The following compounds were synthesized referring to the synthetic procedure of compound 47 in Example 3 and compound 63 in Example 6, using corresponding reagents:

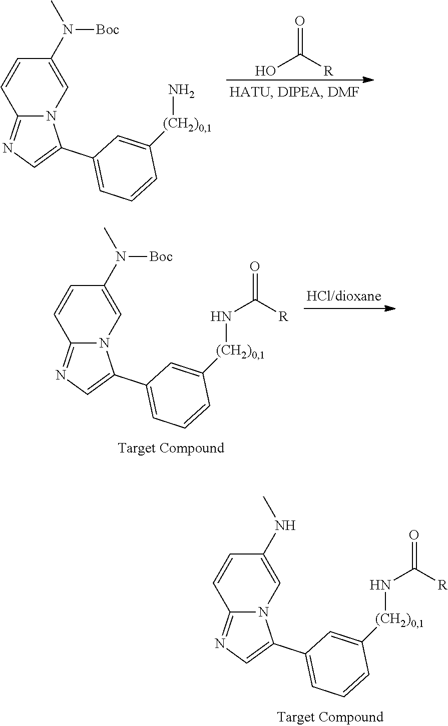

| Number | Structural formula | Mass spectrometry and NMR |
|---|---|---|
| Compound 236 | 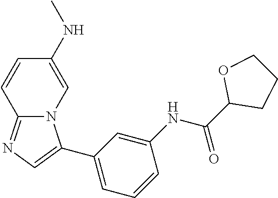 [SMILES: CNc1ccc2ncc(-c3cccc(NC(=O)C4CCCO4)c3)n2c1] | LC_MS: (ES+): m/z 337.16 [M + H]⁺. |
| Compound 237 | 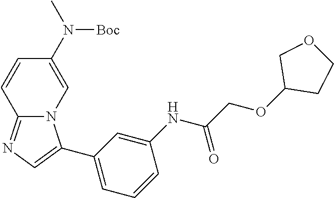 [SMILES: CN(C(=O)OC(C)(C)C)c1ccc2ncc(-c3cccc(NC(=O)COC4CCOC4)c3)n2c1] | LC_MS: (ES+): m/z 467.22 [M + H]⁺. |
| Compound 238 | 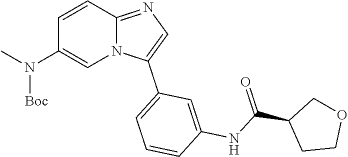 [SMILES: CN(C(=O)OC(C)(C)C)c1ccc2ncc(-c3cccc(NC(=O)[C@@H]4CCOC4)c3)n2c1] | LC_MS: (ES+): m/z 437.21 [M + H]⁺. ¹H NMR (400 MHz, CDCl₃): δ 10.24 (s, 1H), 8.50 (s, 1H), 7.92 (s, 1H), 7.77 (s, 1H), 7.66 (dd, J = 18.5, 8.8 Hz, 2H), 7.49 (t, J = 7.8 Hz, 1H), 7.34 (d, J = 7.8 Hz, 2H), 3.96 (t, J = 8.2 Hz, 1H), 3.75 (dt, J = 15.3, 7.6 Hz, 3H), 3.22 (s, 3H), 3.17 (d, J = 7.1 Hz, 1H), 2.10 (dd, J = 14.0, 6.9 Hz, 2H), 1.38 (s, 9H). |
| Compound 239 | 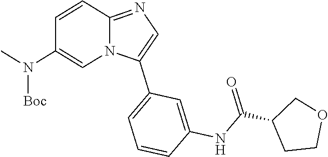 [SMILES: CN(C(=O)OC(C)(C)C)c1ccc2ncc(-c3cccc(NC(=O)[C@H]4CCOC4)c3)n2c1] | LC_MS: (ES+): m/z 437.21 [M + H]+. ¹H NMR (400 MHz, CDCl3): δ δ 10.23 (s, 1H), 8.50 (s, 1H), 7.92 (s, 1H), 7.77 (s, 1H), 7.65 (dd, J = 16.3, 8.8 Hz, 2H), 7.49 (t, J = 7.9 Hz, 1H), 7.34 (d, J = 7.7 Hz, 2H), 3.96 (t, J = 8.1 Hz, 1H), 3.75 (tt, J = 15.2, 7.4 Hz, 3H), 3.22 (s, 3H), 3.17 (d, J = 7.3 Hz, 1H), 2.12-2.05 (m, 2H), 1.38 (s, 9H). |
| Compound 240 | 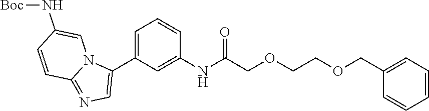 [SMILES: CC(C)(C)OC(=O)Nc1ccc2ncc(-c3cccc(NC(=O)COCCOCc4ccccc4)c3)n2c1] | LC_MS: (ES+): m/z 517.24 [M + H]+. |
| Compound 241 | 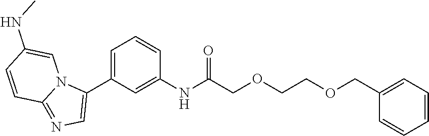 [SMILES: CNc1ccc2ncc(-c3cccc(NC(=O)COCCOCc4ccccc4)c3)n2c1] | LC_MS: (ES+): m/z 431.20 [M + H]+. |
| Compound 242 | 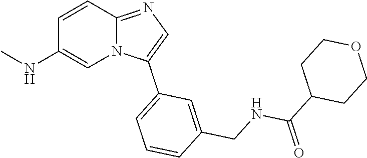 [SMILES: CNc1ccc2ncc(-c3cccc(CNC(=O)C4CCOCC4)c3)n2c1] | LC_MS: (ES+): m/z 365.19 [M + H]+. |
| Compound 243 | 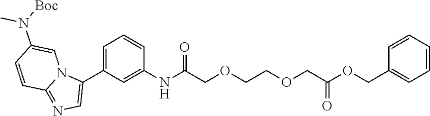 [SMILES: CN(C(=O)OC(C)(C)C)c1ccc2ncc(-c3cccc(NC(=O)COCCOCC(=O)OCc4ccccc4)c3)n2c1] | LC_MS: (ES+): m/z 589.26[M + H]+. |
| Compound 244 | 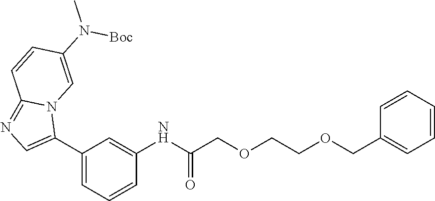 [SMILES: CN(C(=O)OC(C)(C)C)c1ccc2ncc(-c3cccc(NC(=O)COCCOCc4ccccc4)c3)n2c1] | LC_MS: (ES+): m/z 531.25[M + H]+. |
| Compound 245 | 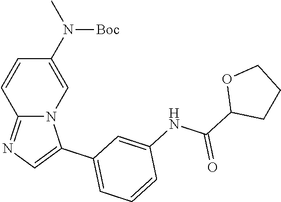 [SMILES: CN(C(=O)OC(C)(C)C)c1ccc2ncc(-c3cccc(NC(=O)C4CCCO4)c3)n2c1] | LC_MS: (ES+): m/z 437.21[M + H]+. ¹H NMR (400 MHz, DMSO) δ 9.86 (s, 1H), 8.53 (s, 1H), 7.98 (s, 1H), 7.77 (d, J = 6.0 Hz, 2H), 7.67 (d, J = 9.4 Hz, 1H), 7.49 (t, J = 7.9 Hz, 1H), 7.35 (t, J = 9.3 Hz, 2H), 4.42 (dd, J = 8.1, 5.6 Hz, 1H), 4.00 (dd, J = 14.5, 6.8 Hz, 1H), 3.84 (dt, J = 14.3, 7.1 Hz, 1H), 3.22 (s, 3H), 2.21 (dt, J = 19.9, 7.7 Hz, 1H), 2.01 (dt, J = 12.5, 6.6 Hz, 1H), 1.92-1.83 (m, 2H), 1.37 (s, 9H). |
| Compound 246 | 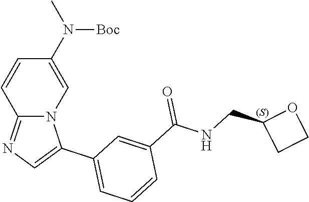 | LC_MS: (ES+): m/z 437.21[M + H]+. |
| Compound 247 | 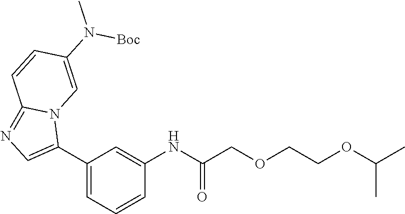 [SMILES: CC(C)OCCOCC(=O)Nc1cccc(-c2cnc3ccc(N(C)C(=O)OC(C)(C)C)cn23)c1] | LC_MS: (ES+): m/z 483.25[M + H]+. ¹H NMR (400 MHz, CDCl3) δ 8.96 (s, 1H), 8.35 (s, 1H), 7.94 (s, 1H), 7.73 (d, J = 28.7 Hz, 2H), 7.59 (d, J = 8.2 Hz, 1H), 7.51 (t, J = 7.9 Hz, 1H), 7.33 (d, J = 7.6 Hz, 1H), 7.24 (s, 1H), 4.16 (s, 2H), 3.81-3.78 (m, 2H), 3.74-3.70 (m, 1H), 3.69-3.67 (m, 2H), 3.30 (s, 3H), 1.46 (s, 9H), 1.24 (s, 3H), 1.22 (s, 3H). |
| Compound 248 | 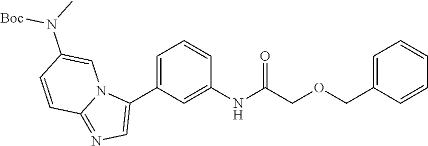 [SMILES: CN(C(=O)OC(C)(C)C)c1ccc2ncc(-c3cccc(NC(=O)COCc4ccccc4)c3)n2c1] | LC_MS: (ES+): m/z 487.23[M + H]+. |
| Compound 249 | 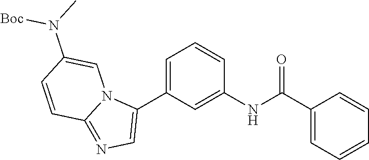 [SMILES: CN(C(=O)OC(C)(C)C)c1ccc2ncc(-c3cccc(NC(=O)c4ccccc4)c3)n2c1] | LC_MS: (ES+): m/z 443.20[M + H]+. |
| Compound 250 | 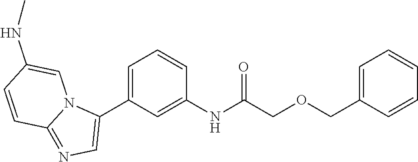 [SMILES: CNc1ccc2ncc(-c3cccc(NC(=O)COCc4ccccc4)c3)n2c1] | LC_MS: (ES+): m/z 387.17[M + H]+. |
| Compound 251 | 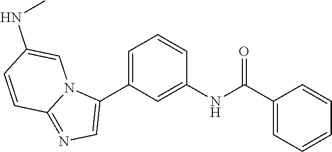 [SMILES: CNc1ccc2ncc(-c3cccc(NC(=O)c4ccccc4)c3)n2c1] | LC_MS: (ES+): m/z 343.15[M + H]+. |
| Compound 252 | 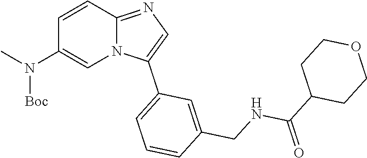 [SMILES: CN(C(=O)OC(C)(C)C)c1ccc2ncc(-c3cccc(CNC(=O)C4CCOCC4)c3)n2c1] | LC_MS: (ES+): m/z 465.24[M + H]+. ¹H NMR (400 MHz, DMSO) δ 8.46 (s, 1H), 8.38 (t, J = 5.9 Hz, 1H), 7.76 (s, 1H), 7.64 (d, J = 9.6 Hz, 1H), 7.51 (dd, J = 8.8, 5.1 Hz, 3H), 7.34-7.29 (m, 2H), 4.39-4.34 (m, 2H), 3.89-3.84 (m, 2H), 3.31-3.27 (m, 2H), 3.22 (s, 3H), 2.46- 2.40 (m, 1H), 1.62 (dd, J = 8.9, 5.2 Hz, 4H), 1.36 (d, J = 15.6 Hz, 9H). |
| Compound 253 | 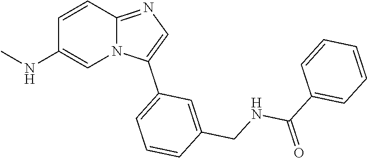 [SMILES: CNc1ccc2ncc(-c3cccc(CNC(=O)c4ccccc4)c3)n2c1] | LC_MS: (ES+): m/z 357.16[M + H]+. |

The following compounds were synthesized referring to the synthetic route of Compound 2 in Example 2 and Compound 63 in Example 6, using corresponding reagents:

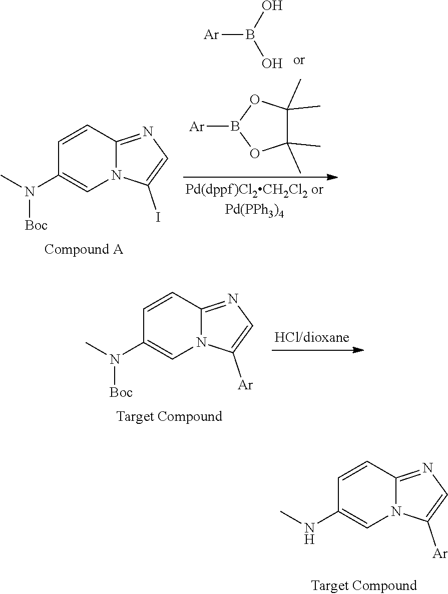

| Number | Structural formula | Mass spectrometry and NMR |
|---|---|---|
| Compound 254 | 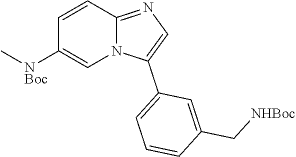 [SMILES: CN(C(=O)OC(C)(C)C)c1ccc2ncc(-c3cccc(CNC(=O)OC(C)(C)C)c3)n2c1] | LC_MS: (ES+): m/z 453.24 [M + H] |
| Compound 255 | 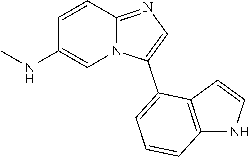 | LC_MS: (ES+): m/z 299.10 [M + H]+. ¹H NMR (400 MHz, DMSO-d6): δ 14.37 (brs, 1H), 11.60 (s, 1H), 8.25 (m, 1H), 7.85-7.83 (m, 1H), 7.67-7.65 (m, 1H), 7.56-7.54 (m, 2H), 7.34-7.32 (m, 2H), 7.22 (m, 1H), 6.29 (m, 1H), 2.53 (s, 3H). |
| Compound 256 | 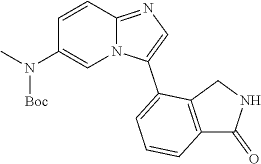 [SMILES: CN(C(=O)OC(C)(C)C)c1ccc2ncc(-c3cccc4c3CNC4=O)n2c1] | LC_MS: (ES+): m/z 379.17 [M + H]+. |
| Compound 257 | 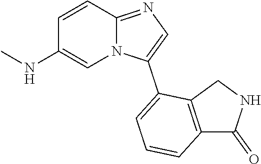 | LC_MS: (ES+): m/z 315.09 [M + H]+. |
| Compound 258 | 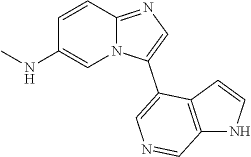 [SMILES: CNc1ccc2ncc(-c3cncc4[nH]ccc34)n2c1] | LC_MS: (ES+): m/z 264.12 [M + H]+. |
| Compound 259 | 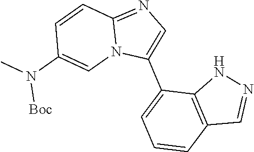 [SMILES: CN(C(=O)OC(C)(C)C)c1ccc2ncc(-c3cccc4cn[nH]c34)n2c1] | LC_MS: (ES+): m/z 364.17 [M + H]+. |
| Compound 260 | 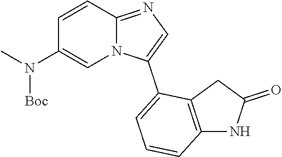 [SMILES: CN(C(=O)OC(C)(C)C)c1ccc2ncc(-c3cccc4c3CC(=O)N4)n2c1] | LC_MS: (ES+): m/z 379.17 [M + H]+. |
| Compound 261 | 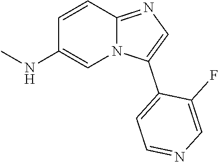 | LC_MS: (ES+): m/z 371.12 [M + H]+. ¹H NMR (400 MHZ, DMSO-d6): δ 8.75 (d, J = 2.4 Hz, 1H), 8.56-8.55 (m, 1H), 7.82-7.75 (m, 2H), 7.55 (d, J = 9.6 Hz, 1H), 7.29 (brs, 1H), 7.06-7.03 (m, 1H), 5.81-5.80 (m, 1H), 2.65 (d, J = 4.4 Hz, 3H). |
| Compound 262 | 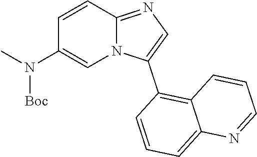 [SMILES: CN(C(=O)OC(C)(C)C)c1ccc2ncc(-c3cccc4ncccc34)n2c1] | LC_MS: (ES+): m/z 375.17 [M + H]+. |
| Compound 263 | 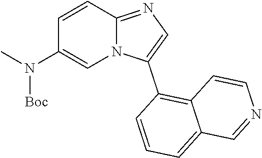 [SMILES: CN(C(=O)OC(C)(C)C)c1ccc2ncc(-c3cccc4cnccc34)n2c1] | LC_MS: (ES+): m/z 375.17 [M + H]⁺. ¹H NMR (400 MHz, CDCl₃): δ 9.47 (s, 1H), 8.49 (d, J = 5.9 Hz, 1H), 8.33 (d, J = 8.2 Hz, 1H), 8.05-7.99 (m, 2H), 7.90-7.85 (m, 2H), 7.84-7.78 (m, 1H), 7.45-7.38 (m, 2H), 3.09 (s, 3H), 1.30 (s, 9H). |
| Compound 264 | 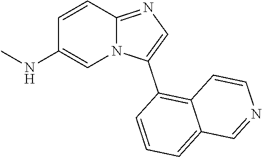 [SMILES: CNc1ccc2ncc(-c3cccc4cnccc34)n2c1] | LC_MS: (ES+): m/z 275.12 [M + H]⁺. ¹H NMR (400 MHz, CDCl₃): δ 10.12 (s, 1H), 8.83 (d, J = 8.3 Hz, 1H), 8.71 (d, J = 6.6 Hz, 1H), 8.45 (d, J = 7.0 Hz, 1H), 8.38 (s, 1H), 8.23 (t, J = 7.7 Hz, 1H), 8.16 (d, J = 6.6 Hz, 1H), 7.91 (d, J = 9.7 Hz, 1H), 7.60 (d, J = 9.7 Hz, 1H), 6.94 (s, 1H), 2.45 (s, 3H). |
| Compound 265 | 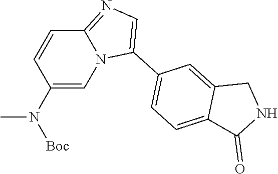 [SMILES: CN(C(=O)OC(C)(C)C)c1ccc2ncc(-c3ccc4c(c3)CNC4=O)n2c1] | LC_MS: (ES+): m/z 379.17 [M + H]⁺. ¹H NMR (400 MHz, CDCl₃): δ 8.63 (d, J = 12.9 Hz, 2H), 7.89 (d, J = 3.6 Hz, 2H), 7.82 (d, J = 7.9 Hz, 1H), 7.77 (d, J = 7.9 Hz, 1H), 7.70 (d, J = 9.6 Hz, 1H), 7.37 (d, J = 9.6 Hz, 1H), 4.47 (s, 2H), 3.22 (s, 3H), 1.38 (s, 9H). |
| Compound 266 | 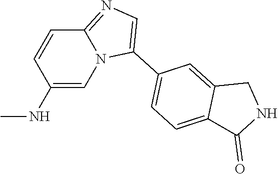 [SMILES: CNc1ccc2ncc(-c3ccc4c(c3)CNC4=O)n2c1] | LC_MS: (ES+): m/z 279.12 [M + H]+. ¹H NMR (400 MHz, CDCl3): δ 8.80 (s, 1H), 8.30 (s, 1H), 7.96 (s, 1H), 7.91 (d, J = 7.8 Hz, 1H), 7.84 (d, J = 9.4 Hz, 2H), 7.57 (d, J = 9.7 Hz, 1H), 7.51 (s, 1H), 4.50 (s, 2H), 2.68 (s, 3H). |
| Compound 267 | 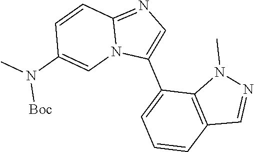 [SMILES: CN(C(=O)OC(C)(C)C)c1ccc2ncc(-c3cccc4cnn(C)c34)n2c1] | LC_MS: (ES+): m/z 378.19 [M + H]+. ¹H NMR (400 MHz, CDCl3): δ 8.11 (s, 1H), 9.94-7.92 (m, 2H), 7.66 (s, 1H), 7.41-7.38 (m, 2H), 7.30-7.28 (m, 2H), 3.50 (s, 3H), 3.17 (s, 3H), 1.36 (s, 9H). |
| Compound 268 | 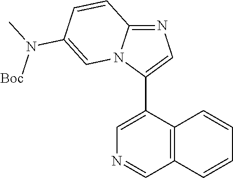 [SMILES: CN(C(=O)OC(C)(C)C)c1ccc2ncc(-c3cncc4ccccc34)n2c1] | LC_MS: (ES+): m/z 375.17 [M + H]+. ¹H NMR (400 MHz, CDCl3): δ 9.44 (s, 1H), 8.69 (s, 1H), 8.20-8.15 (m, 1H), 7.98 (s, 2H), 7.75 (dd, J = 9.5, 4.8 Hz, 3H), 7.64-7.59 (m, 1H), 7.39 (d, J = 8.8 Hz, 1H), 3.17 (s, 3H), 1.40 (s, 9H). |
| Compound 269 | 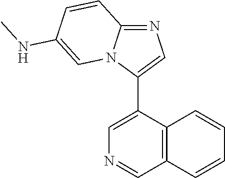 [SMILES: CNc1ccc2ncc(-c3cncc4ccccc34)n2c1] | LC_MS: (ES+): m/z 275.12 [M + H]+. |
| Compound 270 | 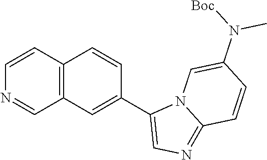 [SMILES: CN(C(=O)OC(C)(C)C)c1ccc2ncc(-c3ccc4ccncc4c3)n2c1] | LC_MS: (ES+): m/z 374.17 [M + H]+. ¹H NMR (400 MHz, CDCl3): δ 9.43 (s 1H), 9.43 (s, 1H), 8.75 (d, J = 1.2 Hz, 1H), 8.56 (d, J = 5.7 Hz, 1H), 8.45 (s, 1H), 8.15 (d, J = 8.6 Hz, 1H), 8.08 (dd, J = 8.6, 1.7 Hz, 1H), 8.00 (s, 1H), 7.70 (d, J = 9.6 Hz, 1H), 7.38 (dd, J = 9.6, 1.9 Hz, 1H), 3.24 (s, 3H), 1.39 (s, 9H). |
| Compound 271 | 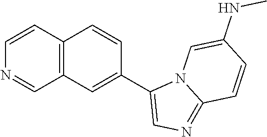 [SMILES: CNc1ccc2ncc(-c3ccc4ccncc4c3)n2c1] | LC_MS: (ES+): m/z 275.12 [M + H]+. |
| Compound 272 | 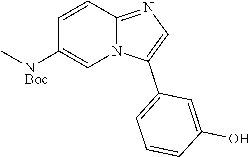 [SMILES: CN(C(=O)OC(C)(C)C)c1ccc2ncc(-c3cccc(O)c3)n2c1] | LC_MS: (ES+): m/z 340.16 [M + H]+. ¹H NMR (400 MHz, CDCl3): δ 9.70 (s, 1H), 8.44 (d, J = 0.8 Hz, 1H), 7.73 (s, 1H), 7.64 (d, J = 9.6 Hz, 1H), 7.37-7.30 (m, 2H), 7.08-7.00 (m, 2H), 6.86-6.84 (m, 1H), 3.20 (s, 3H), 1.38 (s, 9H). |
| Compound 273 | 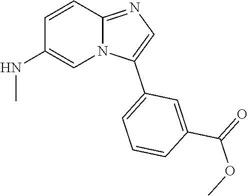 [SMILES: CNc1ccc2ncc(-c3cccc(C(=O)OC)c3)n2c1] | LC_MS: (ES+): m/z 282.13[M + H]+. |
| Compound 274 | 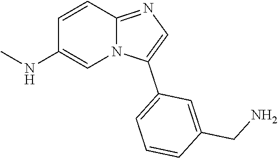 [SMILES: CNc1ccc2ncc(-c3cccc(CN)c3)n2c1] | LC_MS: (ES+): m/z 253.14[M + H]+. ¹H NMR (400 MHz, DMSO) δ 8.77 (s, 3H), 8.25 (s, 1H), 8.00 (s, 1H), 7.85 (d, J = 9.6 Hz, 1H), 7.75-7.65 (m, 4H), 7.59 (dd, J = 9.6, 1.8 Hz, 1H), 4.16-4.11 (m, 2H), 2.72 (s, 3H). |
| Compound 275 | 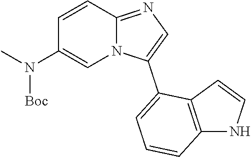 [SMILES: CN(C(=O)OC(C)(C)C)c1ccc2ncc(-c3cccc4[nH]ccc34)n2c1] | LC_MS: (ES+): m/z 363.17[M + H]+. ¹H NMR (400 MHz, DMSO-d6): δ 11.43 (s, 1H), 8.19 (s, 1H), 7.82 (s, 1H), 7.70- 7.67 (d, J = 9.6 Hz, 1H), 7.55-7.53 (d, J = 7.6 Hz, 1H), 7.47-7.46 (t, J = 2.8 Hz, 1H), 7.32-7.23 (m, 3H) , 6.24 (m, 1H), 3.14 (s, 3H) , 1.35 (s, 9H). |
| Compound 276 | 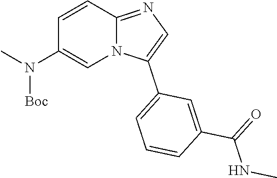 | LC_MS: (ES+): m/z 381.18[M + H]+. |
| Compound 277 | 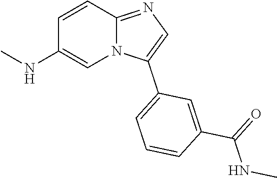 | LC_MS: (ES+): m/z 281.13[M + H]+. |
| Compound 278 | 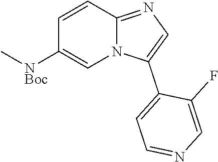 [SMILES: CN(C(=O)OC(C)(C)C)c1ccc2ncc(-c3ccncc3F)n2c1] | LC_MS: (ES+): m/z 343.15[M + H]+. ¹H NMR (400 MHz, CDCl3): δ 8.67 (d, J = 2.0 Hz, 1H), 8.58 (d, J = 4.8 Hz, 1H), 8.07 (brs, 1H), 7.93 (s, 1H), 7.78-7.75 (m, 1H), 7.51 (t, J = 5.6 Hz, 1H), 7.32 (d, J = 9.2 Hz, 1H), 3.29 (s, 3H), 1.45 (s, 9H). |
| Compound 279 | 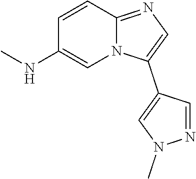 [SMILES: CNc1ccc2ncc(-c3cnn(C)c3)n2c1] | LC_MS: (ES+): m/z 228.12[M + H]+. ¹H NMR (400 MHz, DMSO-d6): δ 8.21 (s, 1H), 7.86(s, 1H), 7.56 (s, 1H), 7.43 (d, J = 9.6 Hz, 1H), 7.25 (d, J = 1.6 Hz, 1H), 6.96-6.93 (m, 1H), 5.75(s, 1H), 3.94 (s, 3H), 2.69 (s, 3H). |
| Compound 280 | 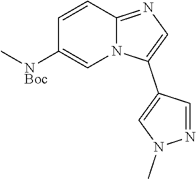 [SMILES: CN(C(=O)OC(C)(C)C)c1ccc2ncc(-c3cnn(C)c3)n2c1] | LC_MS: (ES+): m/z 328.17[M + H]+. ¹H NMR (400 MHz, DMSO-d6): δ 8.38 (d, J = 1.2 Hz, 1H), 8.30 (s, 1H), 7.89 (s, 1H), 7.72 (s, 1H), 7.58 (d, J = 9.2 Hz, 1H), 7.24-7.21 (m, 1H), 3.94 (s, 3H), 3.23 (s, 3H), 1.38 (s, 9H). |
| Compound 281 | 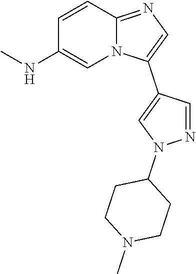 [SMILES: CNc1ccc2ncc(-c3cnn(C4CCN(C)CC4)c3)n2c1] | LC_MS: (ES+): m/z 311.19[M + H]+. ¹H NMR (400 MHz, DMSO-d6): δ 8.25 (s, 1H), 7.94 (s, 1H), 7.69 (s, 1H), 7.54 (d, J = 9.6 Hz, 1H), 7.40 (d, J = 4.6 Hz, 1H), 7.28-7.25 (m, 1H), 4.72-4.64 (m, 1H), 3.63 (d, J = 11.6 Hz, 2H), 3.28-3.21 (m, 2H), 2.91 (s, 3H), 2.79 (s, 3H), 2.45- 2.41 (m, 4H). |
| Compound 282 | 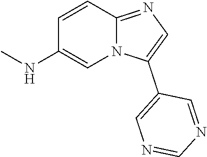 [SMILES: CNc1ccc2ncc(-c3cncnc3)n2c1] | LC_MS: (ES+): m/z 226.10[M + H]+. ¹H NMR (400 MHz, DMSO) δ 9.39 (s, 1H), 9.21 (s, 2H), 8.38 (s, 1H), 7.85 (d, J = 10.4 Hz, 1H), 7.61-7.56 (m, 2H), 2.70 (s, 3H). |
| Compound 283 | 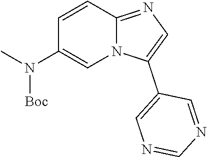 [SMILES: CN(C(=O)OC(C)(C)C)c1ccc2ncc(-c3cncnc3)n2c1] | LC_MS: (ES+): m/z 326.15[M + H]+. ¹H NMR (400 MHz, CDCl3) δ 9.32 (s, 1H), 9.00 (s, 2H), 8.29 (s, 1H), 7.90 (d, J = 15.8 Hz, 2H), 7.38 (d, J = 8.6 Hz, 1H), 3.30 (s, 3H), 1.48 (s, 9H). |
| Compound 284 | 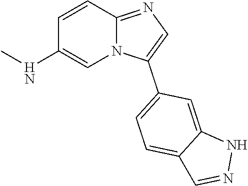 [SMILES: CNc1ccc2ncc(-c3ccc4cn[nH]c4c3)n2c1] | LC_MS: (ES+): m/z 264.12[M + H]+. |
| Compound 285 | 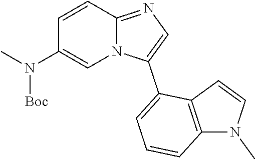 | LC_MS: (ES+): m/z 377.19[M + H]+. ¹H NMR (400 MHz, DMSO-d6): δ 8.20 (s, 1H), 7.82 (s, 1H), 7.73 (d, J = 9.6 Hz, 1H), 7.60 (d, J = 8.0 Hz, 1H), 7.46 (d, J = 3.2 Hz, 1H), 7.37-7.28 (m, 3H), 6.22 (d, J = 2.8 Hz, 1H), 3.87 (s, 3H), 3.14 (s, 3H), 1.35 (s, 9H). |
| Compound 286 | 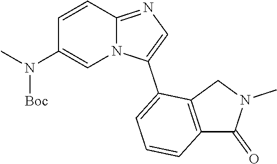 | LC_MS: (ES+): m/z 393.18[M + H]+. ¹H NMR (400 MHz, CDCl3): δ 8.08 (s, 1H), 7.99-7.97 (m, 1H), 7.85-7.84 (m, 1H), 7.67-7.66 (m, 2H), 7.39-7.36 (m, 1H), 7.27-7.26 (m, 1H), 4.28 (s, 2H), 3.25 (s, 3H), 3.21 (s, 3H), 1.45 (s, 9H). |
| Compound 287 | 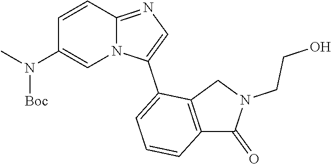 [SMILES: CN(C(=O)OC(C)(C)C)c1ccc2ncc(-c3cccc4c3CN(CCO)C4=O)n2c1] | LC_MS: (ES+): m/z 423.20[M + H]+. |
| Compound 288 | 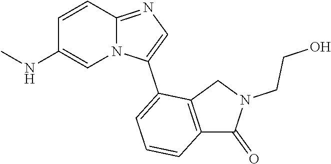 [SMILES: CNc1ccc2ncc(-c3cccc4c3CN(CCO)C4=O)n2c1] | LC_MS: (ES+): m/z 323.14[M + H]+. |
| Compound 289 | 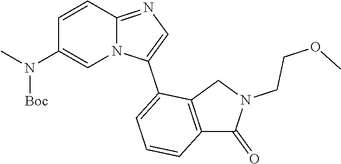 | LC_MS: (ES+): m/z 437.21[M + H]+. |
| Compound 290 | 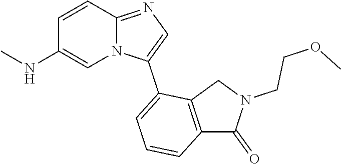 | LC_MS: (ES+): m/z 337.16[M + H]+. |
| Compound 291 | 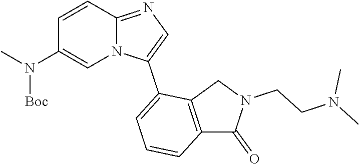 | LC_MS: (ES+): m/z 450.24[M + H]+. ¹H NMR (400 MHz, DMSO) δ 8.38 (s, 1H), 7.95 (s, 1H), 7.86 (d, J = 7.5 Hz, 1H), 7.79 (d, J = 7.4 Hz, 1H), 7.70 (dd, J = 8.4, 6.5 Hz, 2H), 7.35 (d, J = 9.5 Hz, 1H), 4.57 (s, 2H), 3.70 (t, J = 5.7 Hz, 2H), 3.19 (s, 3H), 2.72 (s, 2H), 2.33 (s, 6H), 1.37 (s, 9H). |
| Compound 292 | 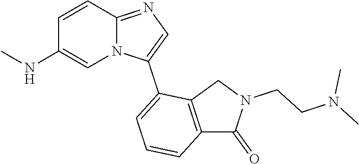 | LC_MS: (ES+): m/z 350.19[M + H]+. |
| Compound 293 | 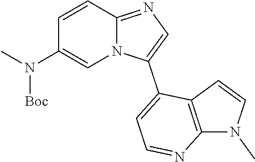 | LC_MS: (ES+): m/z 378.19 [M + H]⁺. ¹H NMR (400 MHz, CDCl₃): δ 8.48 (d, J = 4.8 Hz, 1H), 8.29 (s, 1H), 8.02 (brs, 1H), 7.64-7.53 (m, 1H), 7.44-7.37 (m, 1H), 7.30 (d, J = 3.6 Hz, 1H), 7.24 (d, J = 4.8 Hz, 1H), 6.39 (d, J = 3.2 Hz, 1H), 3.98 (s, 3H), 3.23 (s, 3H), 1.44 (s, 9H). |
| Compound 294 | 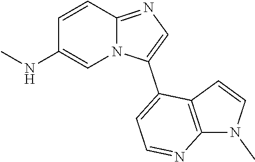 | LC_MS: (ES+): m/z 277.13 [M + H]+. ¹H NMR (400 MHz, DMSO-d6): δ 8.40 (d, J = 4.4 Hz, 1H), 7.79 (s, 1H), 7.67- 7.56 (m, 2H), 7.47-7.39 (m, 2H), 7.04 (d, J = 8.4 Hz, 1H), 6.45 (d, J = 1.6 Hz, 1H), 5.83-5.76 (m, 1H), 3.89 (s, 3H), 2.59 (s, 3H). |
| Compound 295 | 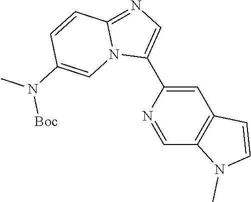 [SMILES: CN(C(=O)OC(C)(C)C)c1ccc2ncc(-c3cc4ccn(C)c4cn3)n2c1] | LC_MS: (ES+): m/z 378.19[M + H]+. ¹H NMR (400 MHz, CD3OD): δ 9.57 (d, J = 1.6 Hz, 1H), 8.91 (s, 1H), 8.02 (d, J = 4.0 Hz, 2H), 7.61 (d, J = 9.2 Hz, 1H), 7.49 (d, J = 3.2 Hz, 1H), 7.38-7.35 (m, 1H), 6.60 (d, J = 2.4 Hz, 1H), 3.99 (s, 3H), 3.31 (s, 3H), 1.45 (s, 9H). |
| Compound 296 | 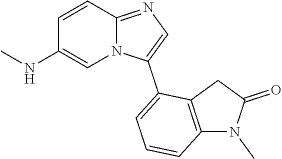 [SMILES: CNc1ccc2ncc(-c3cccc4c3CC(=O)N4C)n2c1] | LC_MS: (ES+): m/z 293.13[M + H]⁺. ¹H NMR (400 MHz, CDCl₃): δ 7.57 (brs, 2H), 7.45 (t, J = 8.0 Hz, 1H), 7.26- 7.23 (m, 1H), 7.18 (m, 1H), 6.91-6.81 (d, J = 8.0 Hz, 1H), 6.85-6.82 (m, 1H), 3.50 (s, 2H), 3.28 (s, 3H), 2.75 (s, 3H). |
| Compound 297 | 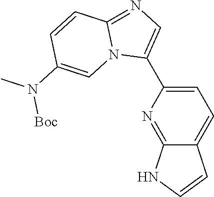 [SMILES: CN(C(=O)OC(C)(C)C)c1ccc2ncc(-c3ccc4cc[nH]c4n3)n2c1] | LC_MS: (ES+): m/z 364.17 [M + H]⁺. ¹H NMR (400 MHz, CDCl₃): δ 10.00 (s, 1H), 9.02 (s, 1H), 8.21 (s, 1H), 8.02 (d, J = 8.2 Hz, 1H), 7.53 (d, J = 8.3 Hz, 1H), 7.39 (dd, J = 6.7, 4.0 Hz, 2H), 6.60-6.57 (m, 1H), 3.37 (s, 3H), 1.48 (s, 9H). |
| Compound 298 | 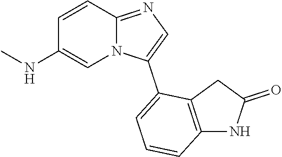 [SMILES: CNc1ccc2ncc(-c3cccc4c3CC(=O)N4)n2c1] | LC_MS: (ES+): m/z 279.12[M + H]+. |

Compound 299 and compound 300 were synthesized referring to the synthetic routes of compound 2 in Example 2 and compound 47 in Example 3, using corresponding reagents:

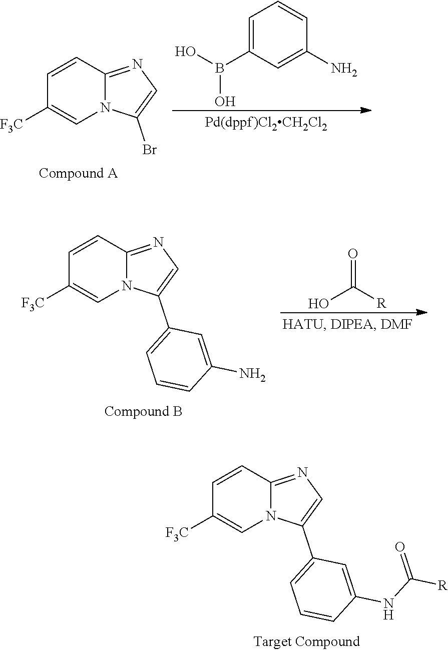

| Number | Structural formula | Mass spectrometry and NMR |
|---|---|---|
| Compound 299 | 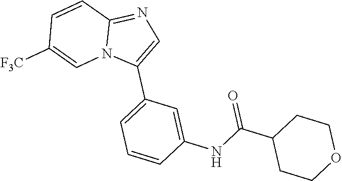 [SMILES: O=C(Nc1cccc(-c2cnc3ccc(C(F)(F)F)cn23)c1)C1CCOCC1] | LC_MS: (ES+): m/z 390.14 [M + H]+. |
| Compound 300 | 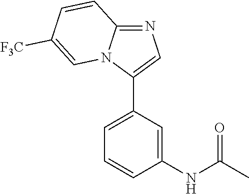 [SMILES: CC(=O)Nc1cccc(-c2cnc3ccc(C(F)(F)F)cn23)c1] | LC_MS: (ES+): m/z 320.09 [M + H]+. |

The synthetic route of compounds 301-304 was as follows:

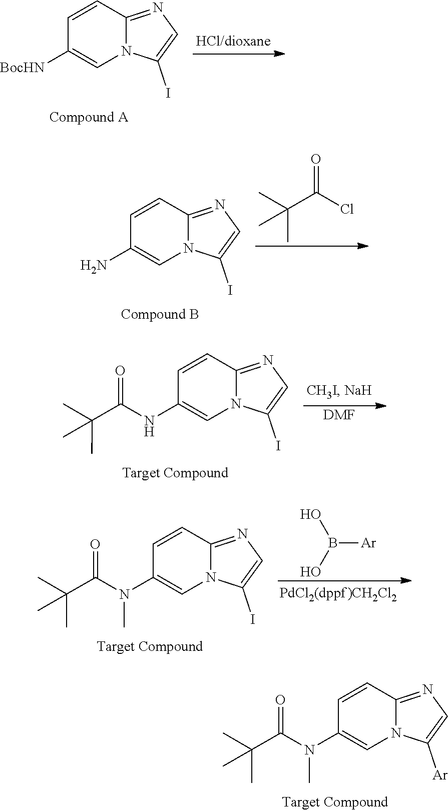

Step 1: The synthesis was performed referring to the synthesis process of compound 63 in Example 6 using corresponding reagents.

Step 2: The synthesis was performed referring to the second step of the synthesis process of compound 129 in Example 31 using corresponding reagents.

Step 3: The synthesis was performed referring to the fifth step of the synthesis process of intermediate BB6 using corresponding reagents.

Step 4: The synthesis was performed referring to the synthesis process of compound 2 in Example 2 using corresponding reagents.

| Number | Structural formula | Mass spectrometry and NMR |
|---|---|---|
| Compound 301 | 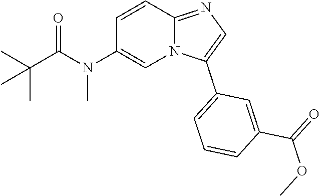 [SMILES: COC(=O)c1cccc(-c2cnc3ccc(N(C)C(=O)C(C)(C)C)cn23)c1] | LC_MS: (ES+): m/z 366.17 [M + H]+. |
| Compound 302 | 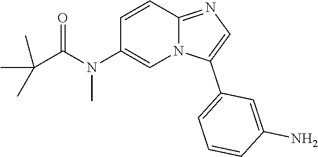 [SMILES: CN(C(=O)C(C)(C)C)c1ccc2ncc(-c3cccc(N)c3)n2c1] | LC_MS: (ES+): m/z 323.18[M + H]+. |
| Compound 303 | 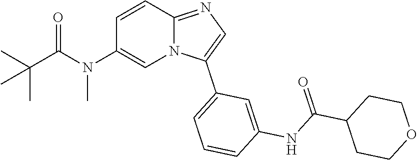 [SMILES: CN(C(=O)C(C)(C)C)c1ccc2ncc(-c3cccc(NC(=O)C4CCOCC4)c3)n2c1] | LC_MS: (ES+): m/z 435.23[M + H]+. |
| Compound 304 | 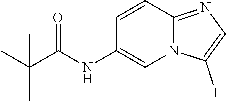 [SMILES: CC(C)(C)C(=O)Nc1ccc2ncc(I)n2c1] | LC_MS: (ES+): m/z 344.02[M + H]+. |

Compounds 305-307 were synthesized according to the following route:

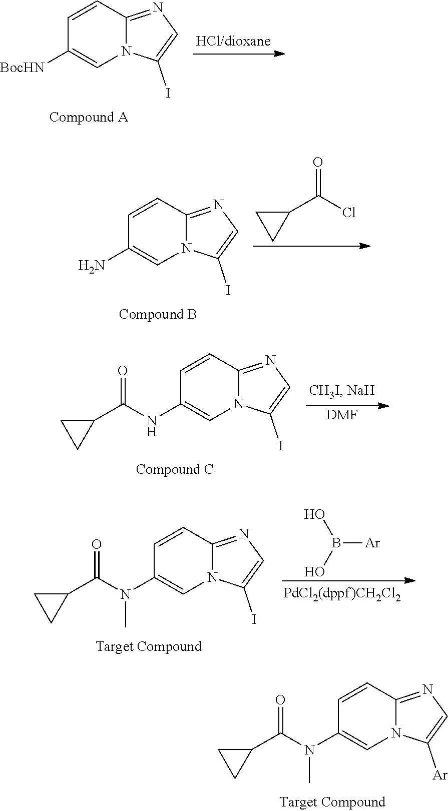

Step 1: The synthesis was performed referring to the synthesis process of compound 63 in Example 6 using corresponding reagents.

Step 2: The synthesis was performed referring to the second step of the synthesis process of compound 129 in Example 31 using corresponding reagents.

Step 3: The synthesis was performed referring to the fifth step of the synthesis process of intermediate BB6 using corresponding reagents.

Step 4: The synthesis was performed referring to the synthesis process of compound 2 in Example 2 using corresponding reagents.

| Number | Structural formula | Mass spectrometry and NMR |
|---|---|---|
| Compound 305 | 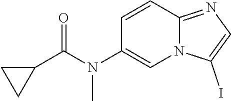 [SMILES: CN(C(=O)C1CC1)c1ccc2ncc(I)n2c1] | LC_MS: (ES+): m/z 342.00 [M + H]+. |
| Compound 306 | 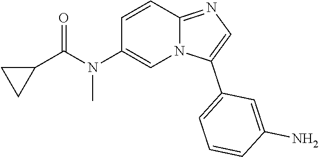 [SMILES: CN(C(=O)C1CC1)c1ccc2ncc(-c3cccc(N)c3)n2c1] | LC_MS: (ES+): m/z 307.15 [M + H]+. |
| Compound 307 | 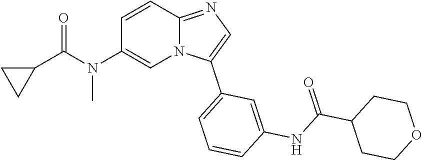 [SMILES: CN(C(=O)C1CC1)c1ccc2ncc(-c3cccc(NC(=O)C4CCOCC4)c3)n2c1] | LC_MS: (ES+): m/z 419.20 [M + H]+. |

Compounds 308-310 were synthesized according to the following route:

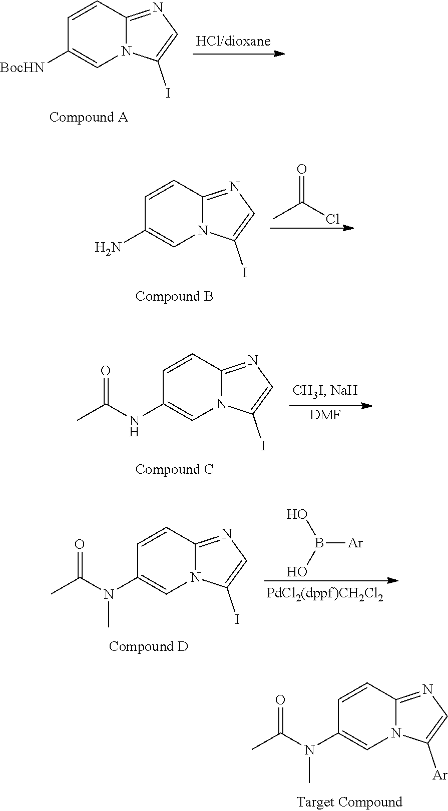

Step 1: The synthesis was performed referring to the synthesis process of compound 63 in Example 6 using corresponding reagents.

Step 2: The synthesis was performed referring to the second step of the synthesis process of compound 129 in Example 31 using corresponding reagents.

Step 3: The synthesis was performed referring to the fifth step of the synthesis process of intermediate BB6 using corresponding reagents.

Step 4: The synthesis was performed referring to the synthesis procedure of compound 2 in Example 2 using corresponding reagents.

| Number | Structural formula | Mass spectrometry and NMR |
|---|---|---|
| Compound 308 | 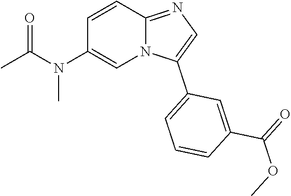 [SMILES: COC(=O)c1cccc(-c2cnc3ccc(N(C)C(C)=O)cn23)c1] | LC_MS: (ES+): m/z 324.13[M + H]+. |
| Compound 309 | 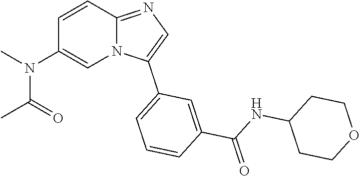 [SMILES: CC(=O)N(C)c1ccc2ncc(-c3cccc(C(=O)NC4CCOCC4)c3)n2c1] | LC_MS: (ES+): m/z 393.18[M + H]+. ¹H NMR (400 MHz, DMSO) δ 8.78 (s, 1H), 8.41 (d, J = 7.7 Hz, 1H), 8.09 (s, 1H), 7.90 (d, J = 9.5 Hz, 2H), 7.86 (d, J = 7.6 Hz, 1H), 7.76 (d, J = 9.5 Hz, 1H), 7.65 (t, J = 7.7 Hz, 1H), 7.37 (d, J = 9.2 Hz, 1H), 4.04 (qd, J = 11.3, 5.8 Hz, 1H), 3.89 (d, J = 10.2 Hz, 2H), 3.42-3.36 (m, 2H), 3.16 (s, 3H), 1.87 (s, 3H), 1.79 (dd, J = 12.6, 2.3 Hz, 2H), 1.58 (qd, J = 12.0, 4.4 Hz, 2H). |
| Compound 310 | 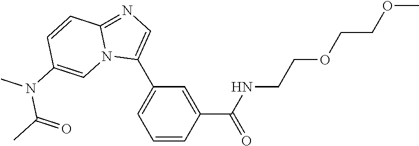 | LC_MS: (ES+): m/z 411.20[M + H]+. |

The following compounds were synthesized referring to the synthetic procedure of compound 83, compound 84, and compound 85 in Example 9, using corresponding reagents:

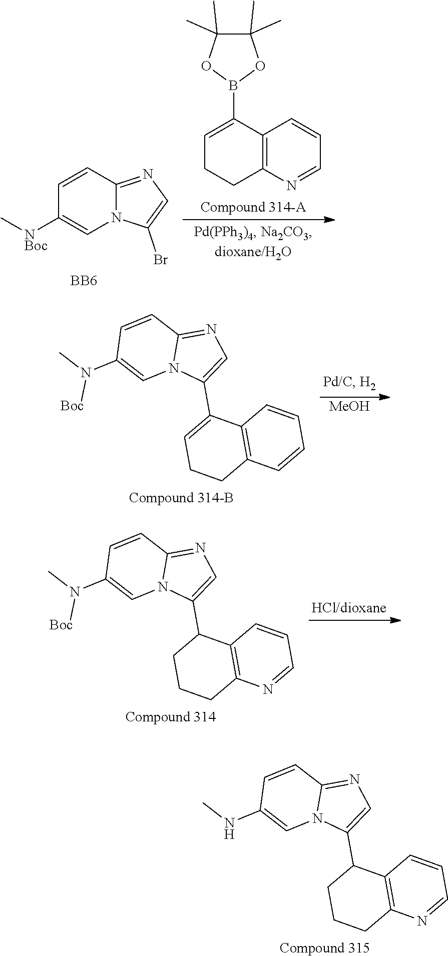

| Number | Structural formula | Mass spectrometry and NMR |
|---|---|---|
| Compound 311 | 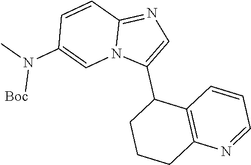 [SMILES: CN(C(=O)OC(C)(C)C)c1ccc2ncc(C3CCCc4ncccc43)n2c1] | LC_MS: (ES+): m/z 379.21 [M + H]+. |
| Compound 312 | 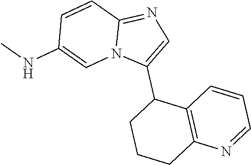 [SMILES: CNc1ccc2ncc(C3CCCc4ncccc43)n2c1] | LC_MS: (ES+): m/z 279.15[M + H]+. |

### Biological Activity Examples

### Biological Activity Example 1: Preparation of SARM1 and Assay of NAD Enzymatic Activity

### Preparation of Compounds to be Tested:

Compounds to be tested were stocked at 200 μM or 10 mM (in DMSO), and further diluted to the desired compound concentration for the in vitro SARM1 enzyme assay and inhibitor screening.

### Expression and Purification of SARM1 Protein

### (1) Plasmid Construction

In this example, the gene sequence of dN-SARM1 was amplified using PCR, the N-terminal mitochondrial localization signal peptide of SARM1 was removed, and the PCR amplification product was constructed into the pLenti-CMV-puro-dest plasmid (addgene catalog #17452). The details were as follows:

BC2T-TEV polypeptide gene fragment, dN-SARM1-F and dN-SARM1-R were synthesized by Sangon Biotech (Shanghai) Co., Ltd. The BC2T-TEV polypeptide gene fragment had a sequence set forth in SEQ ID No.: 1, dN-SARM1-F had a sequence set forth in SEQ ID No.: 2, and dN-SARM1-R had a sequence set forth in SEQ ID No.: 3.

SEQ ID No.: 1:
5′-CTCATGccagacagaaaagcggctgttagtcactggcagcaaGATAT
CGGCGGAGGCGGATCTGGCGGAGGCGGATCTGGCGGAGGCGGATCTgaga
atttgtattttcagggtGGCGGAGGCGGAGGTACCCTG-3′
SEQ ID No.: 2:
5′-GGTACCCTGGCGGTGCCTGGGCCAG-3′
SEQ ID No.: 3:
5′-GCGGCCGCCTAGGTTGGACCCATGGGTGCAGCACCC-3′

The synthesized BC2T-TEV polypeptide gene fragment was connected into the pENTR vector pENTR1A-GFP-N2 (addgene: catalog #19364) through HindIII/KpnI restriction sites. The dN-SARM1 gene fragment was amplified using primers dN-SARM1-F and dN-SARM1-R. The dN-SARM1 gene fragments obtained from amplification were constructed into pENTR vector with BC2T-TEV through KpnI and NotI restriction sites. All endonucleases used in this example were purchased from Thermo.

The dN-SARM1 gene fragment obtained by PCR amplification had a sequence set forth in SEQ ID No.: 4.

SEQ ID No.: 4:
GGTACCCTGGCGGTGCCTGGGCCAGATGGGGGCGGTGGCACGGGCCCATG
GTGGGCTGCGGGTGGCCGCGGGCCCCGCGAAGTGTCGCCGGGGGCAGGCA
CCGAGGTGCAGGACGCCCTGGAGCGCGCGCTGCCGGAGCTGCAGCAGGCC
TTGTCCGCGCTGAAGCAGGCGGGCGGCGCGCGGGCCGTGGGCGCCGGCCT
GGCCGAGGTCTTCCAACTGGTGGAGGAGGCCTGGCTGCTGCCGGCCGTGG
GCCGCGAGGTAGCCCAGGGTCTGTGCGACGCCATCCGCCTCGATGGCGGC
CTCGACCTGCTGTTGCGGCTGCTGCAGGCGCCGGAGTTGGAGACGCGTGT
GCAGGCCGCGCGCCTGCTGGAGCAGATCCTGGTGGCTGAGAACCGAGACC
GCGTGGCGCGCATTGGGCTGGGCGTGATCCTGAACCTGGCGAAGGAACGC
GAACCCGTAGAGCTGGCGCGGAGCGTGGCAGGCATCTTGGAGCACATGTT
CAAGCATTCGGAGGAGACATGCCAGAGGCTGGTGGCGGCCGGCGGCCTGG
ACGCGGTGCTGTATTGGTGCCGCCGCACGGACCCCGCGCTGCTGCGCCAC
TGCGCGCTGGCGCTGGGCAACTGCGCGCTGCACGGGGGCCAGGCGGTGCA
GCGACGCATGGTAGAGAAGCGCGCAGCCGAGTGGCTCTTCCCGCTCGCCT
TCTCCAAGGAGGACGAGCTGCTTCGGCTGCACGCCTGCCTCGCAGTAGCG
GTGTTGGCGACTAACAAGGAGGTGGAGCGCGAGGTGGAGCGCTCGGGCAC
GCTGGCGCTCGTGGAGCCGCTTGTGGCCTCGCTGGACCCTGGCCGCTTCG
CCCGCTGTCTGGTGGACGCCAGCGACACAAGCCAGGGCCGCGGGCCCGAC
GACCTGCAGCGCCTCGTGCCGTTGCTCGACTCTAACCGCTTGGAGGCGCA
GTGCATCGGGGCTTTCTACCTCTGCGCCGAGGCTGCCATCAAGAGCCTGC
AAGGCAAGACCAAGGTGTTCAGCGACATCGGCGCCATCCAGAGCCTGAAA
CGCCTGGTTTCCTACTCTACCAATGGCACTAAGTCGGCGCTGGCCAAGCG
CGCGCTGCGCCTGCTGGGCGAGGAGGTGCCACGGCCCATCCTGCCCTCCG
TGCCCAGCTGGAAGGAGGCCGAGGTTCAGACGTGGCTGCAGCAGATCGGT
TTCTCCAAGTACTGCGAGAGCTTCCGGGAGCAGCAGGTGGATGGCGACCT
GCTTCTGCGGCTCACGGAGGAGGAACTCCAGACCGACCTGGGCATGAAAT
CGGGCATCACCCGCAAGAGGTTCTTTAGGGAGCTCACGGAGCTCAAGACC
TTCGCCAACTATTCTACGTGCGACCGCAGCAACCTGGCGGACTGGCTGGG
CAGCCTGGACCCGCGCTTCCGCCAGTACACCTACGGCCTGGTCAGCTGCG
GCCTGGACCGCTCCCTGCTGCACCGCGTGTCTGAGCAGCAGCTGCTGGAA
GACTGCGGCATCCACCTGGGCGTGCACCGCGCCCGCATCCTCACGGCGGC
CAGAGAAATGCTACACTCCCCGCTGCCCTGTACTGGTGGCAAACCCAGTG
GGGACACTCCAGATGTCTTCATCAGCTACCGCCGGAACTCAGGTTCCCAG
CTGGCCAGTCTCCTGAAGGTGCACCTGCAGCTGCATGGCTTCAGTGTCTT
CATTGATGTGGAGAAGCTGGAAGCAGGCAAGTTCGAGGACAAACTCATCC
AGAGTGTCATGGGTGCCCGCAACTTTGTGTTGGTGCTATCACCTGGAGCA
CTGGACAAGTGCATGCAAGACCATGACTGCAAGGATTGGGTGCATAAGGA
GATTGTGACTGCTTTAAGCTGCGGCAAGAACATTGTGCCCATCATTGATG
GCTTCGAGTGGCCTGAGCCCCAGGTCCTGCCTGAGGACATGCAGGCTGTG
CTTACTTTCAACGGTATCAAGTGGTCCCACGAATACCAGGAGGCCACCAT
TGAGAAGATCATCCGCTTCCTGCAGGGCCGCTCCTCCCGGGACTCATCTG
CAGGCTCTGACACCAGTTTGGAGGGTGCTGCACCCATGGGTCCAACCTAG

The PCR amplification reaction system comprised: 10 μL of 5× PrimeSTAR Buffer (Mg²⁺ plus), 4 μL of dNTP Mixture (2.5 mM each), dN-SARM1-F with a final concentration of 0.2 μmol/L, dN-SARM1-R with a final concentration of 0.2 μmol/L, 100 ng of DNA template, and 0.5 μL of PrimeSTAR HS DNA Polymerase (2.5 U/μL), and the system was finally added with sterilized ddH₂O to 50 μL. The full-length SARM1 was synthesized into the pUC57 plasmid by WZ Biosciences Inc., and PCR was performed using pUC57-SARM1 as a DNA template.

The PCR amplification products were electrophoresed using agarose gel, and then recovered and purified using Omega gel recovery kit D2500-02. The specific steps of gel cutting and recovery were performed referring to the kit instruction. The recovered and purified PCR amplification product was constructed into the pENTR vector with BC2T-TEV

A recombinant plasmid was constructed as follows:

Enzyme digestion reaction system comprised 800 ng of PCR amplification recovery product or plasmid, 1 μL each of endonuclease (Fastdigest) and 1 μL of buffer, and the system was added with sterilized water to a volume of 10 μL. The enzyme digestion reaction was performed at a constant temperature of 37° C. for 30 minutes.

Plasmid ligation: After the enzyme digestion reaction was completed, 300 ng of the enzyme-digested PCR amplification recovery product and 50 ng of the enzyme-digested plasmid were evenly mixed with 1 μL of T4 DNA ligase and 1 μL of T4 DNA ligase buffer, and the mixture was added with sterilized water to a volume of 20 μL. The ligation was performed at a constant temperature of 16° C. overnight.

The ligation product was subjected to agarose gel electrophoresis, and then recovered and purified using Omega gel recovery kit D2500-02. The recovered and purified product was the recombinant plasmid of this example, labeled as pENTR1A-BC2T-dN-SARM1.

After the pENTR1A-BC2T-dN-SARM1 plasmid was constructed, dN-SARM1 was recombined into pLenti-CMV-puro-dest through LR reaction.

Recombinant reaction system comprised 150 ng of pENTR1A-BC2T-dN-SARM1, 50 ng of pLenti-CMV-puro-dest and 1 μL of 5× LR Clonase™ reaction buffer, and the system was added with sterilized water to a total volume of 5 μL.

### (2) Transfection

In this example, the constructed pLenti-CMV-puro-dest and viral packaging plasmid psPAX2, pMD2.G (addgene psPAX2: #12260, pMD2.G: #12259) were co-transfected into HEK293T cells (ATCC) through liposomes Lipofectamine 2000 (Life Technologies), to prepare viruses with dN-SARM1 reading frame. The details were as follows:

1×10⁶ cells were plated in a 3.5 cm dish, and transfect the next day.

A plasmid mixture comprised 1.7 μg of pLenti-dN-SARM1, 1.7 μg of psPAX2, 0.6 μg of pMD2.G, and 8 μL of lipofectamine 2000 transfection reagent. Transfection was carried out according to the instruction. The medium was changed after 8 hours, and the virus was collected after 48 hours.

### (3) Cell Screening

HEK293T cells obtained in the “(2) Transfection” step were infected with dN-SARM1 virus, and cells stably expressing dN-SARM1 protein were screened by adding puromycin. The details were as follows:

Virus: 2×10⁵ cells were infected with virus at 80 μL/3.5 cm. After 48 hours of infection, 2 μg/mL puromycin was added for screening. After 48 hours of screening, cells not infected with the virus were completely dead. Most of the virus-infected cells survived. 2 μg/mL puromycin was added for secondary screening for 48 hours.

### (4) Protein Extraction

Cells stably expressing dN-SARM1 protein obtained in the “(3) Cell Screening” step were cultured and collected. The dN-SARM1 protein expressed in the cytoplasm was obtained through digitonin cleavage, which was used for in vitro activity assay. The details were as follows:

Cells were cultured in DMEM in a 10 cm dish, digested with trypsin-EDTA, then centrifuged at 1000 rpm for 5 minutes, washed once with PBS, then resuspended with PBS containing 100 μM digitonin at 0.6 mL PBS/10 cm cells, and lysed for 5 minutes. The cells were added with trypan blue and observed under a microscope. More than 90% of the cells had been lysed. The cells were centrifuged at 5000 rpm for 10 minutes, and the supernatant of dN-SARM1 protein was collected.

### Biological Activity Example 2: In Vitro Biochemical Assay for Inhibiting SARM1 Enzymatic Activity (% Inhibition Rate)

The compounds were subjected to PC6 fluorescence method measurement [Chinese Patent 202010528147.3] using the dN-SARM1 protein obtained through “(4) Protein extraction” in “Expression and purification of SARM1 protein” in the above biological activity example 1.

### Reaction Conditions:

First, 0.05 μg/ml dN-SARM1 and 20 μM compound were incubated in 50 mM Tris-HCl (pH 7.5) solution for 10 min, and then added with 50 μM NAD and 50 μM PC6 as substrate and 50 μM NMN as activator for 30 minutes of reaction at room temperature. The concentration of each component was the final concentration in the reaction system.

During the reaction, PC6 fluorescence spectrum dynamics were detected by a microplate reader, wherein the excitation wavelength and emission wavelength for detection were 390 nm and 520 nm, respectively. Finally, the reaction rate was used to express the activity of the protein. The higher the reaction rate, the stronger the activity of the protein and the lower the inhibitory efficiency of the compound.

Table 1 below provides the inhibition rate interval of SARM1 enzymatic activity by some compounds at 20 μM: A>90%; B: 50-89%; C: 25-49%;

TABLE 1

| Compound | Inhibition rate of |
|---|---|
| number | enzymatic activity |
| 1 | A |
| 19 | A |
| 20 | B |
| 22 | B |
| 24 | A |
| 25 | A |
| 26 | A |
| 27 | B |
| 28 | B |
| 32 | A |
| 33 | A |
| 34 | A |
| 35 | B |
| 37 | B |
| 40 | A |
| 41 | A |
| 44 | B |
| 46 | A |
| 48 | B |
| 49 | A |
| 50 | A |
| 53 | A |
| 54 | A |
| 55 | A |
| 56 | A |
| 57 | A |
| 58 | B |
| 75 | A |
| 77 | A |
| 78 | A |
| 79 | A |
| 90 | A |
| 94 | A |
| 95 | B |
| 105 | A |
| 108 | B |
| 109 | B |
| 110 | A |
| 115 | A |
| 122 | A |
| 123 | A |
| 124 | A |
| 125 | A |
| 126 | A |
| 127 | B |
| 128 | A |
| 129 | A |
| 130 | A |
| 131 | A |
| 132 | A |
| 133 | B |
| 134 | A |
| 135 | B |
| 136 | A |
| 139 | B |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |

### Biological Activity Example 3: In Vitro Biochemical Assay for Inhibiting SARM1 Enzymatic Activity (IC₅₀)

200 μM compound was first added to a solution of 0.05 μg/ml dN-SARM1 in 50 mM Tris-HCl (pH 7.5). Half of the mixture was added to an equal volume of a solution of 0.05 μg/ml dN-SARM1 in 50 mM Tris-HCl (pH 7.5) and mixed. The drug was diluted 6 times in this manner, with the final concentrations being 200, 100, 50, 25, 12.5, 6.25, 3.125 μM, or 200, 50, 12.5, 3.125, 0.78, 0.195, 0.049 μM, respectively. The protein without inhibitors added, being used as control group, was incubated at room temperature for 10 minutes.

50 μM NAD, 50 μM PC6 as substrate and 50 μM NMN as activator were added to the dN-SARM1 protein incubated with the inhibitor and reacted for 30 minutes at room temperature. The concentration of each component was the final concentration in the reaction system.

During the reaction, PC6 fluorescence spectrum dynamics were detected by a microplate reader, wherein the excitation wavelength and emission wavelength for detection were 390 nm and 520 nm, respectively. Finally, the reaction rate was used to express the activity of the protein, and the half inhibitory concentration was calculated. The higher the reaction rate, the stronger the protein activity, and the lower the inhibitory efficiency of the compound.

The dose curve of compounds inhibiting SARM1 enzymatic activity was determined using the above method.

IC₅₀ intervals of these compounds in the assay are provided in Table 2 below:

IC₅₀ intervals for inhibiting SARM1 enzymatic activity: A<1.0 μM; B: 1-10 μM; C: 10-25 μM

TABLE 2

| Compound Number | IC₅₀ |
|---|---|
| 2 | A |
| 5 | B |
| 6 | B |
| 7 | B |
| 8 | B |
| 9 | B |
| 10 | B |
| 11 | B |
| 12 | B |
| 13 | A |
| 14 | B |
| 15 | B |
| 16 | B |
| 17 | B |
| 18 | B |
| 20 | A |
| 23 | B |
| 24 | B |
| 25 | B |
| 26 | A |
| 27 | B |
| 32 | B |
| 33 | B |
| 34 | B |
| 37 | B |
| 40 | B |
| 41 | B |
| 42 | A |
| 43 | B |
| 44 | B |
| 46 | B |
| 47 | A |
| 49 | B |
| 50 | B |
| 53 | B |
| 54 | A |
| 55 | B |
| 56 | A |
| 57 | A |
| 58 | A |
| 59 | B |
| 60 | B |
| 61 | B |
| 62 | B |
| 63 | B |
| 64 | B |
| 65 | A |
| 67 | B |
| 68 | B |
| 69 | B |
| 70 | B |
| 72 | B |
| 73 | B |
| 74 | B |
| 75 | B |
| 76 | B |
| 77 | B |
| 78 | A |
| 79 | A |
| 80 | B |
| 81 | B |
| 82 | B |
| 83 | B |
| 84 | B |
| 85 | B |
| 86 | A |
| 87 | A |
| 89 | A |
| 90 | B |
| 91 | B |
| 92 | B |
| 94 | A |
| 96 | A |
| 99 | B |
| 105 | B |
| 108 | B |
| 110 | A |
| 113 | B |
| 114 | A |
| 115 | A |
| 116 | A |
| 117 | B |
| 118 | B |
| 119 | A |
| 120 | A |
| 121 | A |
| 122 | A |
| 123 | A |
| 124 | B |
| 125 | B |
| 128 | B |
| 129 | A |
| 130 | A |
| 131 | B |
| 132 | B |
| 134 | A |
| 136 | A |
| 143 | B |
| 144 | A |
| 145 | A |
| 146 | B |
| 149 | B |
| 150 | B |
| 151 | B |
| 152 | B |
| 153 | B |
| 154 | A |
| 156 | B |
| 157 | B |
| 158 | B |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | B |
| 174 | A |
| 175 | A |
| 176 | B |
| 177 | B |
| 178 | A |
| 179 | A |
| 180 | B |
| 181 | B |
| 182 | B |
| 183 | B |
| 184 | B |
| 185 | B |
| 186 | B |
| 191 | B |
| 192 | B |
| 193 | B |
| 194 | B |
| 195 | B |
| 196 | B |
| 197 | B |
| 199 | B |
| 200 | B |
| 201 | B |
| 202 | B |
| 203 | A |
| 204 | B |
| 205 | B |
| 207 | B |
| 208 | B |
| 209 | B |
| 210 | B |
| 211 | B |
| 212 | B |
| 213 | B |
| 217 | B |
| 218 | B |
| 219 | B |
| 220 | B |
| 221 | B |
| 222 | B |
| 223 | A |
| 226 | B |
| 227 | B |
| 229 | B |
| 230 | B |
| 231 | B |
| 232 | B |
| 234 | B |
| 235 | B |
| 236 | A |
| 237 | A |
| 238 | A |
| 239 | A |
| 240 | A |
| 241 | A |
| 242 | A |
| 244 | B |
| 245 | B |
| 246 | B |
| 247 | B |
| 248 | B |
| 249 | B |
| 250 | B |
| 251 | B |
| 253 | B |
| 254 | A |
| 255 | A |
| 256 | A |
| 257 | A |
| 258 | A |
| 259 | A |
| 260 | A |
| 261 | A |
| 262 | A |
| 263 | A |
| 264 | A |
| 265 | A |
| 266 | B |
| 267 | A |
| 268 | A |
| 269 | B |
| 270 | A |
| 271 | A |
| 272 | B |
| 273 | B |
| 274 | B |
| 275 | B |
| 276 | B |
| 278 | B |
| 283 | B |
| 284 | B |
| 285 | B |
| 286 | B |
| 290 | B |
| 291 | B |
| 292 | B |
| 293 | A |
| 294 | B |
| 295 | B |
| 296 | B |
| 297 | B |
| 298 | B |
| 301 | A |
| 302 | B |
| 304 | B |
| 305 | A |
| 306 | A |
| 307 | A |
| 308 | B |
| 309 | B |
| 310 | B |
| 311 | A |
| 312 | B |

### Biological Activity Example 4: Assay of Inhibitory Activity of Drugs in Inducible SARM1 Overexpressing Cell Line

(1) Preparation of iSARM1 Cell Line

In this example, the gene sequence of SARM1 was amplified using PCR, and constructed into pInducer20-neo plasmid. pInducer20-SARM1 virus was packaged in a liposome and infected HEK293 to obtain an inducible SARM1-overexpressing cell line, which was labeled as iSARM1 (HEK293). The specific preparation was as follows:

In this example, the SARM1 gene sequence was amplified by PCR using the primers comprising sequences set forth in SEQ ID No.: 5 and SEQ ID No.: 6. The recovery of PCR amplification products, enzyme digestion, recombinant plasmid construction, transfection and cell screening were all performed in the same manner as dN-SARM1 in “Expression and purification of SARM1 protein”, except that when performing “(3) Cell screening”, “2 μg/mL puromycin” was replaced with 2 mg/mL neomycin. The rest were the same, which will not be repeated here.

SEQ ID No.: 5:
5′-TCTAGAGCCACCATGGTCCTGACGCTGCTTC-3′
SEQ ID No.: 6:
5′-GAATTCTTAGGTTGGACCCATGGGTG-3′

### (2) Determination of Inhibitory Activity of the Inhibitors on SARM1 Protein in Cell Lines

First, a 96-well culture plate was treated with 0.05 mg/ml polylysine for 5 minutes and washed once with PBS. 3×10⁴ iSARM1 (HEK293) cells were plated into the 96-well plate and cultured overnight in a 37° C. and 5% incubator. The next day, the cells were added with an inhibitor with a final concentration of 50 μM, incubated in the incubator for 1.5 hours, added with activator CZ-48 with a final concentration of 100 μM, and incubated for 16 hours. Meanwhile, the cells without adding CZ-48 or without adding drug were set as control group. Finally, the intracellular cADPR level was detected to represent the activity of SARM1, and the inhibition rate of SARM1 in cells by 50 μM inhibitor was calculated.

The cADPR measurement was performed as follows. First, the cells were washed once with PBS, and added with 150 μl of pre-cooled 0.6M perchloric acid (PCA) to quickly lyse the cells and precipitate the protein. The PCA supernatant was transferred to a 1.5 ml centrifuge tube. The protein in the culture medium was dissolved again with 100 μl of 1M NaOH. The supernatant was added with 0.5 ml of organic reagent mixture (trioctylamine:chloroform=1:3) to extract PCA from water. After sufficient shaking, the mixture was centrifuged at 12,000 rpm for 10 minutes. The solution was divided into 3 layers: the upper aqueous phase, containing the small molecules of interest; the lower organic phase, in which PCA was dissolved; and a thin protein layer between the upper and lower layers. The upper layer was transferred to a new centrifuge tube, added with 1M Tris-Mg at a ratio of 1:100 (1M Tris (pH 8.0): 1M MgCl2=9:1), added with NADase at a ratio of 1:250, and treated at 37° C. overnight to remove NAD⁺ from the mixed solution. After the treatment was completed, the mixed solution was filtered using Millipore's 10 K 96-well filter plate to remove NADase.

The cADPR content in the solution was determined through Cycling analysis method. The specific steps were as follows: 20 μl of the sample to be tested or the cADPR standard was taken and added to a 96-well opaque white plate. Preparation of reaction solution: 9.6 ml of PBS (pH 7.4), 200 μl of ethanol, 150 μl of 1 mg/ml AD, 10 μl of 10 mM FMN, 5 μl of 18 mg/ml Diaphorase, 10 μl of 10 mM Resazurin, and 100 μl of 1M Nam. Half of the reaction solution was divided and added with 0.2 μg/ml cyclase. The reaction solution without adding cyclase was used as control experiment. Each sample was divided into two groups, 3 replicates in each group. The reaction solutions containing or not containing cyclase were added to each group to perform the reaction. The reaction kinetic curve within 30 minutes was recorded (Ex:Em=544/599). The average slope of the reaction was calculated. Conversion was performed according to cADPR standard to obtain the accurate cADPR content. A curve of intracellular cADPR content vs corresponding inhibitor concentration was plotted. The half inhibitory concentration of the inhibitor was calculated.

Using the above method, the drug inhibitory activity (EC50) in the inducible overexpressing SARM1 cell line is shown in Table 3 below: cell activity EC50 interval: A<1.0 μM; B: 1-10 μM; C: 10-25 μM

TABLE 3

| Compound Number | EC₅₀ |
|---|---|
| 2 | B |
| 6 | A |
| 7 | B |
| 8 | B |
| 9 | B |
| 10 | A |
| 11 | A |
| 12 | A |
| 13 | B |
| 14 | B |
| 15 | B |
| 17 | B |
| 20 | B |
| 25 | B |
| 32 | B |
| 33 | B |
| 34 | B |
| 40 | B |
| 44 | B |
| 47 | A |
| 49 | A |
| 50 | B |
| 63 | B |
| 65 | B |
| 68 | B |
| 69 | B |
| 70 | B |
| 75 | A |
| 78 | B |
| 79 | B |
| 84 | A |
| 87 | B |
| 90 | B |
| 96 | B |
| 99 | B |
| 105 | B |
| 113 | B |
| 114 | B |
| 115 | B |
| 116 | A |
| 118 | A |
| 119 | B |
| 121 | A |
| 122 | A |
| 123 | A |
| 129 | B |
| 130 | B |
| 155 | A |
| 156 | B |
| 157 | B |
| 158 | A |
| 159 | B |
| 160 | A |
| 161 | B |
| 162 | A |
| 164 | B |
| 167 | A |
| 168 | B |
| 169 | B |
| 171 | A |
| 181 | B |
| 183 | B |
| 185 | B |
| 201 | A |
| 205 | A |
| 211 | A |
| 212 | A |
| 213 | A |
| 215 | A |
| 219 | B |
| 220 | B |
| 221 | A |
| 227 | A |
| 230 | B |
| 231 | B |
| 234 | B |
| 236 | B |
| 237 | A |
| 238 | A |
| 239 | A |
| 240 | A |
| 241 | A |
| 244 | A |
| 245 | A |
| 246 | B |
| 247 | A |
| 248 | A |
| 249 | A |
| 251 | B |
| 253 | B |
| 254 | B |
| 255 | B |
| 256 | B |
| 259 | B |
| 260 | B |
| 262 | A |
| 272 | B |
| 273 | B |
| 274 | B |
| 275 | B |
| 276 | B |
| 278 | B |
| 301 | B |
| 307 | B |

1. A compound represented by formula I: 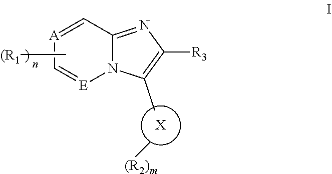
or a pharmaceutically acceptable salt or stereoisomer thereof,
wherein,
A represents CH or N;
E represents CH or N;
R₁ is independently selected from the group consisting of hydrogen, halogen, CF₃, CN, C₁-C₆ alkyl, C₁-C₆ alkoxy, C₃-C₆ cycloalkyl, C₃-C₆ heterocycloalkyl, amino, CF₃C(O)—NH—, CF₃C(O)—N(CH₃)—, C₁-C₆ alkylamino, C₃-C₆ cycloalkylamino, C₆-C₁₄ aryl, C₅-C₁₄ heteroaryl, C₆-C₁₄ arylamino, C₆-C₁₄ heteroarylamino, —OH, C₆-C₁₄ aryloxy, —CONH₂, —SO₂NH₂, C₁-C₆ alkyl-C(O)NR₅—, C₃-C₆ cycloalkyl-C(O)NR₅—, C₃-C₆ heterocycloalkyl-C(O)NR₅—, C₁-C₆ alkyl-OC(O)NR₅—, C₃-C₆ cycloalkyl-OC(O)NR₅—, C₃-C₆ heterocycloalkyl-OC(O)NR₅—, C₁-C₆ alkyl-OC(O)NR₅—(C₁-C₄ alkyl)-, C₁-C₆ alkyl-C(O)NR₅—(C₁-C₄ alkyl)-, C₃-C₆ cycloalkyl-OC(O)NR₅—(C₁-C₄ alkyl)-, C₃-C₆ cycloalkyl-C(O)NR₅—(C₁-C₄ alkyl)-, (C₆-C₁₄ aryl)-(C₁-C₆ alkyl)-CO—N(R₅)—(C₆-C₁₄ aryl)-(C₃-C₆ alkyl)-N(R₅)—, and (C₆-C₁₄ aryl)-(C₃-C₆ alkenyl)-N(R₅)—; wherein, in the above C₁-C₆ alkyl, one carbon atom can be replaced by a heteroatom selected from the group consisting of N, O and S atoms; preferably, R₁ is independently selected from the group consisting of C₁-C₆ alkylamino, C₃-C₆ heterocycloalkylamino, C₁-C₆ alkylacylamino, 1-morpholinyl, and C₁-C₆ alkyl-OC(O)NR₅—;
X represents a cyclic structure, selected from the group consisting of C₃-C₆ cycloalkyl, C₃-C₆ cycloalkenyl, C₃-C₆ heterocycloalkyl, C₆-C₁₄ aryl and C₅-C₁₄ heteroaryl, or X is absent; preferably, X is selected from the group consisting of phenyl, pyridyl, methoxy-substituted pyridyl, thiazolyl, cyclohexyl and cyclohexenyl, wherein the phenyl can be substituted by the following substituents: —SO₂—NH₂, —NH—COCH₃, —NH₂, —CO—NH₂, —OCH₃, halogen, C₁-C₄ alkyl, —SO₂—N(BoC)CH₃ or C₁-C₄ alkyl-NH—SO₂—;
R₂ is independently selected from the group consisting of hydrogen, halogen, —NH₂, —N(R₅)—CO—R, —CO—N(R₅)—R, —N(R₅)—SO₂—R, —SO₂—N(R₅)—R, —COOR, —COR, —(C₁-C₄ alkyl)-OR, —(C₁-C₄ alkyl)-N(CH₃)₂, NH—(C₁-C₄ alkyl)R—, —N(R₅)—R, —NHCO—(C₃-C₆ cycloalkyl)-(C₃-C₆ heterocycloalkyl), —OR, —O—(C₁-C₄ alkyl)-R and R;
R is selected from the group consisting of C₁-C₄ alkoxy, C₁-C₁₂ alkyl, —CONH₂, —SO₂—NH₂, C₃-C₆ cycloalkyl, C₃-C₆ heterocycloalkyl, —(C₁-C₁₂ alkyl)-(C₆-C₁₄) aryl, C₆-C₁₄ aryl and C₅-C₁₄ heteroaryl, wherein the C₁-C₁₂ alkyl, C₃-C₆ cycloalkyl, C₃-C₆ heterocycloalkyl, C₆-C₁₄ aryl and C₅-C₁₄ heteroaryl are optionally substituted by 1, 2 or 3 halogens, and 1 to 4 —CH₂— units in the C₁-C₁₂ alkyl are optionally replaced by O atom, S atom, —CO— or —NH—;
R₅ is selected from the group consisting of H, C₁-C₄ alkyl, C₃-C₆ cycloalkyl, C₃-C₆ heterocycloalkyl, C₁-C₄ alkoxy, C₆-C₁₄ aryl and C₅-C₁₄ heteroaryl;
R₃ is independently selected from the group consisting of hydrogen, C₁-C₄ alkyl and C₁-C₄ alkoxycarbonyl;
wherein the above C₃-C₆ heterocycloalkyl and C₅-C₁₄ heteroaryl contain 1 or 2 heteroatoms selected from the group consisting of N, O and S atoms;
R₁ and R₂ can be connected through a carbon-carbon bond or an ether bond to form a 14- to 16-membered ring, which contains 1-4 heteroatoms selected from the group consisting of N, O and S, preferably, the ring contains 3-4 N atoms and 1-2 O or S atoms;
m and n are positive integers selected from the group consisting of 1, 2 and 3.

2. The compound represented by formula I or a pharmaceutically acceptable salt or stereoisomer thereof according to claim 1, wherein the compound represented by formula I has the following structure of formula II: 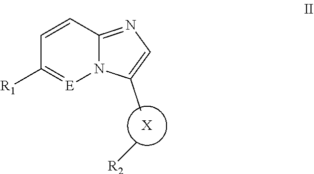
wherein E, R₁, R₂ and X are defined as in claim 1.

3. The compound represented by formula I or pharmaceutically acceptable salt or stereoisomer thereof according to claim 1, wherein the compound represented by formula I has the following structure of formula III: 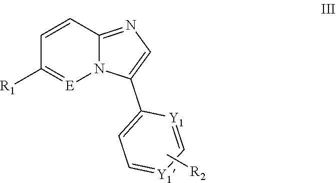
wherein E, R₁ and R₂ are defined as in claim 1;
Y₁ and Y₁′ are independently CH or N.

4. The compound represented by formula I or a pharmaceutically acceptable salt or stereoisomer thereof according to claim 1, wherein the compound represented by formula I has the following structure of formula IV: 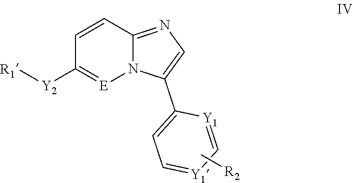
wherein E and R₂ are defined as in claim 1;
Y₁ and Y₁′ are independently CH or N;
Y₂ is selected from the group consisting of —O—, —NH—, —NR₅—, —NR₅—(C₁-C₄ alkyl)- and —NR₅ (C₃-C₆ cycloalkyl)-, or Y₂ is absent;
R₁′ is selected from the group consisting of R, —C(═O)—R, —SO₂—R, —C(═O)—OR and —SO₂NHR; wherein R₅ and R are defined as in claim 1.

5. The compound represented by formula I or a pharmaceutically acceptable salt or stereoisomer thereof according to claim 4, wherein the compound represented by formula I has the following structure of formula V: 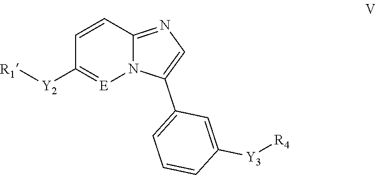
wherein,
E, R₁′ and Y₂ are defined as in claim 4;
Y₃ is selected from the group consisting of —N(R₅)CO—, —CO—N(R₅)—, —N(R₅)—SO₂—, —SO₂—N(R₅)—, —CO₂—, —CO—, —NH—(C₁-C₄ alkyl)-, —N(R₅)—, —O—(C₁-C₄ alkyl)- and —O—, or Y₃ is absent;
R₄ is selected from the group consisting of C₁-C₁₂ alkyl, C₃-C₆ cycloalkyl, C₃-C₆ heterocycloalkyl, C₆-C₁₄ aryl and C₅-C₁₄ heteroaryl, wherein the C₁-C₁₂ alkyl, C₃-C₆ cycloalkyl, C₃-C₆ heterocycloalkyl, C₆-C₁₄ aryl and C₅-C₁₄ heteroaryl are optionally substituted by 1, 2 or 3 halogens; the C₃-C₆ heterocycloalkyl and C₅-C₁₄ heteroaryl contain 1 or 2 heteroatoms selected from the group consisting of N, O and S atoms; and 1 to 4 —CH₂— units in the C₁-C₁₂ alkyl are optionally replaced by O atom, S atom, —CO— or —NH—.

6. A compound represented by formula VI, or a pharmaceutically acceptable salt or stereoisomer thereof: 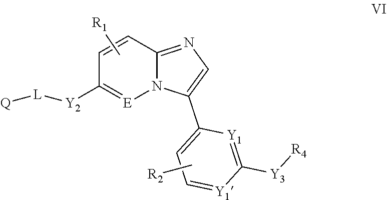
wherein,
E, R₁, R₂, Y₁, Y₁′, Y₂, Y₃ and R₄ are defined as in claim 5;
L is C₂-C₁₂ alkylene, wherein 1, 2, 3 or 4 —CH₂— units in the C₂-C₁₂ alkylene are optionally replaced by 1, 2, 3 or 4 O atom, N atom, —CO—, —CONH— or —NHCO—;
Q is an E3 ligase ligand, preferably a VHL ligand 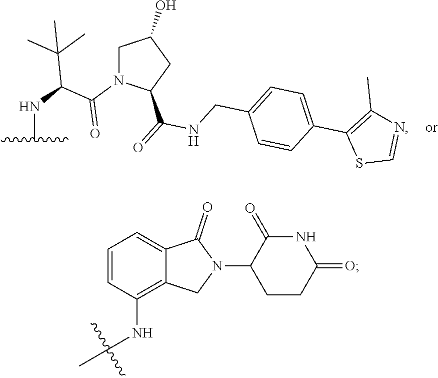 [SMILES: *N[C@H](C(=O)N1C[C@H](O)C[C@H]1C(=O)NCc1ccc(-c2scnc2C)cc1)C(C)(C)C ; *Nc1cccc2c1CN(C1CCC(=O)NC1=O)C2=O]
or Q is a structural unit selected from the group consisting of: 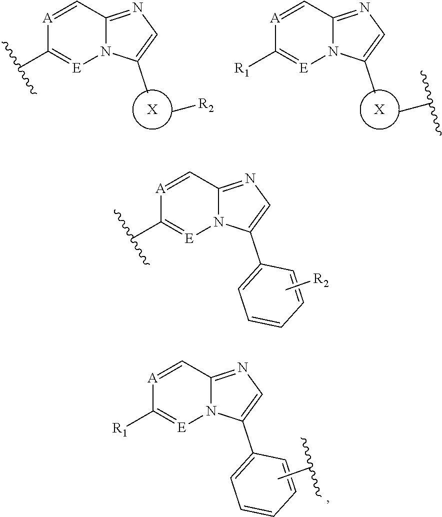
wherein A represents CH or N;
E represents CH or N;
R₁ is independently selected from the group consisting of hydrogen, halogen, CF₃, CN, C₁-C₆ alkyl, C₁-C₆ alkoxy, C₃-C₆ cycloalkyl, C₃-C₆ heterocycloalkyl, amino, CF₃C(O)—NH—, CF₃C(O)—N(CH₃)—, C₁-C₆ alkylamino, C₃-C₆ cycloalkylamino, C₆-C₁₄ aryl, C₅-C₁₄ heteroaryl, C₆-C₁₄ arylamino, C₆-C₁₄ heteroarylamino, —OH, C₆-C₁₄ aryloxy, —CONH₂, —SO₂NH₂, C₁-C₆ alkyl-C(O)NR₅—, C₃-C₆ cycloalkyl-C(O)NR₅—, C₃-C₆ heterocycloalkyl-C(O)NR₅—, C₁-C₆ alkyl-OC(O)NR₅—, C₃-C₆ cycloalkyl-OC(O)NR₅—, C₃-C₆ heterocycloalkyl-OC(O)NR₅—, C₁-C₆ alkyl-OC(O)NR₅—(C₁-C₄ alkyl)-, C₁-C₆ alkyl-C(O)NR₅—(C₁-C₄ alkyl)-, C₃-C₆ cycloalkyl-OC(O)NR₅—(C₁-C₄ alkyl)-, C₃-C₆ cycloalkyl-C(O)NR₅—(C₁-C₄ alkyl)-, (C₆-C₁₄ aryl)-(C₁-C₆ alkyl)-CO—N(R₅)—, (C₆-C₁₄ aryl)-(C₃-C₆ alkyl)-N(R₅)—, and (C₆-C₁₄ aryl)-(C₃-C₆ alkenyl)-N(R₅)—; wherein, in the above C₁-C₆ alkyl, one carbon atom can be replaced by a heteroatom selected from the group consisting of N, O and S atoms; preferably, R₁ is independently selected from the group consisting of C₁-C₆ alkylamino, C₃-C₆ heterocycloalkylamino, C₁-C₆ alkylacylamino, 1-morpholinyl, and C₁-C₆ alkyl-OC(O)NR₅—;
X represents a cyclic structure, selected from the group consisting of C₃-C₆ cycloalkyl, C₃-C₆ cycloalkenyl, C₃-C₆ heterocycloalkyl, C₆-C₁₄ aryl and C₅-C₁₄ heteroaryl, or X is absent; preferably, X is selected from the group consisting of phenyl, pyridyl, methoxy-substituted pyridyl, thiazolyl, cyclohexyl and cyclohexenyl, wherein the phenyl can be substituted by the following substituents: —SO₂—NH₂, —NH—COCH₃, —NH₂, —CO—NH₂, —OCH₃, halogen, C₁-C₄ alkyl, —SO₂—N(BoC)CH₃ or C₁-C₄ alkyl-NH—SO₂—;
R₂ is independently selected from the group consisting of hydrogen, halogen, —NH₂, —N(R₅)—CO—R, —CO—N(R₅)—R, —N(R₅)—SO₂—R, —SO₂—N(R₅)—R, —COOR, —COR, —(C₁-C₄ alkyl)-OR, —(C₁-C₄ alkyl)-N(CH₃)₂, NH—(C₁-C₄ alkyl)R—, —N(R₅)—R, —NHCO—(C₃-C₆ cycloalkyl)-(C₃-C₆ heterocycloalkyl), —OR, —O—(C₁-C₄ alkyl)-R and R;
R is selected from the group consisting of C₁-C₄ alkoxy, C₁-C₁₂ alkyl, —CONH₂, —SO₂—NH₂, C₃-C₆ cycloalkyl, C₃-C₆ heterocycloalkyl, —(C₁-C₁₂ alkyl)-(C₆-C₁₄)aryl, C₆-C₁₄ aryl and C₅-C₁₄ heteroaryl, wherein the C₁-C₁₂ alkyl, C₃-C₆ cycloalkyl, C₃-C₆ heterocycloalkyl, C₆-C₁₄ aryl and C₅-C₁₄ heteroaryl are optionally substituted by 1, 2 or 3 halogens, and 1 to 4 —CH₂— units in the C₁-C₁₂ alkyl are optionally replaced by O atom, S atom, —CO— or —NH—:
R₅ is selected from the group consisting of H, C₁-C₄ alkyl, C₃-C₆ cycloalkyl, C₃-C₆ heterocycloalkyl, C₁-C₄ alkoxy, C₆-C₁₄ aryl and C₅-C₁₄ heteroaryl;
or Q is

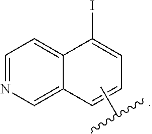

7. A compound represented by formula VII, or a pharmaceutically acceptable salt or stereoisomer thereof: 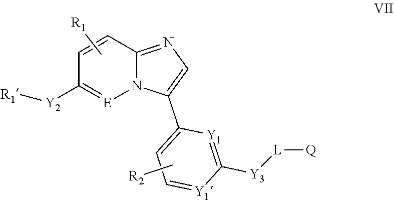
wherein,
E, R₁, R₂, R₁′, Y₁, Y₁′ and Y₂ are defined as in claim 5;
Y₃ is selected from the group consisting of —N(R₅)CO—, —CO—N(R₅)—, —N(R₅)—SO₂—, —SO₂—N(R₅)—, —CO₂—, —CO—, —NH—(C₁-C₄ alkyl)-, —N(R₅)—, —O—(C₁-C₄ alkyl)-, and —O—, or Y₃ is absent;
L is C₂-C₁₂ alkylene, wherein 1, 2, 3 or 4 —CH₂— units in the C₂-C₁₂ alkylene are optionally replaced by 1, 2, 3 or 4 O atoms, N atoms, —CO—, —CONH— or —NHCO—;
Q is an E3 ligase ligand, preferably a VHL ligand 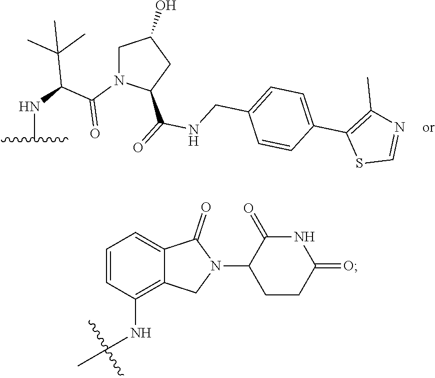 [SMILES: *N[C@H](C(=O)N1C[C@H](O)C[C@H]1C(=O)NCc1ccc(-c2scnc2C)cc1)C(C)(C)C ; *Nc1cccc2c1CN(C1CCC(=O)NC1=O)C2=O]
or Q is a structural unit selected from the group consisting of: 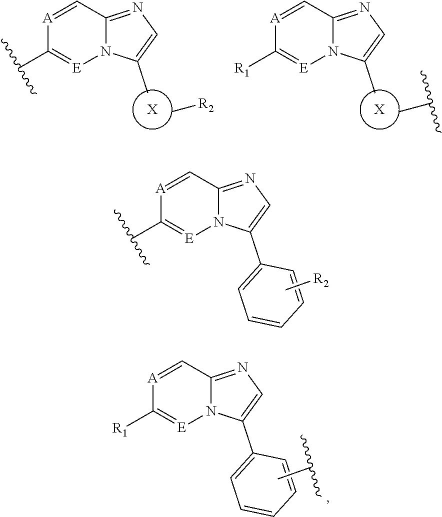
wherein, A represents CH or N;
E represents CH or N;
R₁ is independently selected from the group consisting of hydrogen, halogen, CF₃, CN, C₁-C₆ alkyl, C₁-C₆ alkoxy, C₃-C₆ cycloalkyl, C₃-C₆ heterocycloalkyl, amino, CF₃C(O)—NH—, CF₃C(O)—N(CH₃)—, C₁-C₆ alkylamino, C₃-C₆ cycloalkylamino, C₆-C₁₄ aryl, C₅-C₁₄ heteroaryl, C₆-C₁₄ arylamino, C₆-C₁₄ heteroarylamino, —OH, C₆-C₁₄ aryloxy, —CONH₂, —SO₂NH₂, C₁-C₆ alkyl-C(O)NR₅—, C₃-C₆ cycloalkyl-C(O)NR₅—, C₃-C₆ heterocycloalkyl-C(O)NR₅—, C₁-C₆ alkyl-OC(O)NR₅—, C₃-C₆ cycloalkyl-OC(O)NR₅—, C₃-C₆ heterocycloalkyl-OC(O)NR₅—, C₁-C₆ alkyl-OC(O)NR₅—(C₁-C₄ alkyl)-, C₁-C₆ alkyl-C(O)NR₅—(C₁-C₄ alkyl)-, C₃-C₆ cycloalkyl-OC(O)NR₅—(C₁-C₄ alkyl)-, C₃-C₆ cycloalkyl-C(O)NR₅—(C₁-C₄ alkyl)-, (C₆-C₁₄ aryl)- (C₁-C₆ alkyl)-CO—N(R₅)—, (C₆-C₁₄ aryl)-(C₃-C₆ alkyl)-N(R₅)—, and (C₆-C₁₄ aryl)-(C₃-C₆ alkenyl)-N(R₅)—; wherein, in the above C₁-C₆ alkyl, one carbon atom can be replaced by a heteroatom selected from the group consisting of N, O and S atoms; preferably, R₁ is independently selected from the group consisting of C₁-C₆ alkylamino, C₃-C₆ heterocycloalkylamino, C₁-C₆ alkylacylamino, 1-morpholinyl, and C₁-C₆ alkyl-OC(O)NR₅—;
X represents a cyclic structure, selected from the group consisting of C₃-C₆ cycloalkyl, C₃-C₆ cycloalkenyl, C₃-C₆ heterocycloalkyl, C₆-C₁₄ aryl and C₅-C₁₄ heteroaryl, or X is absent; preferably, X is selected from the group consisting of phenyl, pyridyl, methoxy-substituted pyridyl, thiazolyl, cyclohexyl and cyclohexenyl, wherein the phenyl can be substituted by the following substituents: —SO₂—NH₂, —NH—COCH₃, —NH₂, —CO—NH₂, —OCH₃, halogen, C₁-C₄ alkyl, —SO₂—N(BoC)CH₃ or C₁-C₄ alkyl-NH—SO₂—;
R₂ is independently selected from the group consisting of hydrogen, halogen, —NH₂, —N(R₅)—CO—R, —CO—N(R₅)—R, —N(R₅)—SO₂—R, —SO₂—N(R₅)—R, —COOR, —COR, —(C₁-C₄ alkyl)-OR, —(C₁-C₄ alkyl)-N(CH₃)₂, NH—(C₁-C₄ alkyl)R—, —N(R₅)—R, —NHCO—(C₃-C₆ cycloalkyl)-(C₃-C₆ heterocycloalkyl), —OR, —O—(C₁-C₄ alkyl)-R and R;
R is selected from the group consisting of C₁-C₄ alkoxy, C₁-C₁₂ alkyl, —CONH₂, —SO₂—NH₂, C₃-C₆ cycloalkyl, C₃-C₆ heterocycloalkyl, —(C₁-C₁₂ alkyl)-(C₆-C₁₄)aryl, C₆-C₁₄ aryl and C₅-C₁₄ heteroaryl, wherein the C₁-C₁₂ alkyl, C₃-C₆ cycloalkyl, C₃-C₆ heterocycloalkyl, C₆-C₁₄ aryl and C₅-C₁₄ heteroaryl are optionally substituted by 1, 2 or 3 halogens, and 1 to 4 —CH₂— units in the C₁-C₁₂ alkyl are optionally replaced by O atom, S atom, —CO— or —NH—;
R₅ is selected from the group consisting of H, C₁-C₄ alkyl, C₃-C₆ cycloalkyl, C₃-C₆ heterocycloalkyl, C₁-C₄ alkoxy, C₆-C₁₄ aryl and C₅-C₁₄ heteroaryl;
or Q is

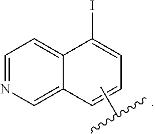

8. The compound or a pharmaceutically acceptable salt or stereoisomer thereof according to claim 1, wherein the compound is selected from the group consisting of:

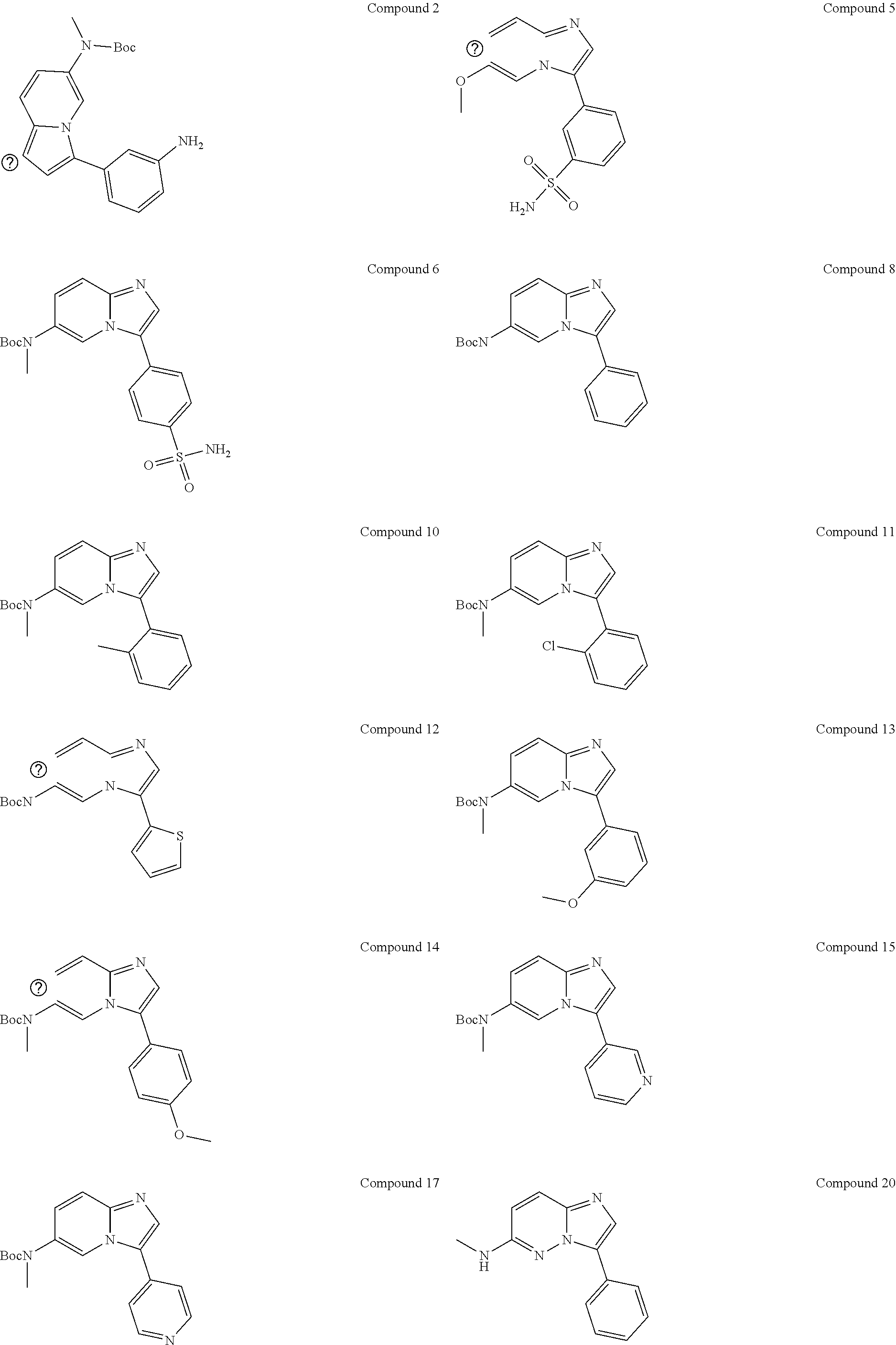

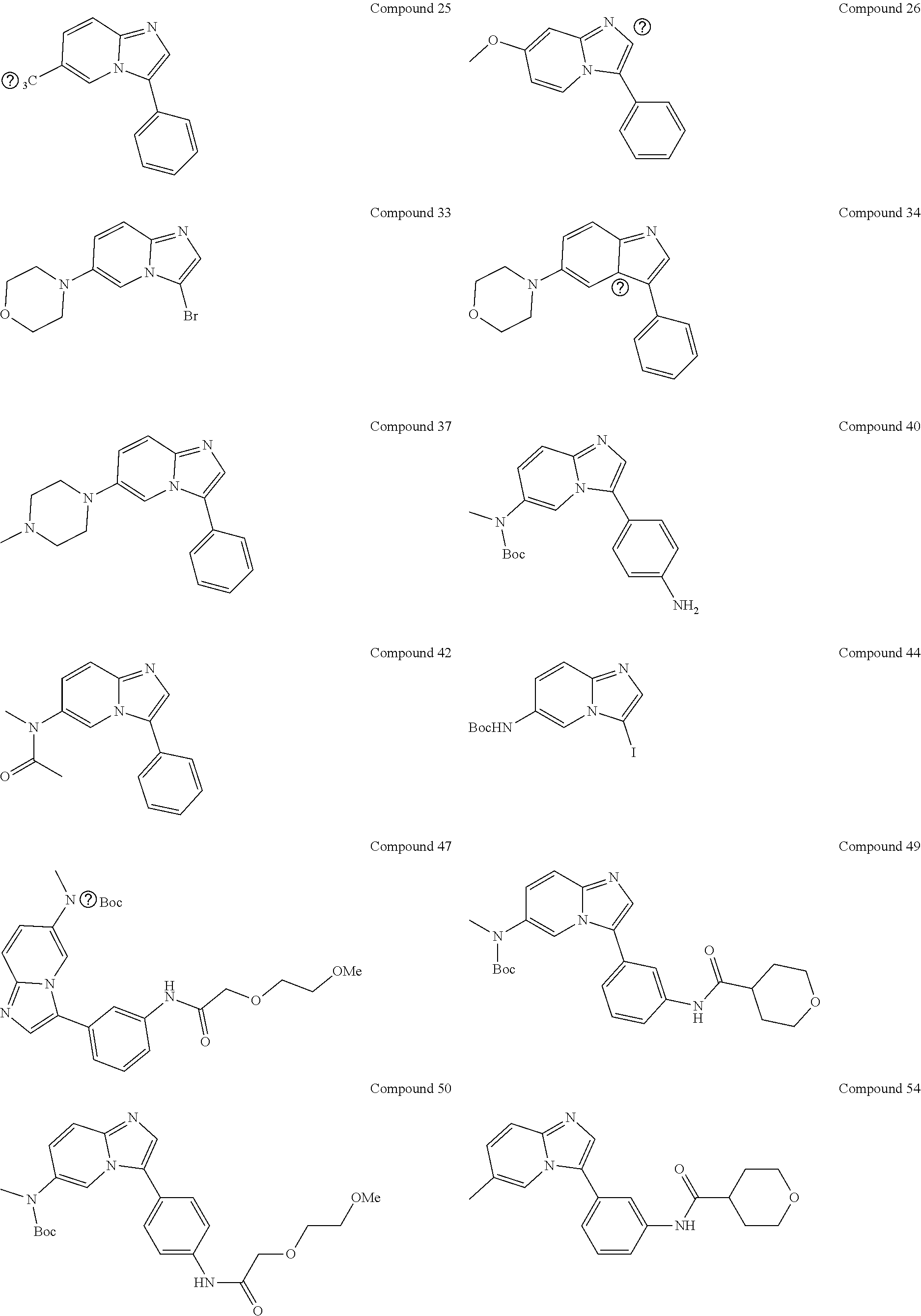

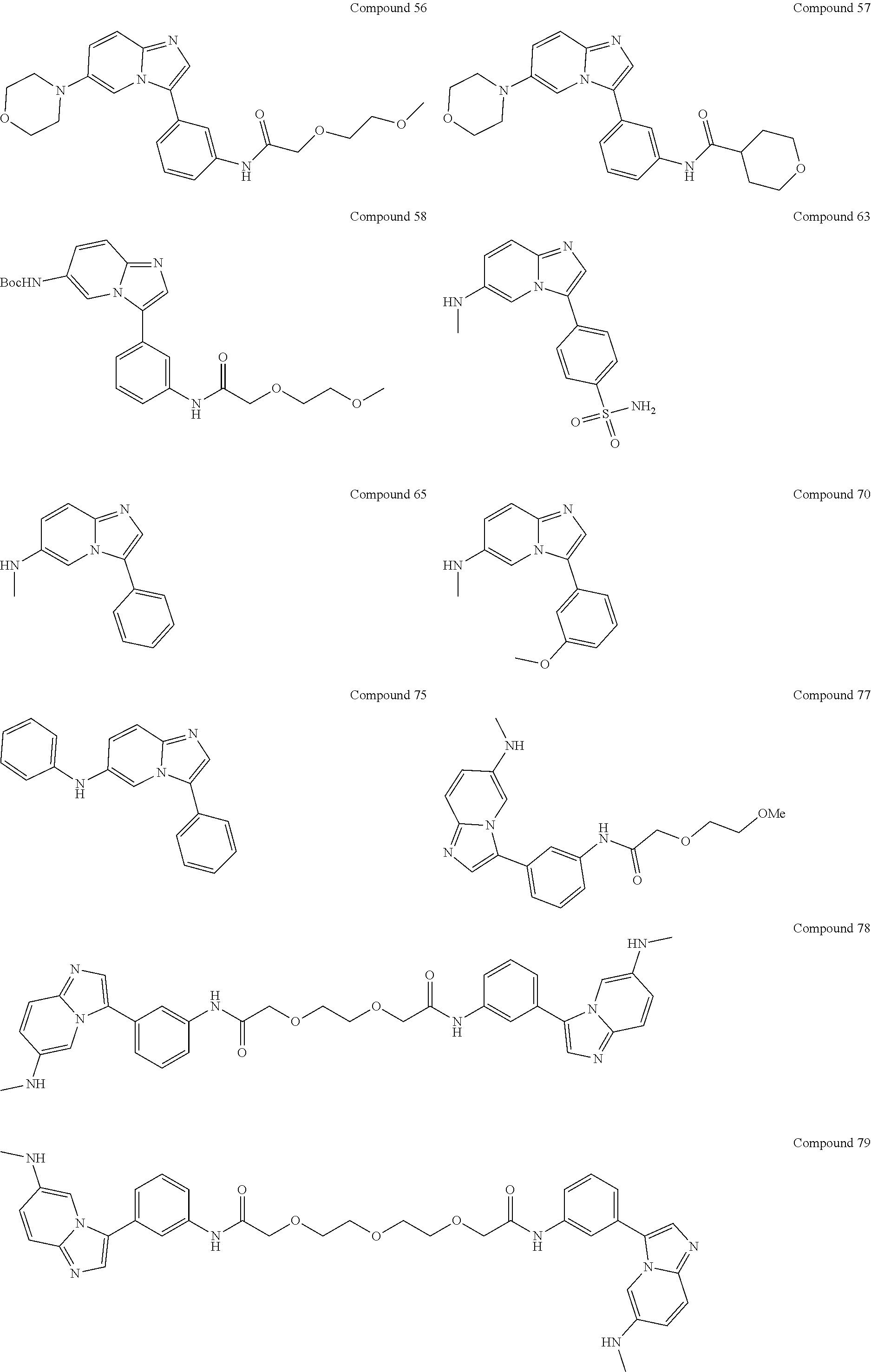

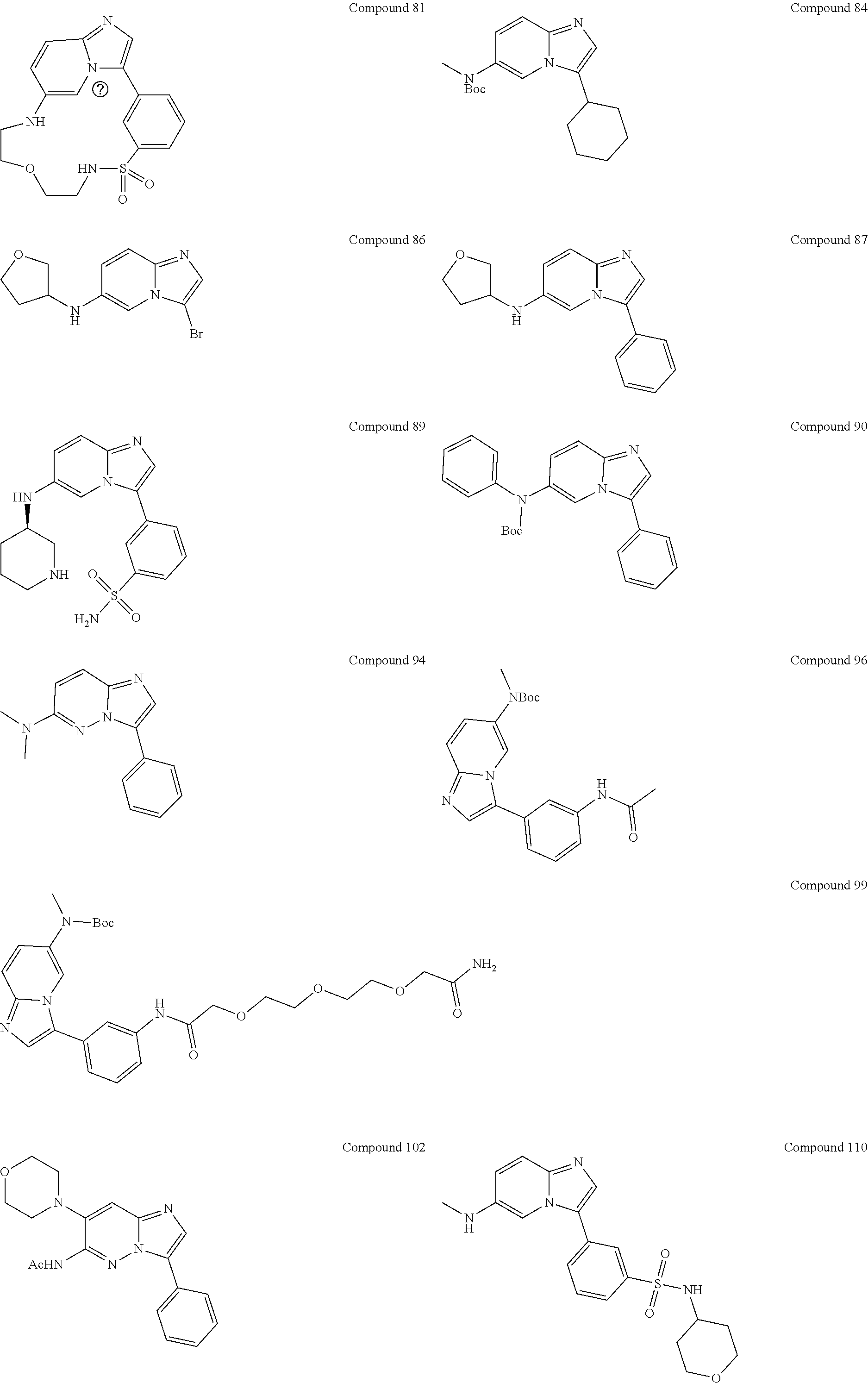

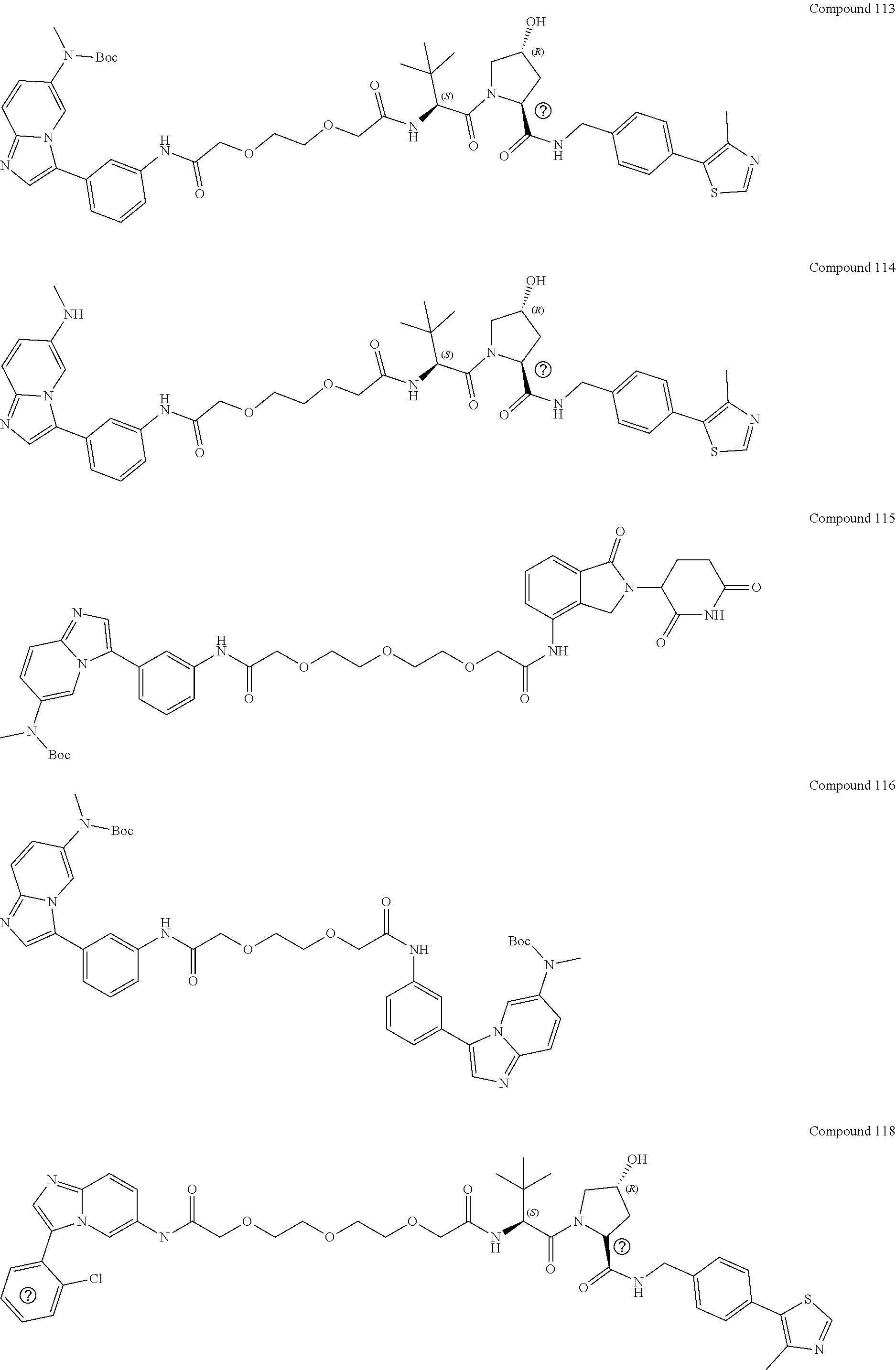

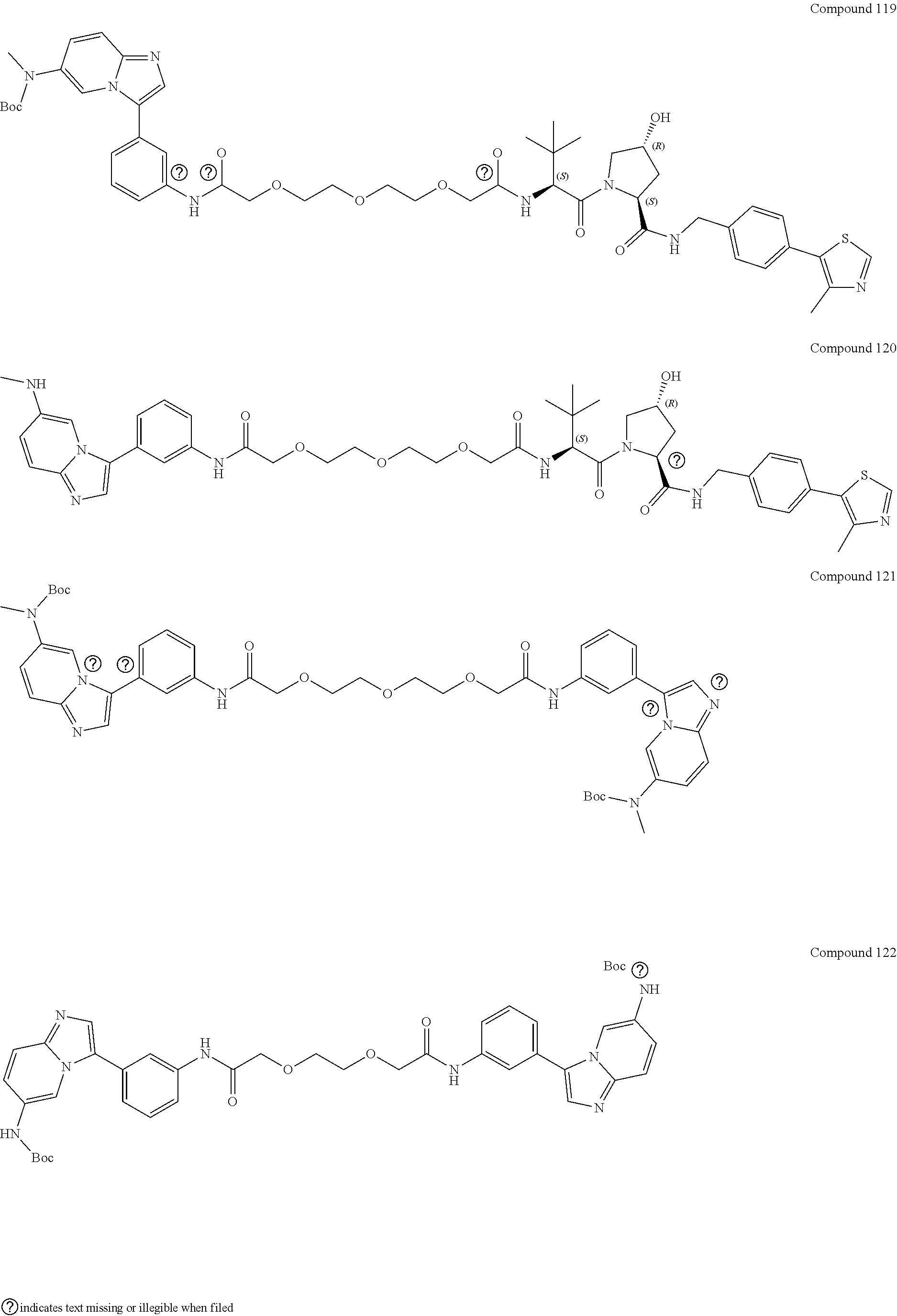

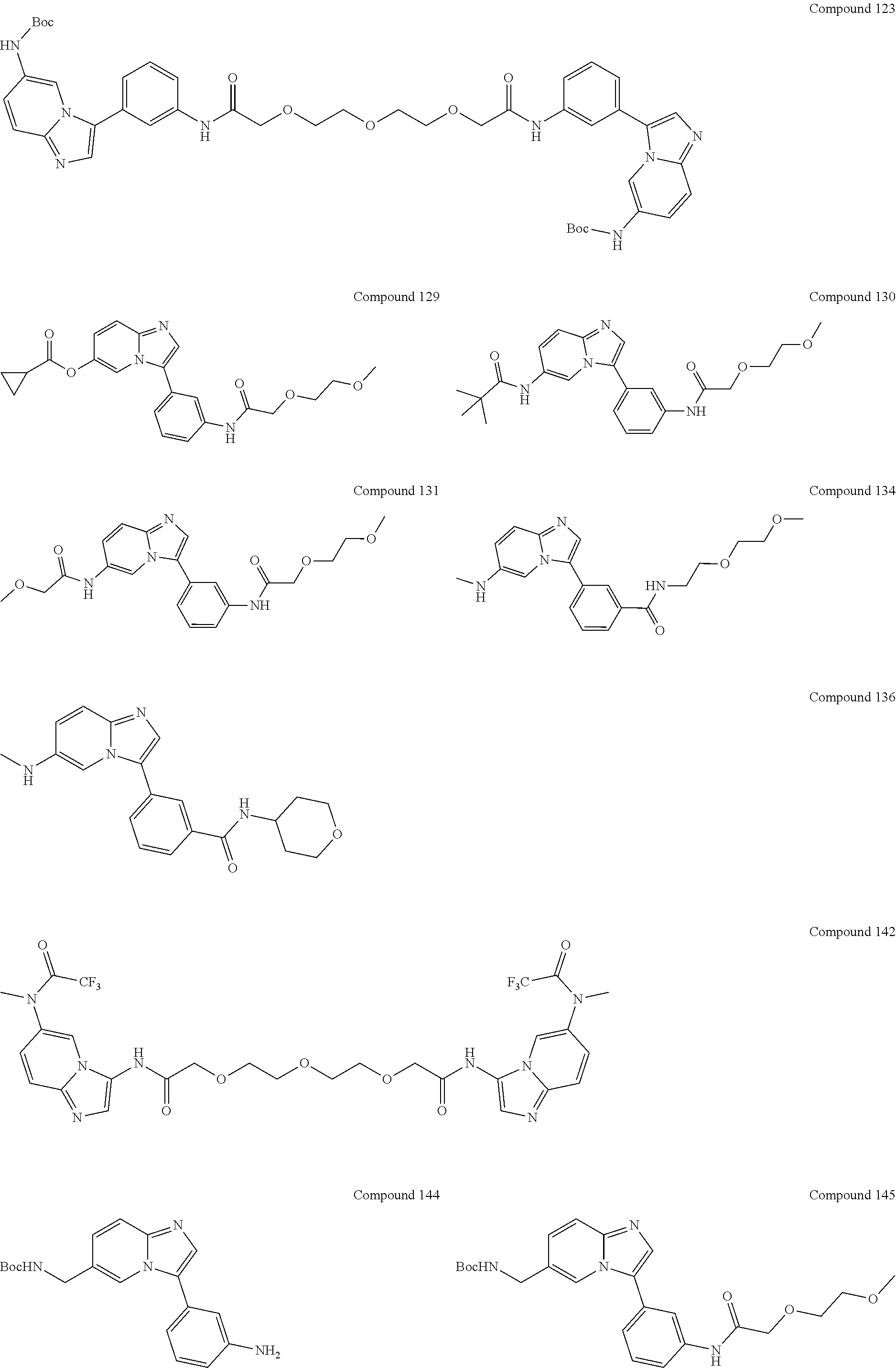

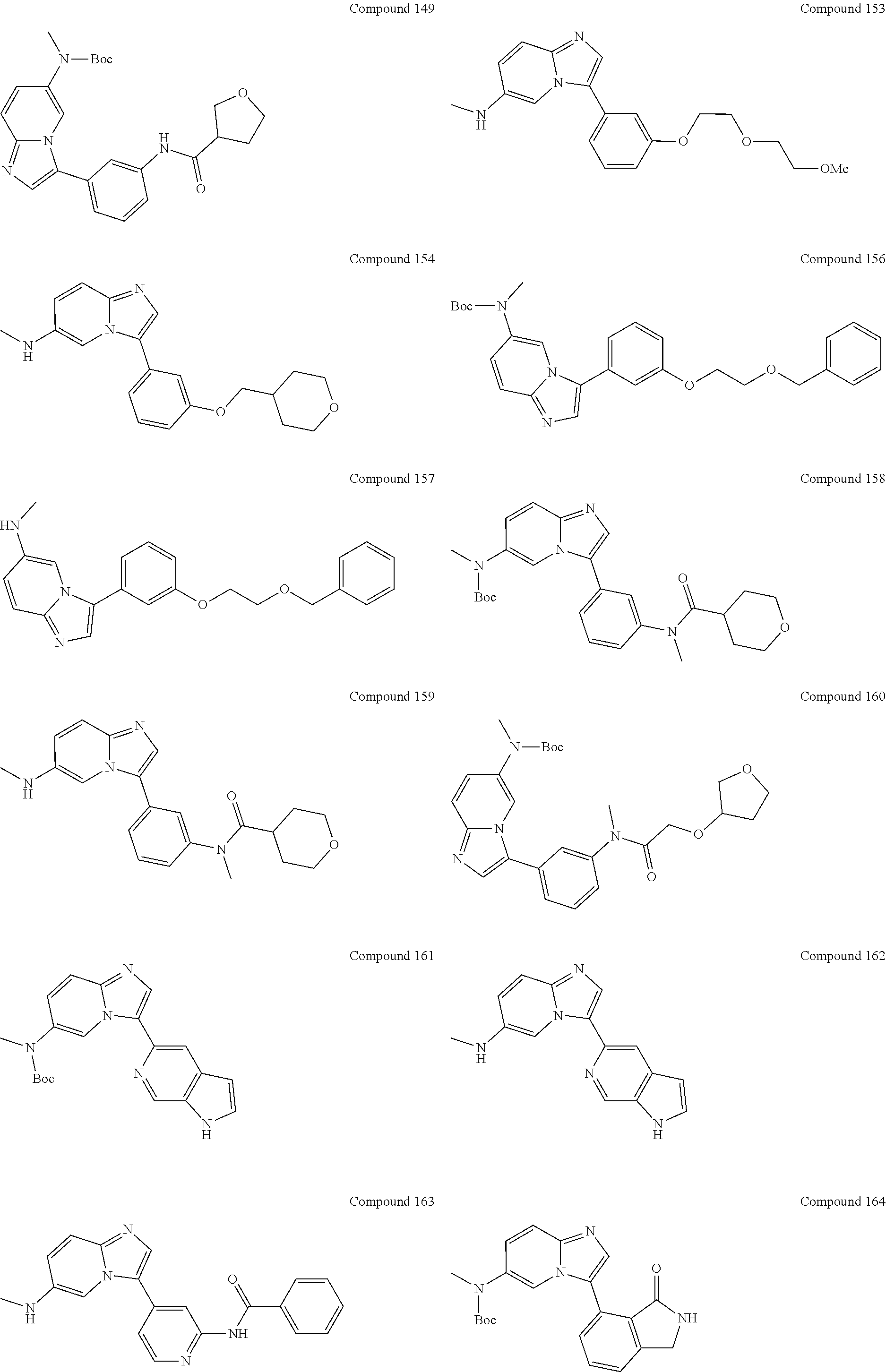

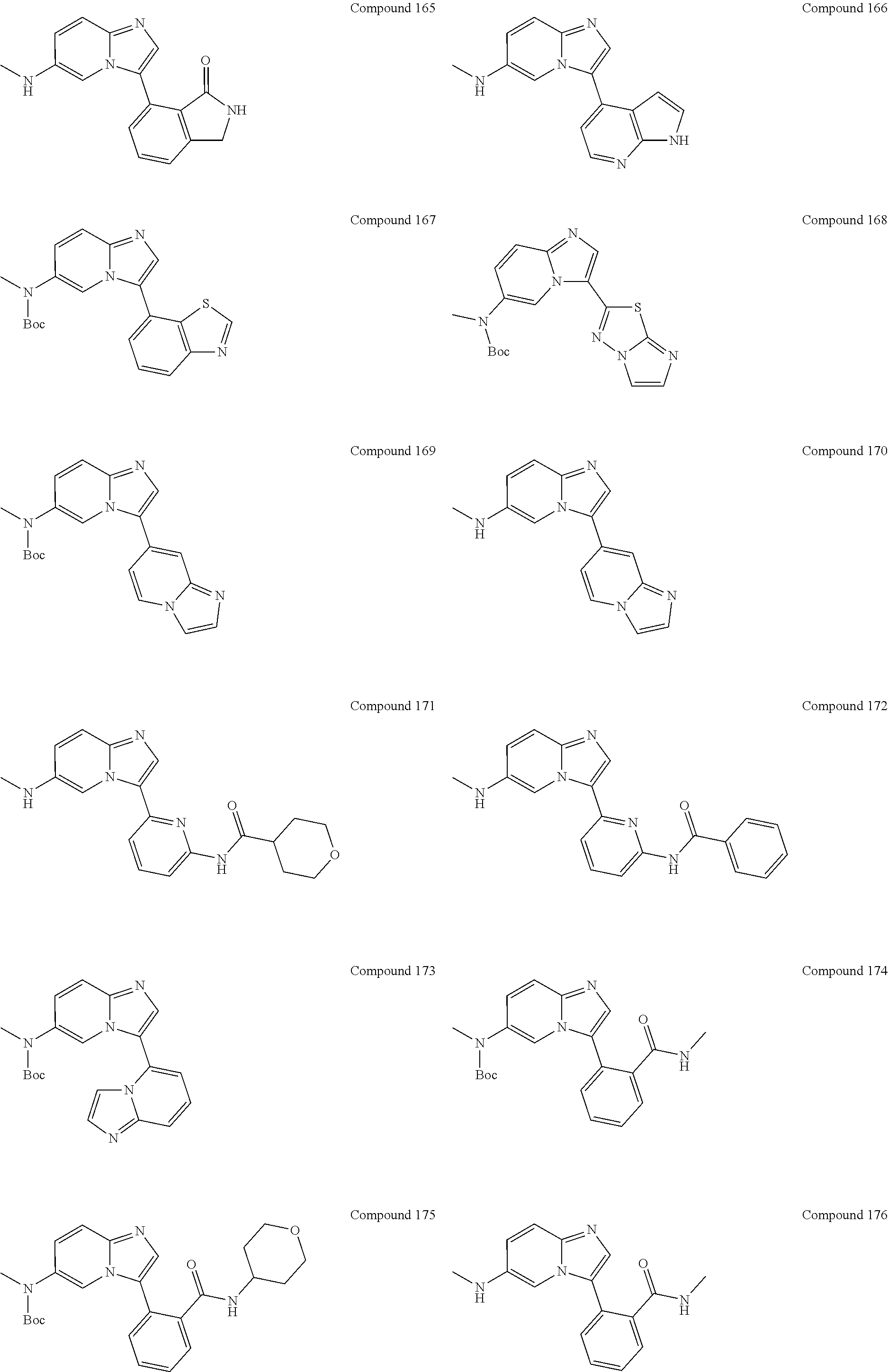

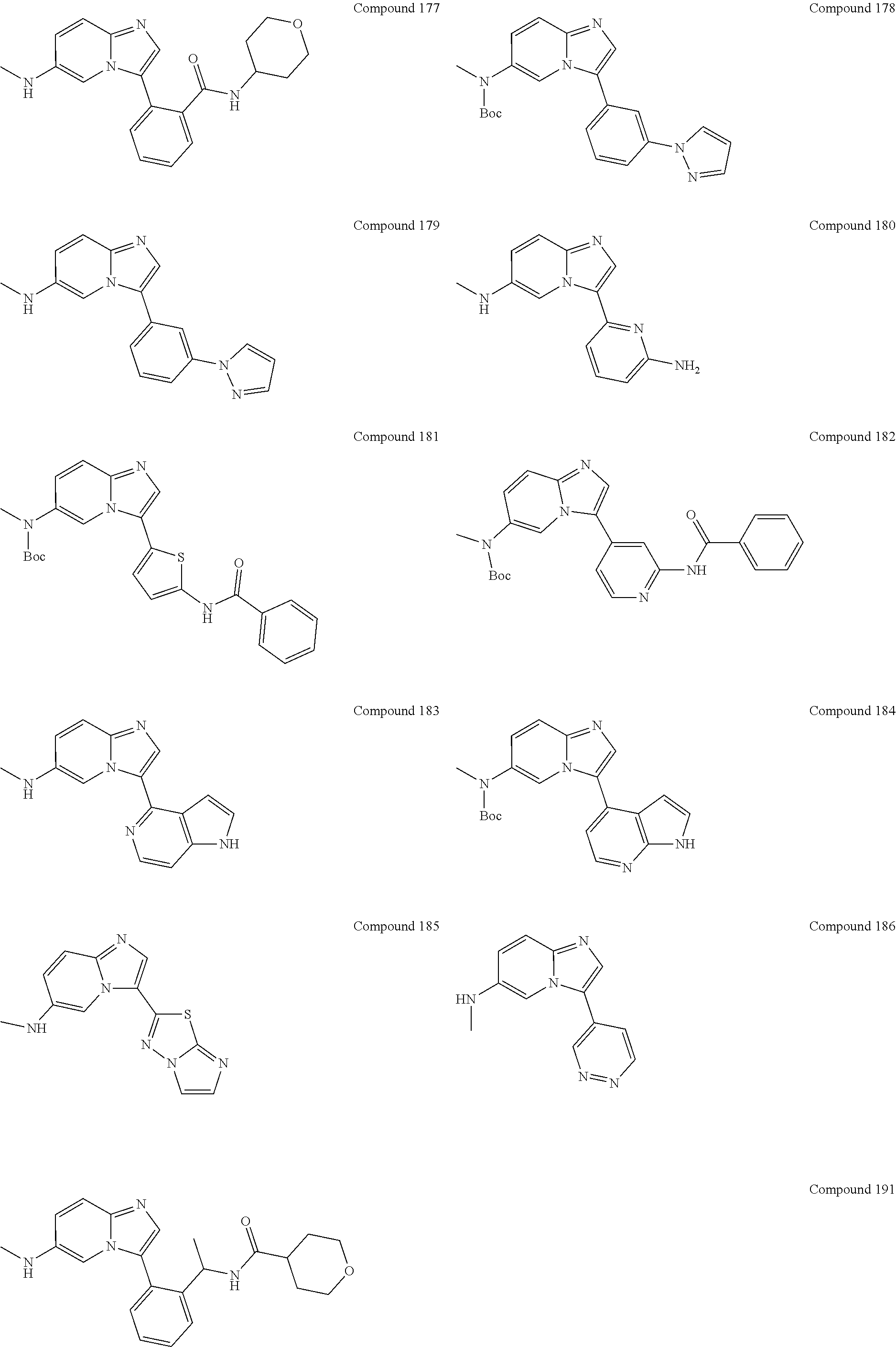

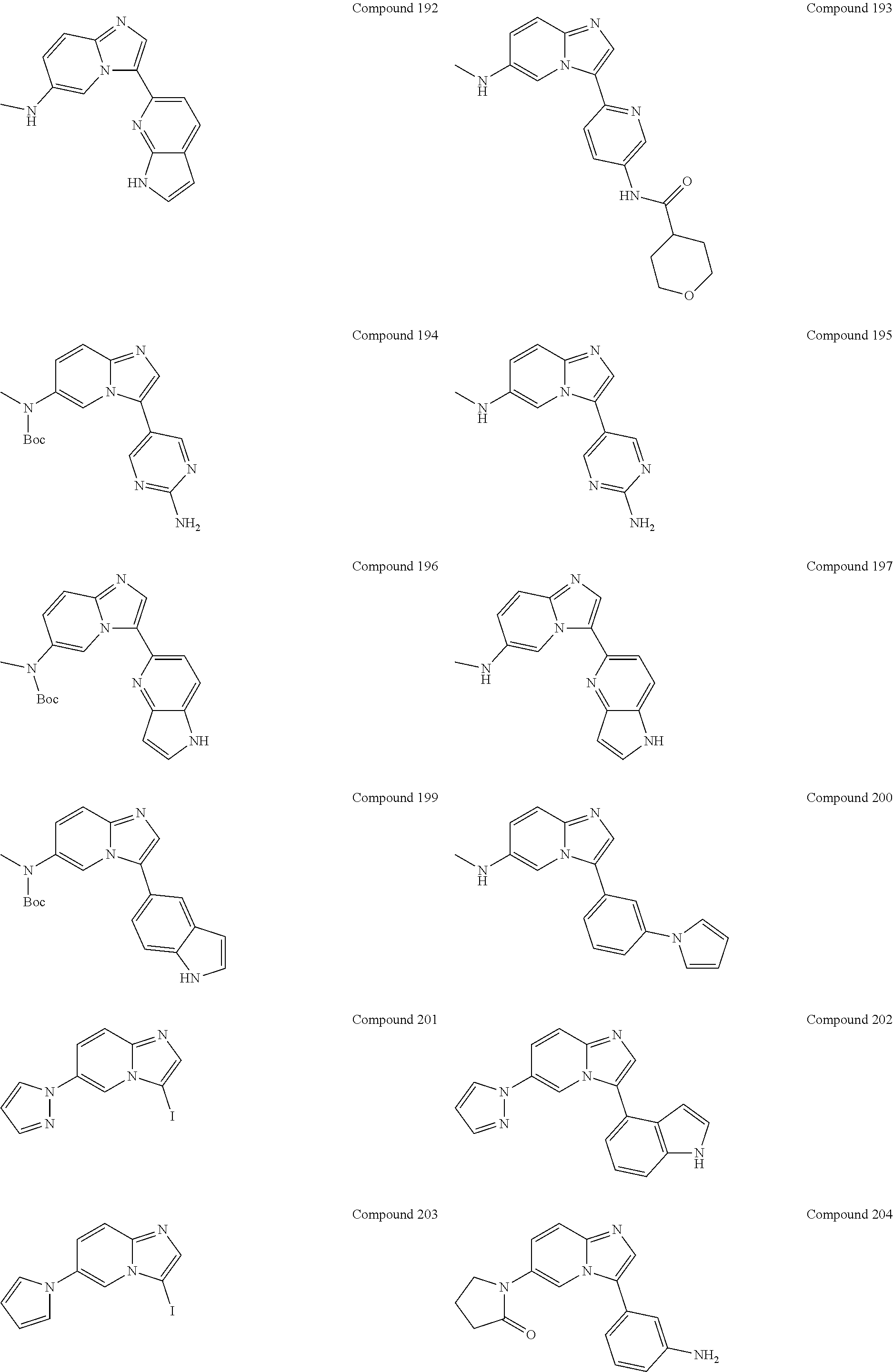

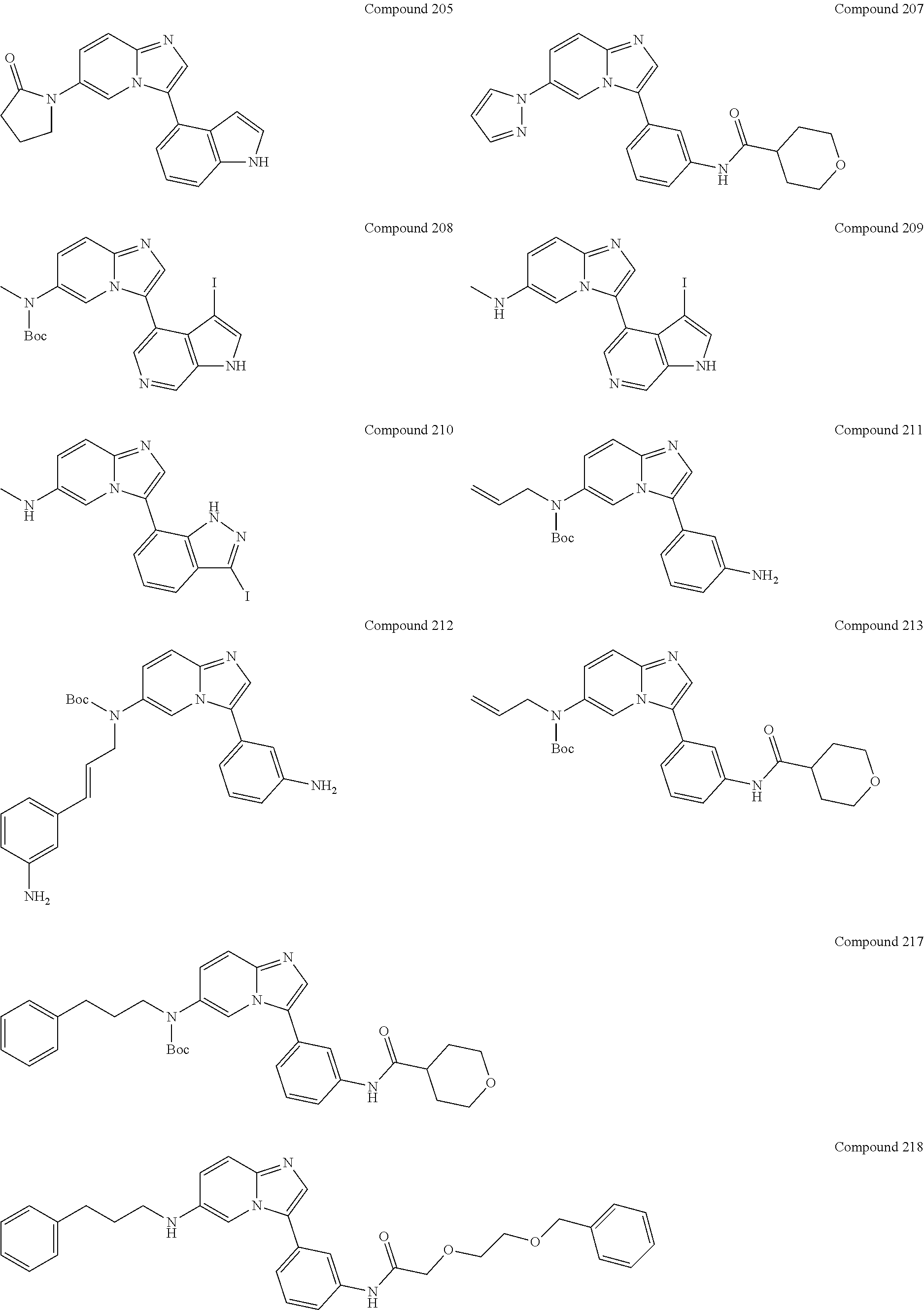

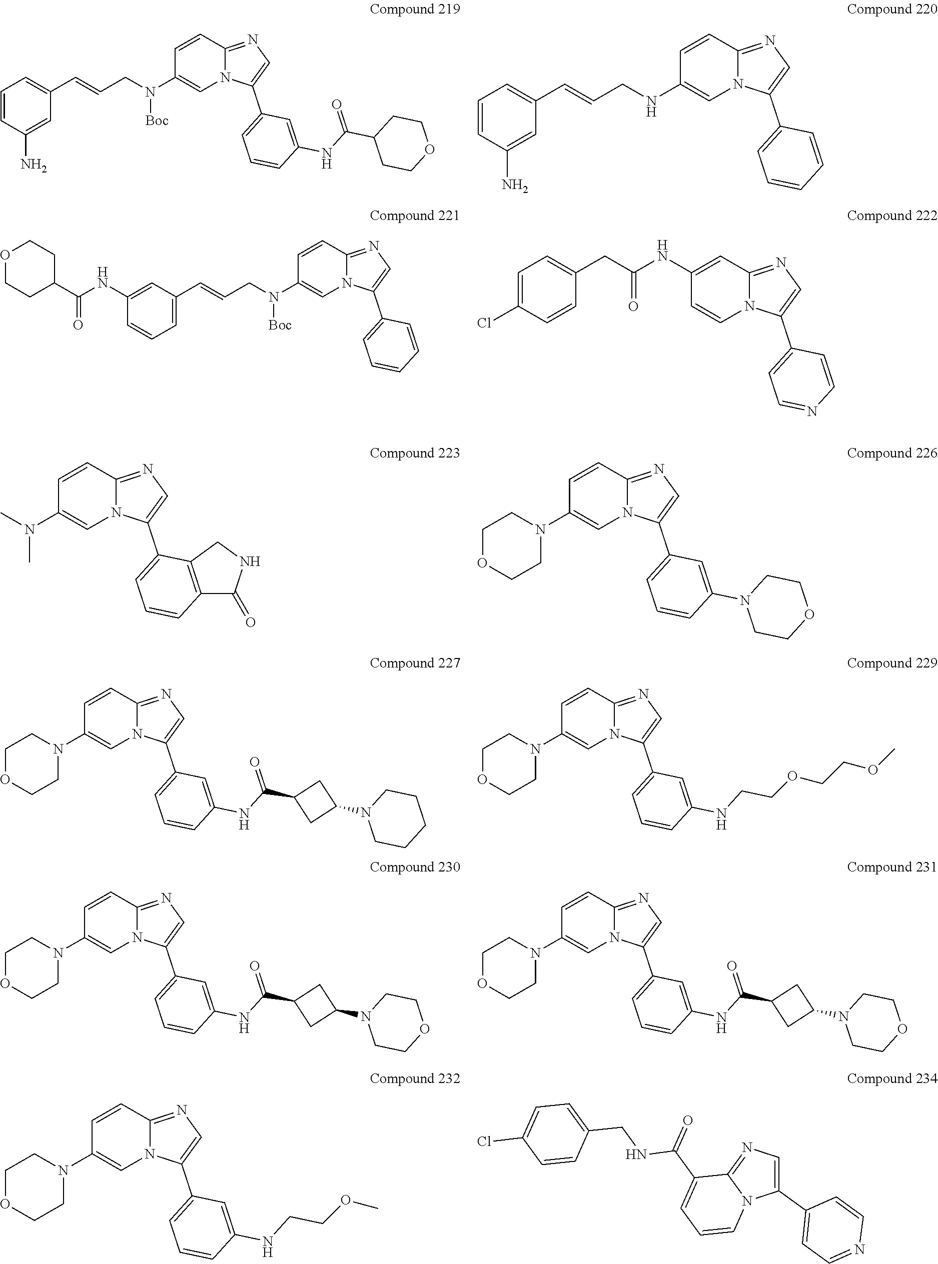

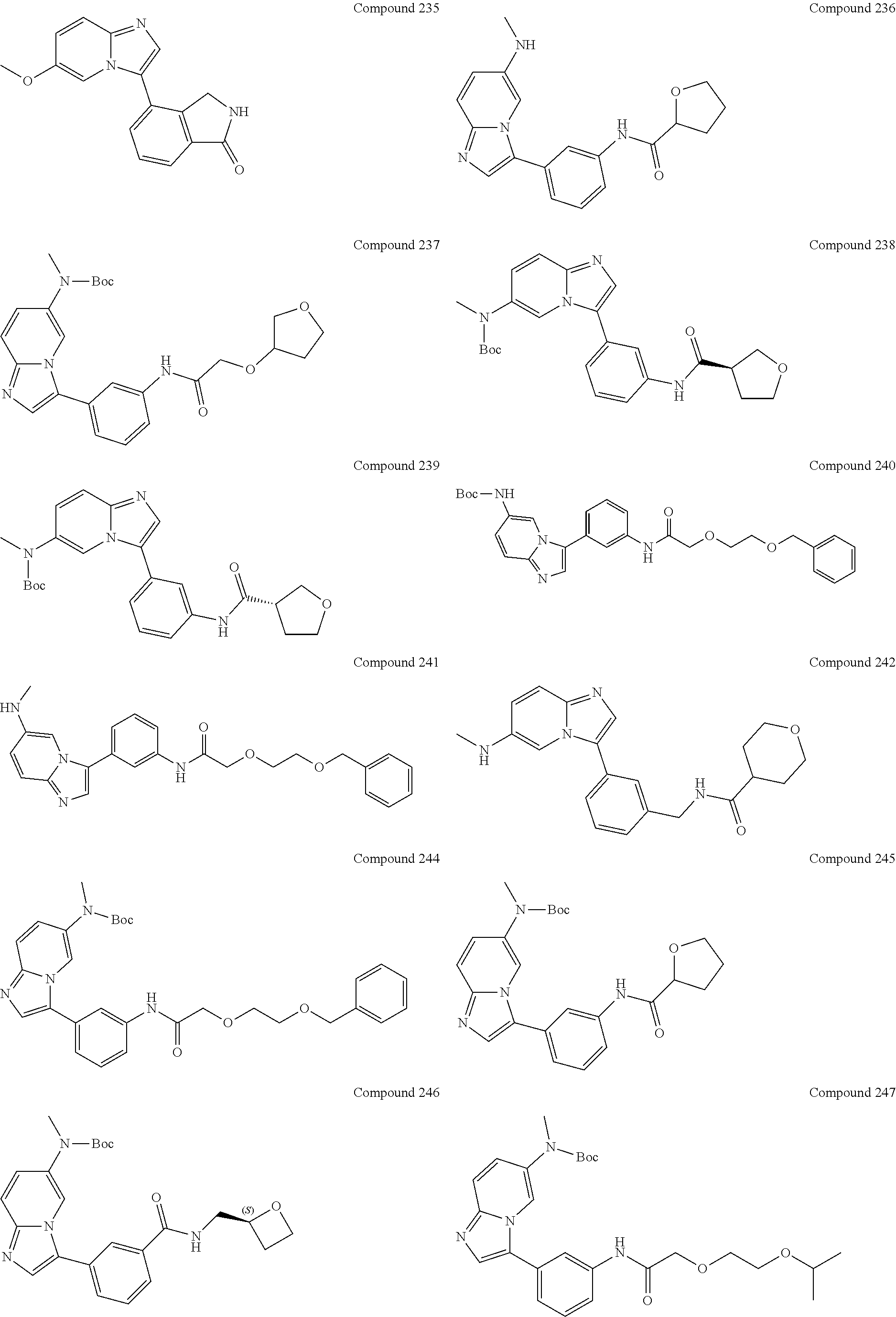

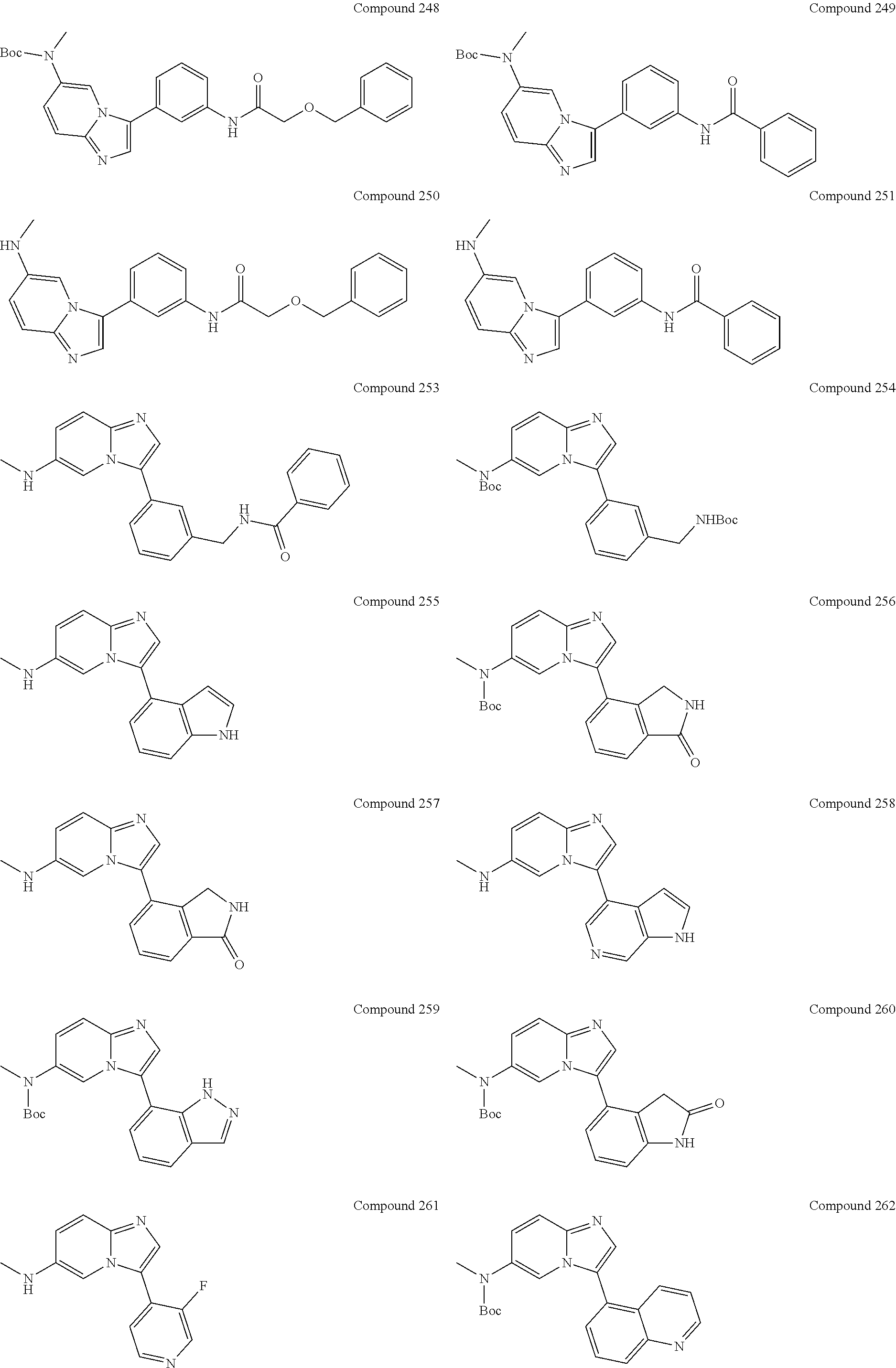

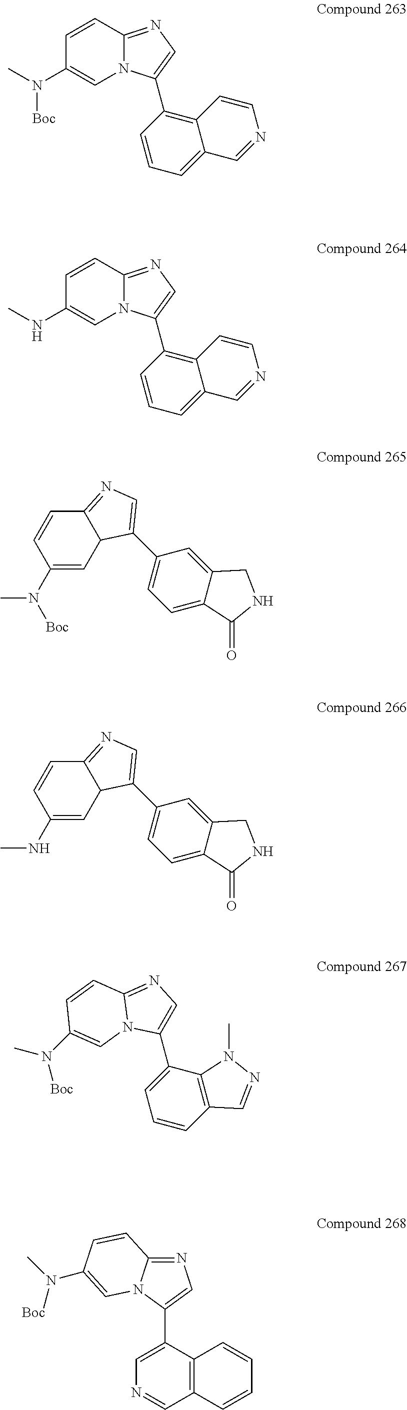 [SMILES: CN(C(=O)OC(C)(C)C)c1ccc2ncc(-c3cccc4cnccc34)n2c1 ; CNc1ccc2ncc(-c3cccc4cnccc34)n2c1 ; CN(C(=O)OC(C)(C)C)C1=CC2C(c3ccc4c(c3)CNC4=O)=CN=C2C=C1 ; CNC1=CC2C(c3ccc4c(c3)CNC4=O)=CN=C2C=C1 ; CN(C(=O)OC(C)(C)C)c1ccc2ncc(-c3cccc4cnn(C)c34)n2c1 ; CN(C(=O)OC(C)(C)C)c1ccc2ncc(-c3cncc4ccccc34)n2c1]

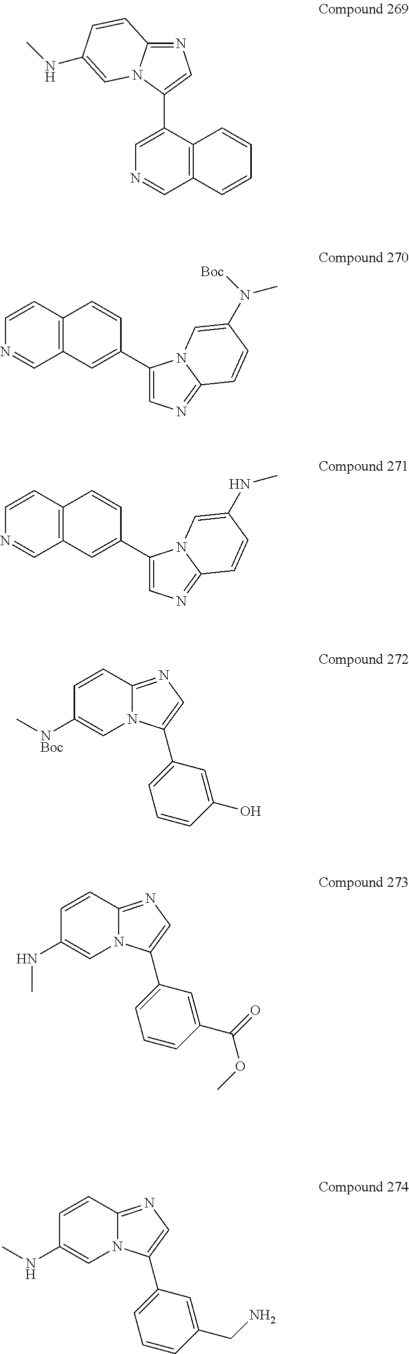

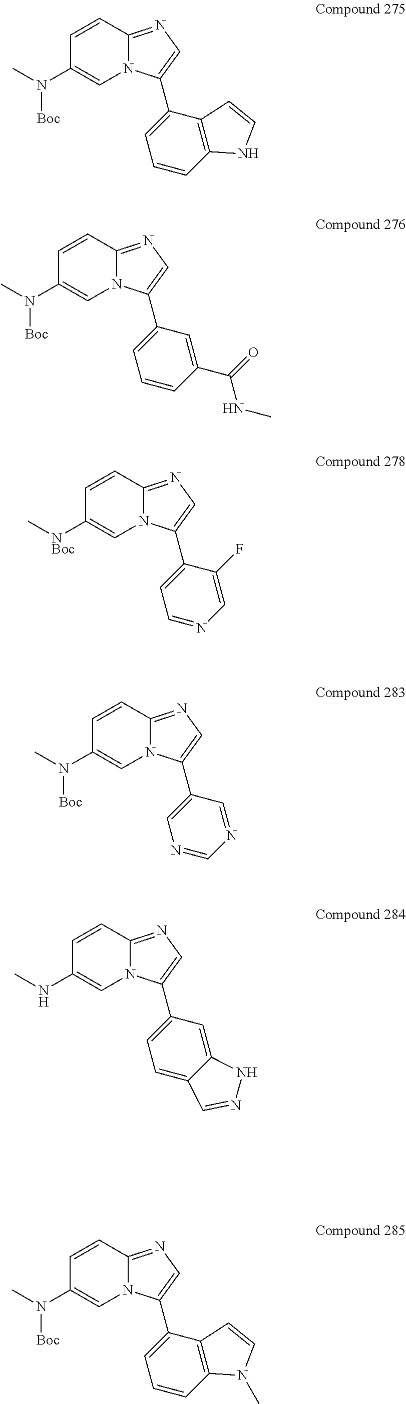

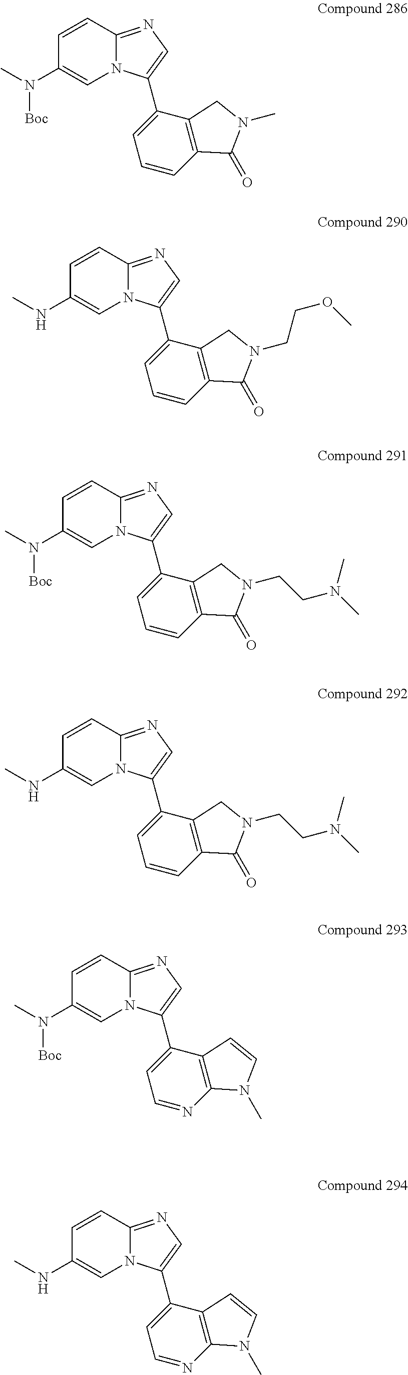

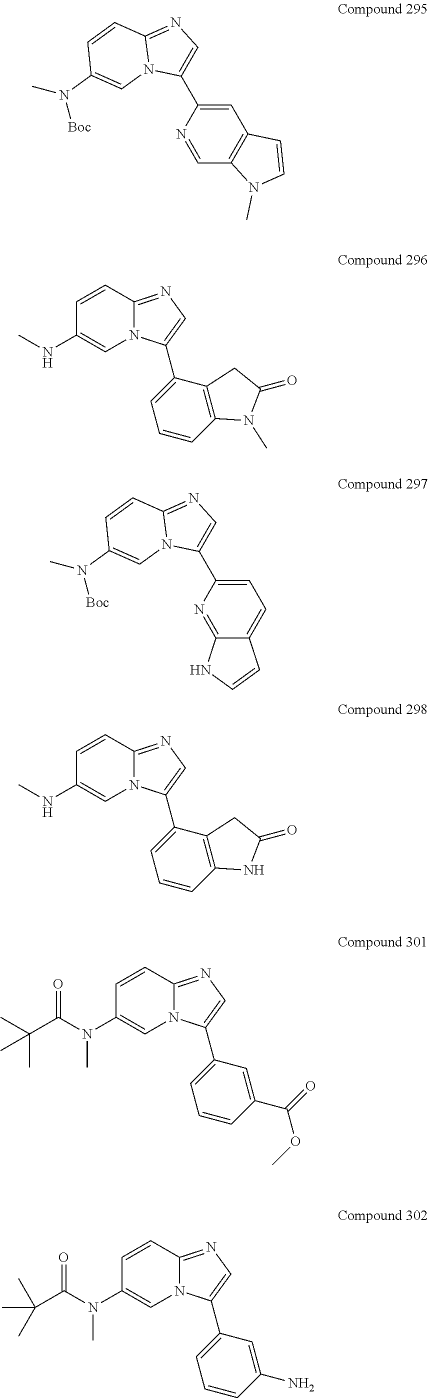 [SMILES: CN(C(=O)OC(C)(C)C)c1ccc2ncc(-c3cc4ccn(C)c4cn3)n2c1 ; CNc1ccc2ncc(-c3cccc4c3CC(=O)N4C)n2c1 ; CN(C(=O)OC(C)(C)C)c1ccc2ncc(-c3ccc4cc[nH]c4n3)n2c1 ; CNc1ccc2ncc(-c3cccc4c3CC(=O)N4)n2c1 ; COC(=O)c1cccc(-c2cnc3ccc(N(C)C(=O)C(C)(C)C)cn23)c1 ; CN(C(=O)C(C)(C)C)c1ccc2ncc(-c3cccc(N)c3)n2c1]

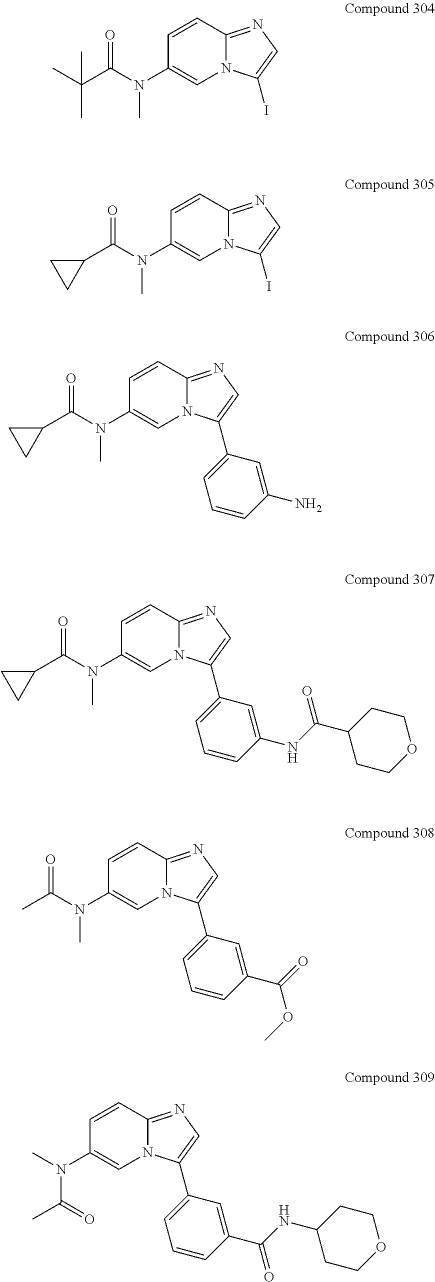 [SMILES: CN(C(=O)C(C)(C)C)c1ccc2ncc(I)n2c1 ; CN(C(=O)C1CC1)c1ccc2ncc(I)n2c1 ; CN(C(=O)C1CC1)c1ccc2ncc(-c3cccc(N)c3)n2c1 ; CN(C(=O)C1CC1)c1ccc2ncc(-c3cccc(NC(=O)C4CCOCC4)c3)n2c1 ; COC(=O)c1cccc(-c2cnc3ccc(N(C)C(C)=O)cn23)c1 ; CC(=O)N(C)c1ccc2ncc(-c3cccc(C(=O)NC4CCOCC4)c3)n2c1]

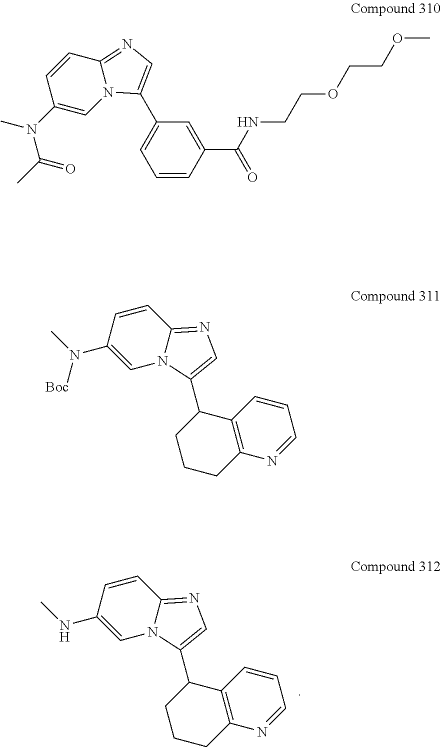

9. A method of treating or preventing a neurodegenerative disease or neurological disease or condition, comprising administering the compound or pharmaceutically acceptable salt or stereoisomer thereof according to claim 1 to a subject in need thereof.

10. A method of inhibiting SARM1 enzymatic activity, comprising administering the compound or a pharmaceutically acceptable salt or stereoisomer thereof according to claim 1 to a subject in need thereof.

11. A method of treating or preventing an axonal degeneration-related disease or condition, comprising administering the compound or a pharmaceutically acceptable salt or stereoisomer thereof according to claim 1 to a subject in need thereof.

12. The method according to claim 9, wherein the neurodegenerative disease or neurological disease or condition is selected from the group consisting of Alzheimer's disease, Parkinson's disease, multiple sclerosis, amyotrophic lateral sclerosis and peripheral neuropathy.

13. The method according to claim 11, wherein the axonal degeneration-related disease or condition is selected from the group consisting of Alzheimer's disease, Parkinson's disease, multiple sclerosis, amyotrophic lateral sclerosis and peripheral neuropathy.


SEQUENCE LISTING (6 sequences)

SEQ ID NO: 1 - DNA, 135 bp, synthetic construct
  misc_feature 1..135: note = BC2T-TEV
  source 1..135: mol_type = other DNA
ctcatgccagacagaaaagcggctgttagtcactggcagcaagatatcggcggaggcggatctggcggaggcggatctggcggaggcggatctgagaatttgtattttcagggtggcggaggcggaggtaccctg

SEQ ID NO: 2 - DNA, 25 bp, synthetic construct
  misc_feature 1..25: note = dN-SARM1-F
  source 1..25: mol_type = other DNA
ggtaccctggcggtgcctgggccag

SEQ ID NO: 3 - DNA, 36 bp, synthetic construct
  misc_feature 1..36: note = dN-SARM1-R
  source 1..36: mol_type = other DNA
gcggccgcctaggttggacccatgggtgcagcaccc

SEQ ID NO: 4 - DNA, 2100 bp, synthetic construct
  misc_feature 1..2100: note = dN-SARM1
  source 1..2100: mol_type = other DNA
ggtaccctggcggtgcctgggccagatgggggcggtggcacgggcccatggtgggctgcgggtggccgcgggccccgcgaagtgtcgccgggggcaggcaccgaggtgcaggacgccctggagcgcgcgctgccggagctgcagcaggccttgtccgcgctgaagcaggcgggcggcgcgcgggccgtgggcgccggcctggccgaggtcttccaactggtggaggaggcctggctgctgccggccgtgggccgcgaggtagcccagggtctgtgcgacgccatccgcctcgatggcggcctcgacctgctgttgcggctgctgcaggcgccggagttggagacgcgtgtgcaggccgcgcgcctgctggagcagatcctggtggctgagaaccgagaccgcgtggcgcgcattgggctgggcgtgatcctgaacctggcgaaggaacgcgaacccgtagagctggcgcggagcgtggcaggcatcttggagcacatgttcaagcattcggaggagacatgccagaggctggtggcggccggcggcctggacgcggtgctgtattggtgccgccgcacggaccccgcgctgctgcgccactgcgcgctggcgctgggcaactgcgcgctgcacgggggccaggcggtgcagcgacgcatggtagagaagcgcgcagccgagtggctcttcccgctcgccttctccaaggaggacgagctgcttcggctgcacgcctgcctcgcagtagcggtgttggcgactaacaaggaggtggagcgcgaggtggagcgctcgggcacgctggcgctcgtggagccgcttgtggcctcgctggaccctggccgcttcgcccgctgtctggtggacgccagcgacacaagccagggccgcgggcccgacgacctgcagcgcctcgtgccgttgctcgactctaaccgcttggaggcgcagtgcatcggggctttctacctctgcgccgaggctgccatcaagagcctgcaaggcaagaccaaggtgttcagcgacatcggcgccatccagagcctgaaacgcctggtttcctactctaccaatggcactaagtcggcgctggccaagcgcgcgctgcgcctgctgggcgaggaggtgccacggcccatcctgccctccgtgcccagctggaaggaggccgaggttcagacgtggctgcagcagatcggtttctccaagtactgcgagagcttccgggagcagcaggtggatggcgacctgcttctgcggctcacggaggaggaactccagaccgacctgggcatgaaatcgggcatcacccgcaagaggttctttagggagctcacggagctcaagaccttcgccaactattctacgtgcgaccgcagcaacctggcggactggctgggcagcctggacccgcgcttccgccagtacacctacggcctggtcagctgcggcctggaccgctccctgctgcaccgcgtgtctgagcagcagctgctggaagactgcggcatccacctgggcgtgcaccgcgcccgcatcctcacggcggccagagaaatgctacactccccgctgccctgtactggtggcaaacccagtggggacactccagatgtcttcatcagctaccgccggaactcaggttcccagctggccagtctcctgaaggtgcacctgcagctgcatggcttcagtgtcttcattgatgtggagaagctggaagcaggcaagttcgaggacaaactcatccagagtgtcatgggtgcccgcaactttgtgttggtgctatcacctggagcactggacaagtgcatgcaagaccatgactgcaaggattgggtgcataaggagattgtgactgctttaagctgcggcaagaacattgtgcccatcattgatggcttcgagtggcctgagccccaggtcctgcctgaggacatgcaggctgtgcttactttcaacggtatcaagtggtcccacgaataccaggaggccaccattgagaagatcatccgcttcctgcagggccgctcctcccgggactcatctgcaggctctgacaccagtttggagggtgctgcacccatgggtccaacctag

SEQ ID NO: 5 - DNA, 31 bp, synthetic construct
  misc_feature 1..31: note = PCR
  source 1..31: mol_type = other DNA
tctagagccaccatggtcctgacgctgcttc

SEQ ID NO: 6 - DNA, 26 bp, synthetic construct
  misc_feature 1..26: note = PCR
  source 1..26: mol_type = other DNA
gaattcttaggttggacccatgggtg